(19) United States
(12) Patent Application Publication
(10) Pub. No.: US 2024/0148890 A1
(43) Pub. Date: May 9, 2024

(54) ANTI-FOLATE RECEPTOR CONJUGATE THERAPY FOR CANCER TREATMENT

(71) Applicants: Sutro Biopharma, Inc., South San Francisco, CA (US); Fred Hutchinson Cancer Center, Seattle, WA (US)

(72) Inventors: Arturo MOLINA, Los Altos, CA (US); Soheil Meshinchi, Seattle, WA (US)

(21) Appl. No.: 18/491,656

(22) Filed: Oct. 20, 2023

Related U.S. Application Data
(60) Provisional application No. 63/380,400, filed on Oct. 21, 2022.
(60) Provisional application No. 63/482,698, filed on Feb. 1, 2023.

(51) Int. Cl. A61K 47/68; A61K 31/395; A61P 35/02; C07K 5/078; C07K 16/28

(52) U.S. Cl. CPC A61K 47/6811; A61K 31/395; A61K 47/6889; A61P 35/02; C07K 5/06156; C07K 16/28

(57) ABSTRACT

The present disclosure relates to therapies for FOLR1 expressing cancers, such as pediatric acute myeloid leukemia, (e.g., CBF/GLIS acute myeloid leukemia), with antibody conjugates with binding specificity for folate receptor alpha (FOLR1) and its isoforms and homologs. Also provided are methods of using the combinations with antibody conjugates and compositions thereof, such as in therapeutic and palliative methods.

### CROSS REFERENCE TO RELATED APPLICATIONS

This application claims priority to U.S. Provisional Patent Application No. 63/380,400 filed Oct. 21, 2022, entitled “Anti-Folate Receptor Conjugate Therapy for Pediatric Acute Myeloid Leukemia” and U.S. Provisional Patent Application No. 63/482,698 filed Feb. 1, 2023, entitled “Anti-Folate Receptor Conjugate Therapy for Pediatric Acute Myeloid Leukemia.” The referenced applications are incorporated herein in their entirety.

### REFERENCE TO SEQUENCE LISTING

A computer readable xml file, entitled “108843_00469_SEQ.xml” created on Oct. 20, 2023, with a file size of 568,356 bytes, contains the sequence listing for this application and is hereby incorporated by reference in its entirety.

### FIELD OF INVENTION

Provided herein are cancer therapies with antibody conjugates having binding specificity for folate receptor alpha (FolRα or FOLR1) and compositions for administering the same, including pharmaceutical compositions. The therapies are useful in methods of treatment, prevention, and palliation of pediatric acute myeloid leukemia, for instance CBF/GLIS acute myeloid leukemia.

### BACKGROUND

Cancer remains a leading cause of death worldwide, and additional treatments that improve outcome are needed.

Pediatric acute myeloid leukemia (AML) is a rare cancer with an incidence of about 7 cases per million children under the age of 15 (Creutzig et al., 2012, Blood 120(16):3187-3205). In children under 5 years of age, pediatric AML is commonly associated with chromosomal alterations. One chromosomal aberration seen in children under three years of age is the fusion protein CBFAT3::GLS2 (CBF/GLIS), which is characterized by the cryptic chromosomal translocation inv(16)(p13.3;q24.3) (Smith et al., 2020, Clin. Cancer Res. 26(3):726-737).

CBF/GLIS oncogenic fusion is the underlying genetic cause of the “RAM” phenotype AML, a megakaryoblastic subtype seen in infants and young children. AIL with CBF-GLIS is a rare but uniformly refractory disease with high mortality. In one study, the five-year event-free survival rate for AML with CBF-GLIS was 27% (Masetti et al., 2013, Blood 121(17):3469-3472). In another study, the estimated overall survival rate was less than 15% despite intensive and myeloablative therapies (Smith et al.). Laboratory data demonstrate that this fusion is sufficient for leukemic transformation without recurrent secondary variants. Studies have linked CBF/GLIS fusion to induction of folate receptor alpha (FOLR1) expression in transformed cells (Le et al., 2021, Blood 138, Supp. 1:209).

CBF/GLIS AML is a fatal subtype of AML with limited response to conventional chemotherapy. Accordingly, there is a need for therapeutics for the treatment, prevention, or palliation of pediatric AML, particularly CBF/GLIS AML, among other FOLR1 expressing cancers.

### SUMMARY

Provided herein are therapies for treating or palliating FOLR1 expressing cancers, such as pediatric AML (e.g., CBF/GLIS AML). The therapies are based, in part, on the discovery of folate receptor alpha (FolRα or FOLR1) expression in patients. As shown in the examples provided herein, FOLR1 is expressed in CBF/GLIS AML but absent in other AMLs and absent in normal hematopoietic cells. As such, FOLR1 provides a therapeutic target for treating pediatric AML such as CBF/GLIS AML. Accordingly, provided herein are compositions and methods for treating pediatric AML, including CBF/GLIS AML, in human patients with antibody conjugates that selectively bind folate receptor alpha. The antibody conjugates comprise an antibody that binds folate receptor alpha (FolRα or FOLR1) and is linked to one or more payload moieties. The antibody can be linked to the payload directly by a covalent bond or indirectly by way of a linker. Examples of Folate receptor alpha (FolRα or FOLR1) antibodies are described in detail herein, as are useful payload moieties, and useful linkers.

In one aspect, provided herein are methods of using the anti-FOLR1 antibody conjugates and compositions. In certain embodiments, the methods are methods of treatment. In certain embodiments, the methods are methods of prevention. In certain embodiments, the methods are methods of palliation. In certain embodiments, the patients have CBF/GLIS AML. In certain embodiments, the patients have had no prior treatment with a cancer therapy. In certain embodiments, the patients have had one or more prior treatments. In certain embodiments, the patients are relapsed. In certain embodiments, the patients are refractory. In certain embodiments, the patients are relapsed and refractory. In certain embodiments, the compositions are pharmaceutical compositions. Any suitable pharmaceutical composition may be used. In certain embodiments, the pharmaceutical composition for the anti-FOLR1 antibody conjugates is a composition for intravenous (IV) administration.

The methods, kits, and compositions disclosed herein are useful for treating a disease or disorder, such as FOLR1 expressing cancers. In certain embodiments, the disease or disorder is a pediatric AML, such as CBF/GLIS AML.

### BRIEF DESCRIPTION OF THE FIGURES

FIG. 1A provides FOLR1 expression in CBF/GLIS AML.

FIG. 1B provides cell surface expression of FOLR1 on AML blasts by flow cytometry.

FIG. 1C demonstrates AML-restricted expression of FOLR1 on AML cells but not in normal hematopoietic subsets in individual CBF/GLIS patient samples.

FIG. 1D demonstrates that CBF/GLIS-transduced CB HSPCs upregulate FOLR1 expression.

FIG. 1E provides in vitro efficacy of Conjugate P tested against MV4;11 AML cell line engineered to express FOLR1 (MV4;11 FOLR1+) and CBF/GLIS-transduced CB IPSCs.

FIG. 1F provides in vivo efficacy of Conjugate P in MV4;11 FOLRJ+NSG xenograft models.

FIG. 1G demonstrates that anti-leukemia effects of Conjugate P resulted in significant increase in survival (p=0.002).

FIG. 1H provides in vivo efficacy in xenograft mice bearing CBF/GLIS-transduced CB HSPCs.

FIG. 2 provides negative minimal residual disease (MRD) and positive absolute neutrophil count (ANC) in a 2-year-old female with primary refractory, transplant naïve, CBF2AT3-GLIS2+AML.

FIG. 3 provides negative MRD and positive ANC in a 3-year-old female with Ph+& CBF2AT3-GLIS2+AML.

FIG. 4 shows the results of the study described in EXAMPLE 7.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2014) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

It is understood that aspects and embodiments described herein as “comprising” include “consisting of” and “consisting essentially of” its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transitional phrase “consisting essentially of” limits the scope of the embodiment to the specified elements, steps, ingredients or components and to those that do not materially affect the embodiment. A material effect would cause a statistically significant reduction in the ability to treat cancer or bind to folate receptor 1, as described herein.

As used herein, the singular forms “a,” “an,” and “the” include the plural referents unless the context clearly indicates otherwise.

The term “about” indicates and encompasses an indicated value and a range above and below that value. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of 20% of the stated value; +19% of the stated value; +18% of the stated value; +17% of the stated value; +16% of the stated value; +15% of the stated value; ±14% of the stated value; ±13% of the stated value; ±12% of the stated value; 110% of the stated value; +10% of the stated value; +9% of the stated value; +8% of the stated value; +7% of the stated value; ±6% of the stated value; +5% of the stated value; +4% of the stated value; +3% of the stated value; ±2% of the stated value; or ±1% of the stated value. In certain embodiments, the term “about” indicates the designated value±one standard deviation of that value.

The term “AML” refers to acute myeloid leukemia. The term “pediatric AML” or “childhood AML” refers to AML in a pediatric patient. The term “AML-RAM” refers to acute myeloid leukemia with the RAM phenotype, named after a patient's initials, as described in Broderson et al., 2016, Leukemia 30(10):2077-2080. The term “CBF/GLIS AML” or “CBF::GLIS AML” refers to CBFAT3::GLIS2 fusion protein positive acute myeloid leukemia, for instance as described in Gruber et al., 2012, Cancer Cell 22(5):683-697. The term “BCR::ABL AML” or “BCR-ABL AML” refers to BCR::ABL fusion positive acute myeloid leukemia, for instance as described in Neuendorf et al., 2016, Ann. Hematol. 95(8):1211-21.

The term “combinations thereof” includes every possible combination of elements to which the term refers to. For example, a sentence stating that “if α₂ is A, then α₅ is not D; α₅ is not S; or α₆ is not S; or combinations thereof” includes the following combinations when α₂ is A: (1) as is not D; (2) α₅ is not S; (3) α₆ is not S; (4) α₃ is not D; α₅ is not S; and α₆ is not S; (5) α₃ is not D and α₅ is not S; (6) α₃ is not D and α₆ is not S; and (7) α₅ is not S and α₆ is not S.

The terms “folate receptor alpha” and “folate receptor 1” are used interchangeably herein. Folate receptor alpha is also known by synonyms, including FOLR1, FolRα, folate binding protein, FBP, adult folate binding protein, Folbp1, FR-alpha, FRα, KB cells FBP, and ovarian tumor-associated antigen MOv18, among others. Unless specified otherwise, the terms include any variants, isoforms and species homologs of human folate receptor alpha that are naturally expressed by cells, or that are expressed by cells transfected with a folate receptor alpha or FOLR1 gene. Folate receptor alpha proteins include, for example, human folate receptor alpha (SEQ ID NO: 1). In some embodiments, folate receptor alpha proteins include cynomolgus monkey folate receptor alpha (SEQ ID NO: 2). In some embodiments, folate receptor alpha proteins include murine folate receptor alpha (SEQ ID NO: 3).

The term “angiogenesis inhibitor” as used herein refers to a substance that inhibits the formation of new blood vessels.

The term “biologic” as used herein, refers to a drug substance is made by a living organism or derived from a living organism or made through recombinant DNA or controlled gene expression methodologies.

The term “biosimilar” or “follow-on-biologic” as used herein, refers to products that have similar structures and properties to existing biologic products. Thus, the term “biosimilar” is generally used to describe subsequent versions (generally from a different source) of “innovator biopharmaceutical products” that have previously been approved and officially granted marketing authorization. Because biologics have a high degree of molecular complexity, and are generally sensitive to changes in manufacturing processes (e.g. if different cell lines are used in their production), and since subsequent follow-on manufacturers generally do not have access to the originators molecular clone, cell bank, know-how regarding the production process, nor to the active drug substance itself (only the innovator's commercialized drug product), a “biosimilar” may not be exactly the same as the innovator drug product. However, biosimilars must demonstrate that they have no clinically meaningful differences from their reference products in terms of safety and effectiveness. Therefore, because biosimilars are subsequent versions of a known product and must demonstrate that they have no clinically meaningful differences from their reference products, the term “biosimilar” as used herein includes currently known and approved “biosimilars” as well as any “biosimilars” developed in the future.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “folate receptor alpha antibody,” “anti-folate receptor alpha antibody,” “folate receptor alpha Ab,” “folate receptor alpha-specific antibody,” “anti-folate receptor alpha Ab,” “FOLR1 antibody,” “FolRα antibody,” “anti-FOLR1 antibody,” “anti-FolRα antibody,” “FOLR1 Ab,” “FolRα Ab,” “FOLR1-specific antibody,” “FolRα-specific antibody,” “anti-FolRα Ab,” or “anti-FOLR1 Ab” is an antibody, as described herein, which binds specifically to folate receptor alpha or FOLR1. In some embodiments, the antibody binds the extracellular domain of folate receptor alpha (FOLR1).

The V_{H} and V_{L} regions of the binding domains may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3 CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

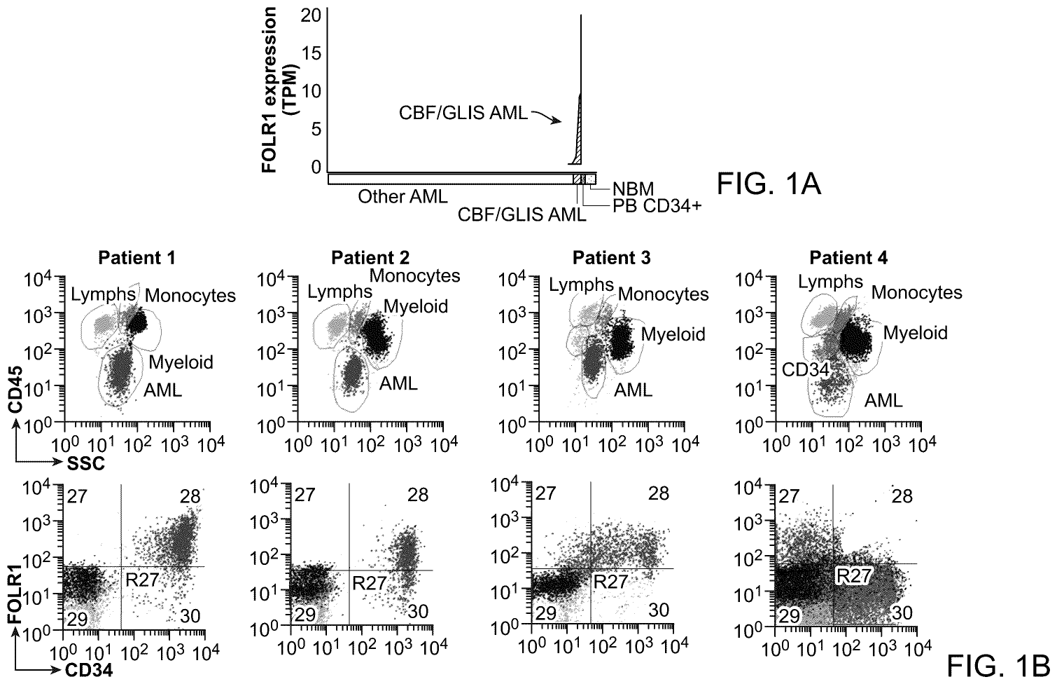

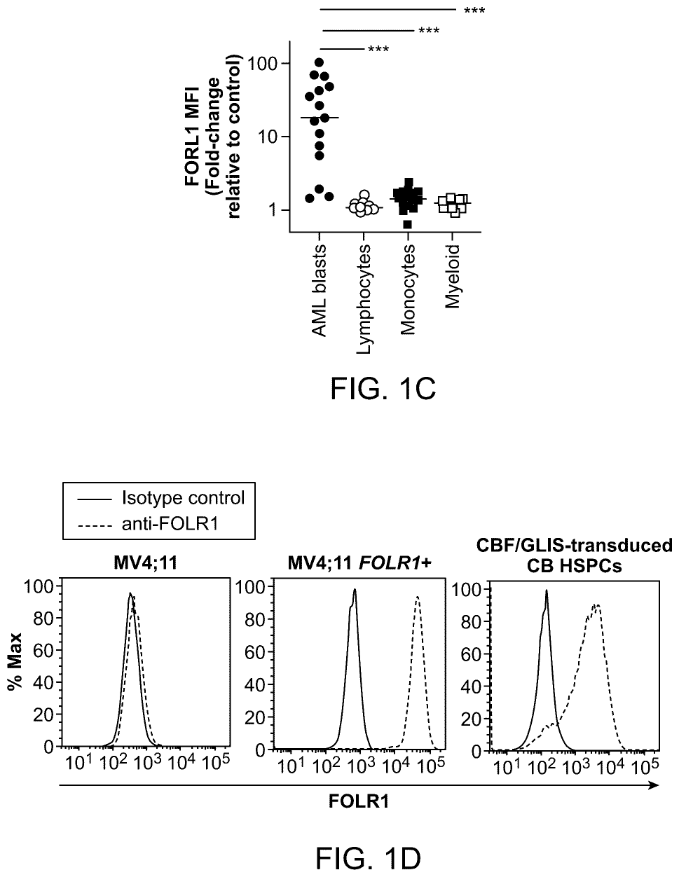

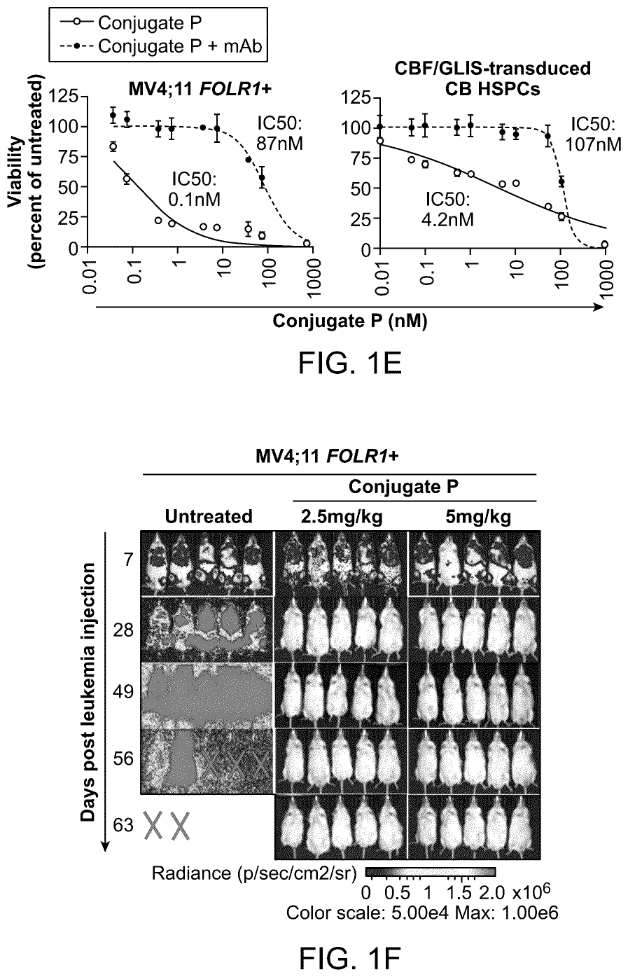

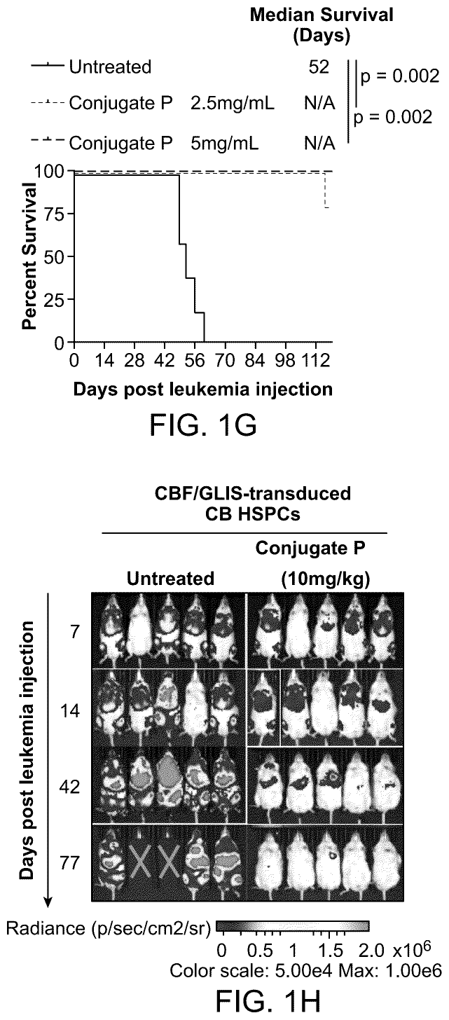

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at www.bioinforg.uk/abs/abnum/, and described in Abhinandan and Martin, Immunology, 2008, 45:3832-3839, and abYsis.org, Swindells et al. 2017, J. Mol. Biol. 429:356-364, each incorporated by reference in its entirety.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} domains are generally linked by a peptide linker. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). See e.g, Bird, et al., Science 242:423-426, 1988; Huston, et al., Proc. Natl. Acad. Sci. USA 85:5879-5883, 1988; Plückthun A. (1994). In some embodiments, the linker is SEQ ID NO: 377. In some embodiments, the linker is SEQ ID NO: 378. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fe domain known in the art or described herein may be used. In some cases, the Fc domain comprises an IgG1 Fc domain. In some embodiments, the IgG1 Fc domain comprises SEQ ID NO: 370, or a portion thereof. SEQ ID NO: 370 provides the sequence of C_{H1}, CH₂, and C_{H3} of the human IgG1 constant region.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies or antibody fragments are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody) and framework regions wherein the amino acid residues including the framework are derived completely or mostly from human germline. The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function.

A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994).

Often, framework residues in the framework regions of humanized antibodies will be substituted with the corresponding residue from the CDR donor antibody to alter, for example improve, cellular marker binding. These framework substitutions are identified by methods well-known in the art, e.g., by modeling of the interactions of the CDR and framework residues to identify framework residues important for cellular marker binding and sequence comparison to identify unusual framework residues at particular positions (See, e.g., U.S. Pat. No. 5,585,089; and Riechmann et al., 1988, Nature, 332:323). For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

The term “binding domain” includes any substance that binds to a cellular marker to form a complex. The choice of binding domain can depend upon the type and number of cellular markers that define the surface of a target cell. Examples of binding domains include cellular marker ligands, receptor ligands, antibodies, peptides, peptide aptamers, receptors (e.g., T cell receptors), or combinations and engineered fragments or formats thereof. The current disclosure provides targeted therapeutics with binding domains that bind FOLR1.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., folate receptor alpha, or FOLR1). In one exemplary assay, FOLR1 is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to variants of folate receptor alpha (FOLR1) with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, R, T, V, |
| | W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn Formation | A, C, D, E, G, H, K, N, Q, R, S, |
| | P, and T |
| Flexible Residues | Q, T, K, S, G, P, D, E, and R |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.” A “functional variant” includes one or more residue additions or substitutions that do not substantially impact the physiological effects of the protein. “Functional fragments” include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling domains) transmit activation or inhibition signals comparable to a wild-type reference when in the activated state of the current disclosure. Functional variants and functional fragments of binding domains bind their cognate antigen or ligand at a level comparable to a wild-type reference.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Naturally encoded amino acids are the proteinogenic amino acids known to those of skill in the art. They include the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and the less common pyrrolysine and selenocysteine. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids and acylated amino acids.

The term “non-natural amino acid” refers to an amino acid that is not a protenogenic amino acid, or a post-translationally modified variant thereof. In particular, the term refers to an amino acid that is not one of the 20 common amino acids or pyrrolysine or selenocysteine, or post-translationally modified variants thereof.

The term “conjugate” or “antibody conjugate” refers to an antibody linked to one or more payload moieties. The antibody can be any antibody described herein. The payload can be any payload described herein. The antibody can be directly linked to the payload via a covalent bond, or the antibody can be linked to the payload indirectly via a linker. Typically, the linker is covalently bonded to the antibody and also covalently bonded to the payload. The term “antibody drug conjugate” or “ADC” refers to a conjugate wherein at least one payload is a therapeutic moiety such as a drug including cytotoxic or therapeutic drugs or agents.

The term “payload” refers to a molecular moiety that can be conjugated to an antibody. In particular embodiments, payloads are selected from the group consisting of cytotoxic moieties, therapeutic moieties, and labelling moieties.

In particular embodiments, ADC refers to targeted chemotherapeutic molecules which combine properties of both binding domains and cytotoxic drugs by targeting potent cytotoxic drugs to antigen-expressing cancer cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & Iyer (Pharm Res. 32(11): 3470-3479, 2015), which describes considerations for the development of ADCs.

The cytotoxic drug moiety of the ADC may include any compound, moiety, or group that has a cytotoxic or cytostatic effect. Cytotoxic drug moieties may impart their cytotoxic and cytostatic effects by mechanisms including tubulin binding, DNA binding or intercalation, and inhibition of RNA polymerase, protein synthesis, and/or topoisomerase. Exemplary drugs include microtubule disrupters and/or anti-mitotic agents (e.g., maytansinoids, hemiasterlins, paclitaxel), alkylating agents (e.g., cisplatin, cyclophosphamide), DNA cross-linking agents (e.g., pyrrolobenzodiazepines (PBDs) or dimers thereof), antitumor antibiotics (e.g., doxorubicin, hedamycin, septacidin), anti-metabolites (e.g., 5-fluorouracil, methotrexate), histone-deacetylase (HDAC) inhibitors (e.g., suberanilohydroxamic acid (SAHA), telomerase inhibitors (e.g., BIBR1542) small molecules that target the SF3b subunit of the spliceosomal U2 small nuclear ribonucleoprotein (snRNP) (Spliceostatin A, meayamycin, and pladienolide B) and/or immunogenic cell death inhibitors (e.g., anthracyclines, platinum based chemotherapeutics, cyclophosphamide, mitoxantrone).

The term “linker” refers to a molecular moiety that is capable of forming at least two covalent bonds. Typically, a linker is capable of forming at least one covalent bond to an antibody and at least another covalent bond to a payload. In certain embodiments, a linker can form more than one covalent bond to an antibody. In certain embodiments, a linker can form more than one covalent bond to a payload or can form covalent bonds to more than one payload. After a linker forms a bond to an antibody, or a payload, or both, the remaining structure, i.e. the residue of the linker after one or more covalent bonds are formed, may still be referred to as a “linker” herein. The term “linker precursor” refers to a linker having one or more reactive groups capable of forming a covalent bond with an antibody or payload, or both. In some embodiments, the linker is a cleavable linker. Cleavable linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the binding domain remains active. For example, a cleavable linker can be one that is released by a bio-labile function, which may or may not be engineered. Alternatively, linkers can be substantially resistant to cleavage (e.g., stable linker or noncleavable linker). In some aspects, the linker is a procharged linker, a hydrophilic linker, or a dicarboxylic acid-based linker. The ADCs selectively deliver an effective dose of a drug to cancer cells whereby greater selectivity, i.e., a lower efficacious dose, may be achieved while increasing the therapeutic index (“therapeutic window”).

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile.

“Excipients” as used herein include pharmaceutically acceptable excipients, carriers, vehicles, or stabilizers that are nontoxic to the cell or mammal being exposed thereto at the dosages and concentrations employed. In some embodiments, the physiologically acceptable excipient is an aqueous pH buffered solution.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder. In some embodiments, a therapeutically effective amount or effective amount refers to an amount of an antibody or composition that when administered to a subject is effective to prevent or ameliorate a disease or the progression of the disease, or result in amelioration of symptoms.

“Palliation” refers to relief of symptoms and suffering caused by a disease such as cancer. Palliation can help a patient feel more comfortable and/or improve quality of life. Palliation does not necessarily cure the disease.

As used herein, the term “inhibits growth” (e.g. referring to cells, such as tumor cells) is intended to include any measurable decrease in cell growth (e.g., tumor cell growth) when contacted with a folate receptor alpha (FOLR1) antibody, as compared to the growth of the same cells not in contact with a FOLR1 antibody. In some embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to antibody internalization, apoptosis, necrosis, and/or effector function-mediated activity.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a disease that can be treated or diagnosed with an antibody provided herein. In some embodiments, the disease is pediatric AML. In some embodiments, the disease is CBF/GLIS AML.

As used herein, the term “cycle” indicates one period of dosing on a dosing schedule. The cycle includes the day of the dose and the following days up to the next dose. Exemplary cycles have lengths of 7 days, 10 days, 14 days, and 21 days.

In some chemical structures illustrated herein, certain substituents, chemical groups, and atoms are depicted with a curvy/wavy line (e.g., [symbol] ) that intersects a bond or bonds to indicate the atom through which the substituents, chemical groups, and atoms are bonded. For example, in some structures, such as but not limited to

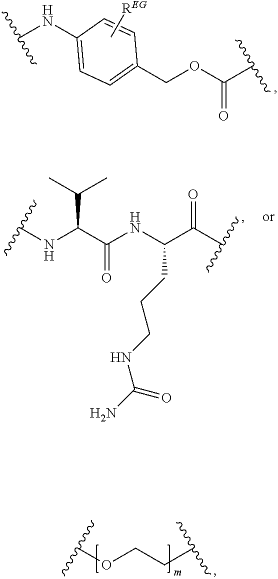

this curvy/wavy line indicates the atoms in the backbone of a conjugate or linker-payload structure to which the illustrated chemical entity is bonded. In some structures, such as but not limited to

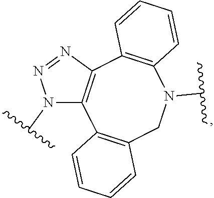 [SMILES: *N1Cc2ccccc2-c2c(nnn2*)-c2ccccc21]

this curvy/wavy line indicates the atoms in the antibody or antibody fragment as well as the atoms in the backbone of a conjugate or linker-payload structure to which the illustrated chemical entity is bonded.

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

### 2. Therapy

Provided herein are methods of treating pediatric AML in a subject in need thereof. In certain embodiments, the pediatric AML is AML-RAM. In certain embodiments, the pediatric AML is CBF/GLIS AML. In certain embodiments, the pediatric AML is BCR::ABL.

The subject can be any subject deemed suitable by the practitioner of skill. In certain embodiments, the subject is 6 months to seven years old. In certain embodiments, the subject is less than three years old. In certain embodiments, the subject has received no prior therapy for AML. In certain embodiments, the subject has received prior therapy for AML. In certain embodiments, the subject has had 1-8 lines of prior therapy. For example, in certain embodiments, the subject has had 1, 2, 3, 4, 5, 6, 7, 8, 9, or 10 lines of prior therapy for AML. In certain embodiments, the subject has had 1-3 lines of prior therapy for AML. In certain embodiments, the subject is relapsed. In certain embodiments, the subject is refractory. In certain embodiments, the subject is relapsed and refractory. In certain embodiments, the subject is not relapsed and not refractory. In certain embodiments, the prior therapy is chemotherapy. In certain embodiments, the prior therapy is stem cell transplant. In certain embodiments, the prior therapy is umbilical cord blood transplant. In certain embodiments, the prior therapy is bone marrow transplant.

In certain embodiments, the methods are for treatment. In certain embodiments, the methods are for treating one or more symptoms of AML. In certain embodiments, the methods are for treating one or more symptoms of AML prior to a subsequent therapy, e.g., bridging methods. In certain embodiments, the subsequent therapy is stem cell transplant. In certain embodiments, the subsequent therapy is umbilical cord blood transplant. In certain embodiments, the subsequent therapy is bone marrow transplant. In certain embodiments, the subsequent therapy is CAR-T. In certain embodiments, the subsequent therapy is donor lymphocyte infusion (DLI). In certain embodiments, the methods are for palliation, for instance to ease pain and suffering and/or to improve quality of life.

### 3. Dosing

The anti-FOLR1 antibody conjugate or composition thereof is suitably administered to the patient over a series of treatments. In certain embodiments, an anti-FOLR1 antibody conjugate described herein is administered to an individual at a fixed dose based on the individual's weight (e.g., mg/kg). In certain embodiments, the first dose is 4.3 mg/kg. In certain embodiments, the first dose is 5.2 mg/kg. In certain embodiments, an anti-FOLR1 antibody conjugate is administered at two different doses. In certain embodiments, the first dose is 4.3 mg/kg, and the second dose is 5.2 mg/kg. In certain embodiments, the first dose is 5.2 mg/kg, and the second dose is 4.3 mg/kg. In certain embodiments, the second dose(s) follow a series of first dose(s).

Each dose can be administered according to a suitable schedule, for example, once, two times, three times, or four times weekly. In certain embodiments, each dose is administered about once every week. In certain embodiments, each dose is administered about once every two to three weeks. In certain embodiments, each dose is administered about once every two weeks. In certain embodiments, each dose is administered about once every three, four, or five weeks. In certain embodiments, each dose is administered about once every three weeks. In certain embodiments, each dose is administered about once every four weeks. In certain embodiments, each dose is administered about once every five weeks. In certain embodiments, each dose is administered daily. In certain embodiments, each dose is separated by a cycle length from the previous dose. In certain embodiments, the cycle length is one day, two days, three days, four days, five days, six days, weekly, ten days, or two weeks, three weeks, four weeks, or more. In particular embodiments the cycle length is 7 days. In particular embodiments the cycle length is 14 days. In particular embodiments the cycle length is 21 days. The cycle length may be 21, 22, 23, 24, 25, 26, 27, 28, 29, 30, 31, 32, 33, 34 or 35 days. In certain embodiments, the clinician or practicing physician will adjust the cycle length. In certain embodiments, the clinician or treating physician will switch from the first dose to the second dose as described herein. Furthermore, it is noted that the clinician or treating physician will know how and when to interrupt, adjust, or terminate therapy in conjunction with subject response. For example, the clinician or treating physician may further adjust dose level in conjunction with subject side effects from treatment or underlying disease for which the subject is receiving treatment.

In certain embodiments, the dose is 4.3 mg/kg on days 1, 3, and 7 of a cycle. In certain embodiments, the dose is 4.3 mg/kg once every 14 days. In certain embodiments, the dose is 4.3 mg/kg once every 21 days. In certain embodiments, the dose is increased to 5.2 mg/kg after one or more initial cycles.

In certain embodiments, the dose is 5.2 mg/kg on days 1, 3, and 7 of a cycle. In certain embodiments, the dose is 5.2 mg/kg once every 14 days. In certain embodiments, the dose is 5.2 mg/kg once every 21 days. In certain embodiments, the dose is decreased to 4.3 mg/kg after one or more initial cycles.

In certain embodiments, the FOLR1 antibody conjugate is administered in combination with a second therapeutic agent. The FOLR1 antibody conjugate may be administered to a patient in conjunction with (e.g., before, simultaneously or following) any number of relevant treatment modalities. In particular embodiments, antibody conjugates may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

In certain embodiments, the second therapeutic agent is a granulocyte colony stimulating factor (GCSF). In certain embodiments, the second therapeutic agent is pegfilgrastim. In certain embodiments, the second therapeutic agent is filgrastim. The second therapeutic agent is administered as described herein, for instance according to labelled instructions. In certain embodiments, the second therapeutic agent lowers the rate of neutropenia in subjects. In certain embodiments, the dose of pegfilgrastim is selected in view of the half-life of the FOLR1 antibody conjugate, for instance on starting on day 8±2 of a therapeutic cycle. In certain embodiments, the dose of pegfilgrastim is selected in view of the half-life of the FOLR1 antibody conjugate, for instance on starting after day 8±2 of a therapeutic cycle and before day 15±2 of a therapeutic cycle. While not intending to be bound by any particular theory of operation, administration of pegfilgrastim on cycle day 8±2 rather than cycle day 1 or 2 (with conventional chemotherapy) was discovered to avoid high levels of exposure of FOLR1 antibody conjugate at the same time as the pegfilgrastim. The half-life of FOLR1 antibody conjugate is longer than conventional small molecule chemotherapy agents. Stimulation of granulopoieses in the presence of high concentrations of a potentially cytotoxic agent (or radiation) can have a deleterious effect on granulocyte production (Viswanathan et al., 2014, Cancer 120(24):3870-83). Further, FOLR1 antibody conjugate-related neutropenia was discovered to occur primarily between cycle day 15 and 21, delayed compared to small molecule cytotoxic agents.

In certain embodiments, the second therapeutic agent is CXCR4 antagonist. In certain embodiments, the second therapeutic agent is plerixafor (Mozobil). The second therapeutic agent is administered as described herein, for instance according to labelled instructions. In certain embodiments, one dose of plerixafor is administered prior to the FOLR1 antibody conjugate. In certain embodiments, one dose of plerixafor is administered after the FOLR1 antibody conjugate. In certain embodiments, doses of plerixafor are administered prior to and after the FOLR1 antibody conjugate, for instance one prior and one after. In certain embodiments, the FOLR1 antibody conjugate is administered in combination with pegfilgrastim and plerixafor.

In certain embodiments, a dose of an antibody conjugate or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

The treatment can continue until the practitioner of skill decides to stop treatment. In certain embodiments, treatment continues until the patient achieves stable disease, decrease in disease burden, partial remission, or complete remission, as understood by the practitioner of skill. In certain embodiments, the therapy continues until the patient reaches remission or partial remission. In certain embodiments, the therapy continues until the clinician or treating physician decides to stop. In certain embodiments, disease burden is evaluated as the percent of bone marrow blasts positive for CBF/GLIS. In certain embodiments, treatment continues until disease burden is less than 50%, less than 25%, less than 15%, less than 10%, less than 5%, less than 3%, or less than 2.5%. In certain embodiments, the therapy continues for one, two, three, four, five, six, seven, eight, nine, ten, fifteen, twenty, twenty-five, or thirty weeks, or more. In certain embodiments, the therapy continues until the patient has a subsequent therapy. In certain embodiments, the subsequent therapy is selected from stem cell transplant, umbilical cord blood transplant, bone marrow transplant, CAR-T, and donor lymphocyte infusion.

### 4. Conjugates

In the methods, anti-FOLR1 antibody conjugate can be any anti-FOLR1 antibody conjugate provided herein. The conjugates comprise an antibody to FOLR1 covalently linked directly or indirectly, via a linker, to a payload. In certain embodiments, the antibody is linked to one payload. In further embodiments, the antibody is linked to more than one payload. In certain embodiments, the antibody is linked to two, three, four, five, six, seven, eight, or more payloads. In certain embodiments, the anti-FOLR1 antibody conjugate is an anti-FOLR1 antibody conjugate described in U.S. Pat. No. 10,596,270, the content of which is hereby incorporated by reference.

The payload can be any payload deemed useful by the practitioner of skill. In certain embodiments, the payload is a therapeutic moiety. In certain embodiments, the payload is a diagnostic moiety, e.g. a label. Useful payloads are described in the sections and examples below.

In certain embodiments, the ADC compounds include a binding domain conjugated, i.e., covalently attached, to the payload (e.g., a drug moiety). In particular embodiments, the binding domain is covalently attached to the drug moiety through a linker. A linker can include any chemical moiety that is capable of linking a binding domain, an antibody, antibody fragment (e.g., antigen binding fragments) or functional equivalent to another moiety, such as a drug moiety. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the binding domain remains active. Alternatively, linkers can be substantially resistant to cleavage (e.g., stable linker or noncleavable linker). In some aspects, the linker is a procharged linker, a hydrophilic linker, or a dicarboxylic acid-based linker. The ADCs selectively deliver an effective dose of a drug to cancer cells whereby greater selectivity, i.e., a lower efficacious dose, may be achieved while increasing the therapeutic index (“therapeutic window”). Useful linkers are described the sections and examples below.

In certain embodiments, the anti-FOLR1 antibody conjugate is according to the formula of Conjugate P, described herein, wherein the antibody is 1848-H01 conjugated through p-azidomethylphenylalanine residues at heavy chain positions Y180 and F404. In certain embodiments, the antibody of the anti-FOLR1 antibody conjugate comprises three heavy chain CDRs of heavy chain SEQ ID NO:362 and three light chain CDRs of light chain SEQ ID NO:367. In certain embodiments, the antibody of the anti-FOLR1 antibody conjugate comprises the three heavy chain CDRs of SEQ ID NOS: 58, 176, and 294 and three light chain CDRs of light chain SEQ ID NO:367. the antibody of the anti-FOLR1 antibody conjugate comprises the three heavy chain CDRs of SEQ ID NOS: 117, 235, and 294 and three light chain CDRs of light chain SEQ ID NO:367. In certain embodiments, the antibody of the anti-FOLR1 antibody conjugate comprises the V_{H} region of heavy chain SEQ ID NO:362 and the V_{L} region of light chain SEQ ID NO:367. In certain embodiments, the antibody of the anti-FOLR1 antibody conjugate comprises heavy chain SEQ ID NO:362 and light chain SEQ ID NO:367. In each of these embodiments, the antibody may comprise Y180 and F404 mutations to p-azidomethylphenylalanine.

In the conjugates provided herein, the antibody can be any antibody with binding specificity for folate receptor alpha (FOLR1 or FolRα). The FOLR1 can be from any species. In certain embodiments, the FOLR1 is a vertebrate FOLR1. In certain embodiments, the FOLR1 is a mammalian FOLR1. In certain embodiments, the FOLR1 is human FOLR1. In certain embodiments, the FOLR1 is mouse FOLR1. In certain embodiments, the FOLR1 is cynomolgus FOLR1.

In certain embodiments, the antibody to folate receptor alpha (FOLR1 or FolRα) competes with an antibody described herein for binding. In certain embodiments, the antibody to FOLR1 binds to the same epitope as an antibody described herein.

The antibody is typically a protein comprising multiple polypeptide chains. In certain embodiments, the antibody is a heterotetramer comprising two identical light (L) chains and two identical heavy (H) chains. Each light chain can be linked to a heavy chain by one covalent disulfide bond. Each heavy chain can be linked to the other heavy chain by one or more covalent disulfide bonds. Each heavy chain and each light chain can also have one or more intrachain disulfide bonds. As is known to those of skill in the art, each heavy chain typically comprises a variable domain (V_{H}) followed by a number of constant domains. Each light chain typically comprises a variable domain at one end (V_{L}) and a constant domain. As is known to those of skill in the art, antibodies typically have selective affinity for their target molecules, i.e. antigens.

The antibodies provided herein can have any antibody form known to those of skill in the art. They can be full-length, or fragments. Exemplary full length antibodies include IgA, IgA1, IgA2, IgD, IgE, IgG, IgG1, IgG2, IgG3, IgG4, IgM, etc. Exemplary fragments include Fv, Fab, Fc, scFv, scFv-Fc, etc.

In certain embodiments, the antibody of the conjugate comprises one, two, three, four, five, or six of the CDR sequences described herein. In certain embodiments, the antibody of the conjugate comprises a heavy chain variable domain (V_{H}) described herein. In certain embodiments, the antibody of the conjugate comprises a light chain variable domain (V_{L}) described herein. In certain embodiments, the antibody of the conjugate comprises a heavy chain variable domain (V_{H}) described herein and a light chain variable domain (V_{L}) described herein. In certain embodiments, the antibody of the conjugate comprises a paired heavy chain variable domain and a light chain variable domain described herein (V_{H}-V_{L} pair).

In particular embodiments, a V_{L} region in a binding domain of the present disclosure is derived from or based on a V_{L} of an antibody disclosed herein and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the V_{L} of the antibody disclosed herein. An insertion, deletion or substitution may be anywhere in the V_{L} region, including at the amino- or carboxy-terminus or both ends of this region, provided that each CDR includes zero changes or at most one, two, or three changes and provided a binding domain containing the modified V_{L} region can still specifically bind its target with an affinity similar to the wild type binding domain.

In particular embodiments, a binding domain V_{H} region of the present disclosure can be derived from or based on a V_{H} of an antibody disclosed herein and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{H} of the antibody disclosed herein. An insertion, deletion or substitution may be anywhere in the V_{H} region, including at the amino- or carboxy-terminus or both ends of this region, provided that each CDR includes zero changes or at most one, two, or three changes and provided a binding domain containing the modified V_{H} region can still specifically bind its target with an affinity similar to the wild type binding domain.

In certain embodiments, the antibody of the conjugate comprises any of the amino acid sequences of the antibodies described above. In certain embodiments, the antibody comprises any of the amino acid sequences above with up to 10 amino acid substitutions. In certain embodiments, the antibody comprises any of the amino acid sequences above with up to 9 amino acid substitutions. In certain embodiments, the antibody comprises any of the amino acid sequences above with up to 8 amino acid substitutions. In certain embodiments, the antibody comprises any of the amino acid sequences above with up to 7 amino acid substitutions. In certain embodiments, the antibody comprises any of the amino acid sequences above with up to 6 amino acid substitutions. In certain embodiments, the antibody comprises any of the amino acid sequences above with up to 5 amino acid substitutions. In certain embodiments, the antibody comprises any of the amino acid sequences above with up to 4 amino acid substitutions. In certain embodiments, the antibody comprises any of the amino acid sequences above with up to 3 amino acid substitutions. In certain embodiments, the antibody comprises any of the amino acid sequences above with up to 2 amino acid substitutions. In certain embodiments, the antibody comprises any of the amino acid sequences above with up to 1 conservative amino acid substitution. In some embodiments, the amino acid substitutions are conservative amino acid substitutions. For example, in certain embodiments, the antibody comprises any of the amino acid sequences above with up to 10 conservative amino acid substitutions. In certain embodiments, the antibody comprises any of the amino acid sequences above with up to 9 conservative amino acid substitutions. In certain embodiments, the antibody comprises any of the amino acid sequences above with up to 8 conservative amino acid substitutions. In certain embodiments, the antibody comprises any of the amino acid sequences above with up to 7 conservative amino acid substitutions. In certain embodiments, the antibody comprises any of the amino acid sequences above with up to 6 conservative amino acid substitutions. In certain embodiments, the antibody comprises any of the amino acid sequences above with up to 5 conservative amino acid substitutions. In certain embodiments, the antibody comprises any of the amino acid sequences above with up to 4 conservative amino acid substitutions. In certain embodiments, the antibody comprises any of the amino acid sequences above with up to 3 conservative amino acid substitutions. In certain embodiments, the antibody comprises any of the amino acid sequences above with up to 2 conservative amino acid substitutions. In certain embodiments, the antibody comprises any of the amino acid sequences above with up to 1 conservative amino acid substitution.

In certain embodiments, the antibody conjugate can be formed from an antibody that comprises one or more reactive groups. In certain embodiments, the antibody conjugate can be formed from an antibody comprising all naturally encoded amino acids. Those of skill in the art will recognize that several naturally encoded amino acids include reactive groups capable of conjugation to a payload or to a linker. These reactive groups include cysteine side chains, lysine side chains, and amino-terminal groups. In these embodiments, the antibody conjugate can comprise a payload or linker linked to the residue of an antibody reactive group. In these embodiments, the payload precursor or linker precursor comprises a reactive group capable of forming a bond with an antibody reactive group. Typical reactive groups include maleimide groups, activated carbonates (including but not limited to, p-nitrophenyl ester), activated esters (including but not limited to, N-hydroxysuccinimide, p-nitrophenyl ester, and aldehydes). Particularly useful reactive groups include maleimide and succinimide, for instance N-hydroxysuccinimide, for forming bonds to cysteine and lysine side chains. Further reactive groups are described in the sections and examples below.

In further embodiments, the antibody comprises one or more modified amino acids having a reactive group, as described herein. Typically, the modified amino acid is not a naturally encoded amino acid. These modified amino acids can comprise a reactive group useful for forming a covalent bond to a linker precursor or to a payload precursor. One of skill in the art can use the reactive group to link the polypeptide to any molecular entity capable of forming a covalent bond to the modified amino acid. Thus, provided herein are conjugates comprising an antibody comprising a modified amino acid residue linked to a payload directly or indirectly via a linker. Exemplary modified amino acids are described in the sections below. Generally, the modified amino acids have reactive groups capable of forming bonds to linkers or payloads with complementary reactive groups.

The non-natural amino acids are positioned at select locations in a polypeptide chain of the antibody. These locations were identified as providing optimum sites for substitution with the non-natural amino acids. Each site is capable of bearing a non-natural amino acid with optimum structure, function and/or methods for producing the antibody.

In certain embodiments, a site-specific position for substitution provides an antibody that is stable. Stability can be measured by any technique apparent to those of skill in the art.

In certain embodiments, a site-specific position for substitution provides an antibody that has optimal functional properties. For instance, the antibody can show little or no loss of binding affinity for its target antigen compared to an antibody without the site-specific non-natural amino acid. In certain embodiments, the antibody can show enhanced binding compared to an antibody without the site-specific non-natural amino acid.

In certain embodiments, a site-specific position for substitution provides an antibody that can be made advantageously. For instance, in certain embodiments, the antibody shows advantageous properties in its methods of synthesis, discussed below. In certain embodiments, the antibody can show little or no loss in yield in production compared to an antibody without the site-specific non-natural amino acid. In certain embodiments, the antibody can show enhanced yield in production compared to an antibody without the site-specific non-natural amino acid. In certain embodiments, the antibody can show little or no loss of tRNA suppression compared to an antibody without the site-specific non-natural amino acid. In certain embodiments, the antibody can show enhanced tRNA suppression in production compared to an antibody without the site-specific non-natural amino acid.

In certain embodiments, a site-specific position for substitution provides an antibody that has advantageous solubility. In certain embodiments, the antibody can show little or no loss in solubility compared to an antibody without the site-specific non-natural amino acid. In certain embodiments, the antibody can show enhanced solubility compared to an antibody without the site-specific non-natural amino acid.

In certain embodiments, a site-specific position for substitution provides an antibody that has advantageous expression. In certain embodiments, the antibody can show little or no loss in expression compared to an antibody without the site-specific non-natural amino acid. In certain embodiments, the antibody can show enhanced expression compared to an antibody without the site-specific non-natural amino acid.

In certain embodiments, a site-specific position for substitution provides an antibody that has advantageous folding. In certain embodiments, the antibody can show little or no loss in proper folding compared to an antibody without the site-specific non-natural amino acid. In certain embodiments, the antibody can show enhanced folding compared to an antibody without the site-specific non-natural amino acid.

In certain embodiments, a site-specific position for substitution provides an antibody that is capable of advantageous conjugation. As described below, several non-natural amino acids have side chains or functional groups that facilitate conjugation of the antibody to a second therapeutic agent, either directly or via a linker. In certain embodiments, the antibody can show enhanced conjugation efficiency compared to an antibody without the same or other non-natural amino acids at other positions. In certain embodiments, the antibody can show enhanced conjugation yield compared to an antibody without the same or other non-natural amino acids at other positions. In certain embodiments, the antibody can show enhanced conjugation specificity compared to an antibody without the same or other non-natural amino acids at other positions.

The one or more non-natural amino acids are located at selected site-specific positions in at least one polypeptide chain of the antibody. The polypeptide chain can be any polypeptide chain of the antibody without limitation, including either light chain or either heavy chain. The site-specific position can be in any domain of the antibody, including any variable domain and any constant domain.

In certain embodiments, the antibodies provided herein comprise one non-natural amino acid at a site-specific position. In certain embodiments, the antibodies provided herein comprise two non-natural amino acids at site-specific positions. In certain embodiments, the antibodies provided herein comprise three non-natural amino acids at site-specific positions. In certain embodiments, the antibodies provided herein comprise more than three non-natural amino acids at site-specific positions.

In certain embodiments, the antibodies provided herein comprise one or more non-natural amino acids each at a position selected from the group consisting of heavy chain or light chain residues HC-F404, HC-K121, HC-Y180, HC-F241, HC-221, LC-T22, LC-S7, LC-N152, LC-K42, LC-E161, LC-D170, HC-S136, HC-S25, HC-A40, HC-S119, HC-S190, HC-K222, HC-R19, HC-Y52, or HC-S70 according to the Kabat, Chothia, or EU numbering scheme, or a post-translationally modified variant thereof. In these designations, HC indicates a heavy chain residue, and LC indicates a light chain residue.

In certain embodiments, the antibody is linked site-specifically to at least one payload. In certain embodiments, the antibody comprises one or more non-natural amino acids at sites selected from the group consisting of: HC F404, HC-K121, HC-Y180, HC-F241, HC-221, LC-T22, LC-S7, LC-N152, LC-K42, LC-E161, LC-D170, HC-S136, HC S25, HC-A40, HC-S119, HC-S190, HC-K222, HC-R19, HC-Y52, or HC-S70, according to the Kabat, Chothia, or EU numbering scheme. In certain embodiments, the antibody comprises one or more non-natural amino acids at sites selected from the group consisting of: HC F404, HC-Y180, and LC-K42, according to the Kabat, Chothia, or EU numbering scheme. In certain embodiments, the antibody comprises a non-natural amino acid at site HC-F404. In certain embodiments, the antibody comprises a non-natural amino acid at site HC-F404 In certain embodiments, the antibody comprises non-natural amino acids at sites HC-F404 and HC-Y180. In certain embodiments, a residue of the one or more non-natural amino acids is linked to the payload moiety via a linker that is hydrolytically stable. In certain embodiments, a residue of the one or more non-natural amino acids is linked to the payload moiety via a linker that is cleavable.

In certain embodiments, the one or more non-natural amino acids is selected from the group consisting of p-acetyl-L-phenylalanine, O-methyl-L-tyrosine, an -3-(2-naphthyl)alanine, 3-methyl-phenylalanine, O-4-allyl-L-tyrosine, 4-propyl-L-tyrosine, a tri-O-acetyl-GlcNAcP-serine, L-Dopa, fluorinated phenylalanine, isopropyl-L-phenylalanine, p-azido-L-phenylalanine, p-azido-methyl-L-phenylalanine, compound 56, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine. In certain embodiments, the non-natural amino acid residue is a residue of compound (30) or compound (56).

In particular embodiments, provided herein are anti-FOLR1 conjugates according to any of Formulas 101a-104b wherein COMP indicates a residue of the non-natural amino acid according to Formula (30), below. In particular embodiments, provided herein are anti-FOLR1 conjugates according to any of Formulas 101a-104b wherein COMP indicates a residue of the non-natural amino acid according to Formula (30), below, at heavy chain position 404 according to the EU numbering system. In particular embodiments, provided herein are anti-FOLR1 conjugates according to any of Formulas 101a-104b wherein COMP indicates a residue of the non-natural amino acid according to Formula (30), below, at heavy chain position 180 according to the EU numbering system. In particular embodiments, provided herein are anti-FOLR1 conjugates according to any of Formulas 101a-104b wherein COMP indicates a residue of the non-natural amino acid according to Formula (30), below, at heavy chain position 241 according to the EU numbering system. In particular embodiments, provided herein are anti-FOLR1 conjugates according to any of Formulas 101a-104b wherein COMP indicates a residue of the non-natural amino acid according to Formula (30), below, at heavy chain position 222 according to the EU numbering system. In particular embodiments, provided herein are anti-FOLR1 conjugates according to any of Formulas 101a-104b wherein COMP indicates a residue of the non-natural amino acid according to Formula (30), below, at light chain position 7 according to the Kabat or Chothia numbering system. In particular embodiments, provided herein are anti-FOLR1 conjugates according to any of Formulas 101a-104b wherein COMP indicates a residue of the non-natural amino acid according to Formula (30), below, at light chain position 42 according to the Kabat or Chothia numbering system. In certain embodiments, PAY is selected from the group consisting of maytansine, hemiasterlin, amanitin, monomethyl auristatin F (MMAF), and monomethyl auristatin E (MMAE). In certain embodiments, the PAY is maytansine. In certain embodiments, PAY is hemiasterlin. In particular embodiments, the hemiasterlin is a cleavable 3-aminophenyl hemiasterlin drug-linker conjugated via a cleavable valine citrulline p-aminobenzyl carbamate linker functionalized with dibenzocyclooctyne (DBCO). In certain embodiments, PAY is amanitin. In certain embodiments, PAY is MMAF. In certain embodiments, PAY is MMAE.

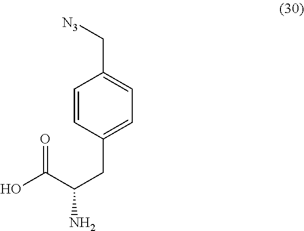 [SMILES: [N-]=[N+]=NCc1ccc(C[C@H](N)C(=O)O)cc1]

In particular embodiments, provided herein are anti-FOLR1 conjugates according to any of Formulas 101a-104b wherein COMP indicates a residue of the non-natural amino acid according to Formula (56), below. In particular embodiments, provided herein are anti-FOLR1 conjugates according to any of Formulas 101a-104b wherein COMP indicates a residue of the non-natural amino acid according to Formula (56), below, at heavy chain position 404 according to the EU numbering system. In particular embodiments, provided herein are anti-FOLR1 conjugates according to any of Formulas 101a-104b wherein COMP indicates a residue of the non-natural amino acid according to Formula (56), below, at heavy chain position 180 according to the EU numbering system. In particular embodiments, provided herein are anti-FOLR1 conjugates according to any of Formulas 101a-104b wherein COMP indicates a residue of the non-natural amino acid according to Formula (56), below, at heavy chain position 241 according to the EU numbering system. In particular embodiments, provided herein are anti-FOLR1 conjugates according to any of Formulas 101a-104b wherein COMP indicates a residue of the non-natural amino acid according to Formula (56), below, at heavy chain position 222 according to the EU numbering system. In particular embodiments, provided herein are anti-FOLR1 conjugates according to any of Formulas 101a-104b wherein COMP indicates a residue of the non-natural amino acid according to Formula (56), below, at light chain position 7 according to the Kabat or Chothia numbering system. In particular embodiments, provided herein are anti-FOLR1 conjugates according to any of Formulas 101a-104b wherein COMP indicates a residue of the non-natural amino acid according to Formula (56), below, at light chain position 42 according to the Kabat or Chothia numbering system. In certain embodiments, PAY is selected from the group consisting of maytansine, hemiasterlin, amanitin, MMAF, and MMAE. In certain embodiments, the PAY is maytansine. In certain embodiments, PAY is hemiasterlin. In particular embodiments, the hemiasterlin is a cleavable 3-aminophenyl hemiasterlin drug-linker conjugated via a cleavable valine citrulline p-aminobenzyl carbamate linker functionalized with dibenzocyclooctyne (DBCO). In certain embodiments, PAY is amanitin. In certain embodiments, PAY is MMAF. In certain embodiments, PAY is MMAE.

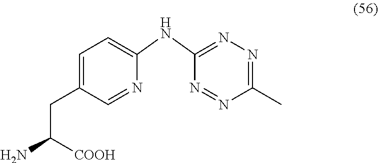 [SMILES: Cc1nnc(Nc2ccc(C[C@H](N)C(=O)O)cn2)nn1]

In particular embodiments, provided herein are anti-FOLR1 conjugates according to any of Formulas 101a-104b wherein COMP indicates a non-natural amino acid residue of para-azido-L-phenylalanine. In particular embodiments, provided herein are anti-FOLR1 conjugates according to any of Formulas 101a-104b wherein COMP indicates the non-natural amino acid residue para-azido-phenylalanine at heavy chain position 404 according to the EU numbering system. In particular embodiments, provided herein are anti-FOLR1 conjugates according to any of Formulas 101a-104b wherein COMP indicates a non-natural amino acid residue of para-azido-L-phenylalanine at heavy chain position 180 according to the EU numbering system. In particular embodiments, provided herein are anti-FOLR1 conjugates according to any of Formulas 101a-104b wherein COMP indicates a non-natural amino acid residue para-azido-L-phenylalanine at heavy chain position 241 according to the EU numbering system. In particular embodiments, provided herein are anti-FOLR1 conjugates according to any of Formulas 101a-104b wherein COMP indicates a non-natural amino acid residue para-azido-L-phenylalanine at heavy chain position 222 according to the EU numbering system. In particular embodiments, provided herein are anti-FOLR1 conjugates according to any of Formulas 101a-104b wherein COMP indicates a non-natural amino acid residue para-azido-L-phenylalanine at light chain position 7 according to the Kabat or Chothia numbering system. In particular embodiments, provided herein are anti-FOLR1 conjugates according to any of Formulas 101a-104b wherein COMP indicates a non-natural amino acid residue para-azido-L-phenylalanine at light chain position 42 according to the Kabat or Chothia numbering system. In certain embodiments, PAY is selected from the group consisting of maytansine, hemiasterlin, amanitin, MMAF, and MMAE. In certain embodiments, the PAY is maytansine. In certain embodiments, PAY is hemiasterlin. In certain embodiments, PAY is amanitin. In certain embodiments, PAY is MMAF. In certain embodiments, PAY is MMAE.

In certain embodiments, the at least one payload moiety is selected from the group consisting of maytansines, hemiasterlins, amanitins, and auristatins. In certain embodiments, the at least one payload moiety is selected from the group consisting of DM1, hemiasterlin, amanitin, MMAF, and MMAE. In certain embodiments, the at least one payload moiety is a hemiasterlin derivative. In certain embodiments, the at least one payload moiety is:

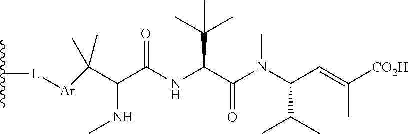

wherein Ar is aryl or heteroaryl, L is a linker, and the wiggly line indicates a bond to the antibody. In certain embodiments, the at least one payload moiety is:

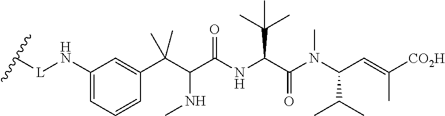

wherein L is a linker, and the wiggly line indicates a bond to the antibody.

In some embodiments, provided herein are anti-FOLR1 conjugates comprising a modified hemiasterlin and linker as described, for example, in PCT Publication No. WO 2016/123582. For example, the conjugate can have a structure comprising any of Formulas 1000-1000b, 1001-1001b, 1002-1002b, and I-XIXb-2, 101-111b, or 1-8b as described in PCT Publication No. WO 2016/2016/123582. In other examples, cleavable 3-aminophenyl hemiasterlin drug-linker is conjugated via a cleavable valine citrulline p-aminobenzyl carbamate linker functionalized with dibenzocyclooctyne (DBCO). Examples of other conjugates comprising a modified hemiasterlin and linker are provided below.

In some embodiments, provided herein are anti-FOLR1 conjugates having the structure of Conjugate M:

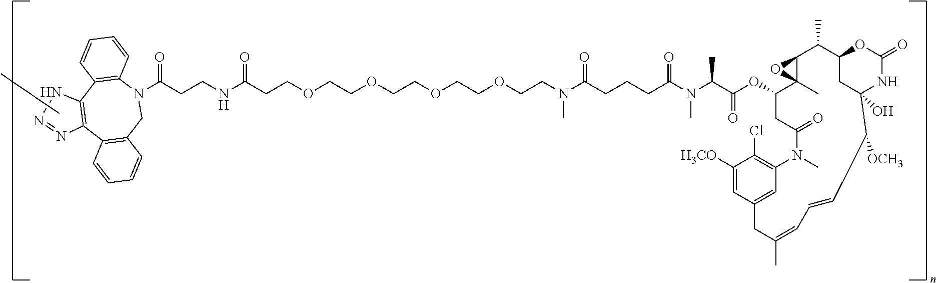

where n is an integer from 1 to 6. In some embodiments, n is an integer from 1 to 4. In some embodiments, n is 2. For example, in some embodiments, the anti-FOLR1 conjugate has the structure:

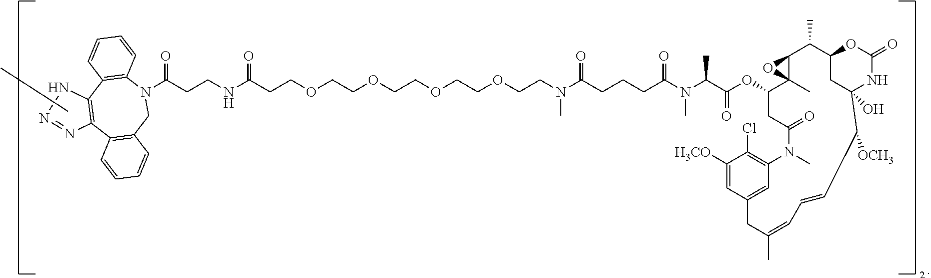

In some embodiments, n is 4. For example, in some embodiments, the anti-FOLR1 conjugate has the structure:

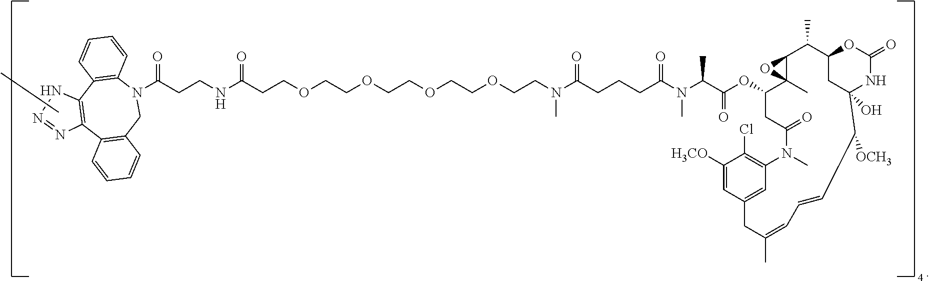

In some embodiments, provided herein are anti-FOLR1 conjugates having the structure of Conjugate P:

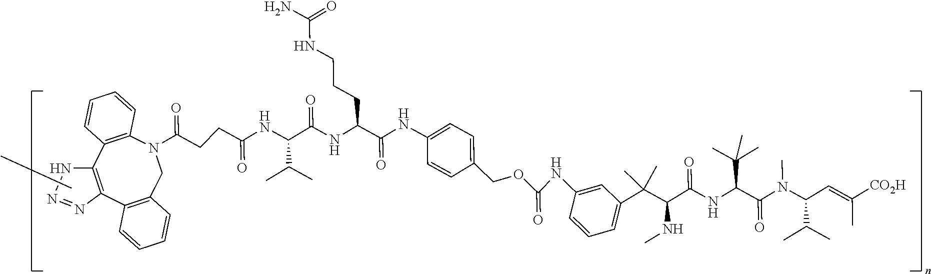

where n is an integer from 1 to 6. In some embodiments, n is an integer from 1 to 4. In some embodiments, n is 2. For example, in some embodiments, the anti-FOLR1 conjugate has the structure:

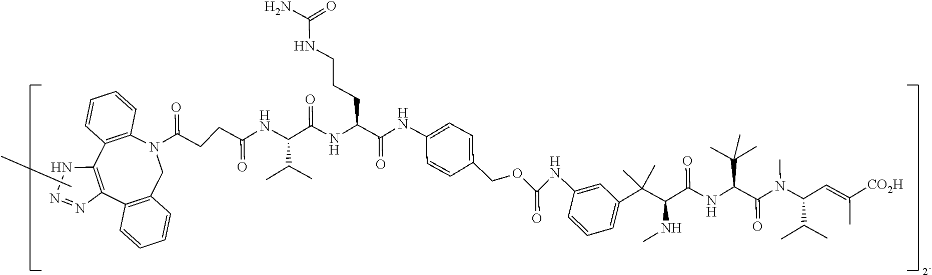

In some embodiments, n is 4. For example, in some embodiments, the anti-FOLR1 conjugate has the structure:

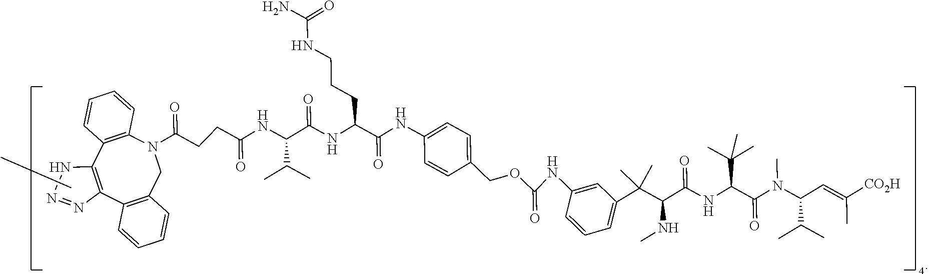

In some embodiments, provided herein are anti-FOLR1 conjugates having the structure of Conjugate Q:

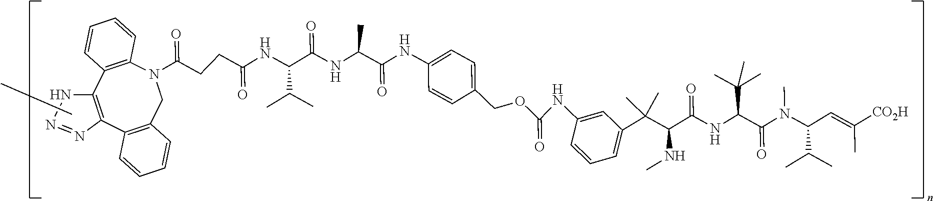

where n is an integer from 1 to 6. In some embodiments, n is an integer from 1 to 4. In some embodiments, n is 2. For example, in some embodiments, the anti-FOLR1 conjugate has the structure:

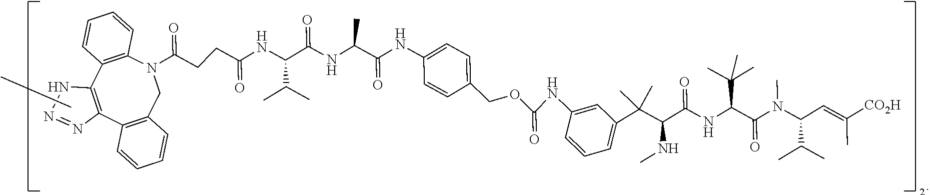

In some embodiments, n is 4. For example, in some embodiments, the anti-FOLR1 conjugate has the structure:

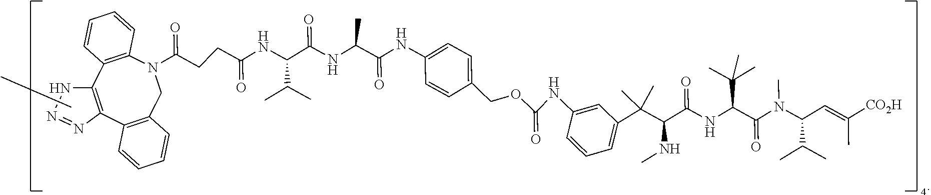

In any of the foregoing embodiments wherein the anti-FOLR1 conjugate has a structure according to Conjugate M, Conjugate P, or Conjugate Q, the bracketed structure can be covalently bonded to one or more non-natural amino acids of the antibody, wherein the one or more non-natural amino acids are located at sites selected from the group consisting of: HC-F404, HC-Y180, and LC-K42 according to the Kabat or EU numbering scheme of Kabat. In some embodiments, the bracketed structure is covalently bonded to one or more non-natural amino acids at site HC-F404 of the antibody. In some embodiments, the bracketed structure is covalently bonded to one or more non-natural amino acids at site HC-Y180 of the antibody. In some embodiments, the bracketed structure is covalently bonded to one or more non-natural amino acids at site LC-K42 of the antibody. In some embodiments, the bracketed structure is covalently bonded to one or more non-natural amino acids at sites HC-F404 and HC-Y180 of the antibody. In some embodiments, at least one bracketed structure is covalently bonded to a non-natural amino acid at site HC-F404 of the antibody, and at least one bracketed structure is covalently bonded a non-natural amino acid at site HC-Y180 of the antibody. In some embodiments, the bracketed structure is covalently bonded to one or more non-natural amino acids at sites HC-Y180 and LC-K42 of the antibody. In some embodiments, at least one bracketed structure is covalently bonded to a non-natural amino acid at site HC-Y180 of the antibody, and at least one bracketed structure is covalently bonded a non-natural amino acid at site LC-K32 of the antibody.

Particular examples of targeted therapeutics disclosed herein include an antibody conjugate including a FOLR1 binding domain linked to the cytotoxic payload. In particular embodiments, the antibody conjugate includes a FOLR1 binding domain conjugated to a cleavable 3-aminophenyl hemiasterlin drug-linker. In particular embodiments, the FOLR1 binding domain includes an FRα human immunoglobulin G1 antibody (H01) or a binding fragment thereof. Particular embodiments include the anti-FolRα human IgG1 antibody H01 conjugated to a cleavable 3-aminophenyl hemiasterlin drug-linker at Y180 and F404 on the antibody heavy chain. H01 was discovered and optimized using a Fab ribosome display selection and has four non-natural amino acid, p-azido-phenylalanine (pAMF) residues incorporated at positions Y180 and F404 on each heavy chain. When H01 is conjugated to a cleavable 3-aminophenyl hemiasterlin drug-linker at Y180 and F404 on the antibody heavy chain, it is referred to as Conjugate A. In particular embodiments, H01 is an antibody described in WO2019055909A1. In particular embodiments, Conjugate A is an anti-Fol1 antibody conjugate described in WO2019055909A1.

### 5. Antibody Specificity

The conjugates comprise antibodies that selectively bind human folate receptor alpha. In some aspects, the antibody selectively binds to the extracellular domain of human folate receptor alpha (human FOLR1).

In some embodiments, the antibody binds to a homolog of human FOLR1. In some aspects, the antibody binds to a homolog of human FOLR1 from a species selected from monkeys, mice, dogs, cats, rats, cows, horses, goats and sheep. In some aspects, the homolog is a cynomolgus monkey homolog. In some aspects, the homolog is a mouse or murine analog.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, or a variant thereof. In some aspects, the variant is a variant with a conservative amino acid substitution.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 13, 14, 15, 16, or 17 residues in length.

In some aspects, the Kabat/Chothia CDR-L1 of the antibody is 11, 12, 13, 14, 15, 16, 17, or 18 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some instances, additional scFvs based on the binding domains described herein and for use in a targeted therapeutic can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies.

Other binding fragments, such as Fv, Fab, Fab′, F(ab′)2, can also be used within the targeted therapeutic disclosed herein. Additional examples of antibody-based binding domain formats for use in a targeted therapeutic include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

In certain embodiments, the antibody comprises: three heavy chain CDRs and three light chain CDRs of a V_{H} region provided herein, and a V_{L} region provided herein. In certain embodiments, the V_{H} region is selected from SEQ ID NOs:308-366. In certain embodiments, the V_{L} region is selected from SEQ ID NOs: 367-369. In particular embodiments, the V_{H} region is according to SEQ ID NO:362, and a V_{L} region is according to SEQ ID NO:367. CDR sequences can be identified by routine techniques well known to those of skill in the art. In certain embodiments, the CDRs are identified according to Kabat numbering. In certain embodiments, the CDRs are identified according to Chothia numbering. In certain embodiments, the CDRs are identified according to Martin numbering. In certain embodiments, the CDRs are identified according to contact numbering. In certain embodiments, the CDRs identified according to AHo numbering. In certain embodiments, the CDRs identified according to IMGT numbering. Tools for identifying CDR sequences are available, for example, abYsis or abnum, supra.

### 5.1 CDR-H3 Sequences

In some embodiments, the antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a V_{H} sequence provided in SEQ ID NOs: 308-366.

In some embodiments, the antibody comprises a CDR-H3 sequence comprising, consisting of, or consisting essentially of a sequence selected from SEQ ID NOs: 240-298. In some aspects, the antibody comprises a CDR-H3 sequence comprising, consisting of, or consisting essentially of SEQ ID NO: 240. In some aspects, the antibody comprises a CDR-H3 sequence comprising, consisting of, or consisting essentially of SEQ ID NO: 241. In some aspects, the antibody comprises a CDR-H3 sequence comprising, consisting of, or consisting essentially of SEQ ID NO: 242. In some aspects, the antibody comprises a CDR-H3 sequence comprising, consisting of, or consisting essentially of SEQ ID NO: 243. In some aspects, the antibody comprises a CDR-H3 sequence comprising, consisting of, or consisting essentially of SEQ ID NO: 244. In some aspects, the antibody comprises a CDR-H3 sequence comprising, consisting of, or consisting essentially of SEQ ID NO: 245. In some aspects, the antibody comprises a CDR-H3 sequence comprising, consisting of, or consisting essentially of SEQ ID NO: 246. In some aspects, the antibody comprises a CDR-H3 sequence comprising, consisting of, or consisting essentially of SEQ ID NO: 247. In some aspects, the antibody comprises a CDR-H3 sequence comprising, consisting of, or consisting essentially of SEQ ID NO: 248. In some aspects, the antibody comprises a CDR-H3 sequence comprising, consisting of, or consisting essentially of SEQ ID NO: 249. In some aspects, the antibody comprises a CDR-H3 sequence comprising, consisting of, or consisting essentially of SEQ ID NO: 250. In some aspects, the antibody comprises a CDR-H3 sequence comprising, consisting of, or consisting essentially of SEQ ID NO: 251. In some aspects, the antibody comprises a CDR-H3 sequence comprising, consisting of, or consisting essentially of SEQ ID NO: 252. In some aspects, the antibody comprises a CDR-H3 sequence comprising, consisting of, or consisting essentially of SEQ ID NO: 253. In some aspects, the antibody comprises a CDR-H3 sequence comprising, consisting of, or consisting essentially of SEQ ID NO: 254. In some aspects, the antibody comprises a CDR-H3 sequence comprising, consisting of, or consisting essentially of SEQ ID NO: 255. In some aspects, the antibody comprises a CDR-H3 sequence comprising, consisting of, or consisting essentially of SEQ ID NO: 256. In some aspects, the antibody comprises a CDR-H3 sequence comprising, consisting of, or consisting essentially of SEQ ID NO: 257. In some aspects, the antibody comprises a CDR-H3 sequence comprising, consisting of, or consisting essentially of SEQ ID NO: 258. In some aspects, the antibody comprises a CDR-H3 sequence comprising, consisting of, or consisting essentially of SEQ ID NO: 259. In some aspects, the antibody comprises a CDR-H3 sequence comprising, consisting of, or consisting essentially of SEQ ID NO: 260. In some aspects, the antibody comprises a CDR-H3 sequence comprising, consisting of, or consisting essentially of SEQ ID NO: 261. In some aspects, the antibody comprises a CDR-H3 sequence comprising, consisting of, or consisting essentially of SEQ ID NO: 262. In some aspects, the antibody comprises a CDR-H3 sequence comprising, consisting of, or consisting essentially of SEQ ID NO: 263. In some aspects, the antibody comprises a CDR-H3 sequence comprising, consisting of, or consisting essentially of SEQ ID NO: 264. In some aspects, the antibody comprises a CDR-H3 sequence comprising, consisting of, or consisting essentially of SEQ ID NO: 265. In some aspects, the antibody comprises a CDR-H3 sequence comprising, consisting of, or consisting essentially of SEQ ID NO: 266. In some aspects, the antibody comprises a CDR-H3 sequence comprising, consisting of, or consisting essentially of SEQ ID NO: 267. In some aspects, the antibody comprises a CDR-H3 sequence comprising, consisting of, or consisting essentially of SEQ ID NO: 268. In some aspects, the antibody comprises a CDR-H3 sequence comprising, consisting of, or consisting essentially of SEQ ID NO: 269. In some aspects, the antibody comprises a CDR-H3 sequence comprising, consisting of, or consisting essentially of SEQ ID NO: 270. In some aspects, the antibody comprises a CDR-H3 sequence comprising, consisting of, or consisting essentially of SEQ ID NO: 271. In some aspects, the antibody comprises a CDR-H3 sequence comprising, consisting of, or consisting essentially of SEQ ID NO: 272. In some aspects, the antibody comprises a CDR-H3 sequence comprising, consisting of, or consisting essentially of SEQ ID NO: 273. In some aspects, the antibody comprises a CDR-H3 sequence comprising, consisting of, or consisting essentially of SEQ ID NO: 274. In some aspects, the antibody comprises a CDR-H3 sequence comprising, consisting of, or consisting essentially of SEQ ID NO: 275. In some aspects, the antibody comprises a CDR-H3 sequence comprising, consisting of, or consisting essentially of SEQ ID NO: 276. In some aspects, the antibody comprises a CDR-H3 sequence comprising, consisting of, or consisting essentially of SEQ ID NO: 277. In some aspects, the antibody comprises a CDR-H3 sequence comprising, consisting of, or consisting essentially of SEQ ID NO: 278. In some aspects, the antibody comprises a CDR-H3 sequence comprising, consisting of, or consisting essentially of SEQ ID NO: 279. In some aspects, the antibody comprises a CDR-H3 sequence comprising, consisting of, or consisting essentially of SEQ ID NO: 280. In some aspects, the antibody comprises a CDR-H3 sequence comprising, consisting of, or consisting essentially of SEQ ID NO: 281. In some aspects, the antibody comprises a CDR-H3 sequence comprising, consisting of, or consisting essentially of SEQ ID NO: 282. In some aspects, the antibody comprises a CDR-H3 sequence comprising, consisting of, or consisting essentially of SEQ ID NO: 283. In some aspects, the antibody comprises a CDR-H3 sequence comprising, consisting of, or consisting essentially of SEQ ID NO: 284. In some aspects, the antibody comprises a CDR-H3 sequence comprising, consisting of, or consisting essentially of SEQ ID NO: 285. In some aspects, the antibody comprises a CDR-H3 sequence comprising, consisting of, or consisting essentially of SEQ ID NO: 286. In some aspects, the antibody comprises a CDR-H3 sequence comprising, consisting of, or consisting essentially of SEQ ID NO: 287. In some aspects, the antibody comprises a CDR-H3 sequence comprising, consisting of, or consisting essentially of SEQ ID NO: 288. In some aspects, the antibody comprises a CDR-H3 sequence comprising, consisting of, or consisting essentially of SEQ ID NO: 289. In some aspects, the antibody comprises a CDR-H3 sequence comprising, consisting of, or consisting essentially of SEQ ID NO: 290. In some aspects, the antibody comprises a CDR-H3 sequence comprising, consisting of, or consisting essentially of SEQ ID NO: 291. In some aspects, the antibody comprises a CDR-H3 sequence comprising, consisting of, or consisting essentially of SEQ ID NO: 292. In some aspects, the antibody comprises a CDR-H3 sequence comprising, consisting of, or consisting essentially of SEQ ID NO: 293. In some aspects, the antibody comprises a CDR-H3 sequence comprising, consisting of, or consisting essentially of SEQ ID NO: 294. In some aspects, the antibody comprises a CDR-H3 sequence comprising, consisting of, or consisting essentially of SEQ ID NO: 295. In some aspects, the antibody comprises a CDR-H3 sequence comprising, consisting of, or consisting essentially of SEQ ID NO: 296. In some aspects, the antibody comprises a CDR-H3 sequence comprising, consisting of, or consisting essentially of SEQ ID NO: 297. In some aspects, the antibody comprises a CDR-H3 sequence comprising, consisting of, or consisting essentially of SEQ ID NO: 298. In some aspects, the antibody comprises a CDR-H3 sequence comprising, consisting of, or consisting essentially of SEQ ID NO: 387. In some aspects, the antibody comprises a CDR-H3 sequence comprising, consisting of, or consisting essentially of SEQ ID NO: 397. In some aspects, the antibody comprises a CDR-H3 sequence comprising, consisting of, or consisting essentially of SEQ ID NO: 407. In some aspects, the antibody comprises a CDR-H3 sequence comprising, consisting of, or consisting essentially of SEQ ID NO: 415. In some aspects, the antibody comprises a CDR-H3 sequence comprising, consisting of, or consisting essentially of SEQ ID NO: 423. In some aspects, the antibody comprises a CDR-H3 sequence comprising, consisting of, or consisting essentially of SEQ ID NO: 431. In some aspects, the antibody comprises a CDR-H3 sequence comprising, consisting of, or consisting essentially of SEQ ID NO: 439.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

### 5.2 CDR-H2 Sequences

In some embodiments, the antibody comprises a CDR-H2 sequence comprising, consisting of, or consisting essentially of a CDR-H2 sequence of an illustrative antibody or V_{H} sequence provided herein. In some aspects, the antibody comprises a CDR-H2 sequence comprising, consisting of, or consisting essentially of SEQ ID NO: 181. In some aspects, the antibody comprises a CDR-H2 sequence comprising, consisting of, or consisting essentially of SEQ ID NO: 182. In some aspects, the antibody comprises a CDR-H2 sequence comprising, consisting of, or consisting essentially of SEQ ID NO: 183. In some aspects, the antibody comprises a CDR-H2 sequence comprising, consisting of, or consisting essentially of SEQ ID NO: 184. In some aspects, the antibody comprises a CDR-H2 sequence comprising, consisting of, or consisting essentially of SEQ ID NO: 185. In some aspects, the antibody comprises a CDR-H2 sequence comprising, consisting of, or consisting essentially of SEQ ID NO: 186. In some aspects, the antibody comprises a CDR-H2 sequence comprising, consisting of, or consisting essentially of SEQ ID NO: 187. In some aspects, the antibody comprises a CDR-H2 sequence comprising, consisting of, or consisting essentially of SEQ ID NO: 188. In some aspects, the antibody comprises a CDR-H2 sequence comprising, consisting of, or consisting essentially of SEQ ID NO: 189. In some aspects, the antibody comprises a CDR-H2 sequence comprising, consisting of, or consisting essentially of SEQ ID NO: 190. In some aspects, the antibody comprises a CDR-H2 sequence comprising, consisting of, or consisting essentially of SEQ ID NO: 191. In some aspects, the antibody comprises a CDR-H2 sequence comprising, consisting of, or consisting essentially of SEQ ID NO: 192. In some aspects, the antibody comprises a CDR-H2 sequence comprising, consisting of, or consisting essentially of SEQ ID NO: 193. In some aspects, the antibody comprises a CDR-H2 sequence comprising, consisting of, or consisting essentially of SEQ ID NO: 194. In some aspects, the antibody comprises a CDR-H2 sequence comprising, consisting of, or consisting essentially of SEQ ID NO: 195. In some aspects, the antibody comprises a CDR-H2 sequence comprising, consisting of, or consisting essentially of SEQ ID NO: 196. In some aspects, the antibody comprises a CDR-H2 sequence comprising, consisting of, or consisting essentially of SEQ ID NO: 197. In some aspects, the antibody comprises a CDR-H2 sequence comprising, consisting of, or consisting essentially of SEQ ID NO: 198. In some aspects, the antibody comprises a CDR-H2 sequence comprising, consisting of, or consisting essentially of SEQ ID NO: 199. In some aspects, the antibody comprises a CDR-H2 sequence comprising, consisting of, or consisting essentially of SEQ ID NO: 200. In some aspects, the antibody comprises a CDR-H2 sequence comprising, consisting of, or consisting essentially of SEQ ID NO: 201. In some aspects, the antibody comprises a CDR-H2 sequence comprising, consisting of, or consisting essentially of SEQ ID NO: 202. In some aspects, the antibody comprises a CDR-H2 sequence comprising, consisting of, or consisting essentially of SEQ ID NO: 203. In some aspects, the antibody comprises a CDR-H2 sequence comprising, consisting of, or consisting essentially of SEQ ID NO: 204. In some aspects, the antibody comprises a CDR-H2 sequence comprising, consisting of, or consisting essentially of SEQ ID NO: 205. In some aspects, the antibody comprises a CDR-H2 sequence comprising, consisting of, or consisting essentially of SEQ ID NO: 206. In some aspects, the antibody comprises a CDR-H2 sequence comprising, consisting of, or consisting essentially of SEQ ID NO: 207. In some aspects, the antibody comprises a CDR-H2 sequence comprising, consisting of, or consisting essentially of SEQ ID NO: 208. In some aspects, the antibody comprises a CDR-H2 sequence comprising, consisting of, or consisting essentially of SEQ ID NO: 209. In some aspects, the antibody comprises a CDR-H2 sequence comprising, consisting of, or consisting essentially of SEQ ID NO: 210. In some aspects, the antibody comprises a CDR-H2 sequence comprising, consisting of, or consisting essentially of SEQ ID NO: 211. In some aspects, the antibody comprises a CDR-H2 sequence comprising, consisting of, or consisting essentially of SEQ ID NO: 212. In some aspects, the antibody comprises a CDR-H2 sequence comprising, consisting of, or consisting essentially of SEQ ID NO: 213. In some aspects, the antibody comprises a CDR-H2 sequence comprising, consisting of, or consisting essentially of SEQ ID NO: 214. In some aspects, the antibody comprises a CDR-H2 sequence comprising, consisting of, or consisting essentially of SEQ ID NO: 215. In some aspects, the antibody comprises a CDR-H2 sequence comprising, consisting of, or consisting essentially of SEQ ID NO: 216. In some aspects, the antibody comprises a CDR-H2 sequence comprising, consisting of, or consisting essentially of SEQ ID NO: 217. In some aspects, the antibody comprises a CDR-H2 sequence comprising, consisting of, or consisting essentially of SEQ ID NO: 218. In some aspects, the antibody comprises a CDR-H2 sequence comprising, consisting of, or consisting essentially of SEQ ID NO: 219. In some aspects, the antibody comprises a CDR-H2 sequence comprising, consisting of, or consisting essentially of SEQ ID NO: 220. In some aspects, the antibody comprises a CDR-H2 sequence comprising, consisting of, or consisting essentially of SEQ ID NO: 221. In some aspects, the antibody comprises a CDR-H2 sequence comprising, consisting of, or consisting essentially of SEQ ID NO: 222. In some aspects, the antibody comprises a CDR-H2 sequence comprising, consisting of, or consisting essentially of SEQ ID NO: 223. In some aspects, the antibody comprises a CDR-H2 sequence comprising, consisting of, or consisting essentially of SEQ ID NO: 224. In some aspects, the antibody comprises a CDR-H2 sequence comprising, consisting of, or consisting essentially of SEQ ID NO: 225. In some aspects, the antibody comprises a CDR-H2 sequence comprising, consisting of, or consisting essentially of SEQ ID NO: 226. In some aspects, the antibody comprises a CDR-H2 sequence comprising, consisting of, or consisting essentially of SEQ ID NO: 227. In some aspects, the antibody comprises a CDR-H2 sequence comprising, consisting of, or consisting essentially of SEQ ID NO: 228. In some aspects, the antibody comprises a CDR-H2 sequence comprising, consisting of, or consisting essentially of SEQ ID NO: 229. In some aspects, the antibody comprises a CDR-H2 sequence comprising, consisting of, or consisting essentially of SEQ ID NO: 295. In some aspects, the antibody comprises a CDR-H2 sequence comprising, consisting of, or consisting essentially of SEQ ID NO: 296. In some aspects, the antibody comprises a CDR-H2 sequence comprising, consisting of, or consisting essentially of SEQ ID NO: 230. In some aspects, the antibody comprises a CDR-H2 sequence comprising, consisting of, or consisting essentially of SEQ ID NO: 231. In some aspects, the antibody comprises a CDR-H2 sequence comprising, consisting of, or consisting essentially of SEQ ID NO: 232. In some aspects, the antibody comprises a CDR-H2 sequence comprising, consisting of, or consisting essentially of SEQ ID NO: 233. In some aspects, the antibody comprises a CDR-H2 sequence comprising, consisting of, or consisting essentially of SEQ ID NO: 234. In some aspects, the antibody comprises a CDR-H2 sequence comprising, consisting of, or consisting essentially of SEQ ID NO: 235. In some aspects, the antibody comprises a CDR-H2 sequence comprising, consisting of, or consisting essentially of SEQ ID NO: 236. In some aspects, the antibody comprises a CDR-H2 sequence comprising, consisting of, or consisting essentially of SEQ ID NO: 237. In some aspects, the antibody comprises a CDR-H2 sequence comprising, consisting of, or consisting essentially of SEQ ID NO: 238. In some aspects, the antibody comprises a CDR-H2 sequence comprising, consisting of, or consisting essentially of SEQ ID NO: 239. In some aspects, the antibody comprises a CDR-H2 sequence comprising, consisting of, or consisting essentially of SEQ ID NO: 386. In some aspects, the antibody comprises a CDR-H2 sequence comprising, consisting of, or consisting essentially of SEQ ID NO: 396. In some aspects, the antibody comprises a CDR-H2 sequence comprising, consisting of, or consisting essentially of SEQ ID NO: 406. In some aspects, the antibody comprises a CDR-H2 sequence comprising, consisting of, or consisting essentially of SEQ ID NO: 414. In some aspects, the antibody comprises a CDR-H2 sequence comprising, consisting of, or consisting essentially of SEQ ID NO: 422. In some aspects, the antibody comprises a CDR-H2 sequence comprising, consisting of, or consisting essentially of SEQ ID NO: 430. In some aspects, the antibody comprises a CDR-H2 sequence comprising, consisting of, or consisting essentially of SEQ ID NO: 438.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

### 5.3 CDR-H1 Sequences

In some embodiments, the antibody comprises a CDR-H1 sequence comprising, consisting of, or consisting essentially of a CDR-H1 sequence of an illustrative antibody or V_{H} sequence provided herein. In some aspects, the antibody comprises a CDR-H1 sequence comprising, consisting of, or consisting essentially of SEQ ID NO: 63. In some aspects, the antibody comprises a CDR-H1 sequence comprising, consisting of, or consisting essentially of SEQ ID NO: 64. In some aspects, the antibody comprises a CDR-H1 sequence comprising, consisting of, or consisting essentially of SEQ ID NO: 65. In some aspects, the antibody comprises a CDR-H1 sequence comprising, consisting of, or consisting essentially of SEQ ID NO: 66. In some aspects, the antibody comprises a CDR-H1 sequence comprising, consisting of, or consisting essentially of SEQ ID NO: 67. In some aspects, the antibody comprises a CDR-H1 sequence comprising, consisting of, or consisting essentially of SEQ ID NO: 68. In some aspects, the antibody comprises a CDR-H1 sequence comprising, consisting of, or consisting essentially of SEQ ID NO: 69. In some aspects, the antibody comprises a CDR-H1 sequence comprising, consisting of, or consisting essentially of SEQ ID NO: 70. In some aspects, the antibody comprises a CDR-H1 sequence comprising, consisting of, or consisting essentially of SEQ ID NO: 71. In some aspects, the antibody comprises a CDR-H1 sequence comprising, consisting of, or consisting essentially of SEQ ID NO: 72. In some aspects, the antibody comprises a CDR-H1 sequence comprising, consisting of, or consisting essentially of SEQ ID NO: 73. In some aspects, the antibody comprises a CDR-H1 sequence comprising, consisting of, or consisting essentially of SEQ ID NO: 74. In some aspects, the antibody comprises a CDR-H1 sequence comprising, consisting of, or consisting essentially of SEQ ID NO: 75. In some aspects, the antibody comprises a CDR-H1 sequence comprising, consisting of, or consisting essentially of SEQ ID NO: 76. In some aspects, the antibody comprises a CDR-H1 sequence comprising, consisting of, or consisting essentially of SEQ ID NO: 77. In some aspects, the antibody comprises a CDR-H1 sequence comprising, consisting of, or consisting essentially of SEQ ID NO: 78. In some aspects, the antibody comprises a CDR-H1 sequence comprising, consisting of, or consisting essentially of SEQ ID NO: 79. In some aspects, the antibody comprises a CDR-H1 sequence comprising, consisting of, or consisting essentially of SEQ ID NO: 80. In some aspects, the antibody comprises a CDR-H1 sequence comprising, consisting of, or consisting essentially of SEQ ID NO: 81. In some aspects, the antibody comprises a CDR-H1 sequence comprising, consisting of, or consisting essentially of SEQ ID NO: 82. In some aspects, the antibody comprises a CDR-H1 sequence comprising, consisting of, or consisting essentially of SEQ ID NO: 83. In some aspects, the antibody comprises a CDR-H1 sequence comprising, consisting of, or consisting essentially of SEQ ID NO: 84. In some aspects, the antibody comprises a CDR-H1 sequence comprising, consisting of, or consisting essentially of SEQ ID NO: 85. In some aspects, the antibody comprises a CDR-H1 sequence comprising, consisting of, or consisting essentially of SEQ ID NO: 86. In some aspects, the antibody comprises a CDR-H1 sequence comprising, consisting of, or consisting essentially of SEQ ID NO: 87. In some aspects, the antibody comprises a CDR-H1 sequence comprising, consisting of, or consisting essentially of SEQ ID NO: 88. In some aspects, the antibody comprises a CDR-H1 sequence comprising, consisting of, or consisting essentially of SEQ ID NO: 89. In some aspects, the antibody comprises a CDR-H1 sequence comprising, consisting of, or consisting essentially of SEQ ID NO: 90. In some aspects, the antibody comprises a CDR-H1 sequence comprising, consisting of, or consisting essentially of SEQ ID NO: 91. In some aspects, the antibody comprises a CDR-H1 sequence comprising, consisting of, or consisting essentially of SEQ ID NO: 92. In some aspects, the antibody comprises a CDR-H1 sequence comprising, consisting of, or consisting essentially of SEQ ID NO: 93. In some aspects, the antibody comprises a CDR-H1 sequence comprising, consisting of, or consisting essentially of SEQ ID NO: 94. In some aspects, the antibody comprises a CDR-H1 sequence comprising, consisting of, or consisting essentially of SEQ ID NO: 95. In some aspects, the antibody comprises a CDR-H1 sequence comprising, consisting of, or consisting essentially of SEQ ID NO: 96. In some aspects, the antibody comprises a CDR-H1 sequence comprising, consisting of, or consisting essentially of SEQ ID NO: 97. In some aspects, the antibody comprises a CDR-H1 sequence comprising, consisting of, or consisting essentially of SEQ ID NO: 98. In some aspects, the antibody comprises a CDR-H1 sequence comprising, consisting of, or consisting essentially of SEQ ID NO: 99. In some aspects, the antibody comprises a CDR-H1 sequence comprising, consisting of, or consisting essentially of SEQ ID NO: 100. In some aspects, the antibody comprises a CDR-H1 sequence comprising, consisting of, or consisting essentially of SEQ ID NO: 101. In some aspects, the antibody comprises a CDR-H1 sequence comprising, consisting of, or consisting essentially of SEQ ID NO: 102. In some aspects, the antibody comprises a CDR-H1 sequence comprising, consisting of, or consisting essentially of SEQ ID NO: 103. In some aspects, the antibody comprises a CDR-H1 sequence comprising, consisting of, or consisting essentially of SEQ ID NO: 104. In some aspects, the antibody comprises a CDR-H1 sequence comprising, consisting of, or consisting essentially of SEQ ID NO: 105. In some aspects, the antibody comprises a CDR-H1 sequence comprising, consisting of, or consisting essentially of SEQ ID NO: 106. In some aspects, the antibody comprises a CDR-H1 sequence comprising, consisting of, or consisting essentially of SEQ ID NO: 107. In some aspects, the antibody comprises a CDR-H1 sequence comprising, consisting of, or consisting essentially of SEQ ID NO: 108. In some aspects, the antibody comprises a CDR-H1 sequence comprising, consisting of, or consisting essentially of SEQ ID NO: 109. In some aspects, the antibody comprises a CDR-H1 sequence comprising, consisting of, or consisting essentially of SEQ ID NO: 110. In some aspects, the antibody comprises a CDR-H1 sequence comprising, consisting of, or consisting essentially of SEQ ID NO: 111. In some aspects, the antibody comprises a CDR-H1 sequence comprising, consisting of, or consisting essentially of SEQ ID NO: 112. In some aspects, the antibody comprises a CDR-H1 sequence comprising, consisting of, or consisting essentially of SEQ ID NO: 113. In some aspects, the antibody comprises a CDR-H1 sequence comprising, consisting of, or consisting essentially of SEQ ID NO: 114. In some aspects, the antibody comprises a CDR-H1 sequence comprising, consisting of, or consisting essentially of SEQ ID NO: 115. In some aspects, the antibody comprises a CDR-H1 sequence comprising, consisting of, or consisting essentially of SEQ ID NO: 116. In some aspects, the antibody comprises a CDR-H1 sequence comprising, consisting of, or consisting essentially of SEQ ID NO: 117. In some aspects, the antibody comprises a CDR-H1 sequence comprising, consisting of, or consisting essentially of SEQ ID NO: 118. In some aspects, the antibody comprises a CDR-H1 sequence comprising, consisting of, or consisting essentially of SEQ ID NO: 119. In some aspects, the antibody comprises a CDR-H1 sequence comprising, consisting of, or consisting essentially of SEQ ID NO: 120. In some aspects, the antibody comprises a CDR-H1 sequence comprising, consisting of, or consisting essentially of SEQ ID NO: 121. In some aspects, the antibody comprises a CDR-H1 sequence comprising, consisting of, or consisting essentially of SEQ ID NO: 385. In some aspects, the antibody comprises a CDR-H1 sequence comprising, consisting of, or consisting essentially of SEQ ID NO: 395. In some aspects, the antibody comprises a CDR-H1 sequence comprising, consisting of, or consisting essentially of SEQ ID NO: 405. In some aspects, the antibody comprises a CDR-H1 sequence comprising, consisting of, or consisting essentially of SEQ ID NO: 413. In some aspects, the antibody comprises a CDR-H1 sequence comprising, consisting of, or consisting essentially of SEQ ID NO: 421. In some aspects, the antibody comprises a CDR-H1 sequence comprising, consisting of, or consisting essentially of SEQ ID NO: 429. In some aspects, the antibody comprises a CDR-H1 sequence comprising, consisting of, or consisting essentially of SEQ ID NO: 437.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

### 5.4 V_{H} Sequences Comprising Illustrative CDRs

In some embodiments, the antibody comprises a V_{H} sequence comprising one or more CDR-H sequences comprising, consisting of, or consisting essentially of one or more illustrative CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 308-366.

5.4.1. V_{H} Sequences Comprising Illustrative Kabat CDRs

In some embodiments, the antibody comprises a V_{H} sequence comprising one or more Kabat CDR-H sequences comprising, consisting of, or consisting essentially of one or more illustrative Kabat CDR-H sequences provided in this disclosure, and variants thereof.

5.4.1.1. Kabat CDR-H3

In some embodiments, the antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a V_{H} sequence provided in SEQ ID NOs: 308-366.

In some embodiments, the antibody comprises a V_{H} sequence comprising a Kabat CDR-H3 sequence comprising, consisting of, or consisting essentially of a sequence selected from SEQ ID NOs: 240-298. In some aspects, the antibody comprises a V_{H} sequence comprising a Kabat CDR-H3 sequence comprising, consisting of, or consisting essentially of SEQ ID NO: 240. In some aspects, the antibody comprises a V_{H} sequence comprising a Kabat CDR-H3 sequence comprising, consisting of, or consisting essentially of SEQ ID NO: 241. In some aspects, the antibody comprises a V_{H} sequence comprising a Kabat CDR-H3 sequence comprising, consisting of, or consisting essentially of SEQ ID NO: 242. In some aspects, the antibody comprises a V_{H} sequence comprising a Kabat CDR-H3 sequence comprising, consisting of, or consisting essentially of SEQ ID NO: 243. In some aspects, the antibody comprises a V_{H} sequence comprising a Kabat CDR-H3 sequence comprising, consisting of, or consisting essentially of SEQ ID NO: 244. In some aspects, the antibody comprises a V_{H} sequence comprising a Kabat CDR-H3 sequence comprising, consisting of, or consisting essentially of SEQ ID NO: 245. In some aspects, the antibody comprises a V_{H} sequence comprising a Kabat CDR-H3 sequence comprising, consisting of, or consisting essentially of SEQ ID NO: 246. In some aspects, the antibody comprises a V_{H} sequence comprising a Kabat CDR-H3 sequence comprising, consisting of, or consisting essentially of SEQ ID NO: 247. In some aspects, the antibody comprises a V_{H} sequence comprising a Kabat CDR-H3 sequence comprising, consisting of, or consisting essentially of SEQ ID NO: 248. In some aspects, the antibody comprises a V_{H} sequence comprising a Kabat CDR-H3 sequence comprising, consisting of, or consisting essentially of SEQ ID NO: 249. In some aspects, the antibody comprises a V_{H} sequence comprising a Kabat CDR-H3 sequence comprising, consisting of, or consisting essentially of SEQ ID NO: 250. In some aspects, the antibody comprises a V_{H} sequence comprising a Kabat CDR-H3 sequence comprising, consisting of, or consisting essentially of SEQ ID NO: 251. In some aspects, the antibody comprises a V_{H} sequence comprising a Kabat CDR-H3 sequence comprising, consisting of, or consisting essentially of SEQ ID NO: 252. In some aspects, the antibody comprises a V_{H} sequence comprising a Kabat CDR-H3 sequence comprising, consisting of, or consisting essentially of SEQ ID NO: 253. In some aspects, the antibody comprises a V_{H} sequence comprising a Kabat CDR-H3 sequence comprising, consisting of, or consisting essentially of SEQ ID NO: 254. In some aspects, the antibody comprises a V_{H} sequence comprising a Kabat CDR-H3 sequence comprising, consisting of, or consisting essentially of SEQ ID NO: 255. In some aspects, the antibody comprises a V_{H} sequence comprising a Kabat CDR-H3 sequence comprising, consisting of, or consisting essentially of SEQ ID NO: 256. In some aspects, the antibody comprises a V_{H} sequence comprising a Kabat CDR-H3 sequence comprising, consisting of, or consisting essentially of SEQ ID NO: 257. In some aspects, the antibody comprises a V_{H} sequence comprising a Kabat CDR-H3 sequence comprising, consisting of, or consisting essentially of SEQ ID NO: 258. In some aspects, the antibody comprises a V_{H} sequence comprising a Kabat CDR-H3 sequence comprising, consisting of, or consisting essentially of SEQ ID NO: 259. In some aspects, the antibody comprises a V_{H} sequence comprising a Kabat CDR-H3 sequence comprising, consisting of, or consisting essentially of SEQ ID NO: 260. In some aspects, the antibody comprises a V_{H} sequence comprising a Kabat CDR-H3 sequence comprising, consisting of, or consisting essentially of SEQ ID NO: 261. In some aspects, the antibody comprises a V_{H} sequence comprising a Kabat CDR-H3 sequence comprising, consisting of, or consisting essentially of SEQ ID NO: 262. In some aspects, the antibody comprises a V_{H} sequence comprising a Kabat CDR-H3 sequence comprising, consisting of, or consisting essentially of SEQ ID NO: 263. In some aspects, the antibody comprises a V_{H} sequence comprising a Kabat CDR-H3 sequence comprising, consisting of, or consisting essentially of SEQ ID NO: 264. In some aspects, the antibody comprises a V_{H} sequence comprising a Kabat CDR-H3 sequence comprising, consisting of, or consisting essentially of SEQ ID NO: 265. In some aspects, the antibody comprises a V_{H} sequence comprising a Kabat CDR-H3 sequence comprising, consisting of, or consisting essentially of SEQ ID NO: 266. In some aspects, the antibody comprises a V_{H} sequence comprising a Kabat CDR-H3 sequence comprising, consisting of, or consisting essentially of SEQ ID NO: 267. In some aspects, the antibody comprises a V_{H} sequence comprising a Kabat CDR-H3 sequence comprising, consisting of, or consisting essentially of SEQ ID NO: 268. In some aspects, the antibody comprises a V_{H} sequence comprising a Kabat CDR-H3 sequence comprising, consisting of, or consisting essentially of SEQ ID NO: 269. In some aspects, the antibody comprises a V_{H} sequence comprising a Kabat CDR-H3 sequence comprising, consisting of, or consisting essentially of SEQ ID NO: 270. In some aspects, the antibody comprises a V_{H} sequence comprising a Kabat CDR-H3 sequence comprising, consisting of, or consisting essentially of SEQ ID NO: 271. In some aspects, the antibody comprises a V_{H} sequence comprising a Kabat CDR-H3 sequence comprising, consisting of, or consisting essentially of SEQ ID NO: 272. In some aspects, the antibody comprises a V_{H} sequence comprising a Kabat CDR-H3 sequence comprising, consisting of, or consisting essentially of SEQ ID NO: 273. In some aspects, the antibody comprises a V_{H} sequence comprising a Kabat CDR-H3 sequence comprising, consisting of, or consisting essentially of SEQ ID NO: 274. In some aspects, the antibody comprises a V_{H} sequence comprising a Kabat CDR-H3 sequence comprising, consisting of, or consisting essentially of SEQ ID NO: 275. In some aspects, the antibody comprises a V_{H} sequence comprising a Kabat CDR-H3 sequence comprising, consisting of, or consisting essentially of SEQ ID NO: 276. In some aspects, the antibody comprises a V_{H} sequence comprising a Kabat CDR-H3 sequence comprising, consisting of, or consisting essentially of SEQ ID NO: 277. In some aspects, the antibody comprises a V_{H} sequence comprising a Kabat CDR-H3 sequence comprising, consisting of, or consisting essentially of SEQ ID NO: 278. In some aspects, the antibody comprises a V_{H} sequence comprising a Kabat CDR-H3 sequence comprising, consisting of, or consisting essentially of SEQ ID NO: 279. In some aspects, the antibody comprises a V_{H} sequence comprising a Kabat CDR-H3 sequence comprising, consisting of, or consisting essentially of SEQ ID NO: 280. In some aspects, the antibody comprises a V_{H} sequence comprising a Kabat CDR-H3 sequence comprising, consisting of, or consisting essentially of SEQ ID NO: 281. In some aspects, the antibody comprises a V_{H} sequence comprising a Kabat CDR-H3 sequence comprising, consisting of, or consisting essentially of SEQ ID NO: 282. In some aspects, the antibody comprises a V_{H} sequence comprising a Kabat CDR-H3 sequence comprising, consisting of, or consisting essentially of SEQ ID NO: 283. In some aspects, the antibody comprises a V_{H} sequence comprising a Kabat CDR-H3 sequence comprising, consisting of, or consisting essentially of SEQ ID NO: 284. In some aspects, the antibody comprises a V_{H} sequence comprising a Kabat CDR-H3 sequence comprising, consisting of, or consisting essentially of SEQ ID NO: 285. In some aspects, the antibody comprises a V_{H} sequence comprising a Kabat CDR-H3 sequence comprising, consisting of, or consisting essentially of SEQ ID NO: 286. In some aspects, the antibody comprises a V_{H} sequence comprising a Kabat CDR-H3 sequence comprising, consisting of, or consisting essentially of SEQ ID NO: 287. In some aspects, the antibody comprises a V_{H} sequence comprising a Kabat CDR-H3 sequence comprising, consisting of, or consisting essentially of SEQ ID NO: 288. In some aspects, the antibody comprises a V_{H} sequence comprising a Kabat CDR-H3 sequence comprising, consisting of, or consisting essentially of SEQ ID NO: 289. In some aspects, the antibody comprises a V_{H} sequence comprising a Kabat CDR-H3 sequence comprising, consisting of, or consisting essentially of SEQ ID NO: 290. In some aspects, the antibody comprises a V_{H} sequence comprising a Kabat CDR-H3 sequence comprising, consisting of, or consisting essentially of SEQ ID NO: 291. In some aspects, the antibody comprises a V_{H} sequence comprising a Kabat CDR-H3 sequence comprising, consisting of, or consisting essentially of SEQ ID NO: 292. In some aspects, the antibody comprises a V_{H} sequence comprising a Kabat CDR-H3 sequence comprising, consisting of, or consisting essentially of SEQ ID NO: 293. In some aspects, the antibody comprises a V_{H} sequence comprising a Kabat CDR-H3 sequence comprising, consisting of, or consisting essentially of SEQ ID NO: 294. In some aspects, the antibody comprises a V_{H} sequence comprising a Kabat CDR-H3 sequence comprising, consisting of, or consisting essentially of SEQ ID NO: 295. In some aspects, the antibody comprises a V_{H} sequence comprising a Kabat CDR-H3 sequence comprising, consisting of, or consisting essentially of SEQ ID NO: 296. In some aspects, the antibody comprises a V_{H} sequence comprising a Kabat CDR-H3 sequence comprising, consisting of, or consisting essentially of SEQ ID NO: 297. In some aspects, the antibody comprises a V_{H} sequence comprising a Kabat CDR-H3 sequence comprising, consisting of, or consisting essentially of SEQ ID NO: 298. In some aspects, the antibody comprises a V_{H} sequence comprising a Kabat CDR-H3 sequence comprising, consisting of, or consisting essentially of SEQ ID NO: 397. In some aspects, the antibody comprises a V_{H} sequence comprising a Kabat CDR-H3 sequence comprising, consisting of, or consisting essentially of SEQ ID NO: 407. In some aspects, the antibody comprises a V_{H} sequence comprising a Kabat CDR-H3 sequence comprising, consisting of, or consisting essentially of SEQ ID NO: 415. In some aspects, the antibody comprises a V_{H} sequence comprising a Kabat CDR-H3 sequence comprising, consisting of, or consisting essentially of SEQ ID NO: 423. In some aspects, the antibody comprises a V_{H} sequence comprising a Kabat CDR-H3 sequence comprising, consisting of, or consisting essentially of SEQ ID NO: 431. In some aspects, the antibody comprises a V_{H} sequence comprising a Kabat CDR-H3 sequence comprising, consisting of, or consisting essentially of SEQ ID NO: 439.

5.4.1.2. Kabat CDR-H2

In some embodiments, the antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of a V_{H} sequence provided in SEQ ID NOs: 308-366.

In some embodiments, the antibody comprises a V_{H} sequence comprising a Kabat CDR-H2 sequence comprising, consisting of, or consisting essentially of a sequence selected from SEQ ID NOs: 181-239. In some aspects, the antibody comprises a V_{H} sequence comprising a Kabat CDR-H2 sequence comprising, consisting of, or consisting essentially of SEQ ID NO: 181. In some aspects, the antibody comprises a V_{H} sequence comprising a Kabat CDR-H2 sequence comprising, consisting of, or consisting essentially of SEQ ID NO: 182. In some aspects, the antibody comprises a V_{H} sequence comprising a Kabat CDR-H2 sequence comprising, consisting of, or consisting essentially of SEQ ID NO: 183. In some aspects, the antibody comprises a V_{H} sequence comprising a Kabat CDR-H2 sequence comprising, consisting of, or consisting essentially of SEQ ID NO: 184. In some aspects, the antibody comprises a V_{H} sequence comprising a Kabat CDR-H2 sequence comprising, consisting of, or consisting essentially of SEQ ID NO: 185. In some aspects, the antibody comprises a V_{H} sequence comprising a Kabat CDR-H2 sequence comprising, consisting of, or consisting essentially of SEQ ID NO: 186. In some aspects, the antibody comprises a V_{H} sequence comprising a Kabat CDR-H2 sequence comprising, consisting of, or consisting essentially of SEQ ID NO: 187. In some aspects, the antibody comprises a V_{H} sequence comprising a Kabat CDR-H2 sequence comprising, consisting of, or consisting essentially of SEQ ID NO: 188. In some aspects, the antibody comprises a V_{H} sequence comprising a Kabat CDR-H2 sequence comprising, consisting of, or consisting essentially of SEQ ID NO: 189. In some aspects, the antibody comprises a V_{H} sequence comprising a Kabat CDR-H2 sequence comprising, consisting of, or consisting essentially of SEQ ID NO: 190. In some aspects, the antibody comprises a V_{H} sequence comprising a Kabat CDR-H2 sequence comprising, consisting of, or consisting essentially of SEQ ID NO: 191. In some aspects, the antibody comprises a V_{H} sequence comprising a Kabat CDR-H2 sequence comprising, consisting of, or consisting essentially of SEQ ID NO: 192. In some aspects, the antibody comprises a V_{H} sequence comprising a Kabat CDR-H2 sequence comprising, consisting of, or consisting essentially of SEQ ID NO: 193. In some aspects, the antibody comprises a V_{H} sequence comprising a Kabat CDR-H2 sequence comprising, consisting of, or consisting essentially of SEQ ID NO: 194. In some aspects, the antibody comprises a V_{H} sequence comprising a Kabat CDR-H2 sequence comprising, consisting of, or consisting essentially of SEQ ID NO: 195. In some aspects, the antibody comprises a V_{H} sequence comprising a Kabat CDR-H2 sequence comprising, consisting of, or consisting essentially of SEQ ID NO: 196. In some aspects, the antibody comprises a V_{H} sequence comprising a Kabat CDR-H2 sequence comprising, consisting of, or consisting essentially of SEQ ID NO: 197. In some aspects, the antibody comprises a V_{H} sequence comprising a Kabat CDR-H2 sequence comprising, consisting of, or consisting essentially of SEQ ID NO: 198. In some aspects, the antibody comprises a V_{H} sequence comprising a Kabat CDR-H2 sequence comprising, consisting of, or consisting essentially of SEQ ID NO: 199. In some aspects, the antibody comprises a V_{H} sequence comprising a Kabat CDR-H2 sequence comprising, consisting of, or consisting essentially of SEQ ID NO: 200. In some aspects, the antibody comprises a V_{H} sequence comprising a Kabat CDR-H2 sequence comprising, consisting of, or consisting essentially of SEQ ID NO: 201. In some aspects, the antibody comprises a V_{H} sequence comprising a Kabat CDR-H2 sequence comprising, consisting of, or consisting essentially of SEQ ID NO: 202. In some aspects, the antibody comprises a V_{H} sequence comprising a Kabat CDR-H2 sequence comprising, consisting of, or consisting essentially of SEQ ID NO: 203. In some aspects, the antibody comprises a V_{H} sequence comprising a Kabat CDR-H2 sequence comprising, consisting of, or consisting essentially of SEQ ID NO: 204. In some aspects, the antibody comprises a V_{H} sequence comprising a Kabat CDR-H2 sequence comprising, consisting of, or consisting essentially of SEQ ID NO: 205. In some aspects, the antibody comprises a V_{H} sequence comprising a Kabat CDR-H2 sequence comprising, consisting of, or consisting essentially of SEQ ID NO: 206. In some aspects, the antibody comprises a V_{H} sequence comprising a Kabat CDR-H2 sequence comprising, consisting of, or consisting essentially of SEQ ID NO: 207. In some aspects, the antibody comprises a V_{H} sequence comprising a Kabat CDR-H2 sequence comprising, consisting of, or consisting essentially of SEQ ID NO: 208. In some aspects, the antibody comprises a V_{H} sequence comprising a Kabat CDR-H2 sequence comprising, consisting of, or consisting essentially of SEQ ID NO: 209. In some aspects, the antibody comprises a V_{H} sequence comprising a Kabat CDR-H2 sequence comprising, consisting of, or consisting essentially of SEQ ID NO: 210. In some aspects, the antibody comprises a V_{H} sequence comprising a Kabat CDR-H2 sequence comprising, consisting of, or consisting essentially of SEQ ID NO: 211. In some aspects, the antibody comprises a V_{H} sequence comprising a Kabat CDR-H2 sequence comprising, consisting of, or consisting essentially of SEQ ID NO: 212. In some aspects, the antibody comprises a V_{H} sequence comprising a Kabat CDR-H2 sequence comprising, consisting of, or consisting essentially of SEQ ID NO: 213. In some aspects, the antibody comprises a V_{H} sequence comprising a Kabat CDR-H2 sequence comprising, consisting of, or consisting essentially of SEQ ID NO: 214. In some aspects, the antibody comprises a V_{H} sequence comprising a Kabat CDR-H2 sequence comprising, consisting of, or consisting essentially of SEQ ID NO: 215. In some aspects, the antibody comprises a V_{H} sequence comprising a Kabat CDR-H2 sequence comprising, consisting of, or consisting essentially of SEQ ID NO: 216. In some aspects, the antibody comprises a V_{H} sequence comprising a Kabat CDR-H2 sequence comprising, consisting of, or consisting essentially of SEQ ID NO: 217. In some aspects, the antibody comprises a V_{H} sequence comprising a Kabat CDR-H2 sequence comprising, consisting of, or consisting essentially of SEQ ID NO: 218. In some aspects, the antibody comprises a V_{H} sequence comprising a Kabat CDR-H2 sequence comprising, consisting of, or consisting essentially of SEQ ID NO: 219. In some aspects, the antibody comprises a V_{H} sequence comprising a Kabat CDR-H2 sequence comprising, consisting of, or consisting essentially of SEQ ID NO: 220. In some aspects, the antibody comprises a V_{H} sequence comprising a Kabat CDR-H2 sequence comprising, consisting of, or consisting essentially of SEQ ID NO: 221. In some aspects, the antibody comprises a V_{H} sequence comprising a Kabat CDR-H2 sequence comprising, consisting of, or consisting essentially of SEQ ID NO: 222. In some aspects, the antibody comprises a V_{H} sequence comprising a Kabat CDR-H2 sequence comprising, consisting of, or consisting essentially of SEQ ID NO: 223. In some aspects, the antibody comprises a V_{H} sequence comprising a Kabat CDR-H2 sequence comprising, consisting of, or consisting essentially of SEQ ID NO: 224. In some aspects, the antibody comprises a V_{H} sequence comprising a Kabat CDR-H2 sequence comprising, consisting of, or consisting essentially of SEQ ID NO: 225. In some aspects, the antibody comprises a V_{H} sequence comprising a Kabat CDR-H2 sequence comprising, consisting of, or consisting essentially of SEQ ID NO: 226. In some aspects, the antibody comprises a V_{H} sequence comprising a Kabat CDR-H2 sequence comprising, consisting of, or consisting essentially of SEQ ID NO: 227. In some aspects, the antibody comprises a V_{H} sequence comprising a Kabat CDR-H2 sequence comprising, consisting of, or consisting essentially of SEQ ID NO: 228. In some aspects, the antibody comprises a V_{H} sequence comprising a Kabat CDR-H2 sequence comprising, consisting of, or consisting essentially of SEQ ID NO: 229. In some aspects, the antibody comprises a V_{H} sequence comprising a Kabat CDR-H2 sequence comprising, consisting of, or consisting essentially of SEQ ID NO: 230. In some aspects, the antibody comprises a V_{H} sequence comprising a Kabat CDR-H2 sequence comprising, consisting of, or consisting essentially of SEQ ID NO: 231. In some aspects, the antibody comprises a V_{H} sequence comprising a Kabat CDR-H2 sequence comprising, consisting of, or consisting essentially of SEQ ID NO: 232. In some aspects, the antibody comprises a V_{H} sequence comprising a Kabat CDR-H2 sequence comprising, consisting of, or consisting essentially of SEQ ID NO: 233. In some aspects, the antibody comprises a V_{H} sequence comprising a Kabat CDR-H2 sequence comprising, consisting of, or consisting essentially of SEQ ID NO: 234. In some aspects, the antibody comprises a V_{H} sequence comprising a Kabat CDR-H2 sequence comprising, consisting of, or consisting essentially of SEQ ID NO: 235. In some aspects, the antibody comprises a V_{H} sequence comprising a Kabat CDR-H2 sequence comprising, consisting of, or consisting essentially of SEQ ID NO: 236. In some aspects, the antibody comprises a V_{H} sequence comprising a Kabat CDR-H2 sequence comprising, consisting of, or consisting essentially of SEQ ID NO: 237. In some aspects, the antibody comprises a V_{H} sequence comprising a Kabat CDR-H2 sequence comprising, consisting of, or consisting essentially of SEQ ID NO: 238. In some aspects, the antibody comprises a V_{H} sequence comprising a Kabat CDR-H2 sequence comprising, consisting of, or consisting essentially of SEQ ID NO: 239. In some aspects, the antibody comprises a V_{H} sequence comprising a Kabat CDR-H2 sequence comprising, consisting of, or consisting essentially of SEQ ID NO: 405. In some aspects, the antibody comprises a V_{H} sequence comprising a Kabat CDR-H2 sequence comprising, consisting of, or consisting essentially of SEQ ID NO: 413. In some aspects, the antibody comprises a V_{H} sequence comprising a Kabat CDR-H2 sequence comprising, consisting of, or consisting essentially of SEQ ID NO: 421. In some aspects, the antibody comprises a V_{H} sequence comprising a Kabat CDR-H2 sequence comprising, consisting of, or consisting essentially of SEQ ID NO: 429. In some aspects, the antibody comprises a V_{H} sequence comprising a Kabat CDR-H2 sequence comprising, consisting of, or consisting essentially of SEQ ID NO:437.

5.4.1.3. Kabat CDR-H1

In some embodiments, the antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of a V_{H} sequence provided in SEQ ID NOs: 308-366.

In some embodiments, the antibody comprises a V_{H} sequence comprising a Kabat CDR-H1 sequence comprising, consisting of, or consisting essentially of a sequence selected from SEQ ID NOs: 63-121. In some aspects, the antibody comprises a V_{H} sequence comprising a Kabat CDR-H1 sequence comprising, consisting of, or consisting essentially of SEQ ID NO: 63. In some aspects, the antibody comprises a V_{H} sequence comprising a Kabat CDR-H1 sequence comprising, consisting of, or consisting essentially of SEQ ID NO: 64. In some aspects, the antibody comprises a V_{H} sequence comprising a Kabat CDR-H1 sequence comprising, consisting of, or consisting essentially of SEQ ID NO: 65. In some aspects, the antibody comprises a V_{H} sequence comprising a Kabat CDR-H1 sequence comprising, consisting of, or consisting essentially of SEQ ID NO: 66. In some aspects, the antibody comprises a V_{H} sequence comprising a Kabat CDR-H1 sequence comprising, consisting of, or consisting essentially of SEQ ID NO: 67. In some aspects, the antibody comprises a V_{H} sequence comprising a Kabat CDR-H1 sequence comprising, consisting of, or consisting essentially of SEQ ID NO: 68. In some aspects, the antibody comprises a V_{H} sequence comprising a Kabat CDR-H1 sequence comprising, consisting of, or consisting essentially of SEQ ID NO: 69. In some aspects, the antibody comprises a V_{H} sequence comprising a Kabat CDR-H1 sequence comprising, consisting of, or consisting essentially of SEQ ID NO: 70. In some aspects, the antibody comprises a V_{H} sequence comprising a Kabat CDR-H1 sequence comprising, consisting of, or consisting essentially of SEQ ID NO: 71. In some aspects, the antibody comprises a V_{H} sequence comprising a Kabat CDR-H1 sequence comprising, consisting of, or consisting essentially of SEQ ID NO: 72. In some aspects, the antibody comprises a V_{H} sequence comprising a Kabat CDR-H1 sequence comprising, consisting of, or consisting essentially of SEQ ID NO: 73. In some aspects, the antibody comprises a V_{H} sequence comprising a Kabat CDR-H1 sequence comprising, consisting of, or consisting essentially of SEQ ID NO: 74. In some aspects, the antibody comprises a V_{H} sequence comprising a Kabat CDR-H1 sequence comprising, consisting of, or consisting essentially of SEQ ID NO: 75. In some aspects, the antibody comprises a V_{H} sequence comprising a Kabat CDR-H1 sequence comprising, consisting of, or consisting essentially of SEQ ID NO: 76. In some aspects, the antibody comprises a V_{H} sequence comprising a Kabat CDR-H1 sequence comprising, consisting of, or consisting essentially of SEQ ID NO: 77. In some aspects, the antibody comprises a V_{H} sequence comprising a Kabat CDR-H1 sequence comprising, consisting of, or consisting essentially of SEQ ID NO: 78. In some aspects, the antibody comprises a V_{H} sequence comprising a Kabat CDR-H1 sequence comprising, consisting of, or consisting essentially of SEQ ID NO: 79. In some aspects, the antibody comprises a V_{H} sequence comprising a Kabat CDR-H1 sequence comprising, consisting of, or consisting essentially of SEQ ID NO: 80. In some aspects, the antibody comprises a V_{H} sequence comprising a Kabat CDR-H1 sequence comprising, consisting of, or consisting essentially of SEQ ID NO: 81. In some aspects, the antibody comprises a V_{H} sequence comprising a Kabat CDR-H1 sequence comprising, consisting of, or consisting essentially of SEQ ID NO: 82. In some aspects, the antibody comprises a V_{H} sequence comprising a Kabat CDR-H1 sequence comprising, consisting of, or consisting essentially of SEQ ID NO: 83. In some aspects, the antibody comprises a V_{H} sequence comprising a Kabat CDR-H1 sequence comprising, consisting of, or consisting essentially of SEQ ID NO: 84. In some aspects, the antibody comprises a V_{H} sequence comprising a Kabat CDR-H1 sequence comprising, consisting of, or consisting essentially of SEQ ID NO: 85. In some aspects, the antibody comprises a V_{H} sequence comprising a Kabat CDR-H1 sequence comprising, consisting of, or consisting essentially of SEQ ID NO: 86. In some aspects, the antibody comprises a V_{H} sequence comprising a Kabat CDR-H1 sequence comprising, consisting of, or consisting essentially of SEQ ID NO: 87. In some aspects, the antibody comprises a V_{H} sequence comprising a Kabat CDR-H1 sequence comprising, consisting of, or consisting essentially of SEQ ID NO: 88. In some aspects, the antibody comprises a V_{H} sequence comprising a Kabat CDR-H1 sequence comprising, consisting of, or consisting essentially of SEQ ID NO: 89. In some aspects, the antibody comprises a V_{H} sequence comprising a Kabat CDR-H1 sequence comprising, consisting of, or consisting essentially of SEQ ID NO: 90. In some aspects, the antibody comprises a V_{H} sequence comprising a Kabat CDR-H1 sequence comprising, consisting of, or consisting essentially of SEQ ID NO: 91. In some aspects, the antibody comprises a V_{H} sequence comprising a Kabat CDR-H1 sequence comprising, consisting of, or consisting essentially of SEQ ID NO: 92. In some aspects, the antibody comprises a V_{H} sequence comprising a Kabat CDR-H1 sequence comprising, consisting of, or consisting essentially of SEQ ID NO: 93. In some aspects, the antibody comprises a V_{H} sequence comprising a Kabat CDR-H1 sequence comprising, consisting of, or consisting essentially of SEQ ID NO: 94. In some aspects, the antibody comprises a V_{H} sequence comprising a Kabat CDR-H1 sequence comprising, consisting of, or consisting essentially of SEQ ID NO: 95. In some aspects, the antibody comprises a V_{H} sequence comprising a Kabat CDR-H1 sequence comprising, consisting of, or consisting essentially of SEQ ID NO: 96. In some aspects, the antibody comprises a V_{H} sequence comprising a Kabat CDR-H1 sequence comprising, consisting of, or consisting essentially of SEQ ID NO: 97. In some aspects, the antibody comprises a V_{H} sequence comprising a Kabat CDR-H1 sequence comprising, consisting of, or consisting essentially of SEQ ID NO: 98. In some aspects, the antibody comprises a V_{H} sequence comprising a Kabat CDR-H1 sequence comprising, consisting of, or consisting essentially of SEQ ID NO: 99. In some aspects, the antibody comprises a V_{H} sequence comprising a Kabat CDR-H1 sequence comprising, consisting of, or consisting essentially of SEQ ID NO: 100. In some aspects, the antibody comprises a V_{H} sequence comprising a Kabat CDR-H1 sequence comprising, consisting of, or consisting essentially of SEQ ID NO: 101. In some aspects, the antibody comprises a V_{H} sequence comprising a Kabat CDR-H1 sequence comprising, consisting of, or consisting essentially of SEQ ID NO: 102. In some aspects, the antibody comprises a V_{H} sequence comprising a Kabat CDR-H1 sequence comprising, consisting of, or consisting essentially of SEQ ID NO: 103. In some aspects, the antibody comprises a V_{H} sequence comprising a Kabat CDR-H1 sequence comprising, consisting of, or consisting essentially of SEQ ID NO: 104. In some aspects, the antibody comprises a V_{H} sequence comprising a Kabat CDR-H1 sequence comprising, consisting of, or consisting essentially of SEQ ID NO: 105. In some aspects, the antibody comprises a V_{H} sequence comprising a Kabat CDR-H1 sequence comprising, consisting of, or consisting essentially of SEQ ID NO: 106. In some aspects, the antibody comprises a V_{H} sequence comprising a Kabat CDR-H1 sequence comprising, consisting of, or consisting essentially of SEQ ID NO: 107. In some aspects, the antibody comprises a V_{H} sequence comprising a Kabat CDR-H1 sequence comprising, consisting of, or consisting essentially of SEQ ID NO: 108. In some aspects, the antibody comprises a V_{H} sequence comprising a Kabat CDR-H1 sequence comprising, consisting of, or consisting essentially of SEQ ID NO: 109. In some aspects, the antibody comprises a V_{H} sequence comprising a Kabat CDR-H1 sequence comprising, consisting of, or consisting essentially of SEQ ID NO: 110. In some aspects, the antibody comprises a V_{H} sequence comprising a Kabat CDR-H1 sequence comprising, consisting of, or consisting essentially of SEQ ID NO: 111. In some aspects, the antibody comprises a V_{H} sequence comprising a Kabat CDR-H1 sequence comprising, consisting of, or consisting essentially of SEQ ID NO: 112. In some aspects, the antibody comprises a V_{H} sequence comprising a Kabat CDR-H1 sequence comprising, consisting of, or consisting essentially of SEQ ID NO: 113. In some aspects, the antibody comprises a V_{H} sequence comprising a Kabat CDR-H1 sequence comprising, consisting of, or consisting essentially of SEQ ID NO: 114. In some aspects, the antibody comprises a V_{H} sequence comprising a Kabat CDR-H1 sequence comprising, consisting of, or consisting essentially of SEQ ID NO: 115. In some aspects, the antibody comprises a V_{H} sequence comprising a Kabat CDR-H1 sequence comprising, consisting of, or consisting essentially of SEQ ID NO: 116. In some aspects, the antibody comprises a V_{H} sequence comprising a Kabat CDR-H1 sequence comprising, consisting of, or consisting essentially of SEQ ID NO: 117. In some aspects, the antibody comprises a V_{H} sequence comprising a Kabat CDR-H1 sequence comprising, consisting of, or consisting essentially of SEQ ID NO: 118. In some aspects, the antibody comprises a V_{H} sequence comprising a Kabat CDR-H1 sequence comprising, consisting of, or consisting essentially of SEQ ID NO: 119. In some aspects, the antibody comprises a V_{H} sequence comprising a Kabat CDR-H1 sequence comprising, consisting of, or consisting essentially of SEQ ID NO: 120. In some aspects, the antibody comprises a V_{H} sequence comprising a Kabat CDR-H1 sequence comprising, consisting of, or consisting essentially of SEQ ID NO: 121. In some aspects, the antibody comprises a V_{H} sequence comprising a Kabat CDR-H1 sequence comprising, consisting of, or consisting essentially of SEQ ID NO: 395. In some aspects, the antibody comprises a V_{H} sequence comprising a Kabat CDR-H1 sequence comprising, consisting of, or consisting essentially of SEQ ID NO: 405. In some aspects, the antibody comprises a V_{H} sequence comprising a Kabat CDR-H1 sequence comprising, consisting of, or consisting essentially of SEQ ID NO: 413. In some aspects, the antibody comprises a V_{H} sequence comprising a Kabat CDR-H1 sequence comprising, consisting of, or consisting essentially of SEQ ID NO: 421. In some aspects, the antibody comprises a V_{H} sequence comprising a Kabat CDR-H1 sequence comprising, consisting of, or consisting essentially of SEQ ID NO: 429. In some aspects, the antibody comprises a V_{H} sequence comprising a Kabat CDR-H1 sequence comprising, consisting of, or consisting essentially of SEQ ID NO: 437.

5.4.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOs: 240-298, and a Kabat CDR-H2 sequence comprising, consisting of, or consisting essentially of a sequence selected from SEQ ID NOs: 181-239. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 308-366.

5.4.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs: 240-298, and a Kabat CDR-H1 sequence comprising, consisting of, or consisting essentially of a sequence selected from SEQ ID NOs: 63-121. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 308-366.

5.4.1.6. Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs: 63-121 and a Kabat CDR-H2 sequence comprising, consisting of, or consisting essentially of a sequence selected from SEQ ID NOs: 181-239. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 308-366.

5.4.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 63-121, a Kabat CDR-H2 sequence comprising, consisting of, or consisting essentially of a sequence selected from SEQ ID NOs: 181-239, and a Kabat CDR-H3 sequence comprising, consisting of, or consisting essentially of a sequence selected from SEQ ID NOs: 240-298. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 308-366.

5.4.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

5.4.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

5.4.2.1. Chothia CDR-H3

In some embodiments, the antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of a V_{H} sequence provided in SEQ ID NOs: 308-366.

In some embodiments, the antibody comprises a V_{H} sequence comprising a Chothia CDR-H3 sequence comprising, consisting of, or consisting essentially of a sequence selected from SEQ ID NOs: 240-298. In some aspects, the antibody comprises a V_{H} sequence comprising a Chothia CDR-H3 sequence comprising, consisting of, or consisting essentially of SEQ ID NO: 240. In some aspects, the antibody comprises a V_{H} sequence comprising a Chothia CDR-H3 sequence comprising, consisting of, or consisting essentially of SEQ ID NO: 241. In some aspects, the antibody comprises a V_{H} sequence comprising a Chothia CDR-H3 sequence comprising, consisting of, or consisting essentially of SEQ ID NO: 242. In some aspects, the antibody comprises a V_{H} sequence comprising a Chothia CDR-H3 sequence comprising, consisting of, or consisting essentially of SEQ ID NO: 243. In some aspects, the antibody comprises a V_{H} sequence comprising a Chothia CDR-H3 sequence comprising, consisting of, or consisting essentially of SEQ ID NO: 244. In some aspects, the antibody comprises a V_{H} sequence comprising a Chothia CDR-H3 sequence comprising, consisting of, or consisting essentially of SEQ ID NO: 245. In some aspects, the antibody comprises a V_{H} sequence comprising a Chothia CDR-H3 sequence comprising, consisting of, or consisting essentially of SEQ ID NO: 246. In some aspects, the antibody comprises a V_{H} sequence comprising a Chothia CDR-H3 sequence comprising, consisting of, or consisting essentially of SEQ ID NO: 247. In some aspects, the antibody comprises a V_{H} sequence comprising a Chothia CDR-H3 sequence comprising, consisting of, or consisting essentially of SEQ ID NO: 248. In some aspects, the antibody comprises a V_{H} sequence comprising a Chothia CDR-H3 sequence comprising, consisting of, or consisting essentially of SEQ ID NO: 249. In some aspects, the antibody comprises a V_{H} sequence comprising a Chothia CDR-H3 sequence comprising, consisting of, or consisting essentially of SEQ ID NO: 250. In some aspects, the antibody comprises a V_{H} sequence comprising a Chothia CDR-H3 sequence comprising, consisting of, or consisting essentially of SEQ ID NO: 251. In some aspects, the antibody comprises a V_{H} sequence comprising a Chothia CDR-H3 sequence comprising, consisting of, or consisting essentially of SEQ ID NO: 252. In some aspects, the antibody comprises a V_{H} sequence comprising a Chothia CDR-H3 sequence comprising, consisting of, or consisting essentially of SEQ ID NO: 253. In some aspects, the antibody comprises a V_{H} sequence comprising a Chothia CDR-H3 sequence comprising, consisting of, or consisting essentially of SEQ ID NO: 254. In some aspects, the antibody comprises a V_{H} sequence comprising a Chothia CDR-H3 sequence comprising, consisting of, or consisting essentially of SEQ ID NO: 255. In some aspects, the antibody comprises a V_{H} sequence comprising a Chothia CDR-H3 sequence comprising, consisting of, or consisting essentially of SEQ ID NO: 256. In some aspects, the antibody comprises a V_{H} sequence comprising a Chothia CDR-H3 sequence comprising, consisting of, or consisting essentially of SEQ ID NO: 257. In some aspects, the antibody comprises a V_{H} sequence comprising a Chothia CDR-H3 sequence comprising, consisting of, or consisting essentially of SEQ ID NO: 258. In some aspects, the antibody comprises a V_{H} sequence comprising a Chothia CDR-H3 sequence comprising, consisting of, or consisting essentially of SEQ ID NO: 259. In some aspects, the antibody comprises a V_{H} sequence comprising a Chothia CDR-H3 sequence comprising, consisting of, or consisting essentially of SEQ ID NO: 260. In some aspects, the antibody comprises a V_{H} sequence comprising a Chothia CDR-H3 sequence comprising, consisting of, or consisting essentially of SEQ ID NO: 261. In some aspects, the antibody comprises a V_{H} sequence comprising a Chothia CDR-H3 sequence comprising, consisting of, or consisting essentially of SEQ ID NO: 262. In some aspects, the antibody comprises a V_{H} sequence comprising a Chothia CDR-H3 sequence comprising, consisting of, or consisting essentially of SEQ ID NO: 263. In some aspects, the antibody comprises a V_{H} sequence comprising a Chothia CDR-H3 sequence comprising, consisting of, or consisting essentially of SEQ ID NO: 264. In some aspects, the antibody comprises a V_{H} sequence comprising a Chothia CDR-H3 sequence comprising, consisting of, or consisting essentially of SEQ ID NO: 265. In some aspects, the antibody comprises a V_{H} sequence comprising a Chothia CDR-H3 sequence comprising, consisting of, or consisting essentially of SEQ ID NO: 266. In some aspects, the antibody comprises a V_{H} sequence comprising a Chothia CDR-H3 sequence comprising, consisting of, or consisting essentially of SEQ ID NO: 267. In some aspects, the antibody comprises a V_{H} sequence comprising a Chothia CDR-H3 sequence comprising, consisting of, or consisting essentially of SEQ ID NO: 268. In some aspects, the antibody comprises a V_{H} sequence comprising a Chothia CDR-H3 sequence comprising, consisting of, or consisting essentially of SEQ ID NO: 269. In some aspects, the antibody comprises a V_{H} sequence comprising a Chothia CDR-H3 sequence comprising, consisting of, or consisting essentially of SEQ ID NO: 270. In some aspects, the antibody comprises a V_{H} sequence comprising a Chothia CDR-H3 sequence comprising, consisting of, or consisting essentially of SEQ ID NO: 271. In some aspects, the antibody comprises a V_{H} sequence comprising a Chothia CDR-H3 sequence comprising, consisting of, or consisting essentially of SEQ ID NO: 272. In some aspects, the antibody comprises a V_{H} sequence comprising a Chothia CDR-H3 sequence comprising, consisting of, or consisting essentially of SEQ ID NO: 273. In some aspects, the antibody comprises a V_{H} sequence comprising a Chothia CDR-H3 sequence comprising, consisting of, or consisting essentially of SEQ ID NO: 274. In some aspects, the antibody comprises a V_{H} sequence comprising a Chothia CDR-H3 sequence comprising, consisting of, or consisting essentially of SEQ ID NO: 275. In some aspects, the antibody comprises a V_{H} sequence comprising a Chothia CDR-H3 sequence comprising, consisting of, or consisting essentially of SEQ ID NO: 276. In some aspects, the antibody comprises a V_{H} sequence comprising a Chothia CDR-H3 sequence comprising, consisting of, or consisting essentially of SEQ ID NO: 277. In some aspects, the antibody comprises a V_{H} sequence comprising a Chothia CDR-H3 sequence comprising, consisting of, or consisting essentially of SEQ ID NO: 278. In some aspects, the antibody comprises a V_{H} sequence comprising a Chothia CDR-H3 sequence comprising, consisting of, or consisting essentially of SEQ ID NO: 279. In some aspects, the antibody comprises a V_{H} sequence comprising a Chothia CDR-H3 sequence comprising, consisting of, or consisting essentially of SEQ ID NO: 280. In some aspects, the antibody comprises a V_{H} sequence comprising a Chothia CDR-H3 sequence comprising, consisting of, or consisting essentially of SEQ ID NO: 281. In some aspects, the antibody comprises a V_{H} sequence comprising a Chothia CDR-H3 sequence comprising, consisting of, or consisting essentially of SEQ ID NO: 282. In some aspects, the antibody comprises a V_{H} sequence comprising a Chothia CDR-H3 sequence comprising, consisting of, or consisting essentially of SEQ ID NO: 283. In some aspects, the antibody comprises a V_{H} sequence comprising a Chothia CDR-H3 sequence comprising, consisting of, or consisting essentially of SEQ ID NO: 284. In some aspects, the antibody comprises a V_{H} sequence comprising a Chothia CDR-H3 sequence comprising, consisting of, or consisting essentially of SEQ ID NO: 285. In some aspects, the antibody comprises a V_{H} sequence comprising a Chothia CDR-H3 sequence comprising, consisting of, or consisting essentially of SEQ ID NO: 286. In some aspects, the antibody comprises a V_{H} sequence comprising a Chothia CDR-H3 sequence comprising, consisting of, or consisting essentially of SEQ ID NO: 287. In some aspects, the antibody comprises a V_{H} sequence comprising a Chothia CDR-H3 sequence comprising, consisting of, or consisting essentially of SEQ ID NO: 288. In some aspects, the antibody comprises a V_{H} sequence comprising a Chothia CDR-H3 sequence comprising, consisting of, or consisting essentially of SEQ ID NO: 289. In some aspects, the antibody comprises a V_{H} sequence comprising a Chothia CDR-H3 sequence comprising, consisting of, or consisting essentially of SEQ ID NO: 290. In some aspects, the antibody comprises a V_{H} sequence comprising a Chothia CDR-H3 sequence comprising, consisting of, or consisting essentially of SEQ ID NO: 291. In some aspects, the antibody comprises a V_{H} sequence comprising a Chothia CDR-H3 sequence comprising, consisting of, or consisting essentially of SEQ ID NO: 292. In some aspects, the antibody comprises a V_{H} sequence comprising a Chothia CDR-H3 sequence comprising, consisting of, or consisting essentially of SEQ ID NO: 293. In some aspects, the antibody comprises a V_{H} sequence comprising a Chothia CDR-H3 sequence comprising, consisting of, or consisting essentially of SEQ ID NO: 294. In some aspects, the antibody comprises a V_{H} sequence comprising a Chothia CDR-H3 sequence comprising, consisting of, or consisting essentially of SEQ ID NO: 295. In some aspects, the antibody comprises a V_{H} sequence comprising a Chothia CDR-H3 sequence comprising, consisting of, or consisting essentially of SEQ ID NO: 296. In some aspects, the antibody comprises a V_{H} sequence comprising a Chothia CDR-H3 sequence comprising, consisting of, or consisting essentially of SEQ ID NO: 297. In some aspects, the antibody comprises a V_{H} sequence comprising a Chothia CDR-H3 sequence comprising, consisting of, or consisting essentially of SEQ ID NO: 298.

5.4.2.2. Chothia CDR-H2

In some embodiments, the antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of a V_{H} sequence provided in SEQ ID NOs: 308-366.

In some embodiments, the antibody comprises a V_{H} sequence comprising a Chothia CDR-H2 sequence comprising, consisting of, or consisting essentially of a sequence selected from SEQ ID NOs: 122-180. In some aspects, the antibody comprises a V_{H} sequence comprising a Chothia CDR-H2 sequence comprising, consisting of, or consisting essentially of SEQ ID NO: 122. In some aspects, the antibody comprises a V_{H} sequence comprising a Chothia CDR-H2 sequence comprising, consisting of, or consisting essentially of SEQ ID NO: 123. In some aspects, the antibody comprises a V_{H} sequence comprising a Chothia CDR-H2 sequence comprising, consisting of, or consisting essentially of SEQ ID NO: 124. In some aspects, the antibody comprises a V_{H} sequence comprising a Chothia CDR-H2 sequence comprising, consisting of, or consisting essentially of SEQ ID NO: 125. In some aspects, the antibody comprises a V_{H} sequence comprising a Chothia CDR-H2 sequence comprising, consisting of, or consisting essentially of SEQ ID NO: 126. In some aspects, the antibody comprises a V_{H} sequence comprising a Chothia CDR-H2 sequence comprising, consisting of, or consisting essentially of SEQ ID NO: 127. In some aspects, the antibody comprises a V_{H} sequence comprising a Chothia CDR-H2 sequence comprising, consisting of, or consisting essentially of SEQ ID NO: 128. In some aspects, the antibody comprises a V_{H} sequence comprising a Chothia CDR-H2 sequence comprising, consisting of, or consisting essentially of SEQ ID NO: 129. In some aspects, the antibody comprises a V_{H} sequence comprising a Chothia CDR-H2 sequence comprising, consisting of, or consisting essentially of SEQ ID NO: 130. In some aspects, the antibody comprises a V_{H} sequence comprising a Chothia CDR-H2 sequence comprising, consisting of, or consisting essentially of SEQ ID NO: 131. In some aspects, the antibody comprises a V_{H} sequence comprising a Chothia CDR-H2 sequence comprising, consisting of, or consisting essentially of SEQ ID NO: 132. In some aspects, the antibody comprises a V_{H} sequence comprising a Chothia CDR-H2 sequence comprising, consisting of, or consisting essentially of SEQ ID NO: 133. In some aspects, the antibody comprises a V_{H} sequence comprising a Chothia CDR-H2 sequence comprising, consisting of, or consisting essentially of SEQ ID NO: 134. In some aspects, the antibody comprises a V_{H} sequence comprising a Chothia CDR-H2 sequence comprising, consisting of, or consisting essentially of SEQ ID NO: 135. In some aspects, the antibody comprises a V_{H} sequence comprising a Chothia CDR-H2 sequence comprising, consisting of, or consisting essentially of SEQ ID NO: 136. In some aspects, the antibody comprises a V_{H} sequence comprising a Chothia CDR-H2 sequence comprising, consisting of, or consisting essentially of SEQ ID NO: 137. In some aspects, the antibody comprises a V_{H} sequence comprising a Chothia CDR-H2 sequence comprising, consisting of, or consisting essentially of SEQ ID NO: 138. In some aspects, the antibody comprises a V_{H} sequence comprising a Chothia CDR-H2 sequence comprising, consisting of, or consisting essentially of SEQ ID NO: 139. In some aspects, the antibody comprises a V_{H} sequence comprising a Chothia CDR-H2 sequence comprising, consisting of, or consisting essentially of SEQ ID NO: 140. In some aspects, the antibody comprises a V_{H} sequence comprising a Chothia CDR-H2 sequence comprising, consisting of, or consisting essentially of SEQ ID NO: 141. In some aspects, the antibody comprises a V_{H} sequence comprising a Chothia CDR-H2 sequence comprising, consisting of, or consisting essentially of SEQ ID NO: 142. In some aspects, the antibody comprises a V_{H} sequence comprising a Chothia CDR-H2 sequence comprising, consisting of, or consisting essentially of SEQ ID NO: 143. In some aspects, the antibody comprises a V_{H} sequence comprising a Chothia CDR-H2 sequence comprising, consisting of, or consisting essentially of SEQ ID NO: 144. In some aspects, the antibody comprises a V_{H} sequence comprising a Chothia CDR-H2 sequence comprising, consisting of, or consisting essentially of SEQ ID NO: 145. In some aspects, the antibody comprises a V_{H} sequence comprising a Chothia CDR-H2 sequence comprising, consisting of, or consisting essentially of SEQ ID NO: 146. In some aspects, the antibody comprises a V_{H} sequence comprising a Chothia CDR-H2 sequence comprising, consisting of, or consisting essentially of SEQ ID NO: 147. In some aspects, the antibody comprises a V_{H} sequence comprising a Chothia CDR-H2 sequence comprising, consisting of, or consisting essentially of SEQ ID NO: 148. In some aspects, the antibody comprises a V_{H} sequence comprising a Chothia CDR-H2 sequence comprising, consisting of, or consisting essentially of SEQ ID NO: 149. In some aspects, the antibody comprises a V_{H} sequence comprising a Chothia CDR-H2 sequence comprising, consisting of, or consisting essentially of SEQ ID NO: 150. In some aspects, the antibody comprises a V_{H} sequence comprising a Chothia CDR-H2 sequence comprising, consisting of, or consisting essentially of SEQ ID NO: 151. In some aspects, the antibody comprises a V_{H} sequence comprising a Chothia CDR-H2 sequence comprising, consisting of, or consisting essentially of SEQ ID NO: 152. In some aspects, the antibody comprises a V_{H} sequence comprising a Chothia CDR-H2 sequence comprising, consisting of, or consisting essentially of SEQ ID NO: 153. In some aspects, the antibody comprises a V_{H} sequence comprising a Chothia CDR-H2 sequence comprising, consisting of, or consisting essentially of SEQ ID NO: 154. In some aspects, the antibody comprises a V_{H} sequence comprising a Chothia CDR-H2 sequence comprising, consisting of, or consisting essentially of SEQ ID NO: 155. In some aspects, the antibody comprises a V_{H} sequence comprising a Chothia CDR-H2 sequence comprising, consisting of, or consisting essentially of SEQ ID NO: 156. In some aspects, the antibody comprises a V_{H} sequence comprising a Chothia CDR-H2 sequence comprising, consisting of, or consisting essentially of SEQ ID NO: 157. In some aspects, the antibody comprises a V_{H} sequence comprising a Chothia CDR-H2 sequence comprising, consisting of, or consisting essentially of SEQ ID NO: 158. In some aspects, the antibody comprises a V_{H} sequence comprising a Chothia CDR-H2 sequence comprising, consisting of, or consisting essentially of SEQ ID NO: 159. In some aspects, the antibody comprises a V_{H} sequence comprising a Chothia CDR-H2 sequence comprising, consisting of, or consisting essentially of SEQ ID NO: 160. In some aspects, the antibody comprises a V_{H} sequence comprising a Chothia CDR-H2 sequence comprising, consisting of, or consisting essentially of SEQ ID NO: 161. In some aspects, the antibody comprises a V_{H} sequence comprising a Chothia CDR-H2 sequence comprising, consisting of, or consisting essentially of SEQ ID NO: 162. In some aspects, the antibody comprises a V_{H} sequence comprising a Chothia CDR-H2 sequence comprising, consisting of, or consisting essentially of SEQ ID NO: 163. In some aspects, the antibody comprises a V_{H} sequence comprising a Chothia CDR-H2 sequence comprising, consisting of, or consisting essentially of SEQ ID NO: 164. In some aspects, the antibody comprises a V_{H} sequence comprising a Chothia CDR-H2 sequence comprising, consisting of, or consisting essentially of SEQ ID NO: 165. In some aspects, the antibody comprises a V_{H} sequence comprising a Chothia CDR-H2 sequence comprising, consisting of, or consisting essentially of SEQ ID NO: 166. In some aspects, the antibody comprises a V_{H} sequence comprising a Chothia CDR-H2 sequence comprising, consisting of, or consisting essentially of SEQ ID NO: 167. In some aspects, the antibody comprises a V_{H} sequence comprising a Chothia CDR-H2 sequence comprising, consisting of, or consisting essentially of SEQ ID NO: 168. In some aspects, the antibody comprises a V_{H} sequence comprising a Chothia CDR-H2 sequence comprising, consisting of, or consisting essentially of SEQ ID NO: 169. In some aspects, the antibody comprises a V_{H} sequence comprising a Chothia CDR-H2 sequence comprising, consisting of, or consisting essentially of SEQ ID NO: 170. In some aspects, the antibody comprises a V_{H} sequence comprising a Chothia CDR-H2 sequence comprising, consisting of, or consisting essentially of SEQ ID NO: 171. In some aspects, the antibody comprises a V_{H} sequence comprising a Chothia CDR-H2 sequence comprising, consisting of, or consisting essentially of SEQ ID NO: 172. In some aspects, the antibody comprises a V_{H} sequence comprising a Chothia CDR-H2 sequence comprising, consisting of, or consisting essentially of SEQ ID NO: 173. In some aspects, the antibody comprises a V_{H} sequence comprising a Chothia CDR-H2 sequence comprising, consisting of, or consisting essentially of SEQ ID NO: 174. In some aspects, the antibody comprises a V_{H} sequence comprising a Chothia CDR-H2 sequence comprising, consisting of, or consisting essentially of SEQ ID NO: 175. In some aspects, the antibody comprises a V_{H} sequence comprising a Chothia CDR-H2 sequence comprising, consisting of, or consisting essentially of SEQ ID NO: 176. In some aspects, the antibody comprises a V_{H} sequence comprising a Chothia CDR-H2 sequence comprising, consisting of, or consisting essentially of SEQ ID NO: 177. In some aspects, the antibody comprises a V_{H} sequence comprising a Chothia CDR-H2 sequence comprising, consisting of, or consisting essentially of SEQ ID NO: 178. In some aspects, the antibody comprises a V_{H} sequence comprising a Chothia CDR-H2 sequence comprising, consisting of, or consisting essentially of SEQ ID NO: 179. In some aspects, the antibody comprises a V_{H} sequence comprising a Chothia CDR-H2 sequence comprising, consisting of, or consisting essentially of SEQ ID NO: 180.

5.4.2.3. Chothia CDR-H1

In some embodiments, the antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of a V_{H} sequence provided in SEQ ID NOs: 308-366.

In some embodiments, the antibody comprises a V_{H} sequence comprising a Chothia CDR-H1 sequence comprising, consisting of, or consisting essentially of a sequence selected from SEQ ID NOs: 4-62. In some aspects, the antibody comprises a V_{H} sequence comprising a Chothia CDR-H1 sequence comprising, consisting of, or consisting essentially of SEQ ID NO: 4. In some aspects, the antibody comprises a V_{H} sequence comprising a Chothia CDR-H1 sequence comprising, consisting of, or consisting essentially of SEQ ID NO: 5. In some aspects, the antibody comprises a V_{H} sequence comprising a Chothia CDR-H1 sequence comprising, consisting of, or consisting essentially of SEQ ID NO: 6. In some aspects, the antibody comprises a V_{H} sequence comprising a Chothia CDR-H1 sequence comprising, consisting of, or consisting essentially of SEQ ID NO: 7. In some aspects, the antibody comprises a V_{H} sequence comprising a Chothia CDR-H1 sequence comprising, consisting of, or consisting essentially of SEQ ID NO: 8. In some aspects, the antibody comprises a V_{H} sequence comprising a Chothia CDR-H1 sequence comprising, consisting of, or consisting essentially of SEQ ID NO: 9. In some aspects, the antibody comprises a V_{H} sequence comprising a Chothia CDR-H1 sequence comprising, consisting of, or consisting essentially of SEQ ID NO: 10. In some aspects, the antibody comprises a V_{H} sequence comprising a Chothia CDR-H1 sequence comprising, consisting of, or consisting essentially of SEQ ID NO: 11. In some aspects, the antibody comprises a V_{H} sequence comprising a Chothia CDR-H1 sequence comprising, consisting of, or consisting essentially of SEQ ID NO: 12. In some aspects, the antibody comprises a V_{H} sequence comprising a Chothia CDR-H1 sequence comprising, consisting of, or consisting essentially of SEQ ID NO: 13. In some aspects, the antibody comprises a V_{H} sequence comprising a Chothia CDR-H1 sequence comprising, consisting of, or consisting essentially of SEQ ID NO: 14. In some aspects, the antibody comprises a V_{H} sequence comprising a Chothia CDR-H1 sequence comprising, consisting of, or consisting essentially of SEQ ID NO: 15. In some aspects, the antibody comprises a V_{H} sequence comprising a Chothia CDR-H1 sequence comprising, consisting of, or consisting essentially of SEQ ID NO: 16. In some aspects, the antibody comprises a V_{H} sequence comprising a Chothia CDR-H1 sequence comprising, consisting of, or consisting essentially of SEQ ID NO: 17. In some aspects, the antibody comprises a V_{H} sequence comprising a Chothia CDR-H1 sequence comprising, consisting of, or consisting essentially of SEQ ID NO: 18. In some aspects, the antibody comprises a V_{H} sequence comprising a Chothia CDR-H1 sequence comprising, consisting of, or consisting essentially of SEQ ID NO: 19. In some aspects, the antibody comprises a V_{H} sequence comprising a Chothia CDR-H1 sequence comprising, consisting of, or consisting essentially of SEQ ID NO: 20. In some aspects, the antibody comprises a V_{H} sequence comprising a Chothia CDR-H1 sequence comprising, consisting of, or consisting essentially of SEQ ID NO: 21. In some aspects, the antibody comprises a V_{H} sequence comprising a Chothia CDR-H1 sequence comprising, consisting of, or consisting essentially of SEQ ID NO: 22. In some aspects, the antibody comprises a V_{H} sequence comprising a Chothia CDR-H1 sequence comprising, consisting of, or consisting essentially of SEQ ID NO: 23. In some aspects, the antibody comprises a V_{H} sequence comprising a Chothia CDR-H1 sequence comprising, consisting of, or consisting essentially of SEQ ID NO: 24. In some aspects, the antibody comprises a V_{H} sequence comprising a Chothia CDR-H1 sequence comprising, consisting of, or consisting essentially of SEQ ID NO: 25. In some aspects, the antibody comprises a V_{H} sequence comprising a Chothia CDR-H1 sequence comprising, consisting of, or consisting essentially of SEQ ID NO: 26. In some aspects, the antibody comprises a V_{H} sequence comprising a Chothia CDR-H1 sequence comprising, consisting of, or consisting essentially of SEQ ID NO: 27. In some aspects, the antibody comprises a V_{H} sequence comprising a Chothia CDR-H1 sequence comprising, consisting of, or consisting essentially of SEQ ID NO: 28. In some aspects, the antibody comprises a V_{H} sequence comprising a Chothia CDR-H1 sequence comprising, consisting of, or consisting essentially of SEQ ID NO: 29. In some aspects, the antibody comprises a V_{H} sequence comprising a Chothia CDR-H1 sequence comprising, consisting of, or consisting essentially of SEQ ID NO: 30. In some aspects, the antibody comprises a V_{H} sequence comprising a Chothia CDR-H1 sequence comprising, consisting of, or consisting essentially of SEQ ID NO: 31. In some aspects, the antibody comprises a V_{H} sequence comprising a Chothia CDR-H1 sequence comprising, consisting of, or consisting essentially of SEQ ID NO: 32. In some aspects, the antibody comprises a V_{H} sequence comprising a Chothia CDR-H1 sequence comprising, consisting of, or consisting essentially of SEQ ID NO: 33. In some aspects, the antibody comprises a V_{H} sequence comprising a Chothia CDR-H1 sequence comprising, consisting of, or consisting essentially of SEQ ID NO: 34. In some aspects, the antibody comprises a V_{H} sequence comprising a Chothia CDR-H1 sequence comprising, consisting of, or consisting essentially of SEQ ID NO: 35. In some aspects, the antibody comprises a V_{H} sequence comprising a Chothia CDR-H1 sequence comprising, consisting of, or consisting essentially of SEQ ID NO: 36. In some aspects, the antibody comprises a V_{H} sequence comprising a Chothia CDR-H1 sequence comprising, consisting of, or consisting essentially of SEQ ID NO: 37. In some aspects, the antibody comprises a V_{H} sequence comprising a Chothia CDR-H1 sequence comprising, consisting of, or consisting essentially of SEQ ID NO: 38. In some aspects, the antibody comprises a V_{H} sequence comprising a Chothia CDR-H1 sequence comprising, consisting of, or consisting essentially of SEQ ID NO: 39. In some aspects, the antibody comprises a V_{H} sequence comprising a Chothia CDR-H1 sequence comprising, consisting of, or consisting essentially of SEQ ID NO: 40. In some aspects, the antibody comprises a V_{H} sequence comprising a Chothia CDR-H1 sequence comprising, consisting of, or consisting essentially of SEQ ID NO: 41. In some aspects, the antibody comprises a V_{H} sequence comprising a Chothia CDR-H1 sequence comprising, consisting of, or consisting essentially of SEQ ID NO: 42. In some aspects, the antibody comprises a V_{H} sequence comprising a Chothia CDR-H1 sequence comprising, consisting of, or consisting essentially of SEQ ID NO: 43. In some aspects, the antibody comprises a V_{H} sequence comprising a Chothia CDR-H1 sequence comprising, consisting of, or consisting essentially of SEQ ID NO: 44. In some aspects, the antibody comprises a V_{H} sequence comprising a Chothia CDR-H1 sequence comprising, consisting of, or consisting essentially of SEQ ID NO: 45. In some aspects, the antibody comprises a V_{H} sequence comprising a Chothia CDR-H1 sequence comprising, consisting of, or consisting essentially of SEQ ID NO: 46. In some aspects, the antibody comprises a V_{H} sequence comprising a Chothia CDR-H1 sequence comprising, consisting of, or consisting essentially of SEQ ID NO: 47. In some aspects, the antibody comprises a V_{H} sequence comprising a Chothia CDR-H1 sequence comprising, consisting of, or consisting essentially of SEQ ID NO: 48. In some aspects, the antibody comprises a V_{H} sequence comprising a Chothia CDR-H1 sequence comprising, consisting of, or consisting essentially of SEQ ID NO: 49. In some aspects, the antibody comprises a V_{H} sequence comprising a Chothia CDR-H1 sequence comprising, consisting of, or consisting essentially of SEQ ID NO: 50. In some aspects, the antibody comprises a V_{H} sequence comprising a Chothia CDR-H1 sequence comprising, consisting of, or consisting essentially of SEQ ID NO: 51. In some aspects, the antibody comprises a V_{H} sequence comprising a Chothia CDR-H1 sequence comprising, consisting of, or consisting essentially of SEQ ID NO: 52. In some aspects, the antibody comprises a V_{H} sequence comprising a Chothia CDR-H1 sequence comprising, consisting of, or consisting essentially of SEQ ID NO: 53. In some aspects, the antibody comprises a V_{H} sequence comprising a Chothia CDR-H1 sequence comprising, consisting of, or consisting essentially of SEQ ID NO: 54. In some aspects, the antibody comprises a V_{H} sequence comprising a Chothia CDR-H1 sequence comprising, consisting of, or consisting essentially of SEQ ID NO: 55. In some aspects, the antibody comprises a V_{H} sequence comprising a Chothia CDR-H1 sequence comprising, consisting of, or consisting essentially of SEQ ID NO: 56. In some aspects, the antibody comprises a V_{H} sequence comprising a Chothia CDR-H1 sequence comprising, consisting of, or consisting essentially of SEQ ID NO: 57. In some aspects, the antibody comprises a V_{H} sequence comprising a Chothia CDR-H1 sequence comprising, consisting of, or consisting essentially of SEQ ID NO: 58. In some aspects, the antibody comprises a V_{H} sequence comprising a Chothia CDR-H1 sequence comprising, consisting of, or consisting essentially of SEQ ID NO: 59. In some aspects, the antibody comprises a V_{H} sequence comprising a Chothia CDR-H1 sequence comprising, consisting of, or consisting essentially of SEQ ID NO: 60. In some aspects, the antibody comprises a V_{H} sequence comprising a Chothia CDR-H1 sequence comprising, consisting of, or consisting essentially of SEQ ID NO: 61. In some aspects, the antibody comprises a V_{H} sequence comprising a Chothia CDR-H1 sequence comprising, consisting of, or consisting essentially of SEQ ID NO: 62.

5.4.2.4. Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOs: 240-298, and a Chothia CDR-H2 sequence comprising, consisting of, or consisting essentially of a sequence selected from SEQ ID NOs: 122-180. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 308-366.

5.4.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs: 240-298, and a Chothia CDR-H1 sequence comprising, consisting of, or consisting essentially of a sequence selected from SEQ ID NOs: 4-62. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 308-366.

5.4.2.6. Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs: 4-62 and a Chothia CDR-H2 sequence comprising, consisting of, or consisting essentially of a sequence selected from SEQ ID NOs: 122-180. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 308-366.

5.4.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 4-62, a Chothia CDR-H2 sequence comprising, consisting of, or consisting essentially of a sequence selected from SEQ ID NOs: 122-180, and a Chothia CDR-H3 sequence comprising, consisting of, or consisting essentially of a sequence selected from SEQ ID NOs: 240-298. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 308-366.

5.4.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 5.5. V_{H} Sequences

In some embodiments, the antibody comprises, consists of, or consists essentially of a V_{H} sequence provided in SEQ ID NOs: 308-366.

In some embodiments, the antibody comprises a V_{H} sequence comprising, consisting of, or consisting essentially of a sequence selected from SEQ ID NOs: 308-366. In some aspects, the antibody comprises a V_{H} sequence comprising, consisting of, or consisting essentially of SEQ ID NO: 308. In some aspects, the antibody comprises a V_{H} sequence comprising, consisting of, or consisting essentially of SEQ ID NO: 309. In some aspects, the antibody comprises a V_{H} sequence comprising, consisting of, or consisting essentially of SEQ ID NO: 310. In some aspects, the antibody comprises a V_{H} sequence comprising, consisting of, or consisting essentially of SEQ ID NO: 311. In some aspects, the antibody comprises a V_{H} sequence comprising, consisting of, or consisting essentially of SEQ ID NO: 312. In some aspects, the antibody comprises a V_{H} sequence comprising, consisting of, or consisting essentially of SEQ ID NO: 313. In some aspects, the antibody comprises a V_{H} sequence comprising, consisting of, or consisting essentially of SEQ ID NO: 314. In some aspects, the antibody comprises a V_{H} sequence comprising, consisting of, or consisting essentially of SEQ ID NO: 315. In some aspects, the antibody comprises a V_{H} sequence comprising, consisting of, or consisting essentially of SEQ ID NO: 316. In some aspects, the antibody comprises a V_{H} sequence comprising, consisting of, or consisting essentially of SEQ ID NO: 317. In some aspects, the antibody comprises a V_{H} sequence comprising, consisting of, or consisting essentially of SEQ ID NO: 318. In some aspects, the antibody comprises a V_{H} sequence comprising, consisting of, or consisting essentially of SEQ ID NO: 319. In some aspects, the antibody comprises a V_{H} sequence comprising, consisting of, or consisting essentially of SEQ ID NO: 320. In some aspects, the antibody comprises a V_{H} sequence comprising, consisting of, or consisting essentially of SEQ ID NO: 321. In some aspects, the antibody comprises a V_{H} sequence comprising, consisting of, or consisting essentially of SEQ ID NO: 322. In some aspects, the antibody comprises a V_{H} sequence comprising, consisting of, or consisting essentially of SEQ ID NO: 323. In some aspects, the antibody comprises a V_{H} sequence comprising, consisting of, or consisting essentially of SEQ ID NO: 324. In some aspects, the antibody comprises a V_{H} sequence comprising, consisting of, or consisting essentially of SEQ ID NO: 325. In some aspects, the antibody comprises a V_{H} sequence comprising, consisting of, or consisting essentially of SEQ ID NO: 326. In some aspects, the antibody comprises a V_{H} sequence comprising, consisting of, or consisting essentially of SEQ ID NO: 327. In some aspects, the antibody comprises a V_{H} sequence comprising, consisting of, or consisting essentially of SEQ ID NO: 328. In some aspects, the antibody comprises a V_{H} sequence comprising, consisting of, or consisting essentially of SEQ ID NO: 329. In some aspects, the antibody comprises a V_{H} sequence comprising, consisting of, or consisting essentially of SEQ ID NO: 330. In some aspects, the antibody comprises a V_{H} sequence comprising, consisting of, or consisting essentially of SEQ ID NO: 331. In some aspects, the antibody comprises a V_{H} sequence comprising, consisting of, or consisting essentially of SEQ ID NO: 332. In some aspects, the antibody comprises a V_{H} sequence comprising, consisting of, or consisting essentially of SEQ ID NO: 333. In some aspects, the antibody comprises a V_{H} sequence comprising, consisting of, or consisting essentially of SEQ ID NO: 334. In some aspects, the antibody comprises a V_{H} sequence comprising, consisting of, or consisting essentially of SEQ ID NO: 335. In some aspects, the antibody comprises a V_{H} sequence comprising, consisting of, or consisting essentially of SEQ ID NO: 336. In some aspects, the antibody comprises a V_{H} sequence comprising, consisting of, or consisting essentially of SEQ ID NO: 337. In some aspects, the antibody comprises a V_{H} sequence comprising, consisting of, or consisting essentially of SEQ ID NO: 338. In some aspects, the antibody comprises a V_{H} sequence comprising, consisting of, or consisting essentially of SEQ ID NO: 339. In some aspects, the antibody comprises a V_{H} sequence comprising, consisting of, or consisting essentially of SEQ ID NO: 340. In some aspects, the antibody comprises a V_{H} sequence comprising, consisting of, or consisting essentially of SEQ ID NO: 341. In some aspects, the antibody comprises a V_{H} sequence comprising, consisting of, or consisting essentially of SEQ ID NO: 342. In some aspects, the antibody comprises a V_{H} sequence comprising, consisting of, or consisting essentially of SEQ ID NO: 343. In some aspects, the antibody comprises a V_{H} sequence comprising, consisting of, or consisting essentially of SEQ ID NO: 344. In some aspects, the antibody comprises a V_{H} sequence comprising, consisting of, or consisting essentially of SEQ ID NO: 345. In some aspects, the antibody comprises a V_{H} sequence comprising, consisting of, or consisting essentially of SEQ ID NO: 346. In some aspects, the antibody comprises a V_{H} sequence comprising, consisting of, or consisting essentially of SEQ ID NO: 347. In some aspects, the antibody comprises a V_{H} sequence comprising, consisting of, or consisting essentially of SEQ ID NO: 348. In some aspects, the antibody comprises a V_{H} sequence comprising, consisting of, or consisting essentially of SEQ ID NO: 349. In some aspects, the antibody comprises a V_{H} sequence comprising, consisting of, or consisting essentially of SEQ ID NO: 350. In some aspects, the antibody comprises a V_{H} sequence comprising, consisting of, or consisting essentially of SEQ ID NO: 351. In some aspects, the antibody comprises a V_{H} sequence comprising, consisting of, or consisting essentially of SEQ ID NO: 352. In some aspects, the antibody comprises a V_{H} sequence comprising, consisting of, or consisting essentially of SEQ ID NO: 353. In some aspects, the antibody comprises a V_{H} sequence comprising, consisting of, or consisting essentially of SEQ ID NO: 354. In some aspects, the antibody comprises a V_{H} sequence comprising, consisting of, or consisting essentially of SEQ ID NO: 355. In some aspects, the antibody comprises a V_{H} sequence comprising, consisting of, or consisting essentially of SEQ ID NO: 356. In some aspects, the antibody comprises a V_{H} sequence comprising, consisting of, or consisting essentially of SEQ ID NO: 357. In some aspects, the antibody comprises a V_{H} sequence comprising, consisting of, or consisting essentially of SEQ ID NO: 358. In some aspects, the antibody comprises a V_{H} sequence comprising, consisting of, or consisting essentially of SEQ ID NO: 359. In some aspects, the antibody comprises a V_{H} sequence comprising, consisting of, or consisting essentially of SEQ ID NO: 360. In some aspects, the antibody comprises a V_{H} sequence comprising, consisting of, or consisting essentially of SEQ ID NO: 361. In some aspects, the antibody comprises a V_{H} sequence comprising, consisting of, or consisting essentially of SEQ ID NO: 362. In some aspects, the antibody comprises a V_{H} sequence comprising, consisting of, or consisting essentially of SEQ ID NO: 363. In some aspects, the antibody comprises a V_{H} sequence comprising, consisting of, or consisting essentially of SEQ ID NO: 364. In some aspects, the antibody comprises a V_{H} sequence comprising, consisting of, or consisting essentially of SEQ ID NO: 365. In some aspects, the antibody comprises a V_{H} sequence comprising, consisting of, or consisting essentially of SEQ ID NO: 366. In some aspects, the antibody comprises a V_{H} sequence comprising, consisting of, or consisting essentially of SEQ ID NO: 391. In some aspects, the antibody comprises a V_{H} sequence comprising, consisting of, or consisting essentially of SEQ ID NO:401. In some aspects, the antibody comprises a V_{H} sequence comprising, consisting of, or consisting essentially of SEQ ID NO:403. In some aspects, the antibody comprises a V_{H} sequence comprising, consisting of, or consisting essentially of SEQ ID NO: 411. In some aspects, the antibody comprises a V_{H} sequence comprising, consisting of, or consisting essentially of SEQ ID NO: 419. In some aspects, the antibody comprises a V_{H} sequence comprising, consisting of, or consisting essentially of SEQ ID NO:427. In some aspects, the antibody comprises a V_{H} sequence comprising, consisting of, or consisting essentially of SEQ ID NO:435. In some aspects, the antibody comprises a V_{H} sequence comprising, consisting of, or consisting essentially of SEQ ID NO:443.

5.5.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.6. CDR-L3 Sequences

In some embodiments, the antibody comprises a CDR-L3 sequence comprising, consisting of, or consisting essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 367-369.

In some embodiments, the antibody comprises a CDR-L3 sequence comprising, consisting of, or consisting essentially of a sequence selected from SEQ ID NOs: 305-307. In some aspects, the antibody comprises a CDR-L3 sequence comprising, consisting of, or consisting essentially of SEQ ID NO: 305. In some aspects, the antibody comprises a CDR-L3 sequence comprising, consisting of, or consisting essentially of SEQ ID NO: 306. In some aspects, the antibody comprises a CDR-L3 sequence comprising, consisting of, or consisting essentially of SEQ ID NO: 307. In some aspects, the antibody comprises a CDR-L3 sequence comprising, consisting of, or consisting essentially of SEQ ID NO: 390. In some aspects, the antibody comprises a CDR-L3 sequence comprising, consisting of, or consisting essentially of SEQ ID NO: 400. In some aspects, the antibody comprises a CDR-L3 sequence comprising, consisting of, or consisting essentially of SEQ ID NO: 410. In some aspects, the antibody comprises a CDR-L3 sequence comprising, consisting of, or consisting essentially of SEQ ID NO: 418. In some aspects, the antibody comprises a CDR-L3 sequence comprising, consisting of, or consisting essentially of SEQ ID NO: 426. In some aspects, the antibody comprises a CDR-L3 sequence comprising, consisting of, or consisting essentially of SEQ ID NO: 434. In some aspects, the antibody comprises a CDR-L3 sequence comprising, consisting of, or consisting essentially of SEQ ID NO: 442.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 5.7 CDR-L2 Sequences

In some embodiments, the antibody comprises a CDR-L2 sequence comprising, consisting of, or consisting essentially of a CDR-L2 sequence of an illustrative antibody or V_{L} sequence provided herein.

In some aspects, the antibody comprises a CDR-L2 sequence comprising, consisting of, or consisting essentially of SEQ ID NO: 303. In some aspects, the antibody comprises a CDR-L2 sequence comprising, consisting of, or consisting essentially of SEQ ID NO: 304. In some aspects, the antibody comprises a CDR-L2 sequence comprising, consisting of, or consisting essentially of SEQ ID NO: 389. In some aspects, the antibody comprises a CDR-L2 sequence comprising, consisting of, or consisting essentially of SEQ ID NO: 399. In some aspects, the antibody comprises a CDR-L2 sequence comprising, consisting of, or consisting essentially of SEQ ID NO: 409. In some aspects, the antibody comprises a CDR-L2 sequence comprising, consisting of, or consisting essentially of SEQ ID NO: 417. In some aspects, the antibody comprises a CDR-L2 sequence comprising, consisting of, or consisting essentially of SEQ ID NO: 425. In some aspects, the antibody comprises a CDR-L2 sequence comprising, consisting of, or consisting essentially of SEQ ID NO: 433. In some aspects, the antibody comprises a CDR-L2 sequence comprising, consisting of, or consisting essentially of SEQ ID NO: 441.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

### 5.8 CDR-L1 Sequences

In some embodiments, the antibody comprises a CDR-L1 sequence comprising, consisting of, or consisting essentially of a CDR-L1 sequence of an illustrative antibody or V_{L} sequence provided herein.

In some aspects, the antibody comprises a CDR-L1 sequence comprising, consisting of, or consisting essentially of SEQ ID NO: 299. In some aspects, the antibody comprises a CDR-L1 sequence comprising, consisting of, or consisting essentially of SEQ ID NO: 300. In some aspects, the antibody comprises a CDR-L1 sequence comprising, consisting of, or consisting essentially of SEQ ID NO: 301. In some aspects, the antibody comprises a CDR-L1 sequence comprising, consisting of, or consisting essentially of SEQ ID NO: 388. In some aspects, the antibody comprises a CDR-L1 sequence comprising, consisting of, or consisting essentially of SEQ ID NO: 398. In some aspects, the antibody comprises a CDR-L1 sequence comprising, consisting of, or consisting essentially of SEQ ID NO: 408. In some aspects, the antibody comprises a CDR-L1 sequence comprising, consisting of, or consisting essentially of SEQ ID NO: 416. In some aspects, the antibody comprises a CDR-L1 sequence comprising, consisting of, or consisting essentially of SEQ ID NO: 424. In some aspects, the antibody comprises a CDR-L1 sequence comprising, consisting of, or consisting essentially of SEQ ID NO: 432. In some aspects, the antibody comprises a CDR-L1 sequence comprising, consisting of, or consisting essentially of SEQ ID NO: 440.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 5.9. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

5.9.1. CDR-L3

In some embodiments, the antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 367-369.

In some embodiments, the antibody comprises a V_{L} sequence comprising a CDR-L3 sequence comprising, consisting of, or consisting essentially of a sequence selected from SEQ ID NOs: 305-307. In some aspects, the antibody comprises a V_{L} sequence comprising a CDR-L3 sequence comprising, consisting of, or consisting essentially of SEQ ID NO: 305. In some aspects, the antibody comprises a V_{L} sequence comprising a CDR-L3 sequence comprising, consisting of, or consisting essentially of SEQ ID NO: 306. In some aspects, the antibody comprises a V_{L} sequence comprising a CDR-L3 sequence comprising, consisting of, or consisting essentially of SEQ ID NO: 307. In some aspects, the antibody comprises a V_{L} sequence comprising a CDR-L3 sequence comprising, consisting of, or consisting essentially of SEQ ID NO: 390.

5.9.2. CDR-L2

In some embodiments, the antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of a V_{L} sequence provided in SEQ ID NOs: 367-369.

In some embodiments, the antibody comprises a V_{L} sequence comprising a CDR-L2 sequence comprising, consisting of, or consisting essentially of a sequence selected from SEQ ID NOs: 302-304. In some aspects, the antibody comprises a V_{L} sequence comprising a CDR-L2 sequence comprising, consisting of, or consisting essentially of SEQ ID NO: 302. In some aspects, the antibody comprises a V_{L} sequence comprising a CDR-L2 sequence comprising, consisting of, or consisting essentially of SEQ ID NO: 303. In some aspects, the antibody comprises a V_{L} sequence comprising a CDR-L2 sequence comprising, consisting of, or consisting essentially of SEQ ID NO: 304. In some aspects, the antibody comprises a V_{L} sequence comprising a CDR-L2 sequence comprising, consisting of, or consisting essentially of SEQ ID NO: 399. In some aspects, the antibody comprises a V_{L} sequence comprising a CDR-L2 sequence comprising, consisting of, or consisting essentially of SEQ ID NO: 409. In some aspects, the antibody comprises a V_{L} sequence comprising a CDR-L2 sequence comprising, consisting of, or consisting essentially of SEQ ID NO: 417. In some aspects, the antibody comprises a V_{L} sequence comprising a CDR-L2 sequence comprising, consisting of, or consisting essentially of SEQ ID NO: 425. In some aspects, the antibody comprises a V_{L} sequence comprising a CDR-L2 sequence comprising, consisting of, or consisting essentially of SEQ ID NO: 433. In some aspects, the antibody comprises a V_{L} sequence comprising a CDR-L2 sequence comprising, consisting of, or consisting essentially of SEQ ID NO: 441.

5.9.3. CDR-L1

In some embodiments, the antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of a V_{L} sequence provided in SEQ ID NOs: 367-369.

In some embodiments, the antibody comprises a V_{L} sequence comprising a CDR-L1 sequence comprising, consisting of, or consisting essentially of a sequence selected from SEQ ID NOs: 299-301. In some aspects, the antibody comprises a V_{L} sequence comprising a CDR-L1 sequence comprising, consisting of, or consisting essentially of SEQ ID NO: 299. In some aspects, the antibody comprises a V_{L} sequence comprising a CDR-L1 sequence comprising, consisting of, or consisting essentially of SEQ ID NO: 300. In some aspects, the antibody comprises a V_{L} sequence comprising a CDR-L1 sequence comprising, consisting of, or consisting essentially of SEQ ID NO: 301. In some aspects, the antibody comprises a V_{L} sequence comprising a CDR-L1 sequence comprising, consisting of, or consisting essentially of SEQ ID NO: 398. In some aspects, the antibody comprises a V_{L} sequence comprising a CDR-L1 sequence comprising, consisting of, or consisting essentially of SEQ ID NO: 408. In some aspects, the antibody comprises a V_{L} sequence comprising a CDR-L1 sequence comprising, consisting of, or consisting essentially of SEQ ID NO: 416. In some aspects, the antibody comprises a V_{L} sequence comprising a CDR-L1 sequence comprising, consisting of, or consisting essentially of SEQ ID NO: 424. In some aspects, the antibody comprises a V_{L} sequence comprising a CDR-L1 sequence comprising, consisting of, or consisting essentially of SEQ ID NO: 432. In some aspects, the antibody comprises a V_{L} sequence comprising a CDR-L1 sequence comprising, consisting of, or consisting essentially of SEQ ID NO: 440.

5.9.4. CDR-L3+CDR-L2

In some embodiments, the antibody comprises a V_{L} sequence comprising a CDR-L3 sequence comprising, consisting of, or consisting essentially of a sequence selected from SEQ ID NOs: 305-307 and a CDR-L2 sequence comprising, consisting of, or consisting essentially of a sequence selected from SEQ ID NOs: 302-304. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 367-369.

5.9.5. CDR-L3+CDR-L1

In some embodiments, the antibody comprises a V_{L} sequence comprising a CDR-L3 sequence comprising, consisting of, or consisting essentially of a sequence selected from SEQ ID NOs: 305-307 and a CDR-L1 sequence comprising, consisting of, or consisting essentially of a sequence selected from SEQ ID NOs: 299-301. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 367-369.

5.9.6. CDR-L1+CDR-L2

In some embodiments, the antibody comprises a V_{L} sequence comprising a CDR-L1 sequence comprising, consisting of, or consisting essentially of a sequence selected from SEQ ID NOs: 299-301 and a CDR-L2 sequence comprising, consisting of, or consisting essentially of a sequence selected from SEQ ID NOs: 302-304. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 367-369.

5.9.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs: 299-301, a CDR-L2 sequence comprising, consisting of, or consisting essentially of a sequence selected from SEQ ID NOs: 302-304, and a CDR-L3 sequence comprising, consisting of, or consisting essentially of a sequence selected from SEQ ID NOs: 305-307. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 367-369.

5.9.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 5.10. V_{L} Sequences

In some embodiments, the antibody comprises, consists of, or consists essentially of a V_{L} sequence provided in SEQ ID NOs: 367-369.

In some embodiments, the antibody comprises a V_{L} sequence comprising, consisting of, or consisting essentially of a sequence selected from SEQ ID NOs: 367-369. In some aspects, the antibody comprises a V_{L} sequence comprising, consisting of, or consisting essentially of SEQ ID NO: 367. In some aspects, the antibody comprises a V_{L} sequence comprising, consisting of, or consisting essentially of SEQ ID NO: 368. In some aspects, the antibody comprises a V_{L} sequence comprising, consisting of, or consisting essentially of SEQ ID NO: 369. In some aspects, the antibody comprises a V_{L} sequence comprising, consisting of, or consisting essentially of SEQ ID NO:394. In some aspects, the antibody comprises a V_{L} sequence comprising, consisting of, or consisting essentially of SEQ ID NO: 404. In some aspects, the antibody comprises a V_{L} sequence comprising, consisting of, or consisting essentially of SEQ ID NO: 402. In some aspects, the antibody comprises a V_{L} sequence comprising, consisting of, or consisting essentially of SEQ ID NO: 412. In some aspects, the antibody comprises a V_{L} sequence comprising, consisting of, or consisting essentially of SEQ ID NO: 420. In some aspects, the antibody comprises a V_{L} sequence comprising, consisting of, or consisting essentially of SEQ ID NO: 428. In some aspects, the antibody comprises a V_{L} sequence comprising, consisting of, or consisting essentially of SEQ ID NO: 436. In some aspects, the antibody comprises a V_{L} sequence comprising, consisting of, or consisting essentially of SEQ ID NO: 444.

5.10.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.11. Pairs

5.11.1. CDR-H3 CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of SEQ ID NOs: 240-298, and the CDR-L3 sequence is a CDR-L3 sequence comprising, consisting of, or consisting essentially of SEQ ID NOs: 305-307.

In some aspects, the CDR-H3-CDR-L3 pairs are selected from SEQ ID NO: 305 and SEQ ID NO: 240; SEQ ID NO: 305 and SEQ ID NO: 241; SEQ ID NO: 305 and SEQ ID NO: 242; SEQ ID NO: 305 and SEQ ID NO: 243; SEQ ID NO: 305 and SEQ ID NO: 244; SEQ ID NO: 305 and SEQ ID NO: 245; SEQ ID NO: 305 and SEQ ID NO: 246; SEQ ID NO: 305 and SEQ ID NO: 247; SEQ ID NO: 305 and SEQ ID NO: 248; SEQ ID NO: 305 and SEQ ID NO: 249; SEQ ID NO: 305 and SEQ ID NO: 250; SEQ ID NO: 305 and SEQ ID NO: 251; SEQ ID NO: 305 and SEQ ID NO: 252; SEQ ID NO: 305 and SEQ ID NO: 253; SEQ ID NO: 305 and SEQ ID NO: 254; SEQ ID NO: 305 and SEQ ID NO: 255; SEQ ID NO: 305 and SEQ ID NO: 256; SEQ ID NO: 305 and SEQ ID NO: 257; SEQ ID NO: 305 and SEQ ID NO: 258; SEQ ID NO: 305 and SEQ ID NO: 259; SEQ ID NO: 305 and SEQ ID NO: 260; SEQ ID NO: 305 and SEQ ID NO: 261; SEQ ID NO: 305 and SEQ ID NO: 262; SEQ ID NO: 305 and SEQ ID NO: 263; SEQ ID NO: 305 and SEQ ID NO: 264; SEQ ID NO: 305 and SEQ ID NO: 265; SEQ ID NO: 305 and SEQ ID NO: 266; SEQ ID NO: 305 and SEQ ID NO: 267; SEQ ID NO: 305 and SEQ ID NO: 268; SEQ ID NO: 305 and SEQ ID NO: 269; SEQ ID NO: 305 and SEQ ID NO: 270; SEQ ID NO: 305 and SEQ ID NO: 271; SEQ ID NO: 305 and SEQ ID NO: 272; SEQ ID NO: 305 and SEQ ID NO: 273; SEQ ID NO: 305 and SEQ ID NO: 274; SEQ ID NO: 305 and SEQ ID NO: 275; SEQ ID NO: 305 and SEQ ID NO: 276; SEQ ID NO: 305 and SEQ ID NO: 277; SEQ ID NO: 305 and SEQ ID NO: 278; SEQ ID NO: 305 and SEQ ID NO: 279; SEQ ID NO: 305 and SEQ ID NO: 280; SEQ ID NO: 305 and SEQ ID NO: 281; SEQ ID NO: 305 and SEQ ID NO: 282; SEQ ID NO: 305 and SEQ ID NO: 283; SEQ ID NO: 305 and SEQ ID NO: 284; SEQ ID NO: 305 and SEQ ID NO: 285; SEQ ID NO: 305 and SEQ ID NO: 286; SEQ ID NO: 305 and SEQ ID NO: 287; SEQ ID NO: 305 and SEQ ID NO: 288; SEQ ID NO: 305 and SEQ ID NO: 289; SEQ ID NO: 305 and SEQ ID NO: 290; SEQ ID NO: 305 and SEQ ID NO: 291; SEQ ID NO: 305 and SEQ ID NO: 292; SEQ ID NO: 305 and SEQ ID NO: 293; SEQ ID NO: 305 and SEQ ID NO: 294; SEQ ID NO: 305 and SEQ ID NO: 295; SEQ ID NO: 305 and SEQ ID NO: 296; SEQ ID NO: 305 and SEQ ID NO: 297; and SEQ ID NO: 305 and SEQ ID NO: 298.

In some aspects, the CDR-H3-CDR-L3 pairs are selected from SEQ ID NO: 306 and SEQ ID NO: 240; SEQ ID NO: 306 and SEQ ID NO: 241; SEQ ID NO: 306 and SEQ ID NO: 242; SEQ ID NO: 306 and SEQ ID NO: 243; SEQ ID NO: 306 and SEQ ID NO: 244; SEQ ID NO: 306 and SEQ ID NO: 245; SEQ ID NO: 306 and SEQ ID NO: 246; SEQ ID NO: 306 and SEQ ID NO: 247; SEQ ID NO: 306 and SEQ ID NO: 248; SEQ ID NO: 306 and SEQ ID NO: 249; SEQ ID NO: 306 and SEQ ID NO: 250; SEQ ID NO: 306 and SEQ ID NO: 251; SEQ ID NO: 306 and SEQ ID NO: 252; SEQ ID NO: 306 and SEQ ID NO: 253; SEQ ID NO: 306 and SEQ ID NO: 254; SEQ ID NO: 306 and SEQ ID NO: 255; SEQ ID NO: 306 and SEQ ID NO: 256; SEQ ID NO: 306 and SEQ ID NO: 257; SEQ ID NO: 306 and SEQ ID NO: 258; SEQ ID NO: 306 and SEQ ID NO: 259; SEQ ID NO: 306 and SEQ ID NO: 260; SEQ ID NO: 306 and SEQ ID NO: 261; SEQ ID NO: 306 and SEQ ID NO: 262; SEQ ID NO: 306 and SEQ ID NO: 263; SEQ ID NO: 306 and SEQ ID NO: 264; SEQ ID NO: 306 and SEQ ID NO: 265; SEQ ID NO: 306 and SEQ ID NO: 266; SEQ ID NO: 306 and SEQ ID NO: 267; SEQ ID NO: 306 and SEQ ID NO: 268; SEQ ID NO: 306 and SEQ ID NO: 269; SEQ ID NO: 306 and SEQ ID NO: 270; SEQ ID NO: 306 and SEQ ID NO: 271; SEQ ID NO: 306 and SEQ ID NO: 272; SEQ ID NO: 306 and SEQ ID NO: 273; SEQ ID NO: 306 and SEQ ID NO: 274; SEQ ID NO: 306 and SEQ ID NO: 275; SEQ ID NO: 306 and SEQ ID NO: 276; SEQ ID NO: 306 and SEQ ID NO: 277; SEQ ID NO: 306 and SEQ ID NO: 278; SEQ ID NO: 306 and SEQ ID NO: 279; SEQ ID NO: 306 and SEQ ID NO: 280; SEQ ID NO: 306 and SEQ ID NO: 281; SEQ ID NO: 306 and SEQ ID NO: 282; SEQ ID NO: 306 and SEQ ID NO: 283; SEQ ID NO: 306 and SEQ ID NO: 284; SEQ ID NO: 306 and SEQ ID NO: 285; SEQ ID NO: 306 and SEQ ID NO: 286; SEQ ID NO: 306 and SEQ ID NO: 287; SEQ ID NO: 306 and SEQ ID NO: 288; SEQ ID NO: 306 and SEQ ID NO: 289; SEQ ID NO: 306 and SEQ ID NO: 290; SEQ ID NO: 306 and SEQ ID NO: 291; SEQ ID NO: 306 and SEQ ID NO: 292; SEQ ID NO: 306 and SEQ ID NO: 293; SEQ ID NO: 306 and SEQ ID NO: 294; SEQ ID NO: 306 and SEQ ID NO: 295; SEQ ID NO: 306 and SEQ ID NO: 296; SEQ ID NO: 306 and SEQ ID NO: 297; and SEQ ID NO: 306 and SEQ ID NO: 298.

In some aspects, the CDR-H3-CDR-L3 pairs are selected from SEQ ID NO: 307 and SEQ ID NO: 240; SEQ ID NO: 307 and SEQ ID NO: 241; SEQ ID NO: 307 and SEQ ID NO: 242; SEQ ID NO: 307 and SEQ ID NO: 243; SEQ ID NO: 307 and SEQ ID NO: 244; SEQ ID NO: 307 and SEQ ID NO: 245; SEQ ID NO: 307 and SEQ ID NO: 246; SEQ ID NO: 307 and SEQ ID NO: 247; SEQ ID NO: 307 and SEQ ID NO: 248; SEQ ID NO: 307 and SEQ ID NO: 249; SEQ ID NO: 307 and SEQ ID NO: 250; SEQ ID NO: 307 and SEQ ID NO: 251; SEQ ID NO: 307 and SEQ ID NO: 252; SEQ ID NO: 307 and SEQ ID NO: 253; SEQ ID NO: 307 and SEQ ID NO: 254; SEQ ID NO: 307 and SEQ ID NO: 255; SEQ ID NO: 307 and SEQ ID NO: 256; SEQ ID NO: 307 and SEQ ID NO: 257; SEQ ID NO: 307 and SEQ ID NO: 258; SEQ ID NO: 307 and SEQ ID NO: 259; SEQ ID NO: 307 and SEQ ID NO: 260; SEQ ID NO: 307 and SEQ ID NO: 261; SEQ ID NO: 307 and SEQ ID NO: 262; SEQ ID NO: 307 and SEQ ID NO: 263; SEQ ID NO: 307 and SEQ ID NO: 264; SEQ ID NO: 307 and SEQ ID NO: 265; SEQ ID NO: 307 and SEQ ID NO: 266; SEQ ID NO: 307 and SEQ ID NO: 267; SEQ ID NO: 307 and SEQ ID NO: 268; SEQ ID NO: 307 and SEQ ID NO: 269; SEQ ID NO: 307 and SEQ ID NO: 270; SEQ ID NO: 307 and SEQ ID NO: 271; SEQ ID NO: 307 and SEQ ID NO: 272; SEQ ID NO: 307 and SEQ ID NO: 273; SEQ ID NO: 307 and SEQ ID NO: 274; SEQ ID NO: 307 and SEQ ID NO: 275; SEQ ID NO: 307 and SEQ ID NO: 276; SEQ ID NO: 307 and SEQ ID NO: 277; SEQ ID NO: 307 and SEQ ID NO: 278; SEQ ID NO: 307 and SEQ ID NO: 279; SEQ ID NO: 307 and SEQ ID NO: 280; SEQ ID NO: 307 and SEQ ID NO: 281; SEQ ID NO: 307 and SEQ ID NO: 282; SEQ ID NO: 307 and SEQ ID NO: 283; SEQ ID NO: 307 and SEQ ID NO: 284; SEQ ID NO: 307 and SEQ ID NO: 285; SEQ ID NO: 307 and SEQ ID NO: 286; SEQ ID NO: 307 and SEQ ID NO: 287; SEQ ID NO: 307 and SEQ ID NO: 288; SEQ ID NO: 307 and SEQ ID NO: 289; SEQ ID NO: 307 and SEQ ID NO: 290; SEQ ID NO: 307 and SEQ ID NO: 291; SEQ ID NO: 307 and SEQ ID NO: 292; SEQ ID NO: 307 and SEQ ID NO: 293; SEQ ID NO: 307 and SEQ ID NO: 294; SEQ ID NO: 307 and SEQ ID NO: 295; SEQ ID NO: 307 and SEQ ID NO: 296; SEQ ID NO: 307 and SEQ ID NO: 297; and SEQ ID NO: 307 and SEQ ID NO: 298.

5.11.1.1. Variants of CDR-H3 CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

5.11.2. CDR-H1 CDR-L1 Pairs

In some embodiments, the antibody comprises a CDR-H1 sequence and a CDR-L1 sequence. In some aspects, the CDR-H1 sequence is part of a V_{H} and the CDR-L1 sequence is part of a V_{L}.

In some aspects, the CDR-H1 sequence is a Chothia CDR-H1 sequence comprising, consisting of, or consisting essentially of SEQ ID NOs: 4-62, and the CDR-L1 sequence is a CDR-L1 sequence comprising, consisting of, or consisting essentially of SEQ ID NOs: 299-301.

In some aspects, the CDR-H1 sequence is a Kabat CDR-H1 sequence comprising, consisting of, or consisting essentially of SEQ ID NOs: 63-121, and the CDR-L1 sequence is a CDR-L1 sequence comprising, consisting of, or consisting essentially of SEQ ID NOs: 299-301.

5.11.2.1. Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

5.11.3. CDR-H2 CDR-L2 Pairs

In some embodiments, the antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of SEQ ID NOs: 122-180, and the CDR-L2 sequence is a CDR-L2 sequence comprising, consisting of, or consisting essentially of SEQ ID NOs: 302-304.

In some aspects, the CDR-H1 sequence is a Kabat CDR-H2 sequence comprising, consisting of, or consisting essentially of SEQ ID NOs: 181-239, and the CDR-L2 sequence is a CDR-L2 sequence comprising, consisting of, or consisting essentially of SEQ ID NOs: 302-304.

5.11.3.1. Variants of CDR-H2 CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

5.11.4. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of SEQ ID NOs: 308-366, and the V_{L} sequence is a V_{L} sequence comprising, consisting of, or consisting essentially of SEQ ID NOs: 367-369.

In some aspects, the V_{H}-V_{L} pairs are selected from SEQ ID NO: 367 and SEQ ID NO: 308; SEQ ID NO: 367 and SEQ ID NO: 309; SEQ ID NO: 367 and SEQ ID NO: 310; SEQ ID NO: 367 and SEQ ID NO: 311; SEQ ID NO: 367 and SEQ ID NO: 312; SEQ ID NO: 367 and SEQ ID NO: 313; SEQ ID NO: 367 and SEQ ID NO: 314; SEQ ID NO: 367 and SEQ ID NO: 315; SEQ ID NO: 367 and SEQ ID NO: 316; SEQ ID NO: 367 and SEQ ID NO: 317; SEQ ID NO: 367 and SEQ ID NO: 318; SEQ ID NO: 367 and SEQ ID NO: 319; SEQ ID NO: 367 and SEQ ID NO: 320; SEQ ID NO: 367 and SEQ ID NO: 321; SEQ ID NO: 367 and SEQ ID NO: 322; SEQ ID NO: 367 and SEQ ID NO: 323; SEQ ID NO: 367 and SEQ ID NO: 324; SEQ ID NO: 367 and SEQ ID NO: 325; SEQ ID NO: 367 and SEQ ID NO: 326; SEQ ID NO: 367 and SEQ ID NO: 327; SEQ ID NO: 367 and SEQ ID NO: 328; SEQ ID NO: 367 and SEQ ID NO: 329; SEQ ID NO: 367 and SEQ ID NO: 330; SEQ ID NO: 367 and SEQ ID NO: 331; SEQ ID NO: 367 and SEQ ID NO: 332; SEQ ID NO: 367 and SEQ ID NO: 333; SEQ ID NO: 367 and SEQ ID NO: 334; SEQ ID NO: 367 and SEQ ID NO: 335; SEQ ID NO: 367 and SEQ ID NO: 336; SEQ ID NO: 367 and SEQ ID NO: 337; SEQ ID NO: 367 and SEQ ID NO: 338; SEQ ID NO: 367 and SEQ ID NO: 339; SEQ ID NO: 367 and SEQ ID NO: 340; SEQ ID NO: 367 and SEQ ID NO: 341; SEQ ID NO: 367 and SEQ ID NO: 342; SEQ ID NO: 367 and SEQ ID NO: 343; SEQ ID NO: 367 and SEQ ID NO: 344; SEQ ID NO: 367 and SEQ ID NO: 345; SEQ ID NO: 367 and SEQ ID NO: 346; SEQ ID NO: 367 and SEQ ID NO: 347; SEQ ID NO: 367 and SEQ ID NO: 348; SEQ ID NO: 367 and SEQ ID NO: 349; SEQ ID NO: 367 and SEQ ID NO: 350; SEQ ID NO: 367 and SEQ ID NO: 351; SEQ ID NO: 367 and SEQ ID NO: 352; SEQ ID NO: 367 and SEQ ID NO: 353; SEQ ID NO: 367 and SEQ ID NO: 354; SEQ ID NO: 367 and SEQ ID NO: 355; SEQ ID NO: 367 and SEQ ID NO: 356; SEQ ID NO: 367 and SEQ ID NO: 357; SEQ ID NO: 367 and SEQ ID NO: 358; SEQ ID NO: 367 and SEQ ID NO: 359; SEQ ID NO: 367 and SEQ ID NO: 360; SEQ ID NO: 367 and SEQ ID NO: 361; SEQ ID NO: 367 and SEQ ID NO: 362; SEQ ID NO: 367 and SEQ ID NO: 363; SEQ ID NO: 367 and SEQ ID NO: 364; SEQ ID NO: 367 and SEQ ID NO: 365; and SEQ ID NO: 367 and SEQ ID NO: 366.

In some aspects, the V_{H}-V_{L} pairs are selected from SEQ ID NO: 368 and SEQ ID NO: 308; SEQ ID NO: 368 and SEQ ID NO: 309; SEQ ID NO: 368 and SEQ ID NO: 310; SEQ ID NO: 368 and SEQ ID NO: 311; SEQ ID NO: 368 and SEQ ID NO: 312; SEQ ID NO: 368 and SEQ ID NO: 313; SEQ ID NO: 368 and SEQ ID NO: 314; SEQ ID NO: 368 and SEQ ID NO: 315; SEQ ID NO: 368 and SEQ ID NO: 316; SEQ ID NO: 368 and SEQ ID NO: 317; SEQ ID NO: 368 and SEQ ID NO: 318; SEQ ID NO: 368 and SEQ ID NO: 319; SEQ ID NO: 368 and SEQ ID NO: 320; SEQ ID NO: 368 and SEQ ID NO: 321; SEQ ID NO: 368 and SEQ ID NO: 322; SEQ ID NO: 368 and SEQ ID NO: 323; SEQ ID NO: 368 and SEQ ID NO: 324; SEQ ID NO: 368 and SEQ ID NO: 325; SEQ ID NO: 368 and SEQ ID NO: 326; SEQ ID NO: 368 and SEQ ID NO: 327; SEQ ID NO: 368 and SEQ ID NO: 328; SEQ ID NO: 368 and SEQ ID NO: 329; SEQ ID NO: 368 and SEQ ID NO: 330; SEQ ID NO: 368 and SEQ ID NO: 331; SEQ ID NO: 368 and SEQ ID NO: 332; SEQ ID NO: 368 and SEQ ID NO: 333; SEQ ID NO: 368 and SEQ ID NO: 334; SEQ ID NO: 368 and SEQ ID NO: 335; SEQ ID NO: 368 and SEQ ID NO: 336; SEQ ID NO: 368 and SEQ ID NO: 337; SEQ ID NO: 368 and SEQ ID NO: 338; SEQ ID NO: 368 and SEQ ID NO: 339; SEQ ID NO: 368 and SEQ ID NO: 340; SEQ ID NO: 368 and SEQ ID NO: 341; SEQ ID NO: 368 and SEQ ID NO: 342; SEQ ID NO: 368 and SEQ ID NO: 343; SEQ ID NO: 368 and SEQ ID NO: 344; SEQ ID NO: 368 and SEQ ID NO: 345; SEQ ID NO: 368 and SEQ ID NO: 346; SEQ ID NO: 368 and SEQ ID NO: 347; SEQ ID NO: 368 and SEQ ID NO: 348; SEQ ID NO: 368 and SEQ ID NO: 349; SEQ ID NO: 368 and SEQ ID NO: 350; SEQ ID NO: 368 and SEQ ID NO: 351; SEQ ID NO: 368 and SEQ ID NO: 352; SEQ ID NO: 368 and SEQ ID NO: 353; SEQ ID NO: 368 and SEQ ID NO: 354; SEQ ID NO: 368 and SEQ ID NO: 355; SEQ ID NO: 368 and SEQ ID NO: 356; SEQ ID NO: 368 and SEQ ID NO: 357; SEQ ID NO: 368 and SEQ ID NO: 358; SEQ ID NO: 368 and SEQ ID NO: 359; SEQ ID NO: 368 and SEQ ID NO: 360; SEQ ID NO: 368 and SEQ ID NO: 361; SEQ ID NO: 368 and SEQ ID NO: 362; SEQ ID NO: 368 and SEQ ID NO: 363; SEQ ID NO: 368 and SEQ ID NO: 364; SEQ ID NO: 368 and SEQ ID NO: 365; and SEQ ID NO: 368 and SEQ ID NO: 366.

In some aspects, the V_{H}-V_{L} pairs are selected from SEQ ID NO: 369 and SEQ ID NO: 308; SEQ ID NO: 369 and SEQ ID NO: 309; SEQ ID NO: 369 and SEQ ID NO: 310; SEQ ID NO: 369 and SEQ ID NO: 311; SEQ ID NO: 369 and SEQ ID NO: 312; SEQ ID NO: 369 and SEQ ID NO: 313; SEQ ID NO: 369 and SEQ ID NO: 314; SEQ ID NO: 369 and SEQ ID NO: 315; SEQ ID NO: 369 and SEQ ID NO: 316; SEQ ID NO: 369 and SEQ ID NO: 317; SEQ ID NO: 369 and SEQ ID NO: 318; SEQ ID NO: 369 and SEQ ID NO: 319; SEQ ID NO: 369 and SEQ ID NO: 320; SEQ ID NO: 369 and SEQ ID NO: 321; SEQ ID NO: 369 and SEQ ID NO: 322; SEQ ID NO: 369 and SEQ ID NO: 323; SEQ ID NO: 369 and SEQ ID NO: 324; SEQ ID NO: 369 and SEQ ID NO: 325; SEQ ID NO: 369 and SEQ ID NO: 326; SEQ ID NO: 369 and SEQ ID NO: 327; SEQ ID NO: 369 and SEQ ID NO: 328; SEQ ID NO: 369 and SEQ ID NO: 329; SEQ ID NO: 369 and SEQ ID NO: 330; SEQ ID NO: 369 and SEQ ID NO: 331; SEQ ID NO: 369 and SEQ ID NO: 332; SEQ ID NO: 369 and SEQ ID NO: 333; SEQ ID NO: 369 and SEQ ID NO: 334; SEQ ID NO: 369 and SEQ ID NO: 335; SEQ ID NO: 369 and SEQ ID NO: 336; SEQ ID NO: 369 and SEQ ID NO: 337; SEQ ID NO: 369 and SEQ ID NO: 338; SEQ ID NO: 369 and SEQ ID NO: 339; SEQ ID NO: 369 and SEQ ID NO: 340; SEQ ID NO: 369 and SEQ ID NO: 341; SEQ ID NO: 369 and SEQ ID NO: 342; SEQ ID NO: 369 and SEQ ID NO: 343; SEQ ID NO: 369 and SEQ ID NO: 344; SEQ ID NO: 369 and SEQ ID NO: 345; SEQ ID NO: 369 and SEQ ID NO: 346; SEQ ID NO: 369 and SEQ ID NO: 347; SEQ ID NO: 369 and SEQ ID NO: 348; SEQ ID NO: 369 and SEQ ID NO: 349; SEQ ID NO: 369 and SEQ ID NO: 350; SEQ ID NO: 369 and SEQ ID NO: 351; SEQ ID NO: 369 and SEQ ID NO: 352; SEQ ID NO: 369 and SEQ ID NO: 353; SEQ ID NO: 369 and SEQ ID NO: 354; SEQ ID NO: 369 and SEQ ID NO: 355; SEQ ID NO: 369 and SEQ ID NO: 356; SEQ ID NO: 369 and SEQ ID NO: 357; SEQ ID NO: 369 and SEQ ID NO: 358; SEQ ID NO: 369 and SEQ ID NO: 359; SEQ ID NO: 369 and SEQ ID NO: 360; SEQ ID NO: 369 and SEQ ID NO: 361; SEQ ID NO: 369 and SEQ ID NO: 362; SEQ ID NO: 369 and SEQ ID NO: 363; SEQ ID NO: 369 and SEQ ID NO: 364; SEQ ID NO: 369 and SEQ ID NO: 365; and SEQ ID NO: 369 and SEQ ID NO: 366.

5.11.4.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.12. Antibodies Comprising all Six CDRs

In some embodiments, the antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 4-62; the CDR-H2 sequence is a Chothia CDR-H2 sequence comprising, consisting of, or consisting essentially of SEQ ID NOs: 122-180; the CDR-H3 sequence is a CDR-H3 sequence comprising, consisting of, or consisting essentially of SEQ ID NOs: 240-298; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Chothia CDR-H1 sequence comprising, consisting of, or consisting essentially of SEQ ID NO: 19; the CDR-H2 sequence is a Chothia CDR-H2 sequence comprising, consisting of, or consisting essentially of SEQ ID NO: 137; the CDR-H3 sequence is a CDR-H3 sequence comprising, consisting of, or consisting essentially of SEQ ID NO: 255; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Chothia CDR-H1 sequence comprising, consisting of, or consisting essentially of SEQ ID NO: 58; the CDR-H2 sequence is a Chothia CDR-H2 sequence comprising, consisting of, or consisting essentially of SEQ ID NO: 176; the CDR-H3 sequence is a CDR-H3 sequence comprising, consisting of, or consisting essentially of SEQ ID NO: 294; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Kabat CDR-H1 sequence comprising, consisting of, or consisting essentially of SEQ ID NOs: 63-121; the CDR-H2 sequence is a Kabat CDR-H2 sequence comprising, consisting of, or consisting essentially of SEQ ID NOs: 181-239; the CDR-H3 sequence is a CDR-H3 sequence comprising, consisting of, or consisting essentially of SEQ ID NOs: 240-298; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Kabat CDR-H1 sequence comprising, consisting of, or consisting essentially of SEQ ID NO: 78; the CDR-H2 sequence is a Kabat CDR-H2 sequence comprising, consisting of, or consisting essentially of SEQ ID NO: 196; the CDR-H3 sequence is a CDR-H3 sequence comprising, consisting of, or consisting essentially of SEQ ID NO: 255; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Kabat CDR-H1 sequence comprising, consisting of, or consisting essentially of SEQ ID NO: 117; the CDR-H2 sequence is a Kabat CDR-H2 sequence comprising, consisting of, or consisting essentially of SEQ ID NO: 235; the CDR-H3 sequence is a CDR-H3 sequence comprising, consisting of, or consisting essentially of SEQ ID NO: 294; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

5.12.1. Variants of Antibodies Comprising all Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In particular embodiments, the FOLR1-binding domain includes the Farletuzumab scFv. In particular embodiments, the Farletuzumab scFv includes the sequence:

(SEQ ID NO: 383)
DIQLTQSPSSLSASVGDRVTITCSVSSSISSNNLHWYQQKPGKAPKPWIY
GTSNLASGVPSRFSGSGSGTDYTFTISSLQPEDIATYYCQQWSSYPYMYT
FGQGTKVEIKGGGGSGGGGSGGGGSGGGGSEVQLVESGGGVVQPGRSLRL
SCSASGFTFSGYGLSWVRQAPGKGLEWVAMISSGGSYTYYADSVKGRFAI
SRDNAKNTLFLQMDSLRPEDTGVYFCARHGDDPAWFAYWGQGTPVTVSS.

In particular embodiments, the FOLR1-binding domain includes the Farletuzumab scFv. In particular embodiments, the Farletuzumab scFv includes the sequence:

(SEQ ID NO: 384)
EVQLVESGGGVVQPGRSLRLSCSASGFTFSGYGLSWVRQAPGKGLEWVAM
ISSGGSYTYYADSVKGRFAISRDNAKNTLFLQMDSLRPEDTGVYFCARHG
DDPAWFAYWGQGTPVTVSSGGGGSGGGGSGGGGSGGGGSDIQLTQSPSSL
SASVGDRVTITCSVSSSISSNNLHWYQQKPGKAPKPWIYGTSNLASGVPS
RFSGSGSGTDYTFTISSLQPEDIATYYCQQWSSYPYMYTFGQGTKVEIK.

In particular embodiments, the FOLR1-binding domain includes the Farletuzumab antibody (MorAb-003). In particular embodiments, the FOLR1-binding domain is a human or humanized binding domain including a variable heavy chain including a CDRH1 sequence including GYGLS (SEQ TD NO: 385), a CDRH2 sequence including MISSGGSYTYYADSVKG (SEQ TD NO: 386), and a CDRH3 sequence including HGDDPAWFAY (SEQ TD NO: 387), and a variable light chain including a CDRL1 sequence including SVSSSISSNNLH (SEQ TD NO: 388), a CDRL2 sequence including GTSNLAS (SEQ TD NO: 389), and a CDRL3 sequence including QQWSSYPYMYT (SEQ ID NO: 390), according to Kabat numbering scheme.

In particular embodiments, the FOLR1-binding domain includes the Farletuzumab antibody. In particular embodiments, a sequence that binds human FOLR1 includes a heavy chain region including sequence:

EVQLVESGGGVVQPGRSLRLSCSASGFTFSGYGLSWVRQAPGKGLEWVAMISSGGSYTY YADSVKGRFAISRDNAKNTLFLQMDSLRPEDTGVYFCARHGDDPAWFAYWGQGTPVTV SS (SEQ TD NO: 391), and a light chain region including sequence:

(SEQ ID NO: 392)
DIQLTQSPSSLSASVGDRVTITCSVSSSISSNNLHWYQQKPGKAPKPWIY
GTSNLASGVPSRFSGSGSGTDYTFTISSLQPEDIATYYCQQWSSYPYMYT
FGQGTKVEIK.

In particular embodiments, the FOLR1-binding domain includes a variable heavy chain region encoded by the sequence:

(SEQ ID NO: 393)
GAGGTACAGCTTGTCGAGAGCGGTGGTGGAGTAGTCCAACCGGGTCGAAG
TCTTAGGCTTTCCTGTAGCGCATCTGGGTTCACTTTTAGTGGCTACGGCC
TCTCCTGGGTGAGACAGGCGCCTGGGAAGGGGCTGGAGTGGGTAGCCATG
ATTTCATCTGGTGGCTCATATACTTATTATGCCGACTCCGTAAAGGGAAG
ATTCGCAATATCACGCGATAACGCTAAAAATACACTCTTCTTGCAGATGG
ATTCTTTGAGACCTGAGGATACCGGGGTTTACTTTTGCGCCAGACACGGG
GATGACCCCGCCTGGTTTGCCTATTGGGGACAGGGAACCCCTGTGACGGT
ATCCTCT, and a variable light chain region encoded
by the sequence:
(SEQ ID NO: 394)
GATATTCAGCTTACTCAAAGTCCGAGTAGTCTGTCTGCCTCAGTTGGCGA
TAGGGTGACCATCACTTGCTCCGTAAGTAGTTCTATTTCTTCCAACAACC
TGCATTGGTATCAACAGAAACCAGGTAAAGCACCTAAGCCGTGGATCTAC
GGAACGTCCAACCTTGCGTCTGGCGTACCAAGCCGGTTCTCCGGGAGTGG
GAGTGGTACAGATTACACATTTACTATCAGTTCTCTTCAACCGGAAGACA
TTGCCACATATTATTGCCAGCAATGGTCATCTTACCCCTATATGTACACA
TTTGGTCAGGGTACAAAGGTTGAAATAAAA.

In particular embodiments, the FOLR1-binding domain includes the huMOV19 (M9346A) antibody. In particular embodiments, the FOLR1-binding domain is a human or humanized binding domain including a variable heavy chain including a CDRH1 sequence including GYFMN (SEQ ID NO: 395), a CDRH2 sequence including RIIIPYDGDTFYNQKFQG (SEQ ID NO: 396), and a CDRH3 sequence including YDGSRAMDY (SEQ ID NO: 397), and a variable light chain including a CDRL1 sequence including KASQSVSFAGTSLMH (SEQ ID NO: 398), a CDRL2 sequence including RASNLEA (SEQ ID NO: 399), and a CDRL3 sequence including QQSREYPYT (SEQ ID NO: 400), according to Kabat numbering scheme.

In particular embodiments, the FOLR1-binding domain includes the huMOV19 version 1.00. In particular embodiments, a sequence that binds human FOLR1 includes a variable heavy chain region including sequence:

QVQLVQSGAEVVKPGASVKISCKASGYTFTGYFMNWVKQSPGQSLEWIGRIIIPYDGDTF YNQKFQGKATLTVDKSSNTAHMELLSLTSEDFAVYYCTRYDGSRAMDYWGQGTTVTVS S (SEQ ID NO: 401), and a variable light chain region including sequence:

(SEQ ID NO: 402)
DIVLTQSPLSLAVSLGQPAIISCKASQSVSFAGTSLMHWYHQKPGQQPRL
LIYRASNLEAGVPDRFSGSGSKTDFTLNISPVEAEDAATYYCQQSREYPY
TFGGGTKLEIKR.

In particular embodiments, the FOLR1-binding domain includes the huMOV19 version 1.60. In particular embodiments, a sequence that binds human FOLR1 includes a variable heavy chain region including sequence:

QVQLVQSGAEVVKPGASVKISCKASGYTFTGYFMNWVKQSPGQSLEWIGRIIIPYDGDTF YNQKFQGKATLTVDKSSNTAHMELLSLTSEDFAVYYCTRYDGSRAMDYWGQGTTVTVS S (SEQ ID NO: 403), and a variable light chain region including sequence:

(SEQ ID NO: 404)
DIVLTQSPLSLAVSLGQPAIISCKASQSVSFAGTSLMHWYHQKPGQQPRL
LIYRASNLEAGVPDRFSGSGSKTDFTLTISPVEAEDAATYYCQQSREYPY
TFGGGTKLEIKR.

In particular embodiments, the FOLR1-binding domain includes the RA15-7 antibody. In particular embodiments, the FOLR1-binding domain is a human or humanized binding domain including a variable heavy chain including a CDRH1 sequence including DFYMN (SEQ ID NO: 405), a CDRH2 sequence including FIRNKANGYTTEFNPSVKG (SEQ ID NO: 406), and a CDRH3 sequence including TLYGYAYYYVMDA (SEQ ID NO: 407), and a variable light chain including a CDRL1 sequence including RTSEDIFRNLA (SEQ ID NO: 408), a CDRL2 sequence including DTNRLAD (SEQ ID NO: 409), and a CDRL3 sequence including QQYDNYPLT (SEQ ID NO: 410), according to Kabat numbering scheme.

In particular embodiments, the FOLR1-binding domain includes the RA15-7 antibody. In particular embodiments, a sequence that binds human FOLR1 includes a variable heavy chain region including sequence:

EVQLVESGGGLVQPGGSLRLSCAASGFTFTDFYMNWVRQPPGKAPEWLGFIRNKANGYT TEFNPSVKGRFTISRDNSKNSLYLQMNSLKTEDTATYYCARTLYGYAYYYVMDAWGQG TLVTVSS (SEQ ID NO: 411), and a variable light chain region including sequence:

(SEQ ID NO: 412)
DIQMTQSPSSLSASLGDRVTITCRTSEDIFRNLAWYQQKPGKAPKLLIYD
TNRLADGVPSRFSGSGSGTDYTLTISSLQPEDFATYFCQQYDNYPLTFGQ
GTKLEIK.

In particular embodiments, the FOLR1-binding domain includes the huFR1-48. In particular embodiments, the FOLR1-binding domain is a human or humanized binding domain including a variable heavy chain including a CDRH1 sequence including NYWMQ (SEQ ID NO: 413), a CDRH2 sequence including AIYPGNGDSRYTQKFQG (SEQ ID NO: 414), and a CDRH3 sequence including RDGNYAAY (SEQ ID NO: 415), and a variable light chain including a CDRL1 sequence including RASENIYSNLA (SEQ ID NO: 416), a CDRL2 sequence including AATNLAD (SEQ ID NO: 417), and a CDRL3 sequence including QHFWASPYT (SEQ ID NO: 418), according to Kabat numbering scheme.

In particular embodiments, the FOLR1-binding domain includes the huFR1-48. In particular embodiments, a sequence that binds human FOLR1 includes a variable heavy chain region including sequence:

QVQLVQSGAEVAKPGASVKLSCKASGYTFTNYWMQWIKQRPGQGLEWIGAIYPGNGDS RYTQKFQGKATLTADKSSSTAYMQVSSLTSEDSAVYYCARRDGNYAAYWGQGTLVTVS A (SEQ ID NO: 419), and a variable light chain region including sequence:

(SEQ ID NO: 420)
DIQMTQSPSSLSVSVGERVTITCRASENIYSNLAWYQQKPGKSPKLLVYA
ATNLADGVPSRFSGSESGTDYSLKINSLQPEDFGSYYCQHFWASPYTFGQ
GTKLEIKR.

In particular embodiments, the FOLR1-binding domain includes the huFR1-49. In particular embodiments, the FOLR1-binding domain is a human or humanized binding domain including a variable heavy chain including a CDRH1 sequence including NYWMY (SEQ ID NO: 421), a CDRH2 sequence including AIYPGNSDTTYNQKFQG (SEQ ID NO: 422), and a CDRH3 sequence including RHDYGAMDY (SEQ ID NO: 423), and a variable light chain including a CDRL1 sequence including RASENIYTNLA (SEQ ID NO: 424), a CDRL2 sequence including TASNLAD (SEQ ID NO: 425), and a CDRL3 sequence including QHFWVSPYT (SEQ ID NO: 426), according to Kabat numbering scheme.

In particular embodiments, the FOLR1-binding domain includes the huFR1-49. In particular embodiments, a sequence that binds human FOLR1 includes a variable heavy chain region including sequence:

QVQLQQSGAVVAKPGASVKMSCKASGYTFTNYWMYWIKQRPGQGLELIGAIYPGNSDT TYNQKFQGKATLTAVTSANTVYMEVSSLTSEDSAVYYCTKRHDYGAMDYWGQGTSVT VSS (SEQ ID NO: 427), and a variable light chain region including sequence:

(SEQ ID NO: 428)
DIQMTQSPSSLSVSVGERVTITCRASENIYTNLAWYQQKPGKSPKLLVYT
ASNLADGVPSRFSGSGSGTDYSLKINSLQPEDFGTYYCQHFWVSPYTFGQ
GTKLEIKR.

In particular embodiments, the FOLR1-binding domain includes the huFR1-57. In particular embodiments, the FOLR1-binding domain is a human or humanized binding domain including a variable heavy chain including a CDRH1 sequence including SFGMH (SEQ ID NO: 429), a CDRH2 sequence including YISSGSSTISYADSVKG (SEQ ID NO: 430), and a CDRH3 sequence including EAYGSSMEY (SEQ ID NO: 431), and a variable light chain including a CDRL1 sequence including RASQNINNNLH (SEQ ID NO: 432), a CDRL2 sequence including YVSQSVS (SEQ ID NO: 433), and a CDRL3 sequence including QQSNSWPHYT (SEQ ID NO: 434), according to Kabat numbering scheme.

In particular embodiments, the FOLR1-binding domain includes the huFR1-57. In particular embodiments, a sequence that binds human FOLR1 includes a variable heavy chain region including sequence:

EVQLVESGGGLVQPGGSRRLSCAASGFTFSSFGMHWVRQAPGKGLEWVAYISSGSSTISY ADSVKGRFTISRDNSKKTLLLQMTSLRAEDTAMYYCAREAYGSSMEYWGQGTLVTVSS (SEQ ID NO: 435), and a variable light chain region including sequence:

(SEQ ID NO: 436)
EIVLTQSPATLSVTPGDRVSLSCRASQNINNNLHWYQQKPGQSPRLLIKY
VSQSVSGIPDRFSGSGSGTDFTLSISSVEPEDFGMYFCQQSNSWPHYTFG
QGTKLEIKR.

In particular embodiments, the FOLR1-binding domain includes the huFR1-65. In particular embodiments, the FOLR1-binding domain is a human or humanized binding domain including a variable heavy chain including a CDRH1 sequence including SYTMH (SEQ ID NO: 437), a CDRH2 sequence including YINPISGYTNYNQKFQG (SEQ ID NO: 438), and a CDRH3 sequence including GGAYGRKPMDY (SEQ ID NO: 439), and a variable light chain including a CDRL1 sequence including KASQNVGPNVA (SEQ ID NO: 440), a CDRL2 sequence including SASYRYS (SEQ ID NO: 441), and a CDRL3 sequence including QQYNSYPYT (SEQ ID NO: 442), according to Kabat numbering scheme.

In particular embodiments, the FOLR1-binding domain includes the huFR1-65. In particular embodiments, a sequence that binds human FOLR1 includes a variable heavy chain region including sequence:

QVQLVQSGAEVAKPGASVKMSCKASGYTFTSYTMHWVKQRPGQGLAWIGYINPISGYT NYNQKFQGKATLTADKSSSTAYMQLNSLTSEDSAVYYCASGGAYGRKPMDYWGQGTS VTVSS (SEQ ID NO: 443), and a variable light chain region including sequence:

(SEQ ID NO: 444)
EIVMTQSPATMSTSPGDRVSVTCKASQNVGPNVAWYQQKPGQSPRALIYS
ASYRYSGVPARFTGSGSGTDFTLTISNMQSEDLAEYFCQQYNSYPYTFGQ
GTKLEIKR.

Additional examples of FOLR1 antibodies include MAB5646, FR-1-21, 60307-1-1, 548908, MA5-43815, AB_2912159, 60307-1-IG, MABS2175, and 26B3.F2. Examples of FOLR1 antibodies also include those known in the art, e.g., those disclosed in U.S. Published Application Nos. 20200325240, 20120009181, and 20120282175; U.S. Provisional Application Nos. 61/695,791 and 61/756,254; and PCT publication WO2011/106528. In certain embodiments, the FOLR1-binding domain includes a sequence having at least 90% sequence identity to SEQ ID NOs: 383-496. In particular embodiments, the FOLR1-binding domain includes a sequence having at least 95%, at least 96%, at least 97%, at least 98%, or at least 99% sequence identity to SEQ ID NOs: 383-496. In certain embodiments, the FOLR1-binding domain includes one or more sequences (e.g., CDRs, V_{L}, and/or V_{H}) set forth SEQ ID NOs: 383-496. Additional examples of FOLR1 antibodies include antibodies described in Table 5.

### 6. Affinity

In some embodiments, the affinity of the antibody for folate receptor alpha as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻⁹ M and 10⁻¹⁰ M.

In some embodiments, the affinity of the antibody for human folate receptor alpha, as determined by surface plasmon resonance at 25° C., and as indicated by K_{D}, is from about 0.36×10⁻⁹ M to about 2.21×10⁻⁹ M. In some embodiments, the affinity of the antibody for human folate receptor alpha, as determined by surface plasmon resonance at 25° C., and as indicated by K_{D}, is from about 8.55×10⁻¹⁰ M to about 1.70×10⁻⁸ M. In some embodiments, the affinity of the antibody for human folate receptor alpha, as determined by surface plasmon resonance at 25° C., and as indicated by K_{D}, is from about 5.71×10⁻¹⁰ M to about 2.58×10⁻⁸ M. In some embodiments, the affinity of the antibody for human folate receptor alpha is about any of the K_{D} values reported for human folate receptor alpha in the examples below.

In some embodiments the antibody has a kₐ of at least about 10⁴ M⁻¹×sec⁻¹. In some embodiments the antibody has a kₐ of at least about 10⁵ M⁻¹×sec⁻¹. In some embodiments the antibody has a kₐ of at least about 10⁶ M⁻¹×sec⁻¹. In some embodiments the antibody has a kₐ of at least about 10⁷ M⁻¹×sec⁻¹. In some embodiments the antibody has a kₐ of at least about 10⁸ M⁻¹×sec⁻¹. In some embodiments the antibody has a kₐ of at least about 10⁹ M⁻¹×sec⁻¹. In some embodiments the antibody has a kₐ of between about 10⁴ M⁻¹×sec⁻¹ and about 10¹⁰ M⁻¹×sec⁻¹. In some embodiments the antibody has a kₐ of between about 10⁵ M⁻¹×sec⁻¹ and about 10¹⁰ M⁻¹×sec⁻¹. In some embodiments the antibody has a kₐ of between about 10⁶ M⁻¹×sec⁻¹ and about 10¹⁰ M⁻¹×sec⁻¹. In some embodiments the antibody has a kₐ of between about 10⁷ M⁻¹×sec⁻¹ and about 10¹⁰ M⁻¹×sec⁻¹.

In some embodiments the antibody has a kₐ when associating with human folate receptor alpha, as determined by surface plasmon resonance at 25° C., of from about 4.44×10⁵ M⁻¹×sec⁻¹ to about 1.61×10⁵ M⁻¹×sec⁻¹. In some embodiments the antibody has a kₐ when associating with human folate receptor alpha, as determined by surface plasmon resonance at 25° C., of from about 2.90×10⁵ M⁻¹×sec⁻¹ to about 9.64×10⁹ M⁻¹×sec⁻¹. In some embodiments the antibody has a kₐ when associating with human folate receptor alpha of about any of the kₐ values reported for human folate receptor alpha in the examples below.

In some embodiments the antibody has a k_{d} of about 10⁻⁵ sec⁻¹ or less. In some embodiments the antibody has a k_{d} of about 10⁻⁴ sec⁻¹ or less. In some embodiments the antibody has a k_{d} of about 10⁻³ sec⁻¹ or less. In some embodiments the antibody has a k_{d} of between about 10⁻² sec⁻¹ and about 10⁻⁵ sec⁻¹. In some embodiments the antibody has a k_{d} of between about 10⁻² sec⁻¹ and about 10⁻⁴ sec⁻¹. In some embodiments the antibody has a k_{d} of between about 10⁻³ sec⁻¹ and about 10⁻⁵ sec⁻¹.

In some embodiments the antibody has a k_{d} when dissociating from human folate receptor alpha, as determined by surface plasmon resonance at 25° C., of from about 8.66×10⁻⁴ sec⁻¹ to about 1.08×10⁻² sec⁻¹. In some embodiments the antibody has a k_{d} when dissociating from human folate receptor alpha, as determined by surface plasmon resonance at 25° C., of from about 2.28×10⁻⁴ sec⁻¹ to about 4.82×10¹ sec⁻¹. In some embodiments the antibody has a k_{d} when dissociating from human folate receptor alpha of about any of the k_{d} values reported for human folate receptor alpha in the examples below.

In some embodiments, the affinity of the antibody for cynomolgus folate receptor alpha, as determined by surface plasmon resonance at 25° C., and as indicated by K_{D}, is from about 0.19×10⁻⁹ M to about 2.84×10⁻⁹ M. In some embodiments, the affinity of the antibody for cynomolgus folate receptor alpha is about any of the K_{D} values reported for cynomolgus folate receptor alpha in the examples below.

In some embodiments, the affinity of the antibody for mouse folate receptor alpha, as determined by surface plasmon resonance at 25° C., and as indicated by K_{D}, is from about 0.5×10⁻⁹ M to about 9.07×10⁻⁸ M. In some embodiments, the affinity of the antibody for mouse folate receptor alpha is about any of the K_{D} values reported for mouse folate receptor alpha in the examples below.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and k_{d} are determined according to the methods described in the Examples provided herein.

### 7. Epitope Bins

In some embodiments, the antibody binds the same epitope as an antibody encompassing any of SEQ ID NOs: 308-366. In some embodiments, the antibody binds the same epitope as an antibody comprising any of the V_{H}-V_{L} pairs, above. In some embodiments, the antibody competes for epitope binding with an antibody encompassing any of SEQ ID NOs: 308-366. In some embodiments, the antibody competes for epitope binding with an antibody comprising any of the V_{H}-V_{L} pairs, above.

### 8. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 9. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc comprises one or more modifications in at least one of the C_{H}3 sequences. In some embodiments, the Fc comprises one or more modifications in at least one of the C_{H}2 sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides. Exemplary modifications in the Fc region are described, for example, in International Patent Application No. PCT/US2017/037545, filed Jun. 14, 2017.

An alteration in in CDC and/or ADCC activity can be confirmed using in vitro and/or in vivo assays. For example, Fc receptor (FcR) binding assays can be conducted to measure FcTR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 10. Preparation of Antibody Conjugates

### 10.1. Antigen Preparation

The FOLR1 protein to be used for isolation of the antibodies may be intact FOLR1 or a fragment of FOLR1. The intact FOLR1 protein, or fragment of FOLR1, may be in the form of an isolated protein or protein expressed by a cell. Other forms of FOLR1 useful for generating antibodies will be apparent to those skilled in the art.

### 10.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 10.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. As provided herein, humanized antibodies or antibody fragments include one or more CDRs from nonhuman immunoglobulin molecules and framework regions wherein the amino acid residues including the framework are derived completely or mostly from human germline. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, cellular marker binding. These framework substitutions are identified by methods well-known in the art, e.g., by modeling of the interactions of the CDR and framework residues to identify framework residues important for cellular marker binding and sequence comparison to identify unusual framework residues at particular positions. (See, e.g., U.S. Pat. No. 5,585,089; and Riechmann et al., 1988, Nature, 332:323). Additional methods of obtaining humanized antibodies include those described in, for example, Winter and Milstein, Nature, 1991, 349:293-299; Rader et al., Proc. Nat. Acad. Sci. U.S.A., 1998, 95:8910-8915; Steinberger et al., J Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

### 10.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 10.5. Conjugation

The antibody conjugates can be prepared by standard techniques, for example, using the techniques described in Doronina et al. (Bioconjugate Chem. 17: 114-124, 2006). In certain embodiments, an antibody is contacted with a payload precursor under conditions suitable for forming a bond from the antibody to the payload to form an antibody-payload conjugate. In certain embodiments, an antibody is contacted with a linker precursor under conditions suitable for forming a bond from the antibody to the linker. The resulting antibody-linker is contacted with a payload precursor under conditions suitable for forming a bond from the antibody-linker to the payload to form an antibody-linker-payload conjugate. In certain embodiments, a payload precursor is contacted with a linker precursor under conditions suitable for forming a bond from the payload to the linker. The resulting payload-linker is contacted with an antibody under conditions suitable for forming a bond from the payload-linker to the antibody to form an antibody-linker-payload conjugate. Suitable linkers for preparing the antibody conjugates are disclosed herein, and exemplary conditions for conjugation are described in the Examples below.

In some embodiments, an anti-FOLR1 conjugate is prepared by contacting an anti-FOLR1 antibody as disclosed herein with a linker precursor having a structure of any of (A)-(L):

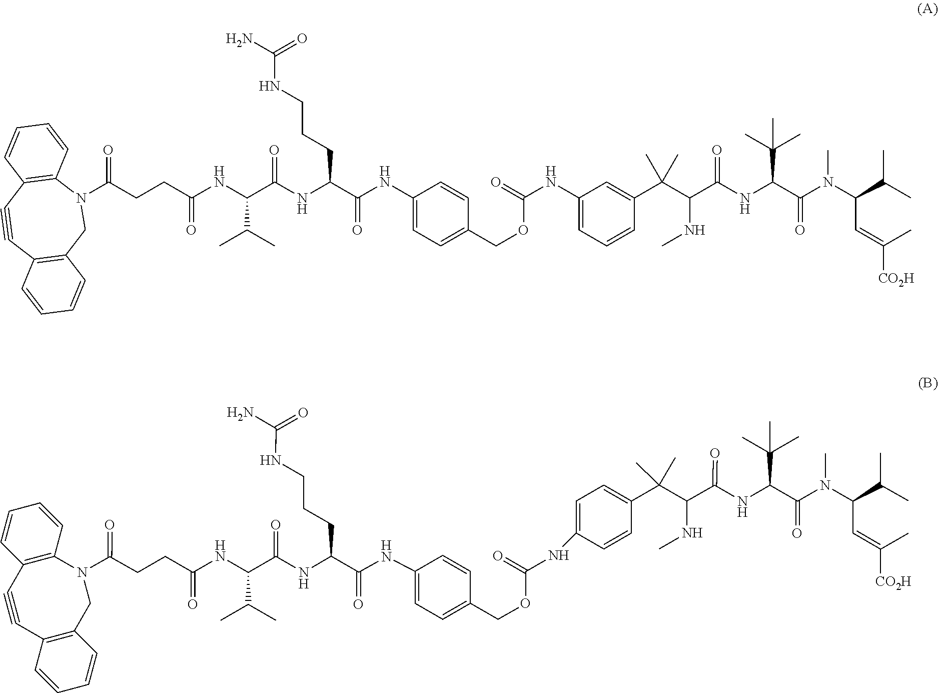 [SMILES: CNC(C(=O)N[C@H](C(=O)N(C)[C@H](/C=C(\C)C(=O)O)C(C)C)C(C)(C)C)C(C)(C)c1cccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c1 ; CNC(C(=O)N[C@H](C(=O)N(C)[C@H](/C=C(\C)C(=O)O)C(C)C)C(C)(C)C)C(C)(C)c1ccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)cc1]

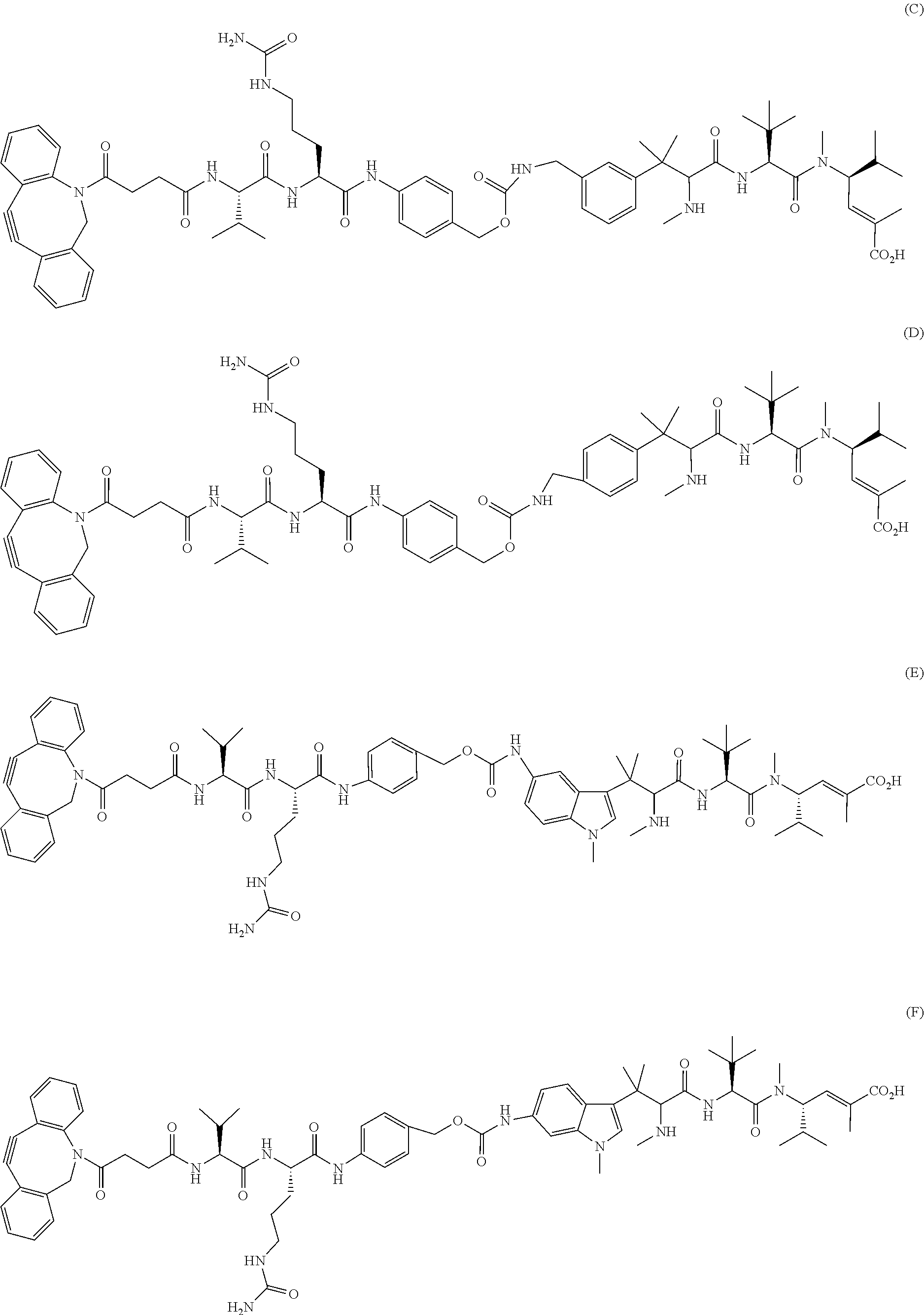

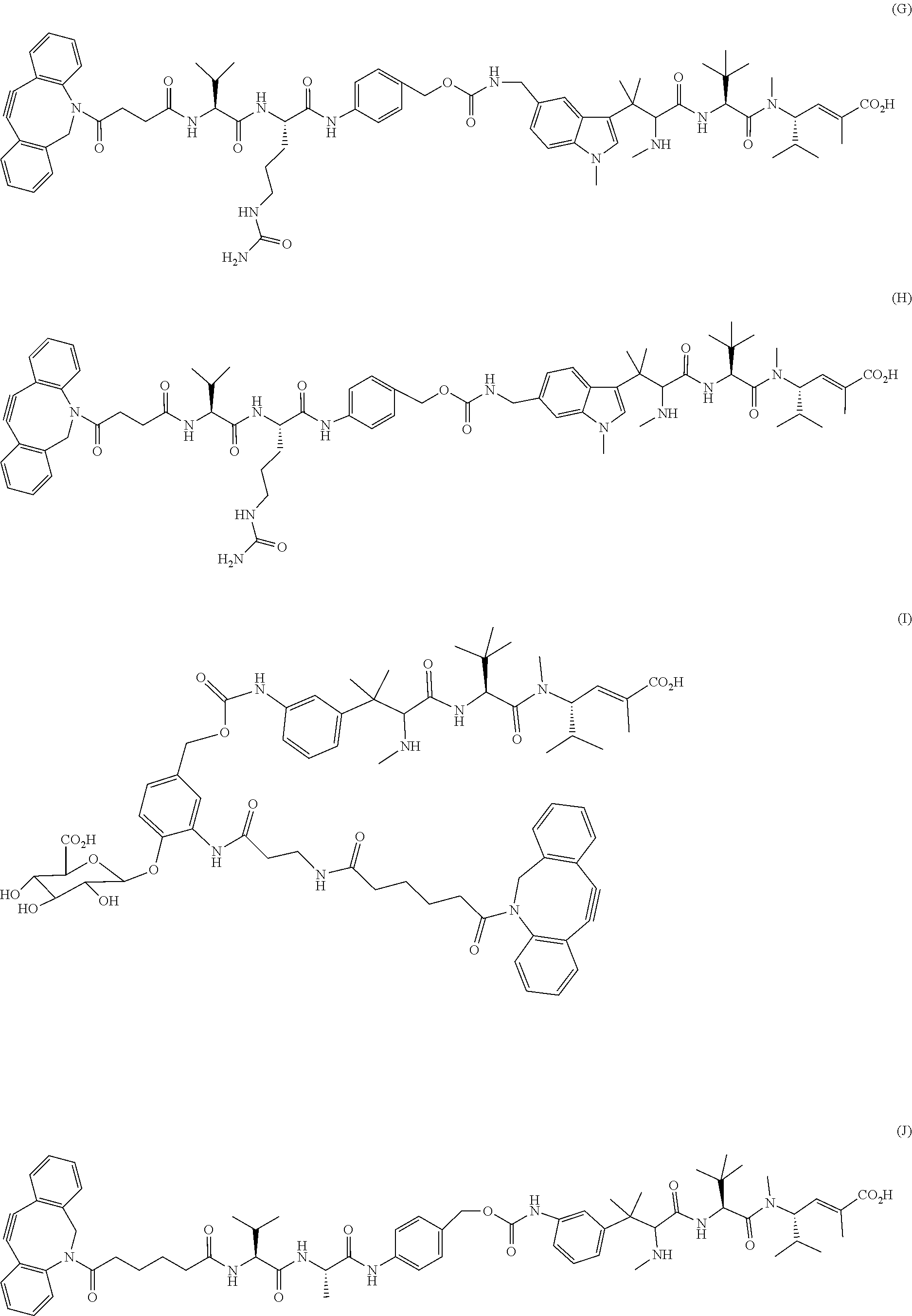

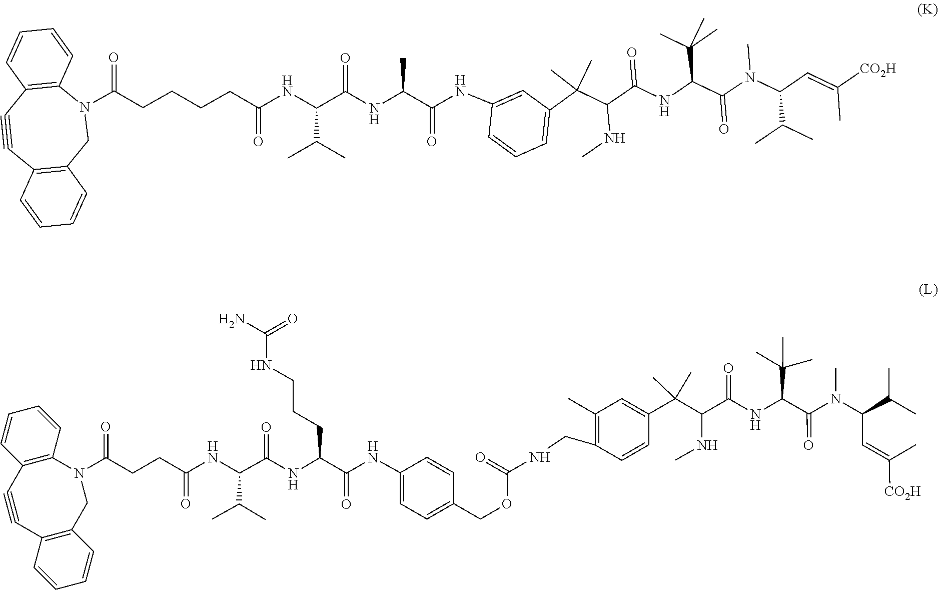 [SMILES: CNC(C(=O)N[C@H](C(=O)N(C)[C@H](/C=C(\C)C(=O)O)C(C)C)C(C)(C)C)C(C)(C)c1cccc(NC(=O)[C@H](C)NC(=O)[C@@H](NC(=O)CCCCC(=O)N2Cc3ccccc3C#Cc3ccccc32)C(C)C)c1 ; CNC(C(=O)N[C@H](C(=O)N(C)[C@H](/C=C(\C)C(=O)O)C(C)C)C(C)(C)C)C(C)(C)c1ccc(CNC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c(C)c1]

In some embodiments, the stereochemistry of the linker precursors identified as (A)-(L) is identified with R and S notation for each chiral center, from left to right as depicted in formulas (A1)-(L1) and (A2)-(L2) illustrated below:

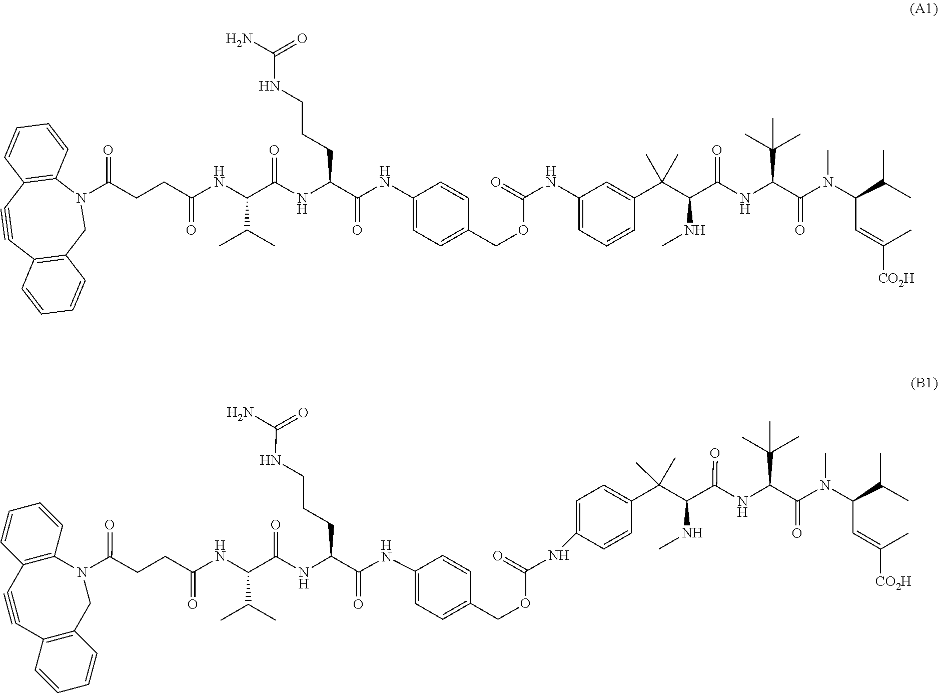 [SMILES: CN[C@H](C(=O)N[C@H](C(=O)N(C)[C@H](/C=C(\C)C(=O)O)C(C)C)C(C)(C)C)C(C)(C)c1cccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c1 ; CN[C@H](C(=O)N[C@H](C(=O)N(C)[C@H](/C=C(\C)C(=O)O)C(C)C)C(C)(C)C)C(C)(C)c1ccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)cc1]

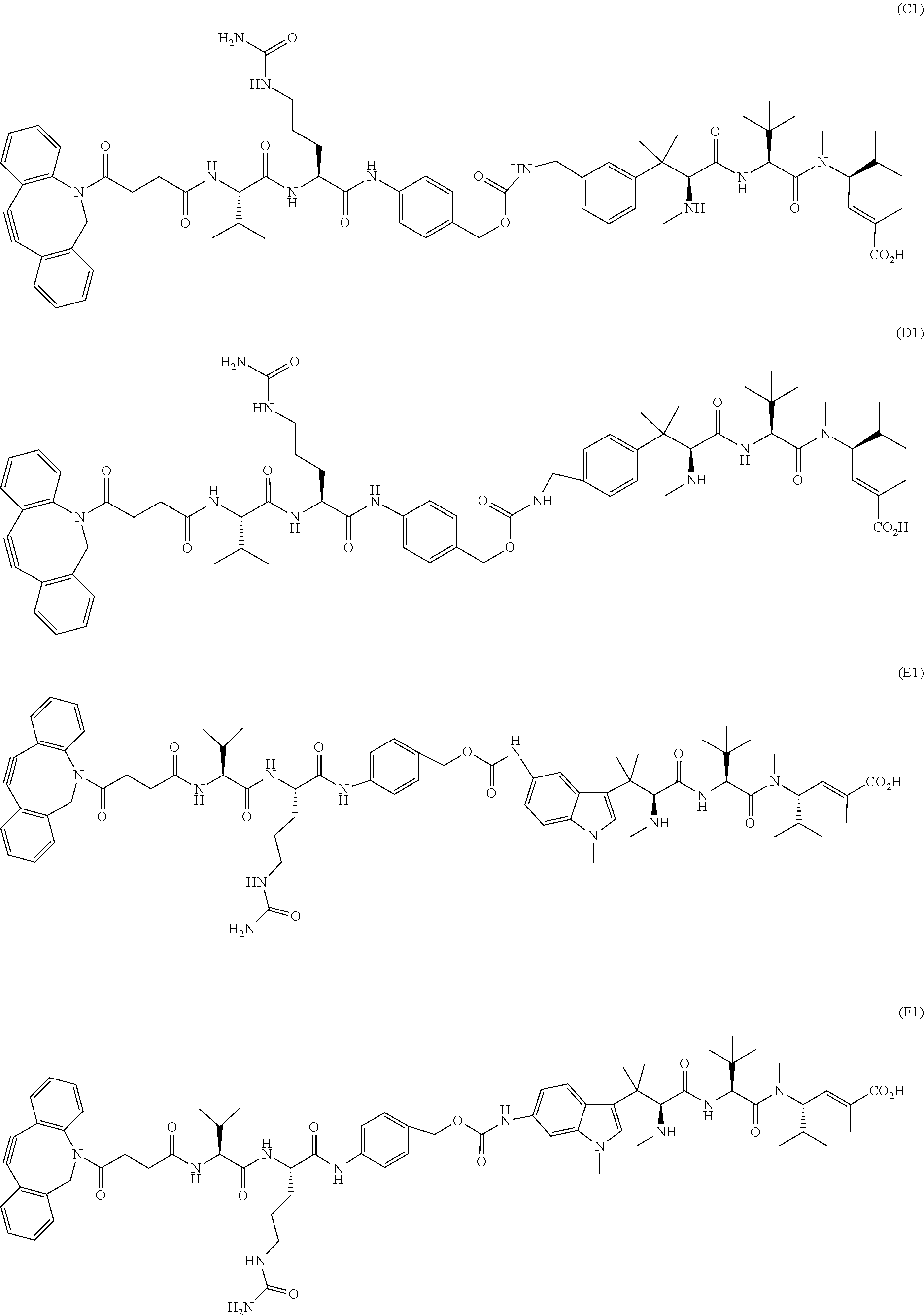

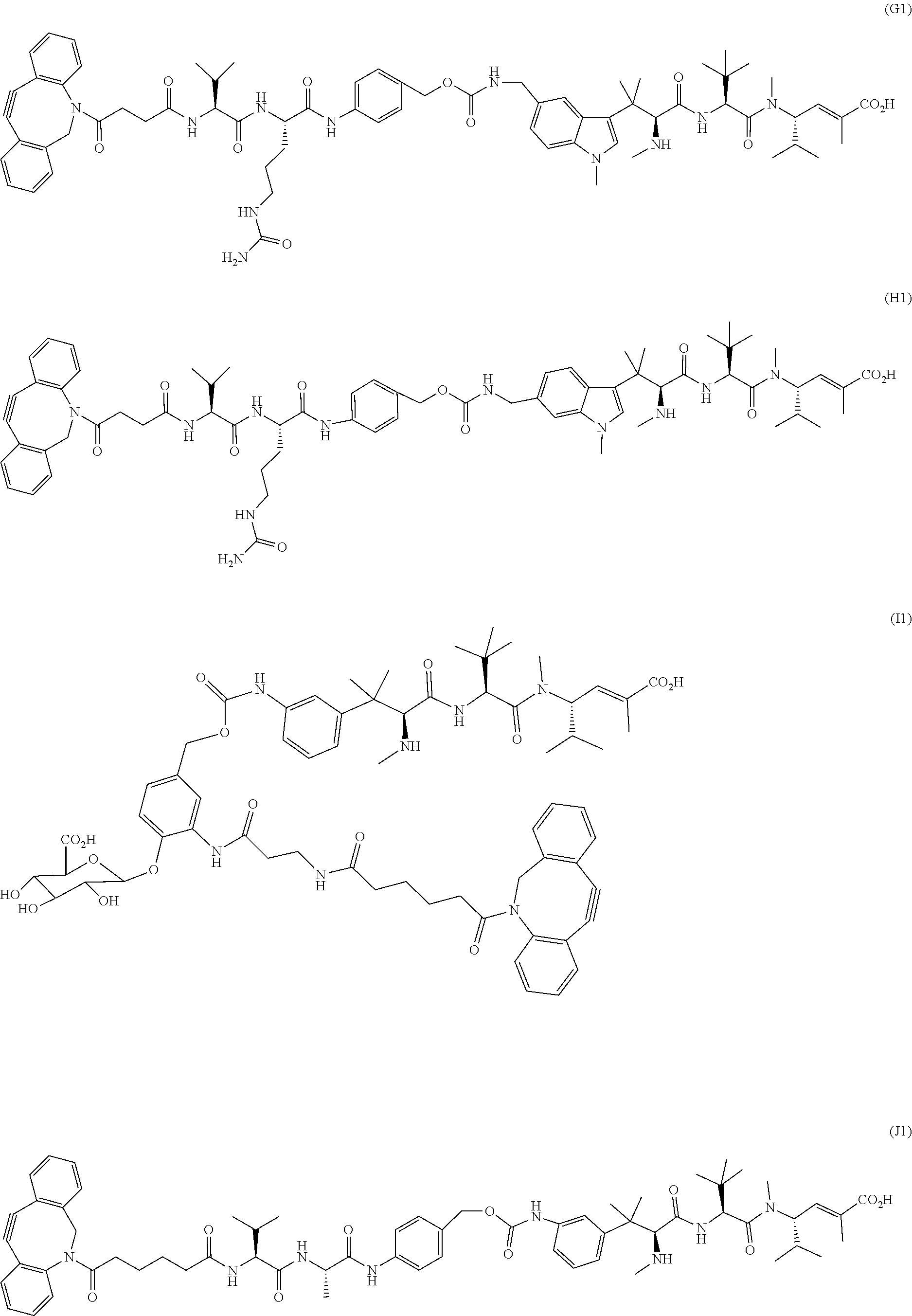

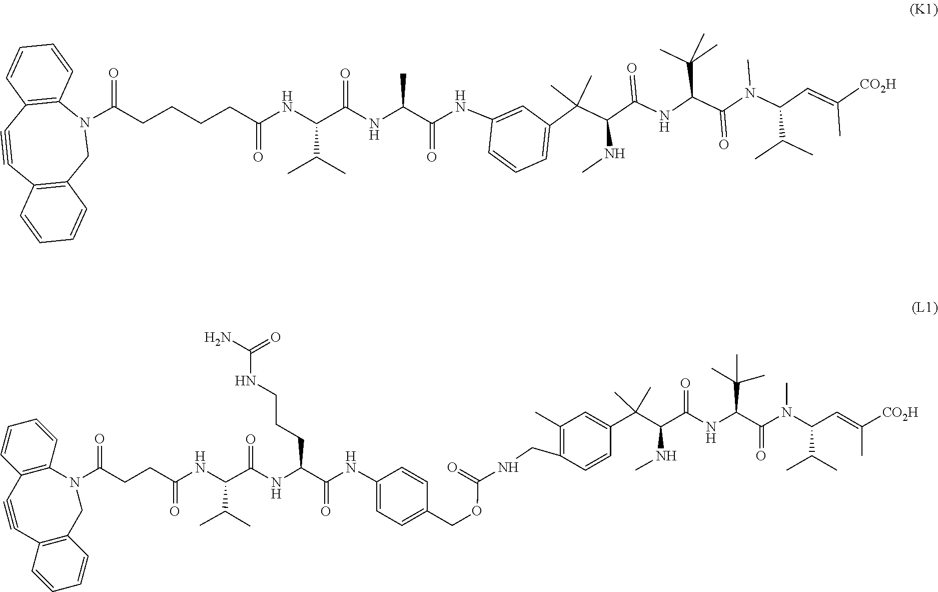 [SMILES: CN[C@H](C(=O)N[C@H](C(=O)N(C)[C@H](/C=C(\C)C(=O)O)C(C)C)C(C)(C)C)C(C)(C)c1cccc(NC(=O)[C@H](C)NC(=O)[C@@H](NC(=O)CCCCC(=O)N2Cc3ccccc3C#Cc3ccccc32)C(C)C)c1 ; CN[C@H](C(=O)N[C@H](C(=O)N(C)[C@H](/C=C(\C)C(=O)O)C(C)C)C(C)(C)C)C(C)(C)c1ccc(CNC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c(C)c1]

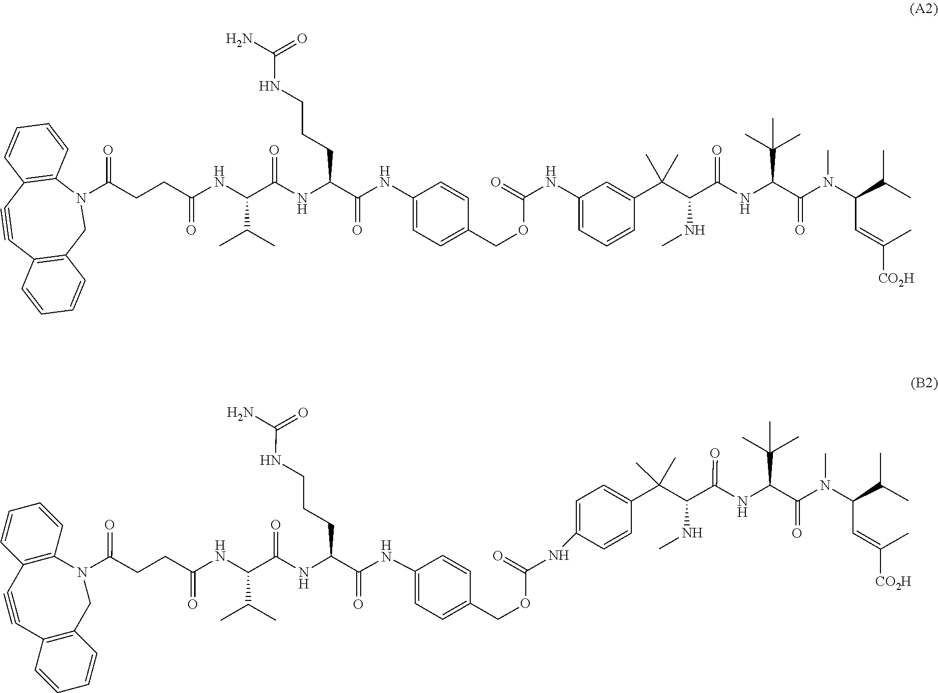 [SMILES: CN[C@@H](C(=O)N[C@H](C(=O)N(C)[C@H](/C=C(\C)C(=O)O)C(C)C)C(C)(C)C)C(C)(C)c1cccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c1 ; CN[C@@H](C(=O)N[C@H](C(=O)N(C)[C@H](/C=C(\C)C(=O)O)C(C)C)C(C)(C)C)C(C)(C)c1ccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)cc1]

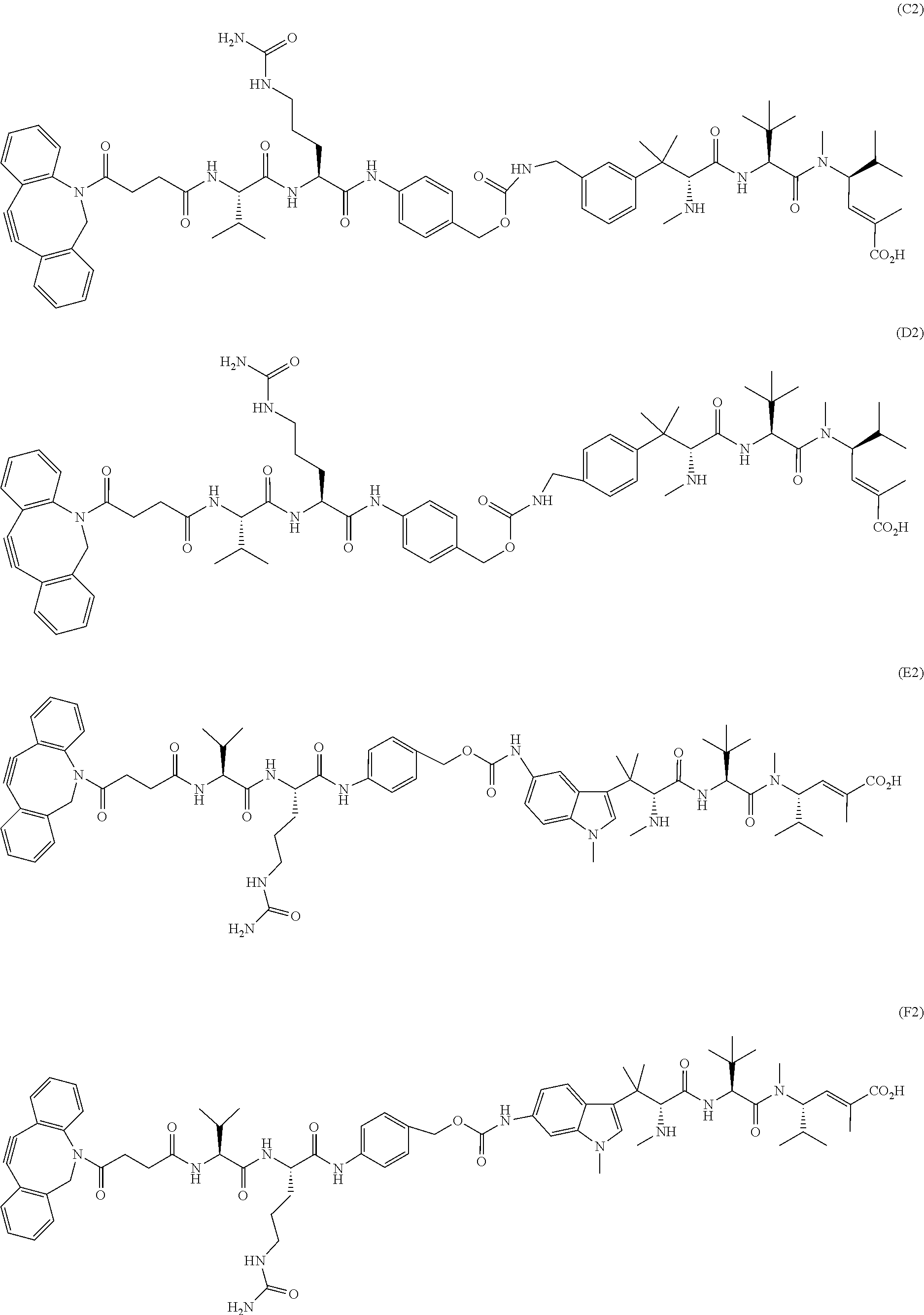

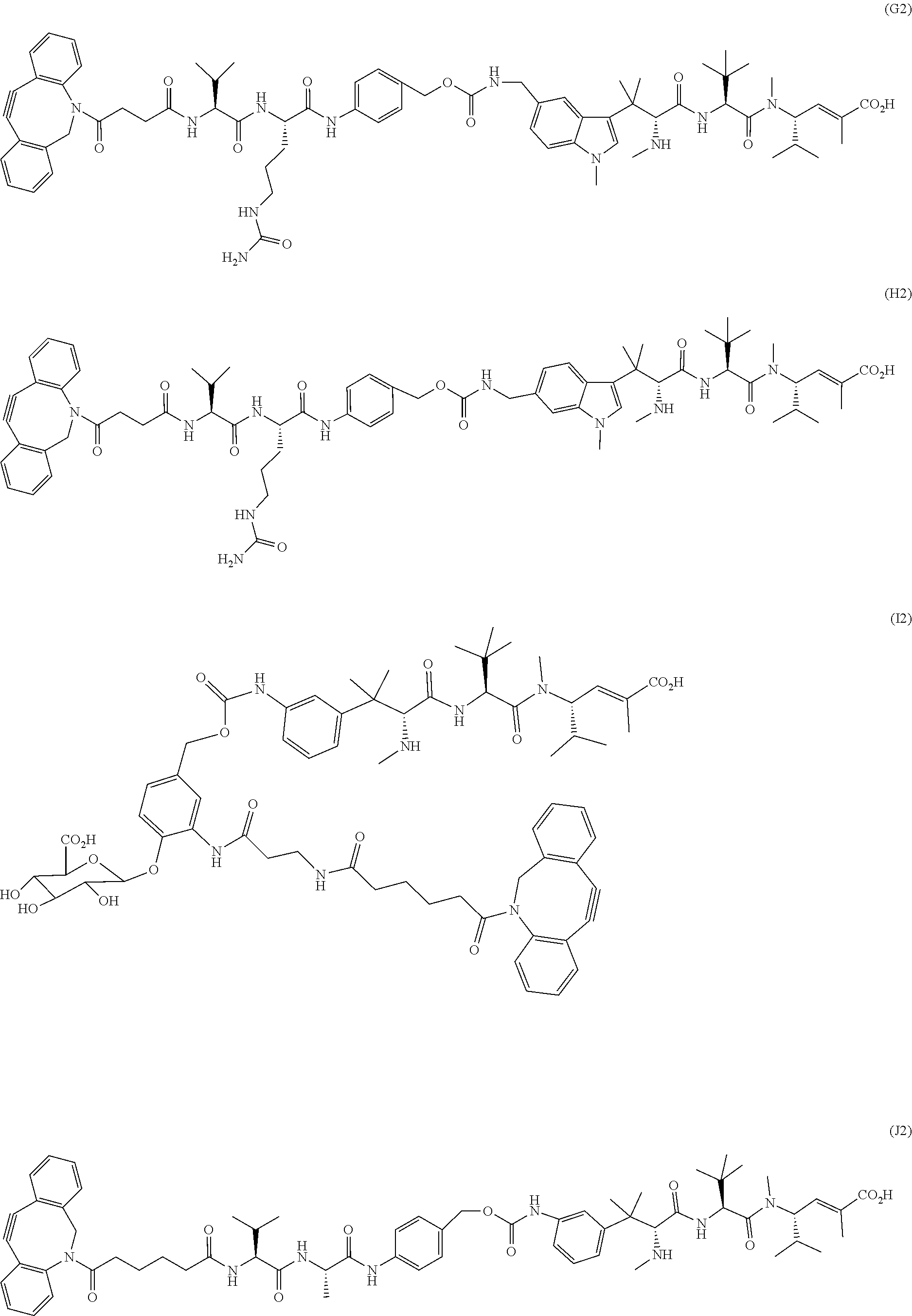

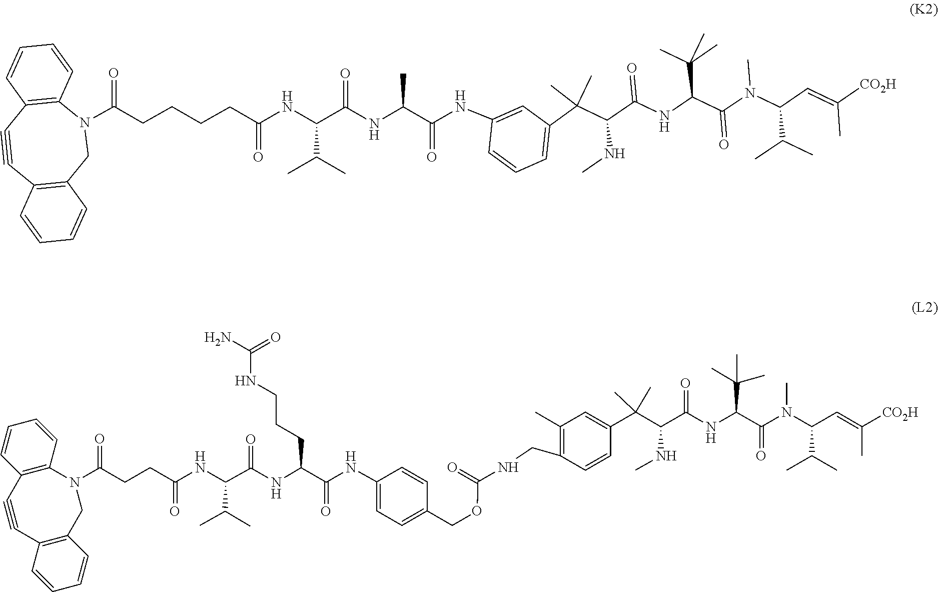 [SMILES: CN[C@@H](C(=O)N[C@H](C(=O)N(C)[C@H](/C=C(\C)C(=O)O)C(C)C)C(C)(C)C)C(C)(C)c1cccc(NC(=O)[C@H](C)NC(=O)[C@@H](NC(=O)CCCCC(=O)N2Cc3ccccc3C#Cc3ccccc32)C(C)C)c1 ; CN[C@@H](C(=O)N[C@H](C(=O)N(C)[C@H](/C=C(\C)C(=O)O)C(C)C)C(C)(C)C)C(C)(C)c1ccc(CNC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c(C)c1]

In some aspects, antibody-drug conjugates (ADC) may be formed. ADCs with multiple (e.g., four) drugs per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of a linker-drug conjugate can be added at 4° C. for 1 hr, and the conjugation reaction can be quenched by addition of a substantial excess, for example 20-fold, of cysteine. The resulting ADC mixture can be purified on SEPHADEX G-25 equilibrated in PBS to remove unreacted linker-drug conjugate, desalted if desired, and purified by size-exclusion chromatography. The resulting ADC can then be sterile filtered, for example, through a 0.2 μm filter, and can be lyophilized if desired for storage.

Immunotoxins include a binding domain (e.g., an antibody or binding fragment thereof) disclosed herein conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of a linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage. Antibody-radioisotope conjugates include a binding domain linked to a radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to binding domains of the present disclosure include actinium-225, iodine-131, arsenic-211, iodine-131, indium-111, yttrium-90, and lutetium-177, as well as alpha-emitting radionuclides such as astatine-211 or bismuth-212 or bismuth-213. Methods for preparing radioimmunoconjugates are established in the art. Examples of radioimmunoconjugates are commercially available, including Zevalin™ (DEC Pharmaceuticals), and similar methods can be used to prepare radioimmunoconjugates using the binding domains of the disclosure.

Examples of radionuclides that are useful for radiation therapy include ²²⁵Ac and ²²⁷Th. ²²⁵Ac is a radionuclide with the half-life of ten days. As ²²⁵Ac decays the daughter isotopes ²²¹Fr, ²¹³Bi, and ²⁰⁹Pb are formed. ²²⁷Th has a half-life of 19 days and forms the daughter isotope 223Ra.

Additional examples of useful radioisotopes include ²²⁸Ac, ¹¹¹Ag, ¹²⁴Am, ⁷⁴As, ²¹¹As ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mRr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb ¹³⁸Nd, ⁵⁷Ni, ⁶⁶Ni, ²³⁴Np, ¹⁵O, ¹⁸²O, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, and/or ⁹⁷Zr.

In particular embodiments, the antibody conjugate includes antibody-nanoparticle conjugates. Antibody-nanoparticle conjugates can function in the targeted delivery of a payload (e.g., small molecules or genetic engineering components) to a cell ex vivo or in vivo that expresses the target cell marker. For example, scFv or other binding fragments can be linked to the surface of nanoparticles to guide delivery to target cells. The linkage can be through, for example, covalent attachment.

Examples of nanoparticles include metal nanoparticles (e.g., gold, platinum, or silver), liposomes, and polymer-based nanoparticles.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467 (1980) and Hope et al., Chem. Phys. Lip. 40:89 (1986). For additional information regarding nanoparticles, see Yetisgin et al., Molecules 2020, 25, 2193.

Examples of polymers that can be used within nanoparticles include polyglutamic acid (PGA); poly(lactic-co-glycolic acid) (PLGA); Polylactic acid (PLA); poly-D-lactic acid (PDLA); PLGA-dimethacrylate; polyamines; polyorganic amines (e.g., polyethyleneimine (PEI), polyethyleneimine celluloses); poly(amidoamines) (PAMAM); polyamino acids (e.g., polylysine (PLL), polyarginine); polysaccharides (e.g., cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins.

In particular embodiments, the nanoparticles can include a coating, particularly when used in vivo. A coating can serve to shield the encapsulated cargo and/or reduce or prevent off-target binding. Off-target binding is reduced or prevented by reducing the surface charge of the nanoparticles to neutral or negative. Coatings can include neutral or negatively charged polymer- and/or liposome-based coatings. In particular embodiments, the coating is a dense surface coating of hydrophilic and/or neutrally charged hydrophilic polymer sufficient to prevent the encapsulated cargo from being exposed to the environment before release into a target cell. In particular embodiments, the coating covers at least 80% or at least 90% of the surface of the nanoparticle.

Examples of neutrally charged polymers that can be used as a nanoparticle coating include polyethylene glycol (PEG); poly(propylene glycol); and polyalkylene oxide copolymers, (PLURONIC®, BASF Corp., Mount Olive, NJ).

The size of particles can vary over a wide range and can be measured in different ways. In preferred embodiments, nanoparticles are <130 nm in size. However, nanoparticles can also have a minimum dimension of equal to or less than 500 nm, less than 150 nm, less than 140 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, nanoparticles are 90 to 130 nm in size.

Dimensions of the particles can be determined using, e.g., conventional techniques, such as dynamic light scattering and/or electron microscopy.

### 11. Vectors, Host Cells, and Recombinant Methods

Embodiments are also directed to the provision of isolated nucleic acids encoding anti-FOLR1 antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (e.g., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. lichenformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-FOLR1 antibody-encoding vectors. Saccharomyces cerevisiae, or common baker's yeast, is a commonly used lower eukaryotic host microorganism. However, a number of other genera, species, and strains are available and useful, such as Spodoptera frugiperda (e.g., SF9), Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-FOLR1 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low. The antibodies produced in a cell-free system may be aglycosylated depending on the source of the cells.

Where the antibody is secreted into the medium, supernatants from such expression systems are generally first concentrated using a commercially available protein concentration filter, for example, an Amicon® or Millipore® Pellicon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxyapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 12. Pharmaceutical Compositions and Methods of Administration

The antibodies and antibody conjugates provided herein can be formulated into pharmaceutical compositions using methods available in the art and those disclosed herein. Any of the antibody conjugates provided herein can be provided in the appropriate pharmaceutical composition and be administered by a suitable route of administration. Antibody conjugates as described herein are also referred to as active ingredients. Salts and/or prodrugs of the active ingredients can also be used.

A pharmaceutically acceptable salt includes any salt that retains the activity of the active ingredients and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

The methods provided herein encompass administering pharmaceutical compositions comprising at least one antibody conjugate provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete), Hank' solution, Ringer's solution, Normosol-R (Abbott Labs), Plasma-LyteA® (Baxter Laboratories, Inc., Morton Grove, IL), saine, buffered saline, physiological saline, glycerol, ethanol, or combinations thereof), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions such as buffered saline and physiological saline and aqueous dextrose and glycerol solutions can also be employed as liquid carriers, particularly for injectable solutions. Additional exemplary pharmaceutically acceptable carriers include any and all absorption delaying agents, antioxidants, binders, buffering agents, bulking agents or fillers, chelating agents, coatings, disintegration agents, dispersion agents, dispersion media, gels, isotonic agents, lubricants, preservatives, salts, solvents or co-solvents, stabilizers, surfactants and/or delivery vehicles. Examples of suitable pharmaceutical carriers further described in Martin, E. W., Remington's Pharmaceutical Sciences.

In clinical practice the pharmaceutical compositions or antibody conjugates provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition or antibody conjugate provided herein is administered parenterally. In some embodiments, a pharmaceutical composition or antibody conjugate provided herein is administered intravenously.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibody conjugates. Therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, stage of cancer, previous or concurrent therapeutic interventions, idiopathy of the subject and route of administration.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, and propylene glycol.

In some embodiments, the pharmaceutical composition comprises an isotonic agent. Exemplary isotonic agents include polyhydric sugar alcohols including trihydric or higher sugar alcohol, such as glycerin, erthritol, arabitol, xylitol, sorbitol, or mannitol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, tartrate, fumarate, gluconate, oxalate, acetate, phosphate, histidine, trymethylamine salts, and/or monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents such as ethylene-diamine-tetra-acetic acid (EDTA), controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

Exemplary preservatives include Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody conjugate, since, in some embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopeia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody or antibody-conjugate will decompose. Such excipients, which are referred to herein as “stabilizers,” include, but are not limited to, antioxidants such as ascorbic acid, pH buffers, or salt buffers.

### 12.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 13. Therapeutic Applications

For therapeutic applications, the conjugates provided herein can be administered to a mammal, generally a human, in a pharmaceutically acceptable dosage form such as those known in the art and those discussed above. For example, the antibody conjugates may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibody conjugates also are suitably administered by peritumoral, intralesional, or perilesional routes, to exert local as well as systemic therapeutic effects. The intravenous route may be particularly useful, for example, in the treatment of ovarian tumors.

The antibody conjugates provided herein may be useful for the treatment of any disease or condition involving folate receptor alpha (FOLR1). In some embodiments, the disease or condition is a disease or condition that can be diagnosed by overexpression of folate receptor alpha. In some embodiments, the disease or condition is a disease or condition that can benefit from treatment with an anti-folate receptor alpha antibody. In some embodiments, the disease or condition is pediatric AML. In some embodiments, the disease or condition is CBF/GLIS AML. Targeted therapeutics disclosed herein that bind FOLR1 can additionally be used to treat other cancers including other leukemias, peritoneal cancer, fallopian tube cancer, ovarian cancer (e.g., epithelial ovarian cancer), endometrial cancer, cervical cancer, breast cancer (e.g., triple-negative breast cancer, HER2-breast cancer), bladder cancer, renal cell carcinoma, pituitary tumors, lung cancer (e.g., lung adenocarcinoma or epithelial lung cancer such as non-small cell lung cancer), uterine cancer, squamous cell carcinoma, ureter cancer, urethral cancer, osteosarcoma, or transitional cell carcinoma. In some embodiments, the disease or condition is relapsed. In some embodiments, the disease or condition is refractory. In some embodiments, the diseases or condition is relapsed and refractory.

The following clinical trials, by Trial Identifier No., provide further support for the efficacy of binding FOL1R in the treatment of various cancer types: GDCT0356356: Indications: Peritoneal Cancer (PC), Fallopian Tube Cancer (FTC), Epithelial Ovarian Cancer (EOC); GDCT0374537: Indications: Ovarian Cancer (OC), EOC, FTC, PC; GDCT0429750: Indications: OC, Solid Tumor, Endometrial Cancer (EC), Non-Small Cell Lung Cancer (NSCLC), FTC, PC, EOC, Triple-Negative Breast Cancer (TNBC); GDCT0026391: Indications: FTC, PC, OC, EOC; GDCT0447204: Indications: EOC, PC, FTC; GDCT0232423: Indications: EOC, PC, FTC, OC; GDCT0229058: Indications: NSCLC; GDCT0445760: Indications: OC; GDCT0001547: Indications: EOC, OC, PC, FTC; GDCT0198047: Indications: NSCLC; GDCT0201658: Indications: TNBC, Breast Cancer (BC), Human Epidermal Growth Factor Receptor 2 Negative Breast Cancer (HER2-Breast Cancer (HER2-BC)); GDCT0043006: Indications: OC; GDCT0423171: Indications: OC; GDCT0002756: Indications: EOC; GDCT0011290: Indications: Solid Tumor; GDCT0198051: Indications: OC; GDCT0286303: Indications: Metastatic BC, TNBC, BC, HER2-BC; GDCT0227787: Indications: OC, EOC, PC, FTC; GDCT0403589: Indications: FTC, PC, EOC; GDCT0004066: Indications: OC; GDCT0007040: Indications: Metastatic Renal Cell Carcinoma (RCC), RCC; GDCT0007042: Indications: OC, EC; GDCT0445900: Indications: OC; GDCT0041274: Indications: Adenomas, Pituitary Tumor; GDCT0016528: Indications: Lung Adenocarcinoma; GDCT0346710: Indications: EC, Uterine Cancer (UC); GDCT0347063: Indications: EC; GDCT0347076: Indications: EC; GDCT0014731: Indications: EOC, FTC, PC, OC; GDCT0144078: Indications: OC; GDCT0048904: Indications: Lung Adenocarcinoma, NSCLC; GDCT0152905: Indications: NSCLC, Squamous Cell Carcinoma; GDCT0010332: Indications: PC, EOC, FTC, OC; GDCT0380005: Indications: EOC, OC, FTC, PC, Metastatic OC; GDCT0433366: Indications: OC, PC, FTC, EOC; GDCT0291846: Indications: Bladder Cancer, Transitional Cell Carcinoma (Urothelial Cell Carcinoma), RCC, Ureter Cancer, Urethral Cancer, Metastatic Transitional (Urothelial) Tract Cancer, Bladder Carcinoma; GDCT0381573: Indications: TNBC, EC, OC, NSCLC, PC, FTC, EOC; GDCT0232424: Indications: EOC, PC, FTC, EC, OC; GDCT0325007: Indications: EC, OC, FTC, PC, Carcinomas, UC, Metastatic BC; GDCT0401603: Indications: BC, Lung Cancer (LC); GDCT0405347: Indications: OC; GDCT0429611: Indications: EOC, EC, Solid Tumor, PC, FTC, NSCLC; GDCT0209078: Indications: Solid Tumor; GDCT0158131: Indications: Solid Tumor, EC, EOC, NSCLC, OC, FTC, PC, Transitional Cell Carcinoma (Urothelial Cell Carcinoma), Cervical Cancer, RCC; GDCT0301058: Indications: Osteosarcoma; GDCT0456860: Indications: NSCLC, RCC, EOC, Solid Tumor, FTC, PC, EC, Squamous Non-Small Cell Lung Carcinoma; GDCT0450501: Indications: PC, NSCLC; GDCT0284998: Indications: OC, EOC, Carcinomas; GDCT0005507: Indications: OC, EOC; GDCT0006565: Indications: Metastatic Cancer Advanced Malignancy; GDCT0250249: Indications: Unspecified Cancer; GDCT0006477: Indications: Metastatic Cancer; GDCT0291176: Indications: Osteosarcoma; GDCT0429953: Indications: OC; GDCT0002840: Indications: Solid Tumor; GDCT0017012: Indications: Solid Tumor Lymphoma; GDCT0007464: Indications: Advanced Malignancy Solid Tumor, Metastatic OC, Unspecified Cancer; GDCT0205391: Indications: Solid Tumor OC, EC, NSCLC, TNBC; GDCT0452526: Indications: Osteosarcoma; GDCT0198062: Indications: Metastatic RCC, OC; GDCT0281679: Indications: TNBC; GDCT0243737: Indications: OC, FTC, Solid Tumor, Malignant Mesothelioma, EC; GDCT0170283: Indications: EC, Advanced Malignancy, Metastatic Cancer, Solid Tumor; GDCT0162335: Indications: Solid Tumor; GDCT0289943: Indications: Solid Tumor EC, TNBC, OC, FTC, NSCLC, PC; GDCT0279766: Indications: OC; GDCT0278064: Indications: FTC, OC, PC, UC, EC, Carcinomas, TNBC, BC; GDCT0250120: Indications: TNBC; GDCT0295131: Indications: OC, LC, TNBC; GDCT0391284: Indications: FTC, EC, EOC, Peritoneal Tumor, OC, PC; GDCT0003999: Indications: Metastatic Cancer, Metastatic RCC; GDCT0011743: Indications: FTC, PC, EOC; GDCT0232969: Indications: Solid Tumor, Hodgkin Lymphoma (B-Cell Hodgkin Lymphoma), Non-Hodgkin Lymphoma, OC, EC; GDCT0327878: Indications: OC, FTC, PC, EOC; GDCT0434405: Indications: Solid Tumor; GDCT0319681: Indications: OC, EC, EOC, FTC, PC, Gynecological Cancer.

Compositions disclosed herein can also be used to treat a complication or disease related to C/G AML. For example, complications relating to AML may include a preceding myelodysplastic syndrome (MDS, formerly known as “preleukemia”), secondary leukemia, in particular secondary AML, high white blood cell count, and absence of Auer rods. Among others, leukostasis and involvement of the central nervous system (CNS), hyperleukocytosis, residual disease, are also considered complications or diseases related to AML.

In certain embodiments, the effectiveness of the methods herein (e.g., method of modulating an immune response in an individual) can be assessed by measuring the biological activity of cancer cells present in a sample isolated from the treated individual.

### 14. Combinations

In certain embodiments, the anti-FOLR1 antibody conjugates are used in combination with one or more second therapeutic agents. The second therapeutic agent can be any agent deemed suitable by the person of skill. In certain embodiments, the second therapeutic agent is a cancer therapeutic. In certain embodiments, the second therapeutic agent modulates angiogenesis.

In particular embodiments, the second therapeutic agent is a VEGF inhibitor. Useful VEGF inhibitors are described herein. In particular embodiments, the second therapeutic agent is a GCSF. Useful GCSFs are described herein. In certain embodiments, the anti-FOLR1 antibody conjugates are used in combination with a VEGF inhibitor and with a GCSF.

Generally, the anti-FOLR1 antibody conjugate and the second therapeutic agent are administered according to their own doses and schedules. Thus, in certain embodiments, the anti-FOLR1 antibody conjugate is administered at a dose and schedule deemed useful by the practitioner of skill. In certain embodiments, the second therapeutic agent is administered at a dose and schedule deemed useful by the practitioner of skill. In particular embodiments, the second therapeutic agent is administered according to its labelled instruction.

In certain embodiments, the anti-FOLR1 antibody conjugate and the additional therapeutic agent are administered consecutively in either order. As used herein, the terms “consecutively,” “serially,” and “sequentially” refer to administration of an anti-FOLR1 antibody conjugate after an additional therapeutic agent, or administration of the additional therapeutic agent after the anti-FOLR1 antibody conjugate. For instance, consecutive administration may involve administration of the anti-FOLR1 antibody conjugate in the absence of the additional therapeutic agent during an induction phase (primary therapy), which is followed by a post-induction treatment phase comprising administration of the additional therapeutic agent. The methods may further comprise a maintenance phase comprising administration of the anti-FOLR1 antibody conjugate or the additional therapeutic agent, or both. Alternatively, consecutive administration may involve administration of the additional therapeutic agent in the absence of the anti-FOLR1 antibody conjugate during an induction phase (primary therapy), which is followed by a post-induction treatment phase comprising administration of the anti-FOLR1 antibody conjugate. The methods may further comprise a maintenance phase comprising administration of the anti-FOLR1 antibody conjugate or the additional therapeutic agent, or both.

In certain embodiments, the anti-FOLR1 antibody conjugate and the additional therapeutic agent are administered concurrently. As used herein, the terms “concurrently,” “simultaneously,” and “in parallel” refer to administration of an anti-FOLR1 antibody conjugate and an additional therapeutic agent during the same doctor visit or during the same phase of treatment. For instance, both the anti-FOLR1 antibody conjugate and the additional therapeutic agent may be administered during one or more of an induction phase, a treatment phase, and a maintenance phase. However, concurrent administration does not require that the anti-FOLR1 antibody conjugate and the additional therapeutic agent be present together in a single formulation or pharmaceutical composition, or that the anti-FOLR1 antibody conjugate and the additional therapeutic agent be administered at precisely the same time.

In certain embodiments, provided are compositions and therapeutic formulations comprising any of the antibody conjugates provided herein in combination with one or more second therapeutic agents, and methods of treatment comprising administering such combinations to subjects in need thereof. In some embodiments, the second therapeutic agent is a VEGF inhibitor. In some embodiments, the one or more VEGF inhibitors comprise an antibody that inhibits VEGF activity. In some embodiments, the one or more VEGF inhibitors are selected from bevacizumab (AVASTIN®) and bevacizumab biosimilars. In some embodiments, the bevacizumab biosimilar is selected from the group consisting of: MVASI (ABP 215, Amgen), Zirabev (Pfizer), Bevax (BEVZ92, mAbxience), Lumiere (Elea), Apotex (Apobiologix), Equidacent (FKB238, AstraZeneca/Centus Biotherapeutics), Avegra (BCD-021, Biocad), BP 01 (Aurobindo Pharma), BCD500 (BIOCND), Krabeva (Biocon), BAT1706 (Bio-Thera Solutions), BXT-2316 (BioXpress Therapeutics), Bevaro (Cadila Pharmaceuticals), BI 695502 (Boehringer Ingelheim), CT-P16 (Celltrion), CHS-5217 (Coherus), DRZ_BZ (Dr Reddy's Laboratories), Cizumab (Hetero/Lupin), Byvasda (IBI305, Innovent Biologics), MIL60 (Mabworks), MYL 14020 (Mylan), ONS-1045 (Oncobiologics/Viropro), HD204 (Prestige Biopharma), Ankeda (QL 1101, Qilu Pharmaceutical), Bevacirel (Reliance Life Sciences), Aybintio (SB8, Samsung Bioepis), Onbevzi (Samsung Bioepis), HLX04 (Shanghai Henlius Biotech), TX16 (Tanvex BioPharma), MB02 (mAbxience), BI 695502 (Boehringer Ingelheim), and Oyavas (STADA).

Examples of second therapeutic agents also include, but are not limited to, Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially uncialamycin, calicheamicin gammall, and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pladienolide B, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

In some embodiments, the second therapeutic agent is a granulocyte colony-stimulating factor (GCSF). In some embodiments, the second therapeutic agent is second therapeutic agent is a pegylated GCSF. In some embodiments, the second therapeutic agent is GCSF is pegfilgrastim or filgrastim. In some embodiments, the second therapeutic agent is pegfilgrastim. Pegfilgrastim may be Neulasta© or a biosimilar. In some embodiments, the second therapeutic agent is filgrastim. Filgrastim may be Neupogen® or a biosimilar.

In some embodiments, the GCSF second therapeutic agent is administered concurrently with or sequentially to the first dose of the antibody conjugate. In some embodiments, the second therapeutic agent is second therapeutic agent is administered at least 7 days after administering the first dose of the antibody conjugate. In some embodiments, the second therapeutic agent is second therapeutic agent is administered at 7, 8, 9, 10 or 11 days after administering the first dose of the antibody conjugate. In some embodiments, the second therapeutic agent is second therapeutic agent is administered at 8±2 days after administering the first dose of the antibody conjugate. In some embodiments, the second therapeutic agent is second therapeutic agent is administered at around 8 days after administering the first dose of the antibody conjugate. In some embodiments, the second therapeutic agent is administered 8 days after administering the first dose of the antibody conjugate. In some embodiments, the second therapeutic agent is second therapeutic agent is administered between 8±2 days and 15±2 days after administering the first dose of the antibody conjugate.

In some embodiments, the GCSF is pegfilgrastim, which is administered as directed or as deemed suitable by the practitioner of skill. In certain embodiments, the pegfilgrastim dose is 6 mg. In some embodiments, pegfilgrastim is administered once every 8-10 days for 1-5 cycles. In some embodiments, pegfilgrastim is administered once every 8-10 days for 1-3 cycles. In some embodiments, pegfilgrastim is administered once every 8-10 days for 1 cycle.

In some embodiments, the GCSF is filgrastim, which is administered as directed or as deemed suitable by the practitioner of skill. In certain embodiments, the first dose is administered at least 24 hours after administering the first dose of the antibody conjugate. In some embodiments, the GCSF is filgrastim and the dose is 5-10 μg/kg/day. In some embodiments, the GCSF is filgrastim and the dose is 5 μg/kg/day. In some embodiments, the GCSF is filgrastim, the dose is 5 μg/kg/day, and the patient is receiving myelosuppressive chemotherapy or induction and/or consolidation chemotherapy for AML. In some embodiments, the GCSF is intravenous filgrastim, the dose is 5 μg/kg/day, and the patient is receiving myelosuppressive chemotherapy or induction and/or consolidation chemotherapy for AML. In some embodiments, the GCSF is subcutaneous filgrastim, the dose is 5 μg/kg/day, and the patient is receiving myelosuppressive chemotherapy or induction and/or consolidation chemotherapy for AML. In some embodiments, the GCSF is filgrastim and the dose is 10 μg/kg/day. In some embodiments, the GCSF is filgrastim, the dose is 10 μg/kg/day, and the patient is undergoing bone marrow transplantation or undergoing autologous peripheral blood progenitor cell collection and therapy. In some embodiments, the GCSF is subcutaneous filgrastim, the dose is 10 μg/kg/day, and the patient is undergoing bone marrow transplantation. In some embodiments, the GCSF is intravenous filgrastim, the dose is 10 μg/kg/day, and the patient is undergoing autologous peripheral blood progenitor cell collection and therapy. In some embodiments, the GCSF is filgrastim and the dose is 6 μg/kg/day. In some embodiments, the GCSF is filgrastim, the dose is 6 μg/kg/day, and the patient has congenital neutropenia. In some embodiments, the GCSF is subcutaneous filgrastim, the dose is 6 μg/kg/day, and the patient has congenital neutropenia. In some embodiments, the GCSF is filgrastim, the dose is 5 μg/kg/day, and the patient has cyclic or idiopathic neutropenia. In some embodiments, the GCSF is subcutaneous filgrastim, the dose is 5 μg/kg/day, and the patient has cyclic or idiopathic neutropenia. In some embodiments, the GCSF is filgrastim, the dose is 10 μg/kg/day, and the patient has been acutely exposed to myelosuppressive doses of radiation. In some embodiments, the GCSF is subcutaneous filgrastim, the dose is 10 μg/kg/day, and the patient has been acutely exposed to myelosuppressive doses of radiation.

In certain embodiments, the second therapeutic agent is CXCR4 antagonist. In certain embodiments, the second therapeutic agent is plerixafor (Mozobil). The second therapeutic agent is administered as described herein, for instance according to labelled instructions. In certain embodiments, the plerixafor dose is 20 mg, for instance in patients under 83 kg. In certain embodiments, the plerixafor dose is 0.24 mg/kg. In certain embodiments, the plerixafor dose is 13 mg, for instance in patients under 83 kg with renal impairment. In certain embodiments, the plerixafor dose is 0.16 mg/kg, for instance in patients with renal impairment. In certain embodiments, the second therapeutic agent lowers the rate of neutropenia in subjects. In certain embodiments, the FOLR1 antibody conjugate is administered in combination with pegfilgrastim and plerixafor.

In certain embodiments, the second therapeutic agent(s) is or are selected from: ADE (cytarabine, daunorubicin, etoposide); low dose cytarabine (20 mg/m²); fudarabine/cytarabine±granulocyte-colony stimulating factor (G-CSF) (FLAG); venetoclax+azacytidine; venetoclax+cytarabine; donor lymphocyte infusion (DLI).

In certain embodiments, the second therapeutic agent is methotrexate. In certain embodiments, the second therapeutic agent is DLI. In certain embodiments, the second therapeutic agents are fludarabine, ara-Cn and G-CSF. In certain embodiments, the second therapeutic agent is venetoclax. In certain embodiments, the second therapeutic agent is azacitidine. In certain embodiments, the second therapeutic agent is decitabine. In certain embodiments, the second therapeutic agents are methotrexate and DLI. In certain embodiments, the second therapeutic agents are methotrexate, and fludarabine, ara-C and G-CSF. In certain embodiments, the second therapeutic agents are methotrexate and venetoclax. In certain embodiments, the second therapeutic agents are venetoclax and azacitidine. In certain embodiments, the second therapeutic agents are venetoclax, azacitidine, and gemtuzumab. In certain embodiments, the second therapeutic agents are plerixafor and methotrexate. In certain embodiments, the second therapeutic agents are methotrexate and calaspargase. In certain embodiments, the second therapeutic agents are decitabine and DLI.

### 15. Kits

In some embodiments, the combination provided herein is provided in the form of a kit, i.e., a packaged combination of reagents in predetermined amounts with instructions for performing a procedure. In other embodiments, the procedure is a therapeutic procedure.

In some embodiments, the kit further comprises a solvent for the reconstitution of the anti-FOLR1 antibody conjugate. In some embodiments, the anti-FOLR1 antibody conjugate is provided in the form of a pharmaceutical composition.

In some embodiments, the kit further comprises a VEGF inhibitor, e.g., bevacizumab or a bevacizumab biosimilar, and instructions for use. In certain embodiments, the kit provides a GCSF, e.g., pegfilgrastim or filgrastim, and instructions for use. In certain embodiments, the kit provides pegfilgrastim and instructions for use. In certain embodiments, the kit provides filgrastim and instructions for use.

In some embodiments, the kit further comprises instructions for use with a VEGF inhibitor, e.g., bevacizumab or a bevacizumab biosimilar. In certain embodiments, the kit provides instructions for use with a GCSF, e.g., pegfilgrastim or filgrastim. In certain embodiments, the kit provides instructions for use with pegfilgrastim. In certain embodiments, the kit provides instructions for use with filgrastim.

In certain embodiments, the pharmaceutical package or kit comprises a container, a composition comprising a folate receptor alpha (FOLR1) antibody conjugate; and a package insert comprising instructions to administer the FOLR1 antibody conjugate according the methods described herein.

### EXAMPLES

### Example 1

### FOLR1 Expression in CBG::GLIS AML

Cryptic inv(16)(p13.3q24.3) leading to the CBFA2T3-GLIS2 (CBF/GLIS) oncogenic fusion is exclusively seen in infants with AML and is associated with adverse outcome. Infants with this fusion are uniformly refractory to conventional therapies and despite intensive and myeloablative therapies, virtually all patients relapse with survival less than 15% (Smith et. al. 2020). In the effort to discover actionable targets for this highly refractory leukemia, the genome and transcriptome of nearly 3,000 AML cases were interrogated, including 45 cases of CBF/GLIS AML, and contrasted this to the transcriptome in normal hematopoietic tissues.

In this study, an expansive target discovery effort through TARGET and Target Pediatric AML (TpAML) was used to discover AML-restricted targets (high expression in AML, silent in normal) that were previously elusive to identify actionable targets for high-risk CBF/GLIS AML. An initial computational effort to identify CBF/GLIS-specific targets focused on genes that are overexpressed in CBF/GLIS AML and silent in normal hematopoietic tissues provided 194 candidate genes. Filtering this library of genes based on cell surface expression and prevalence in target population narrowed the target gene list to six cell surface encoding genes (FOLR1, F4S1, HPSE2, KLRF2, ME.G170 and PCD119). FOLR1 encodes folate receptor alpha and given available therapeutic options with FOLR1-targeting agents, this gene was selected for further studies and therapeutic assessment of available targeted therapies.

Based on RNA-seq data, FOLR1 is uniquely expressed in CBF/GLIS AML as it is absent in other AML and in normal hematopoietic cells (FIG. 1A) providing an opportunity to specifically target leukemia cells while sparing normal hematopoiesis. Cell surface expression of FOLR1 on AML blasts was confirmed by flow cytometry, which further shows AML-restricted expression of FOLR1 on AML cells but not in normal hematopoietic subsets in individual CBF/GLIS patient samples (FIGS. 18, IC). It was previously demonstrated that forced expression of CBF/GLIS fusion transcript in cord blood hematopoietic stem/progenitor cells (CB HSPCs) induces malignant transformation that fully recapitulates primary CBF/GLIS AML (Hylkema et. al. 2019 ASH). It is here demonstrated that CBF/GLIS-transduced CB HSPCs upregulate FOLR1 expression indicating that FOLR1 expression is causally associated with CBF/GLIS expression (FIG. 1D).

Given cell surface expression of FOLR1 on CBF/GLIS AML cells, next investigated was the preclinical efficacy of targeting FOLR1 using FOLR1-directed site-specific antibody-drug Conjugate P (an IgG antibody with VH region SEQ ID NO: 362; VL region SEQ ID NO: 367; para-azidomethylphenylalanine residues substituting for Y180 and F404 of each VH region; according to conjugate formula P, wherein n=4). In vitro efficacy of Conjugate P was tested against MV4;11 AML cell line engineered to express FOLR1 (MV4;11 FOLR1+) and CBF/GLIS-transduced CB HPSCs. Conjugate P exhibited high cytotoxicity against MV4;11 FOLR1+ and CBF/GLIS-transduced CB HPSCs with IC50s of 0.1 nM and 4.2 nM, respectively (FIG. 1E). In vivo efficacy of Conjugate P was evaluated in MV4;11 FOLR1+NSG xenograft models, which showed potent activity that led to complete leukemia clearance after 3 weekly doses of Conjugate P at 2.5 and 5 mg/kg (FIG. 1F). The anti-leukemia effects of Conjugate P resulted in significant increase in survival (p=0.002, FIG. 1G). Conjugate P showed similar in vivo efficacy in the xenograft mice bearing CBF/GLIS-transduced CB HSPCs (FIG. 1H) suggesting highly potent activity of Conjugate P against FOLR1+AML cells.

### Example 2

### CBG::GLIS Case Studies

A total of at least 16 patients have been treated with Conjugate P through Expanded Access. All patients have confirmed Relapsed/Refractory CBF2AT3-GLIS2+AML.

A 2-year-old female with relapsed CBF2AT3-GLIS2+AML received 4 prior lines of therapy including an unrelated donor transplant. After one cycle of Conjugate P, histology staining shows marked reduction in tumor cells.

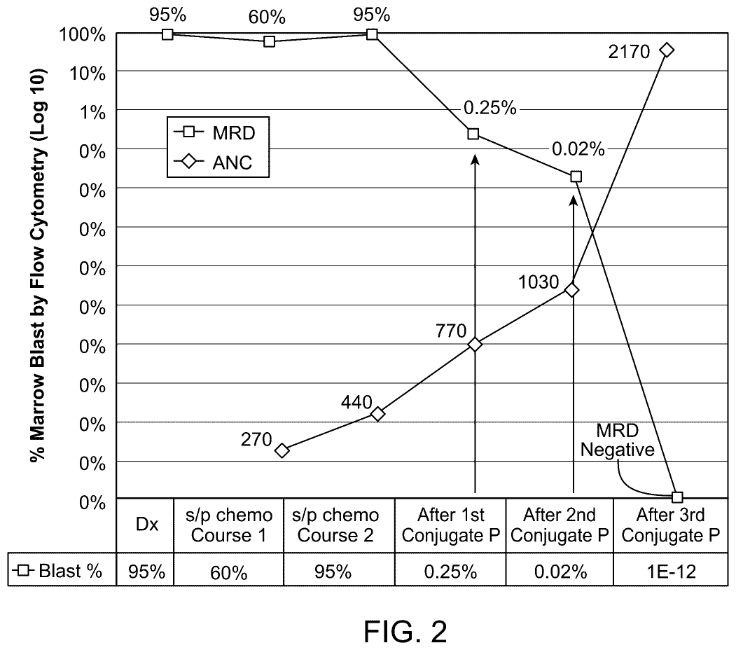

A 2-year-old female with primary refractory, transplant naïve, CBF2AT3-GLIS2+AML received 4 cycles of Conjugate P, monotherapy at 4.3 mg/kg. Patient was bridged to transplant. At Day+100, patient remains disease-free. FIG. 2 shows negative MRD and positive ANC.

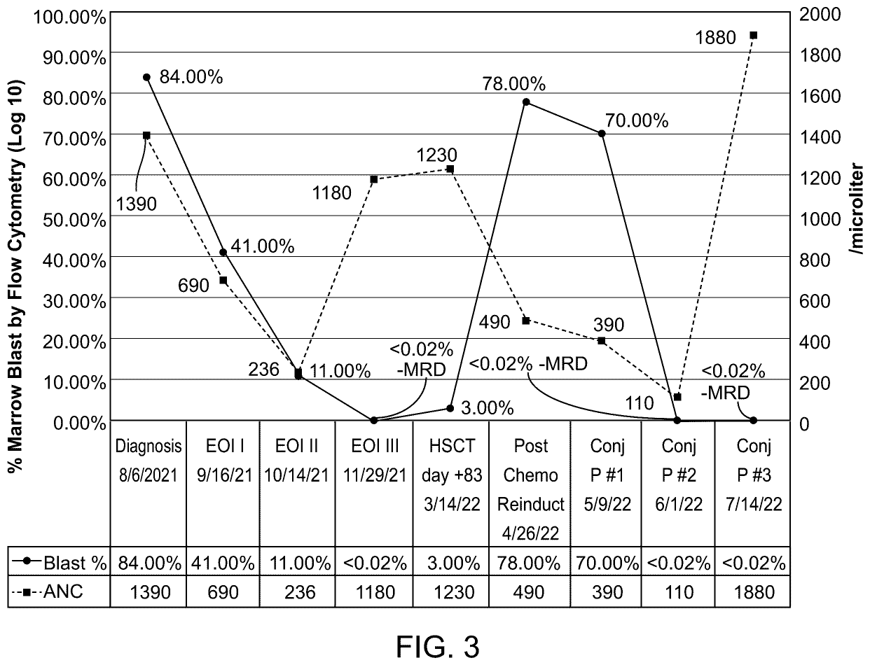

A 3-year-old female with Ph+& CBF2AT3-GLIS2+AML relapsed 3 months post stem cell transplant (Day+83). Patient received 4 cycles of Conjugate P at 4.3 mg/kg with Dasatinib. Patient continues to receive Conjugate P in combination with DLI. FIG. 3 shows negative MRD and positive ANC.

### Example 3

### CBG::GLIS Case Study

A 2-year-old girl of Vietnamese descent received a bone marrow examination for bicytopenia and macrocytosis. Flow cytometry confirmed the poor prognostic RAM immunophenotype AML (CD45 dim, HLA-DR negative, CD38 dim, CD56 bright; Broderson et al., 2016, Leukemia 30(10):2077-2080) and FOLR1 expression. Marrow aspirate smears showed numerous hypercellular spicules and blasts with scant cytoplasm and some blebbing but no granules or Auer rods. Marrow exhibited 100% cellularity. CNS was not involved. Review of diagnostic genomic profile mid induction showed a BCR::ABL minor breakpoint fusion as well as CBF::GLIS fusion.

The child began AML induction on a trial randomized to the experimental arm with CPX-351 (cytarabine and daunorubicin liposome) and gemtuzumab ozogamicin (GO). Due to lack of response to AML therapy at end of Induction (EOI) I, she was taken off protocol and started on modified ALL regimen for Induction II consisting of cytarabine, low dose weekly methotrexate and bimonthly peg-asparaginase with addition of an oral tyrosine kinase inhibitor (TKI), dasatinib. EOI II marrow examination showed improvement with reduction in blasts (11%) and detectable BCR::ABL but undetectable CBF::GLIS by Q-RT-PCR. Induction III included low dose weekly methotrexate, cytarabine, GO, and dasatinib. EOI III marrow examination showed complete morphologic remission (CR) with no evidence of minimal residual disease (MRD) or fusion transcripts of either clone.

Due to availability of a matched sibling donor, the child underwent hematopoietic stem cell transplantation (HSCT); however, was noted to have 3% blasts on day +83 marrow examination despite previously commencing dasatinib post-transplant prophylaxis. BCR::ABL and CBF::GLIS transcripts were again detectable at low levels. Dasatinib dosage was maximized and weekly MTX and bimonthly peg-asparaginase were reintroduced, but the marrow disease burden increased significantly to 78% blasts positive only for CBF::GLIS fusion.

Given availability of FOLR1 directed ADC on single-patient compassionate use basis, patient received single agent Conjugate P on a bi-monthly basis. With rising absolute neutrophil count (ANC), evaluation of marrow after Conjugate P dose remarkably showed CR with MRD negative remission by flow and molecular studies. This time remission was consolidated with monthly Conjugate P and donor lymphocyte infusions (DLI). Patient remained in MRD-negative remission after the 3 of 4 planned Conjugate P/DLI maintenance.

Patient has maintained a 100% Lansky play score and tolerated the regimen with only prolonged cytopenia requiring supportive care as a toxicity.

### Example 4

### CBG::GLIS Treatment Plan

Patient is a 9-month-old girl who was initially diagnosed with acute myeloid leukemia (AML). Immunophenotyping showed the RAM phenotype and molecular studies showed the CBFA2T3-GLIS2 fusion, a very high-risk subtype with poor prognosis.

Patient started chemotherapy per the MRC15 regimen (FLAG-Ida-GO; fludarabine, ara-C, G-CSF, idarubicin, gemtuzumab ozogamicin). Disease response showed morphologic remission, but minimal residual disease (MRD) was detected at 0.06%. While awaiting donor search for bone marrow transplant, patient started a second cycle of chemotherapy (methotrexate, asparaginase), but MRD increased to 0.55%. Patient then received a third cycle of chemotherapy (FLAG-IDA), but disease burden continued to rise, with 10% blasts in the bone marrow.

Despite intensive chemotherapy, patient's disease is persistent and refractory. The best chance of cure is bone marrow transplant (BMT) after achieving a molecular remission. An unrelated BMT donor has been identified. Treatment plan is to induce a molecular remission with Conjugate P followed by BMT.

Conjugate P is administered by intravenous administration at 4.3 mg/kg (dose specifically recommended for this patient) on days 1, 3, and 7 (total dose per cycle of 12.9 mg/kg) or once every 14-21 days. The dose may be increased to 5.2 mg/kg if good tolerability. Each Conjugate P infusion is given over 1 hour. While the infusions are reported to be well-tolerated, there is provider discretion to use acetaminophen and/or diphenhydramine premedication, to be given 1 hour before the infusion.

Additional AML standard of care agents that belong to the class of drugs such as cytarabine and methotrexate, may be combined with the treatment regimen of Conjugate P during any cycle of treatment. Other standard of care agents can be combined. Institutional tumor lysis syndrome (TLS) guidelines will be followed. Growth factors (G-CSF) administration is allowed according to institutional guidelines. G-CSF administration when given prophylactically after Cycle 1 should be between Day 8 and 15 after administration of Conjugate P. A cycle is defined as either 14 or 21 days. Study treatment may be stopped at any time based on clinical judgement for the patient's best clinical interest.

The initial dose of 4.3 mg/kg was selected as this was the lowest dose demonstrated to have efficacy in adult studies. Interpatient incremental dose escalation may be considered for the patient in subsequent cycles if she does not have an adequate response to the drug and is deemed to be tolerating it sufficiently by the treatment team. Currently further dose optimization is being explored in an adult phase 1 dose expansion clinical study with a dose level up to 5.2 mg/kg.

### Example 5

### Combination with Plerixafor for CBG::GLIS

As shown in the Examples above, Conjugate P has in patients with CBF/GLIS AML has been reported with at least 17 patients demonstrating significant response with little to no toxicity (7 patients achieved MRD negative remissions). Plerixafor (Mozobil), a reversible CXCR4 antagonist, mobilizes marrow stem cells and leukemic cells and has been investigated in patients with relapsed/refractory leukemia in combination with chemotherapy with modest response. This Example provides a case of complete morphologic and immunophenotypic remission by targeted combination of Conjugate P and plerixafor in a child with refractory CBF/GLIS AML

Patient is a 2-year-old female with fever, arm pain, and bruising, and diagnosed with RAM phenotype AMKL with FOLR1-positive CBFA2T3::GLIS2 oncogenic fusion. She was enrolled on trial administration of cytarabine/daunorubicin with or without gilteritinib (AAML 1831 Arm A) but induction therapy was unsuccessful as evidenced by 14% residual bone marrow (BM) blasts by flow cytometry.

Patient was transitioned to Conjugate P monotherapy (4.3 mg/kg/dose IV every 2 weeks) as an outpatient for 4 cycles. BM was assessed q2wks prior to each cycle. Flow results post Cycles 1-4 were respectively: 2.7%, 2.02%, 1.6%, and 0.36%. FOLR1 activity, present initially, was absent after Cycle 4 raising concern for lack of FOLR1 expression vs antibody block. Given persistent MRD positive status, plerixafor was added for leukemic cell mobilization with Cycles 5 and 6 (Plerixafor 0.24 mg/kg/dose 4 h prior and 24 h post each Conjugate P dose). After Cycle 5, MRD was 0.013% by flow and 0.4% CBFA2T3::GLIS2 fusion expression. After Cycle 6, Patient achieved 0% MRD by flow and 0% CBFA2T3::GLIS2 fusion expression. Patient had one episode of transient neutropenia after Cycle 5 while RSV+. Blood counts improved with the addition of Plerixafor and no toxicities were noted.

The patient with refractory CBFA2T3::GLIS2-associated AMKL achieved CR with Conjugate P and plerixafor. Conjugate P combined with Plerixafor has not previously been reported and was well-tolerated by the patient.

### Example 6

This example provides an exemplary clinical treatment protocol. A 2-cohort Phase 1/2, open-label study evaluates the efficacy, PK and safety of Conjugate P in infants and children <12 years of age with relapsed/refractory AML. This study also explores tolerability, PK and anti-leukemic activity in newly diagnosed subjects with CBFA2T3::GLIS2 subtype AML. Cohort A will consist of subjects who are CBFA2T3::GLIS2 positive that have achieved a complete remission for their initial treatment but have relapsed or did not achieve remission after their initial induction therapy (refractory). Cohort B will consist of subjects who have been newly diagnosed with CBFA2T3::GLIS2 AML.

Cohort A—Relapsed/Refractory CBFA2T3::GLIS2 subtype AML subjects

Cycle 1 (Dose 1 and 2): Subjects will receive Conjugate P monotherapy at 4.3 mg/kg on Days 1 and 15 of a 28-day cycle. Conjugate P will be administered intravenously (IV) over 1 to 2 hours of infusion time.

Cycle 2 (Dose 3 and 4): If subjects are in complete remission (CR), they will continue with 2 doses of Conjugate P at 4.3 mg/kg IV q2w until disease progression, transplant or other intervening therapy in the absence of disease progression, unacceptable toxicity or withdrawal from study.

If subjects do not achieve a CR prior to the start of Cycle 2, Conjugate P monotherapy will be escalated to 5.2 mg/kg q2w.

Cycle 3 and beyond (Dose 5+): Subjects who are in CR will continue with Conjugate P at their Cycle 2 dose level until disease progression, transplant or other intervening therapy in the absence of disease progression, unacceptable toxicity or withdrawal from study.

Subjects who have not achieved a CR at the conclusion of Cycle 2 will be administered Conjugate P at 4.3 mg/kg IV q2w plus a standard AML treatment regimen until disease progression, unacceptable toxicity, or withdrawal from study.

Cohort B—Newly Diagnosed AML subjects with CBFA2T3::GLIS2 subtype

Two dose levels will be assessed in this Cohort, Dose Level 1 is 4.3 mg/kg q2w. If the 4.3 mg/kg q2w dosing regimen is determined to be tolerable, subsequent subjects will be treated with Dose Level 2 of 5.2 mg/kg q 2w.

Cycle 1 (Dose 1 and 2): Subjects will receive 2 doses of Conjugate P q2w.

Cycle 2 (Dose 3 and 4): Subjects who achieve a CR or exhibit a reduction in leukemia burden (defined as <15% blasts) by Day 28 of Cycle 1, will continue on Conjugate P monotherapy at their assigned dose level until disease progression, transplant or other intervening therapy in the absence of disease progression, unacceptable toxicity, or withdrawal from study.

Subjects who do not achieve a reduction in leukemia burden, defined as >15% of residual leukemic blasts, or do not achieve a CR after Cycle 1 will be administered Conjugate P at 4.3 mg/kg q2w plus a standard AML treatment regimen from Cycle 2 onwards.

The standard AML treatment could include any of the following: ADE (cytarabine, daunorubicin, etoposide); low dose cytarabine (20 mg/m²); fludarabine/cytarabine±granulocyte-colony stimulating factor (G-CSF) (FLAG); venetoclax+azacytidine; venetoclax+cytarabine; donor lymphocyte infusion (DLI; e.g., if patient has had a prior transplant).

### Example 7

This example shows anti-leukemic activity of Luveltamab Tazevibulin (LT, STRO-002), a novel Folate Receptor-α (FR-α)-targeting antibody drug conjugate (ADC) in relapsed/refractory CBF2AT3::GLIS2 AML.

CBFA2T3::GLIS2 (CBF-GLIS) is an oncogenic fusion associated with a lethal and highly refractory childhood myeloid leukemia manifested by the RAM phenotype, expressed in infants and young children. Conventional chemotherapy and stem cell transplant (SCT) result in extraordinarily high rates of induction failure (>80%) and near uniform fatality with 2-year survival <15%. When conventional therapy fails, this leukemia is refractory to all reinduction therapies with post-relapse survival of nearly 0%.

In this study, all patients were required to demonstrate CBF-GLIS fusion by PCR or sequencing. Each investigator in consultation with Sutro Biopharma Inc. defined the treatment plan as to dose, schedule, and combination therapy administered.

Between August 2021 and March 2023, 25 patients with relapsed/refractory CBF-GLIS AML were treated with LT at doses of 4.3 or 5.2 mg/kg every 2-4 weeks (4 received smaller fractionated doses (FD) on days 1, 3, 5 per cycle). Median age at treatment was 2 years (range 0.7-8) with median number of prior therapies of 2 (range 1-7). 10 patients had relapsed after SCT. The remaining 15 had primary refractory, relapsed or MRD+AML. Median time from initial diagnosis was 7.9 months (range 1.5-82.5).

Of the 25 patients who received LT, 18 had >5% blasts (morphologic disease, MD) and 7 had <5% blasts (sub-morphologic disease, SMD). 21 patients initially received LT monotherapy (mono) (15 with MD and 6 with SMD), 9 of which went on to receive combination (combo) with additional therapies. The remaining 4 received LT in combo upfront.

Morphologic or molecular/MRD-response was observed in 12/25 patients (48%). Among those with MD, 7/18 (39%) reached a CR/CRh (5 MRD-) and among those with SMD, 5/7 (71%) reached MRD-CR. Median number of doses received before response was 2.

Responses were observed among patients receiving both mono and combo. Among the 11 MD pts treated with mono (excluding FD), 3 reached CR (27%). Among 10 patients with MD who received combo either initially or following mono, 4 reached CR/CRh (40%). Among the SMD pts, 4 reached MRD—with monotherapy and 1 with combo.

Of the 10 patients with post-SCT MD relapse, 7 patients received non-fractionated LT either as mono or combo therapy. Of these, the CR rate was 71% (5/7).

LT therapy enabled these children to receive potentially definitive therapy. Of the 7 patients with MD in CR, 4 proceeded to SCT, 2 received donor lymphocyte infusions and 1 received consolidation therapy. Of the 5 patients with SMD in MRD-CR, 1 proceeded to SCT, 2 received consolidation therapy in preparation for SCT, and 2 received LT maintenance therapy.

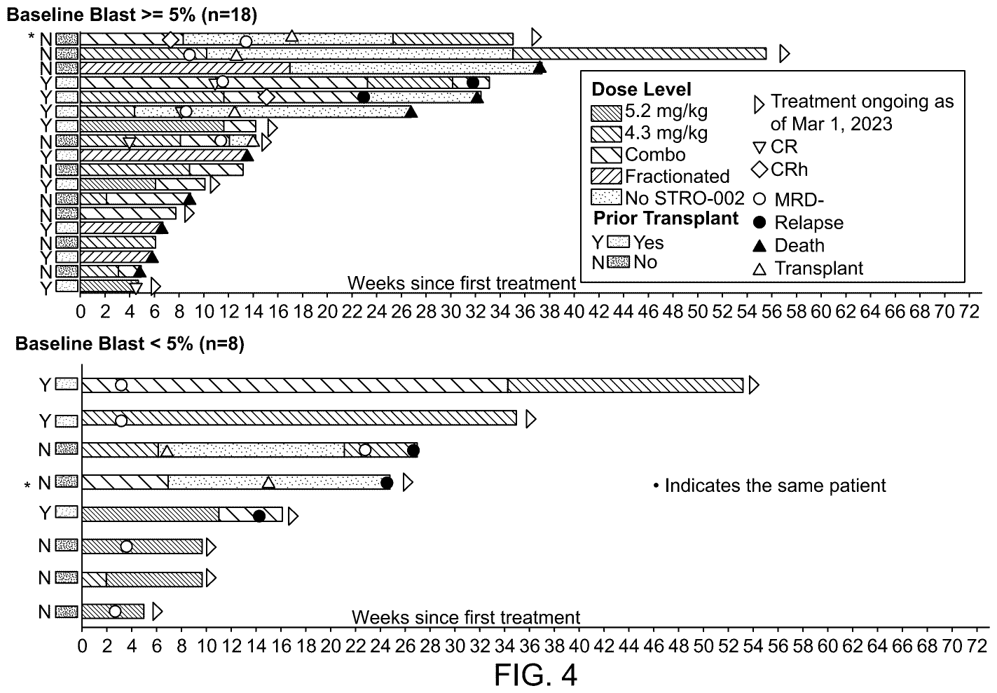

In total, 12 patients remain on treatment. Of the 25 pts who received LT, 17 (63%) are alive, 7 died of progressive disease (5 with relapse post-SCT) and 1 died from SCT-related complications. Of the 10 patients who received LT (non-fractionated) post-transplant, 7 are alive (78%). Patient status and treatment are shown in FIG. 4.

LT was generally well tolerated. TEAEs reported at an incidence of ≥25% included decreased neutrophil, platelet, WBC, or lymphocyte count, anemia, increased AST or ALT, diarrhea, and pyrexia. Grade 3+TEAEs (≥10%) included decreased neutrophil, platelet, WBC, or lymphocyte count, anemia, febrile neutropenia, ALT increased, vomiting and hypoxia. Observed cytopenias were often reported in pts with baseline cytopenia. Grade 5 SAEs were limited to fractionated dosing pts: apnea (n=1) and cardiopulmonary failure (n=1). Neither event was considered related to LT.

The median duration of LT treatment was 9.1 weeks (2.1-60) and 60% of patients received >5 doses. While the initial dose was typically delivered inpatient, approximately 80% of subsequent doses were delivered outpatient.

The results suggest that LT based therapy in CBF-GLIS AML provides clinical and laboratory responses that are highly unusual in this refractory patient population. LT has an acceptable safety profile and does not require inpatient delivery. Importantly, the data suggests that LT can enable these children to receive potential definitive therapy.

### Example 8

Table 5 provides sequences referred to herein.

TABLE 5
Sequences

| SEQ | | | | |
|---|---|---|---|---|
| ID | | | | |
| NO: | Molecule | Region | Scheme | Sequence |
| 1 | Human folate | | | MAQRMTTQLLLLLVWVAVVGEAQT |
| | receptor alpha | | | RIAWARTELLNVCMNAKHHKEKPG |
| | (hFOLR1) | | | PEDKLHEQCRPWRKNACCSTNTSQ |
| | | | | EAHKDVSYLYRFNWNHCGEMAPAC |
| | | | | KRHFIQDTCLYECSPNLGPWIQQV |
| | | | | DQSWRKERVLNVPLCKEDCEQWWE |
| | | | | DCRTSYTCKSNWHKGWNWTSGFNK |
| | | | | CAVGAACQPFHFYFPTPTVLCNEI |
| | | | | WTHSYKVSNYSRGSGRCIQMWFDP |
| | | | | AQGNPNEEVARFYAAAMSGAGPWA |
| | | | | AWPFLLSLALMLLWLLS |
| 2 | Cynomolgus folate | | | MAQRMTTQLLLLLVWVAVVGEAQT |
| | receptor alpha | | | RTARARTELLNVCMNAKHHKEKPG |
| | | | | PEDKLHEQCRPWKKNACCSTNTSQ |
| | | | | EAHKDVSYLYRFNWNHCGEMAPAC |
| | | | | KRHFIQDTCLYECSPNLGPWIQQV |
| | | | | DQSWRKERVLNVPLCKEDCERWWE |
| | | | | DCRTSYTCKSNWHKGWNWTSGFNK |
| | | | | CPVGAACQPFHFYFPTPTVLCNEI |
| | | | | WTYSYKVSNYSRGSGRCIQMWFDP |
| | | | | AQGNPNEEVARFYAAAMSGAGPWA |
| | | | | AWPLLLSLALTLLWLLS |
| 3 | Murine folate | | | MAHLMTVQLLLLVMWMAECAQSRA |
| | receptor alpha | | | TRARTELLNVCMDAKHHKEKPGPE |
| | | | | DNLHDQCSPWKTNSCCSTNTSQEA |
| | | | | HKDISYLYRFNWNHCGTMTSECKR |
| | | | | HFIQDTCLYECSPNLGPWIQQVDQ |
| | | | | SWRKERILDVPLCKEDCQQWWEDC |
| | | | | QSSFTCKSNWHKGWNWSSGHNECP |
| | | | | VGASCHPFTFYFPTSAALCEEIWS |
| | | | | HSYKLSNYSRGSGRCIQMWFDPAQ |
| | | | | GNPNEEVARFYAEAMSGAGFHGTW |
| | | | | PLLCSLSLVLLWVIS |
| 4 | SRP1848-A01 | CDR-H1 | Chothia | GFNITRY |
| 5 | SRP1848-A02 | CDR-H1 | Chothia | GFNISGF |
| 6 | SRP1848-A04 | CDR-H1 | Chothia | GFNIDQS |
| 7 | SRP1848-A06 | CDR-H1 | Chothia | GFNIGNS |
| 8 | SRP1848-A07 | CDR-H1 | Chothia | GFNIGYH |
| 9 | SRP1848-A08 | CDR-H1 | Chothia | GSNIRKH |
| 10 | SRP1848-A09 | CDR-H1 | Chothia | GFNIRKQ |
| 11 | SRP1848-A10 | CDR-H1 | Chothia | GFNIRKY |
| 12 | SRP1848-B01 | CDR-H1 | Chothia | GFNIRNY |
| 13 | SRP1848-B03 | CDR-H1 | Chothia | GFNISMK |
| 14 | SRP1848-B04 | CDR-H1 | Chothia | SFNISNH |
| 15 | SRP1848-B05 | CDR-H1 | Chothia | GFNISNY |
| 16 | SRP1848-B06 | CDR-H1 | Chothia | GFNISNY |
| 17 | SRP1848-B07 | CDR-H1 | Chothia | GFNISRF |
| 18 | SRP1848-B09 | CDR-H1 | Chothia | GFNITNY |
| 19 | SRP1848-B10 | CDR-H1 | Chothia | GFNTTTK |
| 20 | SRP1848-B11 | CDR-H1 | Chothia | GFNIGNN |
| 21 | SRP1848-C01 | CDR-H1 | Chothia | GFNIGNS |
| 22 | SRP1848-C03 | CDR-H1 | Chothia | GFNIGVY |
| 23 | SRP1848-C04 | CDR-H1 | Chothia | GFNIRHY |
| 24 | SRP1848-C05 | CDR-H1 | Chothia | GFNIRKY |
| 25 | SRP1848-C07 | CDR-H1 | Chothia | GFNIRKY |
| 26 | SRP1848-C10 | CDR-H1 | Chothia | GFNIRTY |
| 27 | SRP1848-D02 | CDR-H1 | Chothia | GFNISHN |
| 28 | SRP1848-D03 | CDR-H1 | Chothia | GFNIRYF |
| 29 | SRP1848-D04 | CDR-H1 | Chothia | GFNISHY |
| 30 | SRP1848-D05 | CDR-H1 | Chothia | GFNISIS |
| 31 | SRP1848-D07 | CDR-H1 | Chothia | GFNISKY |
| 32 | SRP1848-D09 | CDR-H1 | Chothia | GFNISNY |
| 33 | SRP1848-D10 | CDR-H1 | Chothia | GFNISRN |
| 34 | SRP1848-E01 | CDR-H1 | Chothia | GFNITNK |
| 35 | SRP1848-E02 | CDR-H1 | Chothia | GFNIGKY |
| 36 | SRP1848-E03 | CDR-H1 | Chothia | GFNIGNY |
| 37 | SRP1848-E05 | CDR-H1 | Chothia | GFNIGVY |
| 38 | SRP1848-E06 | CDR-H1 | Chothia | GFNINRY |
| 39 | SRP1848-E07 | CDR-H1 | Chothia | GFNIRKS |
| 40 | SRP1848-F01 | CDR-H1 | Chothia | GFNIRTY |
| 41 | SRP1848-F02 | CDR-H1 | Chothia | GFNIRTY |
| 42 | SRP1848-F04 | CDR-H1 | Chothia | GFNISNY |
| 43 | SRP1848-F05 | CDR-H1 | Chothia | GFNISKS |
| 44 | SRP1848-F06 | CDR-H1 | Chothia | GFNISLS |
| 45 | SRP1848-F07 | CDR-H1 | Chothia | GFNISNH |
| 46 | SRP1848-F08 | CDR-H1 | Chothia | GFNISNH |
| 47 | SRP1848-F09 | CDR-H1 | Chothia | GFNISNH |
| 48 | SRP1848-F10 | CDR-H1 | Chothia | GFNISNN |
| 49 | SRP1848-F11 | CDR-H1 | Chothia | GFNISNN |
| 50 | SRP1848-G01 | CDR-H1 | Chothia | GFNISRH |
| 51 | SRP1848-G03 | CDR-H1 | Chothia | GFNISTY |
| 52 | SRP1848-G04 | CDR-H1 | Chothia | GFNIHST |
| 53 | SRP1848-G06 | CDR-H1 | Chothia | GFNIRST |
| 54 | SRP1848-G07 | CDR-H1 | Chothia | GFNIHST |
| 55 | SRP1848-G09 | CDR-H1 | Chothia | GFNIRGT |
| 56 | SRP1848-G10 | CDR-H1 | Chothia | GFNIRST |
| 57 | SRP1848-G11 | CDR-H1 | Chothia | GFNISST |
| 58 | SRP1848-H01 | CDR-H1 | Chothia | GFNIRTQ |
| 59 | SRP2060-E10 | CDR-H1 | Chothia | GFSLSTFGM |
| 60 | SRP2060-E05 | CDR-H1 | Chothia | GFSLSTFGM |
| 61 | SRP2060-B01 | CDR-H1 | Chothia | GFSLSTFGM |
| 62 | SRP2060-A06 | CDR-H1 | Chothia | GFSLSTFGM |
| 63 | SRP1848-A01 | CDR-H1 | Kabat | RYSIH |
| 64 | SRP1848-A02 | CDR-H1 | Kabat | GFRIH |
| 65 | SRP1848-A04 | CDR-H1 | Kabat | QSSIH |
| 66 | SRP1848-A06 | CDR-H1 | Kabat | NSYIH |
| 67 | SRP1848-A07 | CDR-H1 | Kabat | YHSIH |
| 68 | SRP1848-A08 | CDR-H1 | Kabat | KHSIH |
| 69 | SRP1848-A09 | CDR-H1 | Kabat | KQSIH |
| 70 | SRP1848-A10 | CDR-H1 | Kabat | KYSIH |
| 71 | SRP1848-B01 | CDR-H1 | Kabat | NYSIH |
| 72 | SRP1848-B03 | CDR-H1 | Kabat | MKYIH |
| 73 | SRP1848-B04 | CDR-H1 | Kabat | NHSIH |
| 74 | SRP1848-B05 | CDR-H1 | Kabat | NYYIH |
| 75 | SRP1848-B06 | CDR-H1 | Kabat | NYYIH |
| 76 | SRP1848-B07 | CDR-H1 | Kabat | RFYIH |
| 77 | SRP1848-B09 | CDR-H1 | Kabat | NYYIH |
| 78 | SRP1848-B10 | CDR-H1 | Kabat | TKSIH |
| 79 | SRP1848-B11 | CDR-H1 | Kabat | NNSIH |
| 80 | SRP1848-C01 | CDR-H1 | Kabat | NSYIH |
| 81 | SRP1848-C03 | CDR-H1 | Kabat | VYSIH |
| 82 | SRP1848-C04 | CDR-H1 | Kabat | HYSIH |
| 83 | SRP1848-C05 | CDR-H1 | Kabat | KYSIH |
| 84 | SRP1848-C07 | CDR-H1 | Kabat | KYSIH |
| 85 | SRP1848-C10 | CDR-H1 | Kabat | TYYIH |
| 86 | SRP1848-D02 | CDR-H1 | Kabat | HNYIH |
| 87 | SRP1848-D03 | CDR-H1 | Kabat | YFSIH |
| 88 | SRP1848-D04 | CDR-H1 | Kabat | HYSIH |
| 89 | SRP1848-D05 | CDR-H1 | Kabat | ISYIH |
| 90 | SRP1848-D07 | CDR-H1 | Kabat | KYYIH |
| 91 | SRP1848-D09 | CDR-H1 | Kabat | NYYIH |
| 92 | SRP1848-D10 | CDR-H1 | Kabat | RNSIH |
| 93 | SRP1848-E01 | CDR-H1 | Kabat | NKYIH |
| 94 | SRP1848-E02 | CDR-H1 | Kabat | KYSIH |
| 95 | SRP1848-E03 | CDR-H1 | Kabat | NYYIH |
| 96 | SRP1848-E05 | CDR-H1 | Kabat | VYYIH |
| 97 | SRP1848-E06 | CDR-H1 | Kabat | RYYIH |
| 98 | SRP1848-E07 | CDR-H1 | Kabat | KSSIH |
| 99 | SRP1848-F01 | CDR-H1 | Kabat | TYSIH |
| 100 | SRP1848-F02 | CDR-H1 | Kabat | TYSIH |
| 101 | SRP1848-F04 | CDR-H1 | Kabat | NYSIH |
| 102 | SRP1848-F05 | CDR-H1 | Kabat | KSSIH |
| 103 | SRP1848-F06 | CDR-H1 | Kabat | LSYIH |
| 104 | SRP1848-F07 | CDR-H1 | Kabat | NHSIH |
| 105 | SRP1848-F08 | CDR-H1 | Kabat | NHSIH |
| 106 | SRP1848-F09 | CDR-H1 | Kabat | NHYIH |
| 107 | SRP1848-F10 | CDR-H1 | Kabat | NNSIH |
| 108 | SRP1848-F11 | CDR-H1 | Kabat | NNYIH |
| 109 | SRP1848-G01 | CDR-H1 | Kabat | RHSIH |
| 110 | SRP1848-G03 | CDR-H1 | Kabat | TYYIH |
| 111 | SRP1848-G04 | CDR-H1 | Kabat | STDIH |
| 112 | SRP1848-G06 | CDR-H1 | Kabat | STDIH |
| 113 | SRP1848-G07 | CDR-H1 | Kabat | STDIH |
| 114 | SRP1848-G09 | CDR-H1 | Kabat | GTDIH |
| 115 | SRP1848-G10 | CDR-H1 | Kabat | STDIH |
| 116 | SRP1848-G11 | CDR-H1 | Kabat | STDIH |
| 117 | SRP1848-H01 | CDR-H1 | Kabat | TQSIH |
| 118 | SRP2060-E10 | CDR-H1 | Kabat | TFGMGVG |
| 119 | SRP2060-E05 | CDR-H1 | Kabat | TFGMGVG |
| 120 | SRP2060-B01 | CDR-H1 | Kabat | TFGMGVG |
| 121 | SRP2060-A06 | CDR-H1 | Kabat | TFGMGVG |
| 122 | SRP1848-A01 | CDR-H2 | Chothia | LPESGG |
| 123 | SRP1848-A02 | CDR-H2 | Chothia | YPESGA |
| 124 | SRP1848-A04 | CDR-H2 | Chothia | YPVDGT |
| 125 | SRP1848-A06 | CDR-H2 | Chothia | TPIDGN |
| 126 | SRP1848-A07 | CDR-H2 | Chothia | FPVDGT |
| 127 | SRP1848-A08 | CDR-H2 | Chothia | YPNDGT |
| 128 | SRP1848-A09 | CDR-H2 | Chothia | FPNDGT |
| 129 | SRP1848-A10 | CDR-H2 | Chothia | FPIDDI |
| 130 | SRP1848-B01 | CDR-H2 | Chothia | YPVDGI |
| 131 | SRP1848-B03 | CDR-H2 | Chothia | TPIDGM |
| 132 | SRP1848-B04 | CDR-H2 | Chothia | YPVDGI |
| 133 | SRP1848-B05 | CDR-H2 | Chothia | SPIDGY |
| 134 | SRP1848-B06 | CDR-H2 | Chothia | TPIDGY |
| 135 | SRP1848-B07 | CDR-H2 | Chothia | SPYDGF |
| 136 | SRP1848-B09 | CDR-H2 | Chothia | TPVDGY |
| 137 | SRP1848-B10 | CDR-H2 | Chothia | YPRDGI |
| 138 | SRP1848-B11 | CDR-H2 | Chothia | SPIDGF |
| 139 | SRP1848-C01 | CDR-H2 | Chothia | TPNDGY |
| 140 | SRP1848-C03 | CDR-H2 | Chothia | YPIDGN |
| 141 | SRP1848-C04 | CDR-H2 | Chothia | YPGPGN |
| 142 | SRP1848-C05 | CDR-H2 | Chothia | FPIDGI |
| 143 | SRP1848-C07 | CDR-H2 | Chothia | FPIDGI |
| 144 | SRP1848-C10 | CDR-H2 | Chothia | SPIDGY |
| 145 | SRP1848-D02 | CDR-H2 | Chothia | TPQDGY |
| 146 | SRP1848-D03 | CDR-H2 | Chothia | FPNDGS |
| 147 | SRP1848-D04 | CDR-H2 | Chothia | YPRDGI |
| 148 | SRP1848-D05 | CDR-H2 | Chothia | SPIDGY |
| 149 | SRP1848-D07 | CDR-H2 | Chothia | SPNDGY |
| 150 | SRP1848-D09 | CDR-H2 | Chothia | SPNDGY |
| 151 | SRP1848-D10 | CDR-H2 | Chothia | SPNDGT |
| 152 | SRP1848-E01 | CDR-H2 | Chothia | TPFDGF |
| 153 | SRP1848-E02 | CDR-H2 | Chothia | YPNDGN |
| 154 | SRP1848-E03 | CDR-H2 | Chothia | TPRDGF |
| 155 | SRP1848-E05 | CDR-H2 | Chothia | TPNDGY |
| 156 | SRP1848-E06 | CDR-H2 | Chothia | TPNDGY |
| 157 | SRP1848-E07 | CDR-H2 | Chothia | FPYDGS |
| 158 | SRP1848-F01 | CDR-H2 | Chothia | FPNDGT |
| 159 | SRP1848-F02 | CDR-H2 | Chothia | FPNDGT |
| 160 | SRP1848-F04 | CDR-H2 | Chothia | YPIDGI |
| 161 | SRP1848-F05 | CDR-H2 | Chothia | YPNDGS |
| 162 | SRP1848-F06 | CDR-H2 | Chothia | SPIDGN |
| 163 | SRP1848-F07 | CDR-H2 | Chothia | YPNDGI |
| 164 | SRP1848-F08 | CDR-H2 | Chothia | YPVDGI |
| 165 | SRP1848-F09 | CDR-H2 | Chothia | SPLDGY |
| 166 | SRP1848-F10 | CDR-H2 | Chothia | FPNDGY |
| 167 | SRP1848-F11 | CDR-H2 | Chothia | TPIDGN |
| 168 | SRP1848-G01 | CDR-H2 | Chothia | APNDGS |
| 169 | SRP1848-G03 | CDR-H2 | Chothia | TPSDGF |
| 170 | SRP1848-G04 | CDR-H2 | Chothia | TPAGGA |
| 171 | SRP1848-G06 | CDR-H2 | Chothia | TPAGGA |
| 172 | SRP1848-G07 | CDR-H2 | Chothia | TPAGGA |
| 173 | SRP1848-G09 | CDR-H2 | Chothia | TPAGGA |
| 174 | SRP1848-G10 | CDR-H2 | Chothia | TPAGGA |
| 175 | SRP1848-G11 | CDR-H2 | Chothia | TPAGGA |
| 176 | SRP1848-H01 | CDR-H2 | Chothia | FPIDGI |
| 177 | SRP2060-E10 | CDR-H2 | Chothia | WWDDD |
| 178 | SRP2060-E05 | CDR-H2 | Chothia | WWDDD |
| 179 | SRP2060-B01 | CDR-H2 | Chothia | WWDDD |
| 180 | SRP2060-A06 | CDR-H2 | Chothia | WWDDD |
| 181 | SRP1848-A01 | CDR-H2 | Kabat | GILPESGGTSYADSVKG |
| 182 | SRP1848-A02 | CDR-H2 | Kabat | GIYPESGATYYADSVKG |
| 183 | SRP1848-A04 | CDR-H2 | Kabat | VIYPVDGTTDYADSVKG |
| 184 | SRP1848-A06 | CDR-H2 | Kabat | GITPIDGNTDYADSVKG |
| 185 | SRP1848-A07 | CDR-H2 | Kabat | EIFPVDGTTDYADSVKG |
| 186 | SRP1848-A08 | CDR-H2 | Kabat | SIYPNDGTTDYADSVKG |
| 187 | SRP1848-A09 | CDR-H2 | Kabat | SIFPNDGTTDYADSVKG |
| 188 | SRP1848-A10 | CDR-H2 | Kabat | DIFPIDDITDYADSVKG |
| 189 | SRP1848-B01 | CDR-H2 | Kabat | EIYPVDGITDYADSVKG |
| 190 | SRP1848-B03 | CDR-H2 | Kabat | GITPIDGMTDYADSVKG |
| 191 | SRP1848-B04 | CDR-H2 | Kabat | EIYPVDGITDYADSVKG |
| 192 | SRP1848-B05 | CDR-H2 | Kabat | GISPIDGYTDYADSMKG |
| 193 | SRP1848-B06 | CDR-H2 | Kabat | GITPIDGYTDYADSVKG |
| 194 | SRP1848-B07 | CDR-H2 | Kabat | GISPYDGFTDYADSVKG |
| 195 | SRP1848-B09 | CDR-H2 | Kabat | GITPVDGYTDYADRVKG |
| 196 | SRP1848-B10 | CDR-H2 | Kabat | EIYPRDGITDYADSVKG |
| 197 | SRP1848-B11 | CDR-H2 | Kabat | DISPIDGFTDYADSVKG |
| 198 | SRP1848-C01 | CDR-H2 | Kabat | GVTPNDGYTDYADSVKG |
| 199 | SRP1848-C03 | CDR-H2 | Kabat | EIYPIDGNTDYADSVKG |
| 200 | SRP1848-C04 | CDR-H2 | Kabat | EIYPGPGNTDYADSVKG |
| 201 | SRP1848-C05 | CDR-H2 | Kabat | DIFPIDGINDYADSVKG |
| 202 | SRP1848-C07 | CDR-H2 | Kabat | DIFPIDGITDYADSVKG |
| 203 | SRP1848-C10 | CDR-H2 | Kabat | GISPIDGYTDYADSMKG |
| 204 | SRP1848-D02 | CDR-H2 | Kabat | GITPQDGYTDYADSVKG |
| 205 | SRP1848-D03 | CDR-H2 | Kabat | DIFPNDGSTDYADSVKG |
| 206 | SRP1848-D04 | CDR-H2 | Kabat | EIYPRDGITDYADSVKG |
| 207 | SRP1848-D05 | CDR-H2 | Kabat | GISPIDGYTDYADSVKG |
| 208 | SRP1848-D07 | CDR-H2 | Kabat | GISPNDGYTDYADSVKG |
| 209 | SRP1848-D09 | CDR-H2 | Kabat | GISPNDGYTDYADSVKG |
| 210 | SRP1848-D10 | CDR-H2 | Kabat | WISPNDGTTDYADSVKG |
| 211 | SRP1848-E01 | CDR-H2 | Kabat | GITPFDGFTDYADSVKG |
| 212 | SRP1848-E02 | CDR-H2 | Kabat | EIYPNDGNTDYADSVKG |
| 213 | SRP1848-E03 | CDR-H2 | Kabat | GITPRDGFTDYADSVKG |
| 214 | SRP1848-E05 | CDR-H2 | Kabat | GITPNDGYTDYADSVKG |
| 215 | SRP1848-E06 | CDR-H2 | Kabat | GITPNDGYTDYADSVEG |
| 216 | SRP1848-E07 | CDR-H2 | Kabat | EIFPYDGSTDYADNVKG |
| 217 | SRP1848-F01 | CDR-H2 | Kabat | SIFPNDGTTDYADSVKG |
| 218 | SRP1848-F02 | CDR-H2 | Kabat | SIFPNDGTTDYADSVKG |
| 219 | SRP1848-F04 | CDR-H2 | Kabat | EIYPIDGITDYADSVKG |
| 220 | SRP1848-F05 | CDR-H2 | Kabat | EIYPNDGSTDYADSVKG |
| 221 | SRP1848-F06 | CDR-H2 | Kabat | GISPIDGNTDYADSVKG |
| 222 | SRP1848-F07 | CDR-H2 | Kabat | EIYPNDGITDYADSVKG |
| 223 | SRP1848-F08 | CDR-H2 | Kabat | EIYPVDGITDYADSVKG |
| 224 | SRP1848-F09 | CDR-H2 | Kabat | GISPLDGYTDYADSVKG |
| 225 | SRP1848-F10 | CDR-H2 | Kabat | SIFPNDGYTDYADSVKG |
| 226 | SRP1848-F11 | CDR-H2 | Kabat | GITPIDGNTDYADSVKG |
| 227 | SRP1848-G01 | CDR-H2 | Kabat | WIAPNDGSTDYADSVKG |
| 228 | SRP1848-G03 | CDR-H2 | Kabat | GITPSDGFTDYADSVKG |
| 229 | SRP1848-G04 | CDR-H2 | Kabat | YITPAGGATFYADSVKG |
| 230 | SRP1848-G06 | CDR-H2 | Kabat | YITPAGGATYYADNVKG |
| 231 | SRP1848-G07 | CDR-H2 | Kabat | YITPAGGATWYADSVKG |
| 232 | SRP1848-G09 | CDR-H2 | Kabat | YITPAGGATFYADSVKG |
| 233 | SRP1848-G10 | CDR-H2 | Kabat | YITPAGGATYYADSVKG |
| 234 | SRP1848-G11 | CDR-H2 | Kabat | YITPAGGATWYADSVKG |
| 235 | SRP1848-H01 | CDR-H2 | Kabat | DIFPIDGITDYADSVKG |
| 236 | SRP2060-E10 | CDR-H2 | Kabat | HIWWDDDKYYHPALKG |
| 237 | SRP2060-E05 | CDR-H2 | Kabat | HIWWDDDKYYHPALKG |
| 238 | SRP2060-B01 | CDR-H2 | Kabat | HIWWDDDKYYHPALKG |
| 239 | SRP2060-A06 | CDR-H2 | Kabat | HIWWDDDKYYYPALKG |
| 240 | SRP1848-A01 | CDR-H3 | | HIYPWDWFSNYVLDY |
| 241 | SRP1848-A02 | CDR-H3 | | HLYVWDWVLDHVLDY |
| 242 | SRP1848-A04 | CDR-H3 | | GAWSWRSGYGYYIDY |
| 243 | SRP1848-A06 | CDR-H3 | | GAWSWRSGYGYYIDY |
| 244 | SRP1848-A07 | CDR-H3 | | GFWAWRSGYGYYLDY |
| 245 | SRP1848-A08 | CDR-H3 | | GSWFWRAGYGYYLDY |
| 246 | SRP1848-A09 | CDR-H3 | | GSWFWRSGYGYFLEY |
| 247 | SRP1848-A10 | CDR-H3 | | GSWSWPSGHSYYLDY |
| 248 | SRP1848-B01 | CDR-H3 | | GFWSWPSGYSYFLDY |
| 249 | SRP1848-B03 | CDR-H3 | | GSWSWPSGYSYYLDY |
| 250 | SRP1848-B04 | CDR-H3 | | GRYSWRAGYSYYLDY |
| 251 | SRP1848-B05 | CDR-H3 | | GSWFWQSGYGYYLDY |
| 252 | SRP1848-B06 | CDR-H3 | | GFWSWPSGYGYYQDY |
| 253 | SRP1848-B07 | CDR-H3 | | GSWSWPAGYGYYQDY |
| 254 | SRP1848-B09 | CDR-H3 | | GAWSWRSGYGYYMDY |
| 255 | SRP1848-B10 | CDR-H3 | | GGWHWRSGYSYYLDY |
| 256 | SRP1848-B11 | CDR-H3 | | GSWSWRAGYGYYLDY |
| 257 | SRP1848-C01 | CDR-H3 | | GSWFWRAGYGYYLDY |
| 258 | SRP1848-C03 | CDR-H3 | | GSWAWRSGYSYYLDY |
| 259 | SRP1848-C04 | CDR-H3 | | GSLSWRAGYGYYLDY |
| 260 | SRP1848-C05 | CDR-H3 | | GSWSWKAGYGYYLDY |
| 261 | SRP1848-C07 | CDR-H3 | | GSWSWPAGYGYYQDY |
| 262 | SRP1848-C10 | CDR-H3 | | GSWSWPAGYGYYLDY |
| 263 | SRP1848-D02 | CDR-H3 | | GAWSWRAGYGYYLDY |
| 264 | SRP1848-D03 | CDR-H3 | | GHWSWPSGYWYYLDY |
| 265 | SRP1848-D04 | CDR-H3 | | GYWFWRSGYGYYLDY |
| 266 | SRP1848-D05 | CDR-H3 | | GSWSWRAGYGYYLDY |
| 267 | SRP1848-D07 | CDR-H3 | | GFWAWRSGYGYYLDY |
| 268 | SRP1848-D09 | CDR-H3 | | GSWSWRHGYGYYLDY |
| 269 | SRP1848-D10 | CDR-H3 | | GAWSWRSGYGYYIDY |
| 270 | SRP1848-E01 | CDR-H3 | | GSWSWPAGYGYYQDY |
| 271 | SRP1848-E02 | CDR-H3 | | GSWSWRSGYGYYLDY |
| 272 | SRP1848-E03 | CDR-H3 | | GSWSWPAGHSYYLDY |
| 273 | SRP1848-E05 | CDR-H3 | | GFWAWRSGYGYYLDY |
| 274 | SRP1848-E06 | CDR-H3 | | GTWSWPSGHSYYLDY |
| 275 | SRP1848-E07 | CDR-H3 | | GAWSWRSGYGYYIDY |
| 276 | SRP1848-F01 | CDR-H3 | | GSWAWRAGYSYYLDY |
| 277 | SRP1848-F02 | CDR-H3 | | GSWSWQAGYGYYLDY |
| 278 | SRP1848-F04 | CDR-H3 | | GSWFWRSGYGYYLDY |
| 279 | SRP1848-F05 | CDR-H3 | | GSWAWRSGYSYFLDY |
| 280 | SRP1848-F06 | CDR-H3 | | GFWAWRSGYGYYLDY |
| 281 | SRP1848-F07 | CDR-H3 | | GSWDWRSGYSYYLDY |
| 282 | SRP1848-F08 | CDR-H3 | | GSWYWQSGYSYYLDY |
| 283 | SRP1848-F09 | CDR-H3 | | GAWSWRSGYGYYIDY |
| 284 | SRP1848-F10 | CDR-H3 | | GSWFWRSGYGYYLDY |
| 285 | SRP1848-F11 | CDR-H3 | | GSWYWRAGYGYYLDY |
| 286 | SRP1848-G01 | CDR-H3 | | GSWAWRSGYSYFLDY |
| 287 | SRP1848-G03 | CDR-H3 | | GSWSWPSGHGYFLDY |
| 288 | SRP1848-G04 | CDR-H3 | | YPYWFAGYMDY |
| 289 | SRP1848-G06 | CDR-H3 | | QPYWFAGYMDY |
| 290 | SRP1848-G07 | CDR-H3 | | YPFWFAGYMDY |
| 291 | SRP1848-G09 | CDR-H3 | | HEYWFSGYMDY |
| 292 | SRP1848-G10 | CDR-H3 | | YPYWFAGYIDY |
| 293 | SRP1848-G11 | CDR-H3 | | YPYWFSGYMDY |
| 294 | SRP1848-H01 | CDR-H3 | | GSWSWPSGMDYYLDY |
| 295 | SRP2060-E10 | CDR-H3 | | NHFPHYYGSSHWYFNV |
| 296 | SRP2060-E05 | CDR-H3 | | NHFPHYYGSSHWYFNV |
| 297 | SRP2060-B01 | CDR-H3 | | NHFPHYYGSSHWYFNV |
| 298 | SRP2060-A06 | CDR-H3 | | NHFPHYYGSSHWYFDV |
| 299 | trastuzumab | CDR-L1 | | RASQDVNTAVA |
| 300 | H6D1-LC4 | CDR-L1 | | KASQDINSYLS |
| 301 | H6D1-LC5 | CDR-L1 | | KASQDINSYLS |
| 302 | trastuzumab | CDR-L2 | | SASFLYS |
| 303 | H6D1-LC4 | CDR-L2 | | RANRLVD |
| 304 | H6D1-LC5 | CDR-L2 | | RANRLVD |
| 305 | trastuzumab | CDR-L3 | | QQHYTTPPT |
| 306 | H6D1-LC4 | CDR-L3 | | LQYDEFPYT |
| 307 | H6D1-LC5 | CDR-L3 | | LQYDEFPYT |
| 308 | SRP1848-A01 | VH | | EVQLVESGGGLVQPGGSLRLSCAA |
| | | | | SGFNITRYSIHWVRQAPGKGLEWV |
| | | | | AGILPESGGTSYADSVKGRFTISA |
| | | | | DTSKNTAYLQMNSLRAEDTAVYYC |
| | | | | ARHIYPWDWFSNYVLDYWGQGTLV |
| | | | | TVSS |
| 309 | SRP1848-A02 | VH | | EVQLVESGGGLVQPGGSLRLSCAA |
| | | | | SGFNISGFRIHWVRQAPGKGLEWV |
| | | | | AGIYPESGATYYADSVKGRFTISA |
| | | | | DTSKNTAYLQMNSLRAEDTAVYYC |
| | | | | ARHLYVWDWVLDHVLDYWGQGTLV |
| | | | | TVSS |
| 310 | SRP1848-A04 | VH | | EVQLVESGGGLVQPGGSLRLSCAA |
| | | | | SGFNIDQSSIHWVRQAPGKGLEWV |
| | | | | GVIYPVDGTTDYADSVKGRFTISA |
| | | | | DTSKNTAYLQMNSLRAEDTAVYYC |
| | | | | ARGAWSWRSGYGYYIDYWGQGTLV |
| | | | | TVSS |
| 311 | SRP1848-A06 | VH | | EVQLVESGGGLVQPGGSLRLSCAA |
| | | | | SGFNIGNSYIHWVRQAPGKGLEWV |
| | | | | GGITPIDGNTDYADSVKGRFTISA |
| | | | | DTSKNTAYLQMNSLRAEDTAVYYC |
| | | | | ARGAWSWRSGYGYYIDYWGQGTLV |
| | | | | TVSS |
| 312 | SRP1848-A07 | VH | | EVQLVESGGGLVQPGGSLRLSCAA |
| | | | | SGFNIGYHSIHWVRQAPGKGLEWV |
| | | | | GEIFPVDGTTDYADSVKGRFTISA |
| | | | | DTSKNTAYLHMNSLRAEDTAVYYC |
| | | | | ARGFWAWRSGYGYYLDYWGQGTLV |
| | | | | TVSS |
| 313 | SRP1848-A08 | VH | | EVQLVESGGGLVQPGGSLRLSCAA |
| | | | | SGSNIRKHSIHWVRQAPGKGLEWV |
| | | | | GSIYPNDGTTDYADSVKGRFTISA |
| | | | | DTSKNTAYLQMNSLRAEDTAVYYC |
| | | | | ARGSWFWRAGYGYYLDYWGQGTLV |
| | | | | TVSS |
| 314 | SRP1848-A09 | VH | | EVQLVESGGGLVQPGGSLRLSCAA |
| | | | | SGFNIRKQSIHWVRQAPGKGLEWV |
| | | | | GSIFPNDGTTDYADSVKGRFTISA |
| | | | | DTSKNTAYLQVNSLRAEDTAVYYC |
| | | | | ARGSWFWRSGYGYFLEYWGQGTLV |
| | | | | TVSS |
| 315 | SRP1848-A10 | VH | | EVQLVESGGGLVQPGGSLRLSCAA |
| | | | | SGFNIRKYSIHWARQAPGKGLEWV |
| | | | | GDIFPIDDITDYADSVKGRFTISA |
| | | | | DTSKNTAYLQMNSLRAEDTAVYYC |
| | | | | ARGSWSWPSGHSYYLDYWGQGTLV |
| | | | | TVSS |
| 316 | SRP1848-B01 | VH | | EVQLVESGGGLVQPGGSLRLSCAA |
| | | | | SGFNIRNYSIHWVRQAPGKGLEWV |
| | | | | GEIYPVDGITDYADSVKGRFTISA |
| | | | | DTSKNTAYLQMNSLRAEDTAVYYC |
| | | | | ARGFWSWPSGYSYFLDYWGQGTLV |
| | | | | TVSS |
| 317 | SRP1848-B03 | VH | | EVQLVESGGGLVQPGGSLRLSCAA |
| | | | | SGFNISMKYIHWVRQAPGKGLEWV |
| | | | | GGITPIDGMTDYADSVKGRFTISA |
| | | | | DTSKNTAYLQMNSLRAEDTAVYYC |
| | | | | ARGSWSWPSGYSYYLDYWGQGTLV |
| | | | | TVSS |
| 318 | SRP1848-B04 | VH | | EVQLVESGGGLVQPGGSLRLSCAA |
| | | | | SSFNISNHSIHWVRQAPGKGLEWV |
| | | | | GEIYPVDGITDYADSVKGRFTISA |
| | | | | DTSKNTAYLQMNSLRAEDTAVYYC |
| | | | | ARGRYSWRAGYSYYLDYWGQGTLV |
| | | | | TVSS |
| 319 | SRP1848-B05 | VH | | EVQLVESGGGLVQPGGSLRLSCAA |
| | | | | SGFNISNYYIHWVRQAPGKGLEWV |
| | | | | GGISPIDGYTDYADSMKGRFTISA |
| | | | | DTSKNTAYLQMSSLRAEDTAVYYC |
| | | | | ARGSWFWQSGYGYYLDYWGQGTLV |
| | | | | TVSS |
| 320 | SRP1848-B06 | VH | | EVQLVESGGGLVQPGGSLRLSCAA |
| | | | | SGFNISNYYIHWVRQAPGKGLEWV |
| | | | | GGITPIDGYTDYADSVKGRFTISA |
| | | | | DTSKNTAYLQMNSLRAEDTAVYYC |
| | | | | ARGFWSWPSGYGYYQDYWGQGTLV |
| | | | | TVSS |
| 321 | SRP1848-B07 | VH | | EVQLVESGGGLVQPGGSLRLSCAA |
| | | | | SGFNISRFYIHWVRQAPGKGLEWV |
| | | | | GGISPYDGFTDYADSVKGRFTISA |
| | | | | DTSKNTAYLQMNSLRAEDTAVYYC |
| | | | | ARGSWSWPAGYGYYQDYWGQGTLV |
| | | | | TVSS |
| 322 | SRP1848-B09 | VH | | EVQLVESGGGLVQPGGSLRLSCAA |
| | | | | GGFNITNYYIHWVRQAPGKGLEWV |
| | | | | GGITPVDGYTDYADRVKGRFTISA |
| | | | | DTSKNTAYLQMNSLRAEDTAVYYC |
| | | | | ARGAWSWRSGYGYYMDYWGQGTLV |
| | | | | TVSS |
| 323 | SRP1848-B10 | VH | | EVQLVESGGGLVQPGGSLRLSCAA |
| | | | | SGFNTTTKSIHWVRQAPGKGLEWV |
| | | | | GEIYPRDGITDYADSVKGRFTISA |
| | | | | DTSKNTAYLQMNSLRAEDTAVYYC |
| | | | | ARGGWHWRSGYSYYLDYWGQGTLV |
| | | | | TVSS |
| 324 | SRP1848-B11 | VH | | EVQLVESGGGLVQPGGSLRLSCAA |
| | | | | SGFNIGNNSIHWVRQAPGKGLEWV |
| | | | | GDISPIDGFTDYADSVKGRFTISA |
| | | | | DTSKNTAYLQMNSLRAEDTAVYYC |
| | | | | ARGSWSWRAGYGYYLDYWGQGTLV |
| | | | | TVSS |
| 325 | SRP1848-C01 | VH | | EVQLVESGGGLVQPGGSLRLSCAA |
| | | | | SGFNIGNSYIHWVRQAPGKGLEWV |
| | | | | GGVTPNDGYTDYADSVKGRFTISA |
| | | | | DTSKNTTYLQMNSLRAEDTAVYYC |
| | | | | ARGSWFWRAGYGYYLDYWGQGALV |
| | | | | TVSS |
| 326 | SRP1848-C03 | VH | | EVQLVESGGGLVQPGGSLRLSCAA |
| | | | | SGFNIGVYSIHWVRQAPGKGLEWV |
| | | | | GEIYPIDGNTDYADSVKGRFTISA |
| | | | | DTSKNTAYLQMNSLRAEDTAVYYC |
| | | | | ARGSWAWRSGYSYYLDYWGQGTLV |
| | | | | TVSS |
| 327 | SRP1848-C04 | VH | | EVQLVESGGGLVQPGGSLRLSCAA |
| | | | | SGFNIRHYSIHWVRQAPGKGLEWV |
| | | | | GEIYPGPGNTDYADSVKGRFTISA |
| | | | | DTSKNTAYLQMNSLRAEDTAVYYC |
| | | | | ARGSLSWRAGYGYYLDYWGQGTLV |
| | | | | TVSS |
| 328 | SRP1848-C05 | VH | | EVQLVESGGGLVQPGGSLRLSCAA |
| | | | | SGFNIRKYSIHWVRQAPGKGLEWV |
| | | | | GDIFPIDGINDYADSVKGRFTISA |
| | | | | DTSKNTAYLQMNSLRAEDTAVYYC |
| | | | | ARGSWSWKAGYGYYLDYWGQGTLV |
| | | | | TVSS |
| 329 | SRP1848-C07 | VH | | EVQLVESGGGLVQPGGSLRLSCAA |
| | | | | SGFNIRKYSIHWVRQAPGKGLEWV |
| | | | | GDIFPIDGITDYADSMKGRFTISA |
| | | | | DTSKNTAYLQMNSLRAEDTAVYYC |
| | | | | ARGSWSWPAGYGYYQDYWGQGTLV |
| | | | | TVSS |
| 330 | SRP1848-C10 | VH | | EVQLVESGGGLVQPGGSLRLSCAA |
| | | | | SGFNIRTYYIHWVRQAPGKGLEWV |
| | | | | GGISPIDGYTDYADSVKGRFTISA |
| | | | | DTSKNTAYLQMNSLRAEDTAVYYC |
| | | | | ARGSWSWPAGYGYYLDYWGQGTLV |
| | | | | TVSS |
| 331 | SRP1848-D02 | VH | | EVQLVESGGGLVQPGGSLRLSCAA |
| | | | | SGFNISHNYIHWVRQAPGKGLEWV |
| | | | | GGITPQDGYTDYADSVKGRFTISA |
| | | | | DTSKNTAYLQMNRLRAEDTAVYYC |
| | | | | ARGAWSWRAGYGYYLDYWGQGTLV |
| | | | | TVSS |
| 332 | SRP1848-D03 | VH | | EVQLVESGGGVVQPGGSLRLSCAA |
| | | | | SGFNIRYFSIHWVRQAPGKGLEWV |
| | | | | GDIFPNDGSTDYADSVKGRFTISA |
| | | | | DTSKNTAYLQMNSLRAEETAVYYC |
| | | | | ARGHWSWPSGYWYYLDYWGQGTLV |
| | | | | TVSS |
| 333 | SRP1848-D04 | VH | | EVQLVESGGGLVQPGGSLRLSCAA |
| | | | | SGFNISHYSIHWVRQAPGKGLEWV |
| | | | | GEIYPRDGITDYADSVKGRFTISA |
| | | | | DTSKNTAYLQMNSLSAEDTAVYYC |
| | | | | ARGYWFWRSGYGYYLDYWGQGTLV |
| | | | | TVSS |
| 334 | SRP1848-D05 | VH | | EVQLVESGGGLVQPGGSLRLSCAA |
| | | | | SGFNISISYIHWVRQAPGKGLEWV |
| | | | | GGISPIDGYTDYADSVKGRFTISA |
| | | | | DTSKNTAYLQMNSLRAEDTAVYYC |
| | | | | ARGSWSWRAGYGYYLDYWGQGTLV |
| | | | | TVSS |
| 335 | SRP1848-D07 | VH | | EVQLVESGGGLVQPGGSLRLSCAA |
| | | | | SGFNISKYYIHWVRQAPGKGLEWV |
| | | | | GGISPNDGYTDYADSVKGRFAISA |
| | | | | DTSKNTAYLQMNSLRAEDTAVYYC |
| | | | | ARGFWAWRSGYGYYLDYWGQGTLV |
| | | | | TVSS |
| 336 | SRP1848-D09 | VH | | EVQLVESGGGLVQPGGSLRLSCAA |
| | | | | SGFNISNYYIHWVRQAPGKGLEWV |
| | | | | GGISPNDGYTDYADSVKGRFTISA |
| | | | | DTSKNTAYLQMNSLRAEDTAVYYC |
| | | | | ARGSWSWRHGYGYYLDYWGQGTLV |
| | | | | TVSS |
| 337 | SRP1848-D10 | VH | | EVQLVESGGGLVQPGGSLRLSCAA |
| | | | | SGFNISRNSIHWVRQAPGKGLEWV |
| | | | | GWISPNDGTTDYADSVKGRFTISA |
| | | | | DGSKNTAYLQMNSLRAEDTAVYYC |
| | | | | ARGAWSWRSGYGYYIDYWGQGTLV |
| | | | | TVSS |
| 338 | SRP1848-E01 | VH | | EVQLVESGGGLVQPGGSLRLSCAA |
| | | | | SGFNITNKYIHWVRQAPGKGLEWV |
| | | | | GGITPFDGFTDYADSVKGRFTISA |
| | | | | DTSKNTAYLQMNSLRAEDTAVYYC |
| | | | | ARGSWSWPAGYGYYQDYWGQGTLV |
| | | | | TVSS |
| 339 | SRP1848-E02 | VH | | EVQLVESGGGLVQPGGSLRLSCAA |
| | | | | SGFNIGKYSIHWVRQAPGKGLEWV |
| | | | | GEIYPNDGNTDYADSVKGRFTISA |
| | | | | DTSKNTAYLQMNSLRAEDTAVYYC |
| | | | | ARGSWSWRSGYGYYLDYWGQGTLV |
| | | | | TVSS |
| 340 | SRP1848-E03 | VH | | EVQLVESGGGLAQPGGSLRLSCAA |
| | | | | SGFNIGNYYIHWVRQAPGKGLEWV |
| | | | | GGITPRDGFTDYADSVKGRFTISA |
| | | | | DTSKNTAYLQVNSLRAEDTAVYYC |
| | | | | ARGSWSWPAGHSYYLDYWGQGTLV |
| | | | | TVSS |
| 341 | SRP1848-E05 | VH | | EVQLVESGGGLVQPGGSLRVSCAA |
| | | | | SGFNIGVYYIHWVRQAPGKGLEWV |
| | | | | GGITPNDGYTDYADSVKGRFTISA |
| | | | | DTSKNTAYLQMNSLRAEDTAVYYC |
| | | | | ARGFWAWRSGYGYYLDYWGQGTLV |
| | | | | TVSS |
| 342 | SRP1848-E06 | VH | | EVQLVESGGGLVQPSGSLRLSCAA |
| | | | | SGFNINRYYIHWVRQAPGKGLEWV |
| | | | | GGITPNDGYTDYADSVEGRFTTSA |
| | | | | DTSKNTAYLQMNSLRAEDTAVYYC |
| | | | | ARGTWSWPSGHSYYLDYWGQGTLV |
| | | | | TVSS |
| 343 | SRP1848-E07 | VH | | EVQLVESGGGLVQPGGSLRLSCAA |
| | | | | SGFNIRKSSIHWVRQAPGKGLEWV |
| | | | | GEIFPYDGSTDYADNVKGRFTISA |
| | | | | DTSKNTAYLQMNSLRAEDTAVYYC |
| | | | | ARGAWSWRSGYGYYIDYWGQGTLV |
| | | | | TVSS |
| 344 | SRP1848-F01 | VH | | EVQLVESGGGLVQPGGSLRLSCAA |
| | | | | SGFNIRTYSIHWVRQAPGKGLEWV |
| | | | | GSIFPNDGTTDYADSVKGRFTISA |
| | | | | DTSKNTAYLQMNSLRAEDTAVYYC |
| | | | | ARGSWAWRAGYSYYLDYWGQGTLV |
| | | | | TVSS |
| 345 | SRP1848-F02 | VH | | EVQLVESGGGLVQPGGSLRLSCAA |
| | | | | SGFNIRTYSIHWVRQAPGKGLEWV |
| | | | | GSIFPNDGTTDYADSVKGRLTISA |
| | | | | DTSKNTAYLQMNSLRAEDTAVYYC |
| | | | | ARGSWSWQAGYGYYLDYWGQGTLV |
| | | | | TVSS |
| 346 | SRP1848-F04 | VH | | EVQLVESGGGLVQPGGSLRLSCAA |
| | | | | SGFNISNYSIHWVRQAPGKGLEWV |
| | | | | GEIYPIDGITDYADSVKGRFTISA |
| | | | | DTSKNTAYLQMNSLRAEDTAVYYC |
| | | | | ARGSWFWRSGYGYYLDYWGQGTLV |
| | | | | TVSS |
| 347 | SRP1848-F05 | VH | | EVQLVESGGGLVQPGGSLRLSCAA |
| | | | | SGFNISKSSIHWVRQAPGKGLEWV |
| | | | | GEIYPNDGSTDYADSVKGRFTISA |
| | | | | DTSKNTAYLQMNSLRAEDTAVYYC |
| | | | | ARGSWAWRSGYSYFLDYWGQGTLV |
| | | | | TVSS |
| 348 | SRP1848-F06 | VH | | EVQLVESGGGLVQPGGSLRLSCAA |
| | | | | SGFNISLSYIHWVRQAPGKGLEWV |
| | | | | GGISPIDGNTDYADSVKGRFTISA |
| | | | | DTSKNTAYLQMNSLRAEDTAVYYC |
| | | | | ARGFWAWRSGYGYYLDYWGQGTLV |
| | | | | TVSS |
| 349 | SRP1848-F07 | VH | | EVQLVESGGGLVQPGGSLRLSCAA |
| | | | | SGFNISNHSIHWVRQAPGKGLEWV |
| | | | | GEIYPNDGITDYADSVKGRFTISA |
| | | | | DTSKNTAYLQMNSLSAEDTAVYYC |
| | | | | ARGSWDWRSGYSYYLDYWGQGTLV |
| | | | | TVSS |
| 350 | SRP1848-F08 | VH | | EVQLVESGGGLVQPGGSLRLSCAA |
| | | | | GGFNISNHSIHWVRQAPGKGVEWV |
| | | | | GEIYPVDGITDYADSVKGRFTISA |
| | | | | DTSKNTAYLRMNSLRAEDTAVYYC |
| | | | | ARGSWYWQSGYSYYLDYWGQGTLV |
| | | | | TVSS |
| 351 | SRP1848-F09 | VH | | EVQLVESGGGLVQPGGSLRLSCAA |
| | | | | SGFNISNHYIHWVRQAPGKGLEWV |
| | | | | GGISPLDGYTDYADSVKGRFTISA |
| | | | | DTSKNTAYLQMNSLRAEDTAVYYC |
| | | | | ARGAWSWRSGYGYYIDYWGQGTLV |
| | | | | TVSS |
| 352 | SRP1848-F10 | VH | | EVQLVESGGGLVQPGGSLRLSCAA |
| | | | | SGFNISNNSIHWVRQAPGKGLEWV |
| | | | | GSIFPNDGYTDYADSVKGRFTISA |
| | | | | DTSKNTAYLQMNSLRAEDTAVYYC |
| | | | | ARGSWFWRSGYGYYLDYWGQGTLV |
| | | | | TVSS |
| 353 | SRP1848-F11 | VH | | EVQLVESGGGLVQPGGSLRLSCAA |
| | | | | SGFNISNNYIHWVRQAPGKGLEWV |
| | | | | GGITPIDGNTDYADSVKGRFTISA |
| | | | | DTSMNTAYLQMNSLRAEDTAVYYC |
| | | | | ARGSWYWRAGYGYYLDYWGQGALV |
| | | | | TVSS |
| 354 | SRP1848-G01 | VH | | EVQLVESGGGLVQPGGSLRLSCAA |
| | | | | SGFNISRHSIHWVRQAPGKGLEWV |
| | | | | GWIAPNDGSTDYADSVKGRFTISA |
| | | | | DTSKNTAYLQMNSLRAEDTAVYYC |
| | | | | ARGSWAWRSGYSYFLDYWGQGTLV |
| | | | | TVSS |
| 355 | SRP1848-G03 | VH | | EVQLVESGGGLVQPGGSLRLSCAA |
| | | | | SGFNISTYYIHWVRQAPGKGLEWV |
| | | | | GGITPSDGFTDYADSVKGRSTISA |
| | | | | DTSKNTAYLQMNSLRAEDTAVYYC |
| | | | | ARGSWSWPSGHGYFLDYWGQGTLV |
| | | | | TVSS |
| 356 | SRP1848-G04 | VH | | EVQLVESGGGLVQPGGSLRLSCAA |
| | | | | SGFNIHSTDIHWVRQAPGKGLEWV |
| | | | | AYITPAGGATFYADSVKGRFTISA |
| | | | | DTSKNTAYLQMNSLRAEDTAVYYC |
| | | | | ARYPYWFAGYMDYWGQGTLVTVSS |
| 357 | SRP1848-G06 | VH | | EVQLVESGGGLVQPGGSLRLSCAA |
| | | | | SGFNIRSTDIHWVRQAPGKGLEWV |
| | | | | AYITPAGGATYYADNVKGRFTISA |
| | | | | DTSKNTAYLQMNSLRAEDTAVYYC |
| | | | | ARQPYWFAGYMDYWGQGTLVTVSS |
| 358 | SRP1848-G07 | VH | | EVQLVESGGGLVQPGGSLRLSCAA |
| | | | | SGFNIHSTDIHWVRQAPGKGLEWV |
| | | | | AYITPAGGATWYADSVKGRFTISA |
| | | | | DTSKNTAYLQMNSLRAEDTAVYYC |
| | | | | ARYPFWFAGYMDYWGQGTLVTVSS |
| 359 | SRP1848-G09 | VH | | EVQLVESGGGLVQPGGSLRLSCAA |
| | | | | SGFNIRGTDIHWVRQAPGKGLEWV |
| | | | | AYITPAGGATFYADSVKGRFTISA |
| | | | | DTSKNTAYLQMNSLRAEDTAVYYC |
| | | | | ARHEYWFSGYMDYWGQGTLVTVSS |
| 360 | SRP1848-G10 | VH | | EVQLVESGGGLVQPGSSLRLSCAA |
| | | | | SGFNIRSTDIHWVRQAPGKGLEWV |
| | | | | AYITPAGGATYYADSVKGRFTISA |
| | | | | DTSKNTAYLQMNSLRAEDTAVYYC |
| | | | | ARYPYWFAGYIDYWGQGTLVTVSS |
| 361 | SRP1848-G11 | VH | | EVQLVESGGGLVQPGGSLRLSCAA |
| | | | | SGFNISSTDIHWVRQAPGKGLEWV |
| | | | | AYITPAGGATWYADSVKGRFTISA |
| | | | | DTSKNTAYLQMNSLRAEDTAVYYC |
| | | | | ARYPYWFSGYMDYWGQGTLVTVSS |
| 362 | SRP1848-H01 | VH | | EVQLVESGGGLVQPGGSLRLSCAA |
| | | | | SGFNIRTQSIHWVRQAPGKGLEWI |
| | | | | GDIFPIDGITDYADSVKGRFTISA |
| | | | | DTSKNTAYLQMNSLRAEDTAVYYC |
| | | | | ARGSWSWPSGMDYYLDYWGQGTLV |
| | | | | TVSS |
| 363 | SRP2060-E10 | VH | | EVQLLESGGGLVQPGGSLRLSCAF |
| | | | | SGFSLSTFGMGVGWVRQAPGKGLE |
| | | | | WVSHIWWDDDKYYHPALKGRFTIS |
| | | | | KDNSKNTVYLQMNSLRAEDTAVYY |
| | | | | CGRNHFPHYYGSSHWYFNVWGQGT |
| | | | | TVTVSS |
| 364 | SRP2060-E05 | VH | | EVQLLESGGGLVQPGGSLRLSCAF |
| | | | | SGFSLSTFGMGVGWVRQAPGKGLE |
| | | | | WVSHIWWDDDKYYHPALKGRFTVS |
| | | | | KDNSKNTVYLQMNSLRAEDTAVYY |
| | | | | CGRNHFPHYYGSSHWYFNVWGQGT |
| | | | | TVTVSS |
| 365 | SRP2060-B01 | VH | | EVQLLESGGGLVQPGGSLRLSCAL |
| | | | | SGFSLSTFGMGVGWVRQATGKGLE |
| | | | | WVSHIWWDDDKYYHPALKGRFTIS |
| | | | | KDNSKNTVHLQMNSLRAEDTAVYY |
| | | | | CGRNHFPHYYGSSHWYFNVWGQGT |
| | | | | TVTVSS |
| 366 | SRP2060-A06 | VH | | EVQLLESGGGLVQPGGSLRLSCAF |
| | | | | SGFSLSTFGMGVGWVRQAPGKGLE |
| | | | | WVGHIWWDDDKYYYPALKGRFTIS |
| | | | | KDNSKNTVYLQMNSLRAEDTAVYY |
| | | | | CGRNHFPHYYGSSHWYFDVWGQGT |
| | | | | TVTVSS |
| 367 | trastuzumab | VL | | DIQMTQSPSSLSASVGDRVTITCR |
| | | | | ASQDVNTAVAWYQQKPGKAPKLLI |
| | | | | YSASFLYSGVPSRFSGSRSGTDFT |
| | | | | LTISSLQPEDFATYYCQQHYTTPP |
| | | | | TFGQGTKVEIK |
| 368 | H6D1-LC4 | VL | | EIVMTQSPATLSLSPGERATLSCK |
| | | | | ASQDINSYLSWYQQKPGQAPRLLI |
| | | | | YRANRLVDGIPARFSGSGSGTDYT |
| | | | | LTISSLEPEDFAVYYCLQYDEFPY |
| | | | | TFGGGTKVEIK |
| 369 | H6D1-LC5 | VL | | DIQMTQSPSTLSASVGDRVTITCK |
| | | | | ASQDINSYLSWYQQKPGKAPKLLI |
| | | | | YRANRLVDGVPSRFSGSGSGTEFT |
| | | | | LTISSLQPDDFATYYCLQYDEFPY |
| | | | | TFGGGTKVEIK |
| 370 | Human IgG1 HC | | | ASTKGPSVFPLAPSSKSTSGGTAA |
| | Constant | | | LGCLVKDYFPEPVTVSWNSGALTS |
| | | | | GVHTFPAVLQSSGLYSLSSVVTVP |
| | | | | SSSLGTQTYICNVNHKPSNTKVDK |
| | | | | KVEPKSCDKTHTCPPCPAPELLGG |
| | | | | PSVFLFPPKPKDTLMISRTPEVTC |
| | | | | VVVDVSHEDPEVKFNWYVDGVEVH |
| | | | | NAKTKPREEQYNSTYRVVSVLTVL |
| | | | | HQDWLNGKEYKCKVSNKALPAPIE |
| | | | | KTISKAKGQPREPQVYTLPPSREE |
| | | | | MTKNQVSLTCLVKGFYPSDIAVEW |
| | | | | ESNGQPENNYKTTPPVLDSDGSFF |
| | | | | LYSKLTVDKSRWQQGNVFSCSVMH |
| | | | | EALHNHYTQKSLSLSPGK |
| 371 | Human IgG LC | | | RTVAAPSVFIFPPSDEQLKSGTAS |
| | Constant Ckappa | | | VVCLLNNFYPREAKVQWKVDNALQ |
| | | | | SGNSQESVTEQDSKDSTYSLSSTL |
| | | | | TLSKADYEKHKVYACEVTHQGLSS |
| | | | | PVTKSFNRGEC |
| 372 | Mouse IgG1 HC | | | AKTTPPSVYPLAPGSAAQTNSMVT |
| | Constant | | | LGCLVKGYFPEPVTVTWNSGSLSS |
| | | | | GVHTFPAVLQSDLYTLSSSVTVPS |
| | | | | STWPSETVTCNVAHPASSTKVDKK |
| | | | | IVPRDCGCKPCICTVPEVSSVFIF |
| | | | | PPKPKDVLTITLTPKVTCVVVDIS |
| | | | | KDDPEVQFSWFVDDVEVHTAQTQP |
| | | | | REEQFNSTFRSVSELPIMHQDWLN |
| | | | | GKEFKCRVNSAAFPAPIEKTISKT |
| | | | | KGRPKAPQVYTIPPPKEQMAKDKV |
| | | | | SLTCMITDFFPEDITVEWQWNGQP |
| | | | | AENYKNTQPIMDTDGSYFVYSKLN |
| | | | | VQKSNWEAGNTFTCSVLHEGLHNH |
| | | | | HTEKSLSHSPG |
| 373 | Mouse IgG LC | | | RADAAPTVSIFPPSSEQLTSGGAS |
| | Constant Ckappa | | | VVCFLNNFYPKDINVKWKIDGSER |
| | | | | QNGVLNSWTDQDSKDSTYSMSSTL |
| | | | | TLTKDEYERHNSYTCEATHKTSTS |
| | | | | PIVKSFNRNEC |
| 374 | Kappa LC | | | HMTVAAPSVFIFPPSDEQLKSGTA |
| | | | | SVVCLLNNFYPREAKVQWKVDNAL |
| | | | | QSGNSQESVTEQDSKDSTYSLSST |
| | | | | LTLSKADYEKHKVYACEVTHQGLS |
| | | | | SPVTKSFNRGEC |
| 375 | Lambda LD | | | GQPKAAPSVTLFPPSSEELQANKA |
| | | | | TLVCLISDFYPGAVTVAWKADSSP |
| | | | | VKAGVETTTPSKQSNNKYAASSYL |
| | | | | SLTPEQWKSHRSYSCQVTHEGSTV |
| | | | | EKTVAPTECS |
| 376 | FlagHis Tag | | | GSGDYKDDDDKGSGHHHHHH |
| 377 | Linker | | | GGGGSGGGGSGGGGS |
| 378 | Linker | | | AAGSDQEPKSS |
| 379 | 1848-B10-VH- | scFv | | MEVQLVESGGGLVQPGGSLRLSCA |
| | (G4S)3-VL | | | ASGFNTTTKSIHWVRQAPGKGLEW |
| | | | | VGEIYPRDGITDYADSVKGRFTIS |
| | | | | ADTSKNTAYLQMNSLRAEDTAVYY |
| | | | | CARGGWHWRSGYSYYLDYWGQGTL |
| | | | | VTVSSGGGGSGGGGSGGGGSDIQM |
| | | | | TQSPSSLSASVGDRVTITCRASQD |
| | | | | VNTAVAWYQQKPGKAPKLLIYSAS |
| | | | | FLYSGVPSRFSGSRSGTDFTLTIS |
| | | | | SLQPEDFATYYCQQHYTTPPTFGQ |
| | | | | GTKVEIK |
| 380 | 1848-B10-VL- | scFv | | MDIQMTQSPSSLSASVGDRVTITC |
| | (G4S)3-VH | | | RASQDVNTAVAWYQQKPGKAPKLL |
| | | | | IYSASFLYSGVPSRFSGSRSGTDF |
| | | | | TLTISSLQPEDFATYYCQQHYTTP |
| | | | | PTFGQGTKVEIKGGGGSGGGGSGG |
| | | | | GGSEVQLVESGGGLVQPGGSLRLS |
| | | | | CAASGFNTTTKSIHWVRQAPGKGL |
| | | | | EWVGEIYPRDGITDYADSVKGRFT |
| | | | | ISADTSKNTAYLQMNSLRAEDTAV |
| | | | | YYCARGGWHWRSGYSYYLDYWGQG |
| | | | | TLVTVSS |
| 381 | 1848-B10-VH- | scFv-Fc | | MEVQLVESGGGLVQPGGSLRLSCA |
| | (G4S)3-VL | | | ASGFNTTTKSIHWVRQAPGKGLEW |
| | | | | VGEIYPRDGITDYADSVKGRFTIS |
| | | | | ADTSKNTAYLQMNSLRAEDTAVYY |
| | | | | CARGGWHWRSGYSYYLDYWGQGTL |
| | | | | VTVSSGGGGSGGGGSGGGGSDIQM |
| | | | | TQSPSSLSASVGDRVTITCRASQD |
| | | | | VNTAVAWYQQKPGKAPKLLIYSAS |
| | | | | FLYSGVPSRFSGSRSGTDFTLTIS |
| | | | | SLQPEDFATYYCQQHYTTPPTFGQ |
| | | | | GTKVEIKAAGSDQEPKSSDKTHTC |
| | | | | PPCPAPELLGGPSVFLFPPKPKDT |
| | | | | LMISRTPEVTCVVVDVSHEDPEVK |
| | | | | FNWYVDGVEVHNAKTKPREEQYNS |
| | | | | TYRVVSVLTVLHQDWLNGKEYKCK |
| | | | | VSNKALPAPIEKTISKAKGQPREP |
| | | | | QVYTLPPSREEMTKNQVSLTCLVK |
| | | | | GFYPSDIAVEWESNGQPENNYKTT |
| | | | | PPVLDSDGSFFLYSKLTVDKSRWQ |
| | | | | QGNVFSCSVMHEALHNHYTQKSLS |
| | | | | LSPGK |
| 382 | 1848-B10-VL- | scFv-Fc | | MDIQMTQSPSSLSASVGDRVTITC |
| | (G4S)3-VH | | | RASQDVNTAVAWYQQKPGKAPKLL |
| | | | | IYSASFLYSGVPSRFSGSRSGTDF |
| | | | | TLTISSLQPEDFATYYCQQHYTTP |
| | | | | PTFGQGTKVEIKGGGGSGGGGSGG |
| | | | | GGSEVQLVESGGGLVQPGGSLRLS |
| | | | | CAASGENTTTKSIHWVRQAPGKGL |
| | | | | EWVGEIYPRDGITDYADSVKGRFT |
| | | | | ISADTSKNTAYLQMNSLRAEDTAV |
| | | | | YYCARGGWHWRSGYSYYLDYWGQG |
| | | | | TLVTVSSAAGSDQEPKSSDKTHTC |
| | | | | PPCPAPELLGGPSVFLFPPKPKDT |
| | | | | LMISRTPEVTCVVVDVSHEDPEVK |
| | | | | FNWYVDGVEVHNAKTKPREEQYNS |
| | | | | TYRVVSVLTVLHQDWLNGKEYKCK |
| | | | | VSNKALPAPIEKTISKAKGQPREP |
| | | | | QVYTLPPSREEMTKNQVSLTCLVK |
| | | | | GFYPSDIAVEWESNGQPENNYKTT |
| | | | | PPVLDSDGSFFLYSKLTVDKSRWQ |
| | | | | QGNVFSCSVMHEALHNHYTQKSLS |
| | | | | LSPGK |
| 383 | Farletuzumab | scFv | | DIQLTQSPSSLSASVGDRVTITCS |
| | | | | VSSSISSNNLHWYQQKPGKAPKPW |
| | | | | IYGTSNLASGVPSRFSGSGSGTDY |
| | | | | TFTISSLQPEDIATYYCQQWSSYP |
| | | | | YMYTFGQGTKVEIKGGGGSGGGGS |
| | | | | GGGGSGGGGSEVQLVESGGGVVQP |
| | | | | GRSLRLSCSASGFTFSGYGLSWVR |
| | | | | QAPGKGLEWVAMISSGGSYTYYAD |
| | | | | SVKGRFAISRDNAKNTLFLQMDSL |
| | | | | RPEDTGVYFCARHGDDPAWFAYWG |
| | | | | QGTPVTVSS |
| 384 | Farletuzumab | scFv | | EVQLVESGGGVVQPGRSLRLSCSA |
| | | | | SGFTFSGYGLSWVRQAPGKGLEWV |
| | | | | AMISSGGSYTYYADSVKGRFAISR |
| | | | | DNAKNTLFLQMDSLRPEDTGVYFC |
| | | | | ARHGDDPAWFAYWGQGTPVTVSSG |
| | | | | GGGSGGGGSGGGGSGGGGSDIQLT |
| | | | | QSPSSLSASVGDRVTITCSVSSSI |
| | | | | SSNNLHWYQQKPGKAPKPWIYGTS |
| | | | | NLASGVPSRFSGSGSGTDYTFTIS |
| | | | | SLQPEDIATYYCQQWSSYPYMYTF |
| | | | | GQGTKVEIK |
| 385 | Farletuzumab | CDRH1 | Kabat | GYGLS |
| 386 | Farletuzumab | CDRH1 | Kabat | MISSGGSYTYYADSVKG |
| 387 | Farletuzumab | CDRH3 | Kabat | HGDDPAWFAY |
| 388 | Farletuzumab | CDRL1 | Kabat | SVSSSISSNNLH |
| 389 | Farletuzumab | CDRL2 | Kabat | GTSNLAS |
| 390 | Farletuzumab | CDRL3 | Kabat | QQWSSYPYMYT |
| 391 | Farletuzumab | VH | | EVQLVESGGGVVQPGRSLRLSCSA |
| | | | | SGFTFSGYGLSWVRQAPGKGLEWV |
| | | | | AMISSGGSYTYYADSVKGRFAISR |
| | | | | DNAKNTLFLQMDSLRPEDTGVYFC |
| | | | | ARHGDDPAWFAYWGQGTPVTVSS |
| 392 | Farletuzumab | VL | | DIQLTQSPSSLSASVGDRVTITCS |
| | | | | VSSSISSNNLHWYQQKPGKAPKPW |
| | | | | IYGTSNLASGVPSRFSGSGSGTDY |
| | | | | TFTISSLQPEDIATYYCQQWSSYP |
| | | | | YMYTFGQGTKVEIK |
| 393 | | VH | | GAGGTACAGCTTGTCGAGAGCGGT |
| | | | | GGTGGAGTAGTCCAACCGGGTCGA |
| | | | | AGTCTTAGGCTTTCCTGTAGCGCA |
| | | | | TCTGGGTTCACTTTTAGTGGCTAC |
| | | | | GGCCTCTCCTGGGTGAGACAGGCG |
| | | | | CCTGGGAAGGGGCTGGAGTGGGTA |
| | | | | GCCATGATTTCATCTGGTGGCTCA |
| | | | | TATACTTATTATGCCGACTCCGTA |
| | | | | AAGGGAAGATTCGCAATATCACGC |
| | | | | GATAACGCTAAAAATACACTCTTC |
| | | | | TTGCAGATGGATTCTTTGAGACCT |
| | | | | GAGGATACCGGGGTTTACTTTTGC |
| | | | | GCCAGACACGGGGATGACCCCGCC |
| | | | | TGGTTTGCCTATTGGGGACAGGGA |
| | | | | ACCCCTGTGACGGTATCCTCT |
| 394 | | VL | | GATATTCAGCTTACTCAAAGTCCG |
| | | | | AGTAGTCTGTCTGCCTCAGTTGGC |
| | | | | GATAGGGTGACCATCACTTGCTCC |
| | | | | GTAAGTAGTTCTATTTCTTCCAAC |
| | | | | AACCTGCATTGGTATCAACAGAAA |
| | | | | CCAGGTAAAGCACCTAAGCCGTGG |
| | | | | ATCTACGGAACGTCCAACCTTGCG |
| | | | | TCTGGCGTACCAAGCCGGTTCTCC |
| | | | | GGGAGTGGGAGTGGTACAGATTAC |
| | | | | ACATTTACTATCAGTTCTCTTCAA |
| | | | | CCGGAAGACATTGCCACATATTAT |
| | | | | TGCCAGCAATGGTCATCTTACCCC |
| | | | | TATATGTACACATTTGGTCAGGGT |
| | | | | ACAAAGGTTGAAATAAAA |
| 395 | huMOV19 | CDRH1 | Kabat | GYFMN |
| 396 | huMOV19 | CDRH2 | Kabat | RIHPYDGDTFYNQKFQG |
| 397 | huMOV19 | CDRH3 | Kabat | YDGSRAMDY |
| 398 | huMOV19 | CDRL1 | Kabat | KASQSVSFAGTSLMH |
| 399 | huMOV19 | CDRL2 | Kabat | RASNLEA |
| 400 | huMOV19 | CDRL3 | Kabat | QQSREYPYT |
| 401 | huMOV19 1.00 | VH | | QVQLVQSGAEVVKPGASVKISCKA |
| | | | | SGYTFTGYFMNWVKQSPGQSLEWI |
| | | | | GRIHPYDGDTFYNQKFQGKATLTV |
| | | | | DKSSNTAHMELLSLTSEDFAVYYC |
| | | | | TRYDGSRAMDYWGQGTTVTVSS |
| 402 | huMOV19 1.00 | VL | | DIVLTQSPLSLAVSLGQPAIISCK |
| | | | | ASQSVSFAGTSLMHWYHQKPGQQP |
| | | | | RLLIYRASNLEAGVPDRFSGSGSK |
| | | | | TDFTLNISPVEAEDAATYYCQQSR |
| | | | | EYPYTFGGGTKLEIKR |
| 403 | huMOV19 1.60 | VH | | QVQLVQSGAEVVKPGASVKISCKA |
| | | | | SGYTFTGYFMNWVKQSPGQSLEWI |
| | | | | GRIHPYDGDTFYNQKFQGKATLTV |
| | | | | DKSSNTAHMELLSLTSEDFAVYYC |
| | | | | TRYDGSRAMDYWGQGTTVTVSS |
| 404 | huMOV19 1.60 | VL | | DIVLTQSPLSLAVSLGQPAIISCK |
| | | | | ASQSVSFAGTSLMHWYHQKPGQQP |
| | | | | RLLIYRASNLEAGVPDRFSGSGSK |
| | | | | TDFTLTISPVEAEDAATYYCQQSR |
| | | | | EYPYTFGGGTKLEIKR |
| 405 | RA15-7 | CDRH1 | Kabat | DFYMN |
| 406 | RA15-7 | CDRH2 | Kabat | FIRNKANGYTTEFNPSVKG |
| 407 | RA15-7 | CDRH3 | Kabat | TLYGYAYYYVMDA |
| 408 | RA15-7 | CDRL1 | Kabat | RTSEDIFRNLA |
| 409 | RA15-7 | CDRL2 | Kabat | DTNRLAD |
| 410 | RA15-7 | CDRL3 | Kabat | QQYDNYPLT |
| 411 | RA15-7 | VH | | EVQLVESGGGLVQPGGSLRLSCAA |
| | | | | SGFTFTDFYMNWVRQPPGKAPEWL |
| | | | | GFIRNKANGYTTEFNPSVKGRFTI |
| | | | | SRDNSKNSLYLQMNSLKTEDTATY |
| | | | | YCARTLYGYAYYYVMDAWGQGTLV |
| | | | | TVSS |
| 412 | RA15-7 | VL | | DIQMTQSPSSLSASLGDRVTITCR |
| | | | | TSEDIFRNLAWYQQKPGKAPKLLI |
| | | | | YDTNRLADGVPSRFSGSGSGTDYT |
| | | | | LTISSLQPEDFATYFCQQYDNYPL |
| | | | | TFGQGTKLEIK |
| 413 | huFR1-48 | CDRH1 | Kabat | NYWMQ |
| 414 | huFR1-48 | CDRH2 | Kabat | AIYPGNGDSRYTQKFQG |
| 415 | huFR1-48 | CDRH3 | Kabat | RDGNYAAY |
| 416 | huFR1-48 | CDRL1 | Kabat | RASENIYSNLA |
| 417 | huFR1-48 | CDRL2 | Kabat | AATNLAD |
| 418 | huFR1-48 | CDRL3 | Kabat | QHFWASPYT |
| 419 | huFR1-48 | VH | | QVQLVQSGAEVAKPGASVKLSCKA |
| | | | | SGYTFTNYWMQWIKQRPGQGLEWI |
| | | | | GAIYPGNGDSRYTQKFQGKATLTA |
| | | | | DKSSSTAYMQVSSLTSEDSAVYYC |
| | | | | ARRDGNYAAYWGQGTLVTVSA |
| 420 | huFR1-48 | VL | | DIQMTQSPSSLSVSVGERVTITCR |
| | | | | ASENIYSNLAWYQQKPGKSPKLLV |
| | | | | YAATNLADGVPSRFSGSESGTDYS |
| | | | | LKINSLQPEDFGSYYCQHFWASPY |
| | | | | TFGQGTKLEIKR |
| 421 | huFR1-49 | CDRH1 | Kabat | NYWMY |
| 422 | huFR1-49 | CDRH2 | Kabat | AIYPGNSDTTYNQKFQG |
| 423 | huFR1-49 | CDRH3 | Kabat | RHDYGAMDY |
| 424 | huFR1-49 | CDRL1 | Kabat | RASENIYTNLA |
| 425 | huFR1-49 | CDRL2 | Kabat | TASNLAD |
| 426 | huFR1-49 | CDRL3 | Kabat | QHFWVSPYT |
| 427 | huFR1-49 | VH | | QVQLQQSGAVVAKPGASVKMSCKA |
| | | | | SGYTFTNYWMYWIKQRPGQGLELI |
| | | | | GAIYPGNSDTTYNQKFQGKATLTA |
| | | | | VTSANTVYMEVSSLTSEDSAVYYC |
| | | | | TKRHDYGAMDYWGQGTSVTVSS |
| 428 | huFR1-49 | VL | | DIQMTQSPSSLSVSVGERVTITCR |
| | | | | ASENIYTNLAWYQQKPGKSPKLLV |
| | | | | YTASNLADGVPSRFSGSGSGTDYS |
| | | | | LKINSLQPEDFGTYYCQHFWVSPY |
| | | | | TFGQGTKLEIKR |
| 429 | huFR1-57 | CDRH1 | Kabat | SFGMH |
| 430 | huFR1-57 | CDRH2 | Kabat | YISSGSSTISYADSVKG |
| 431 | huFR1-57 | CDRH3 | Kabat | EAYGSSMEY |
| 432 | huFR1-57 | CDRL1 | Kabat | RASQNINNNLH |
| 433 | huFR1-57 | CDRL2 | Kabat | YVSQSVS |
| 434 | huFR1-57 | CDRL3 | Kabat | QQSNSWPHYT |
| 435 | huFR1-57 | VH | | EVQLVESGGGLVQPGGSRRLSCAA |
| | | | | SGFTFSSFGMHWVRQAPGKGLEWV |
| | | | | AYISSGSSTISYADSVKGRFTISR |
| | | | | DNSKKTLLLQMTSLRAEDTAMYYC |
| | | | | AREAYGSSMEYWGQGTLVTVSS |
| 436 | huFR1-57 | VL | | EIVLTQSPATLSVTPGDRVSLSCR |
| | | | | ASQNINNNLHWYQQKPGQSPRLLI |
| | | | | KYVSQSVSGIPDRFSGSGSGTDFT |
| | | | | LSISSVEPEDFGMYFCQQSNSWPH |
| | | | | YTFGQGTKLEIKR |
| 437 | huFR1-65 | CDRH1 | Kabat | SYTMH |
| 438 | huFR1-65 | CDRH2 | Kabat | YINPISGYTNYNQKFQG |
| 439 | huFR1-65 | CDRH3 | Kabat | GGAYGRKPMDY |
| 440 | huFR1-65 | CDRL1 | Kabat | KASQNVGPNVA |
| 441 | huFR1-65 | CDRL2 | Kabat | SASYRYS |
| 442 | huFR1-65 | CDRL3 | Kabat | QQYNSYPYT |
| 443 | huFR1-65 | VH | | QVQLVQSGAEVAKPGASVKMSCKA |
| | | | | SGYTFTSYTMHWVKQRPGQGLAWI |
| | | | | GYINPISGYTNYNQKFQGKATLTA |
| | | | | DKSSSTAYMQLNSLTSEDSAVYYC |
| | | | | ASGGAYGRKPMDYWGQGTSVTVSS |
| 444 | huFR1-65 | VL | | EIVMTQSPATMSTSPGDRVSVTCK |
| | | | | ASQNVGPNVAWYQQKPGQSPRALI |
| | | | | YSASYRYSGVPARFTGSGSGTDFT |
| | | | | LTISNMQSEDLAEYFCQQYNSYPY |
| | | | | TFGQGTKLEIKR |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating or palliating pediatric acute myeloid leukemia (AML) in a patient in need thereof, comprising administering to the patient an antibody conjugate comprising an antibody that specifically binds to folate receptor alpha (FOLR1) and is linked site-specifically to at least one payload moiety, in an amount effective to treat or palliate the pediatric AML in the patient.

2. The method of claim 1, wherein the pediatric AML is AML-RAM, CBF/GLIS AML, or BCR::ABL.

3. (canceled)

4. (canceled)

5. The method of claim 1, wherein the patient has relapsed, refractory or residual pediatric AIL.

6. (canceled)

7. (canceled)

8. (canceled)

9. The method of claim 1, wherein the patient has had prior therapy for pediatric AML.

10. (canceled)

11. (canceled)

12. (canceled)

13. (canceled)

14. (canceled)

15. The method of claim 1, wherein the patient has had no prior therapy for pediatric AML.

16. The method of claim 1, wherein the patient has a stem cell transplant subsequent to administrating the antibody conjugate, or an umbilical cord blood transplant subsequent to administrating the antibody conjugate.

17. (canceled)

18. (canceled)

19. (canceled)

20. The method of claim 1, wherein the antibody conjugate is administered at 4.3 mg/kg.

21. The method of claim 20, further comprising administering the antibody conjugate at a dose of 5.2 mg/kg after one or more doses of 4.3 mg/kg.

22. The method of claim 1, wherein the antibody conjugate is administered at 5.2 mg/kg.

23. The method of claim 22, further comprising administering the antibody conjugate at a dose of 4.3 mg/kg after one or more doses of 5.2 mg/kg.

24. (canceled)

25. (canceled)

26. (canceled)

27. The method of claim 1, wherein antibody conjugate is administered at 4.3 mg/kg every four weeks.

28. The method of claim 1, wherein antibody conjugate is administered at 4.3 mg/kg every 2-3 weeks.

29. The method of claim 1, wherein antibody conjugate is administered at 5.2 mg/kg every four weeks.

30. The method of claim 1, wherein antibody conjugate is administered at 5.2 mg/kg every 2-3 weeks.

31. The method of claim 1, wherein the antibody conjugate is administered in combination with methotrexate, intratumoral methotrexate, fludarabine, ara-C, G-CSF, donor lymphocyte infusion, fludarabine, cytarabine, venetoclax, azacitidin, decitabine, dasatanib, gemtuzumab, plerixafor, calaspargase, CAR-T therapy, a CXCR4 antagonist, or any combination thereof.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. The method of claim 31, wherein the G-CSF is administered prophylactically after a first cycle of the antibody conjugate, or between day 8 and day 15 following the administration of the antibody conjugate.

50. (canceled)

51. (canceled)

52. (canceled)

53. The method of claim 1, wherein the FOLR1 antibody conjugate is administered intravenously.

54. The method of claim 1, wherein treatment continues until the patient has stable disease, decrease in disease burden, partial remission, or complete remission, or until the patient undergoes subsequent therapy.

55. The method of claim 1, wherein the antibody comprises one or more non-natural amino acids at sites selected from the group consisting of:—HC F404, HC-K121, HC-Y180, HC-F241, HC-221, LC-T22, LC-S7, LC-N152, LC-K42, LC-E161, LC-D170, HC-S136, HC S25, HC-A40, HC-S119, HC-S190, HC-K222, HC-R19, HC-Y52, and HC-S70, according to the Kabat, Chothia, or EU numbering scheme.

56. (canceled)

57. (canceled)

58. (canceled)

59. (canceled)

60. The method of claim 55, wherein a residue of the one or more non-natural amino acids is linked to the payload moiety via a linker that is hydrolytically stable.

61. The method of claim 55, wherein a residue of the one or more non-natural amino acids is linked to the payload moiety via a linker that is cleavable.

62. The method of claim 55, wherein the one or more non-natural amino acids:
is selected from the group consisting of p-acetyl-L-phenylalanine, O-methyl-L-tyrosine, an -3-(2-naphthyl)alanine, 3-methyl-phenylalanine, O-4-allyl-L-tyrosine, 4-propyl-L-tyrosine, a tri-O-acetyl-GlcNAcP-serine, L-Dopa, fluorinated phenylalanine, isopropyl-L-phenylalanine, p-azido-L-phenylalanine, p-azido-methyl-L-phenylalanine, compound 56, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine, or
is a residue of compound (30) or compound (56).

63. (canceled)

64. The method of claim 1, wherein the at least one payload moiety is selected from the group consisting of maytansines, hemiasterlins, amanitins, and auristatins.

65. The method of claim 1, wherein the at least one payload moiety is selected from the group consisting of DM1, hemiasterlin, amanitin, MMAF, and MMAE.

66. (canceled)

67. The method of claim 1, wherein the at least one payload moiety is 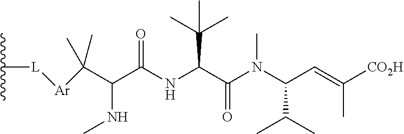
wherein Ar is aryl or heteroaryl, L is a linker, and the wiggly line indicates a bond to the antibody.

68. The method of claim 1, wherein the at least one payload moiety is a hemiasterlin payload.

69. The method of claim 68, wherein the hemiasterlin payload is a 3-aminophenyl hemiasterlin payload.

70. The method of claim 68, wherein the hemiasterlin payload is a cleavable 3-aminophenyl hemiasterlin payload conjugated to the antibody with a cleavable valine citrulline p-aminobenzyl carbamate linker functionalized with dibenzocyclooctyne (DBCO).

71. (canceled)

72. The method of claim 1, wherein the antibody comprises:
(i) three heavy chain CDRs and three light chain CDRs of the V_{H} region SEQ ID NO: 362, and the V_{L} region SEQ ID NO: 367;
(ii) three heavy chain CDRs and three light chain CDRs of the V_{H} region SEQ ID NO: 323, and the V_{L} region SEQ ID NO: 367;
(iii) three heavy chain CDRs and three light chain CDRs of the V_{H} region SEQ ID NO: 308, and the V_{L} region SEQ ID NO: 367;
(iv) three heavy chain CDRs and three light chain CDRs of the V_{H} region SEQ ID NO: 309, and the V_{L} region SEQ ID NO: 367;
(v) three heavy chain CDRs and three light chain CDRs of the V_{H} region SEQ ID NO: 310, and the V_{L} region SEQ ID NO: 367;
(vi) three heavy chain CDRs and three light chain CDRs of the V_{H} region SEQ ID NO: 311, and the V_{L} region SEQ ID NO: 367;
(vii) three heavy chain CDRs and three light chain CDRs of the V_{H} region SEQ ID NO: 312, and the V_{L} region SEQ ID NO: 367;
(viii) three heavy chain CDRs and three light chain CDRs of the V_{H} region SEQ ID NO: 313, and the V_{L} region SEQ ID NO: 367;
(ix) three heavy chain CDRs and three light chain CDRs of the V_{H} region SEQ ID NO: 314, and the V_{L} region SEQ ID NO: 367;
(x) three heavy chain CDRs and three light chain CDRs of the V_{H} region SEQ ID NO: 315, and the V_{L} region SEQ ID NO: 367;
(xi) three heavy chain CDRs and three light chain CDRs of the V_{H} region SEQ ID NO: 316, and the V_{L} region SEQ ID NO: 367;
(xii) three heavy chain CDRs and three light chain CDRs of the V_{H} region SEQ ID NO: 317, and the V_{L} region SEQ ID NO: 367;
(xiii) three heavy chain CDRs and three light chain CDRs of the V_{H} region SEQ ID NO: 318, and the V_{L} region SEQ ID NO: 367;
(xiv) three heavy chain CDRs and three light chain CDRs of the V_{H} region SEQ ID NO: 319, and the V_{L} region SEQ ID NO: 367;
(xv) three heavy chain CDRs and three light chain CDRs of the V_{H} region SEQ ID NO: 320, and the V_{L} region SEQ ID NO: 367;
(xvi) three heavy chain CDRs and three light chain CDRs of the V_{H} region SEQ ID NO: 321, and the V_{L} region SEQ ID NO: 367;
(xvii) three heavy chain CDRs and three light chain CDRs of the V_{H} region SEQ ID NO: 322, and the V_{L} region SEQ ID NO: 367;
(xviii) three heavy chain CDRs and three light chain CDRs of the V_{H} region SEQ ID NO: 324, and the V_{L} region SEQ ID NO: 367;
(xix) three heavy chain CDRs and three light chain CDRs of the V_{H} region SEQ ID NO: 325, and the V_{L} region SEQ ID NO: 367;
(xx) three heavy chain CDRs and three light chain CDRs of the V_{H} region SEQ ID NO: 326, and the V_{L} region SEQ ID NO: 367; or
(xxi) three heavy chain CDRs and three light chain CDRs of the V_{H} region SEQ ID NO: 327, and the V_{L} region SEQ ID NO: 367;
(xxii) three heavy chain CDRs and three light chain CDRs of the V_{H} region SEQ ID NO: 328, and the V_{L} region SEQ ID NO: 367;
(xxiii) three heavy chain CDRs and three light chain CDRs of the V_{H} region SEQ ID NO: 329, and the V_{L} region SEQ ID NO: 367;
(xxiv) three heavy chain CDRs and three light chain CDRs of the V_{H} region SEQ ID NO: 330, and the V_{L} region SEQ ID NO: 367;
(xxv) three heavy chain CDRs and three light chain CDRs of the V_{H} region SEQ ID NO: 331, and the V_{L} region SEQ ID NO: 367;
(xxvi) three heavy chain CDRs and three light chain CDRs of the V_{H} region SEQ ID NO: 332, and the V_{L} region SEQ ID NO: 367;
(xxvii) three heavy chain CDRs and three light chain CDRs of the V_{H} region SEQ ID NO: 333, and the V_{L} region SEQ ID NO: 367;
(xxviii) three heavy chain CDRs and three light chain CDRs of the V_{H} region SEQ ID NO: 334, and the V_{L} region SEQ ID NO: 367;
(xxix) three heavy chain CDRs and three light chain CDRs of the V_{H} region SEQ ID NO: 335, and the V_{L} region SEQ ID NO: 367;
(xxx) three heavy chain CDRs and three light chain CDRs of the V_{H} region SEQ ID NO: 336, and the V_{L} region SEQ ID NO: 367;
(xxxi) three heavy chain CDRs and three light chain CDRs of the V_{H} region SEQ ID NO: 337, and the V_{L} region SEQ ID NO: 367;
(xxxii) three heavy chain CDRs and three light chain CDRs of the V_{H} region SEQ ID NO: 338, and the V_{L} region SEQ ID NO: 367;
(xxxiii) three heavy chain CDRs and three light chain CDRs of the V_{H} region SEQ ID NO: 339, and the V_{L} region SEQ ID NO: 367;
(xxxiv) three heavy chain CDRs and three light chain CDRs of the V_{H} region SEQ ID NO: 340, and the V_{L} region SEQ ID NO: 367;
(xxxv) three heavy chain CDRs and three light chain CDRs of the V_{H} region SEQ ID NO: 341, and the V_{L} region SEQ ID NO: 367;
(xxxvi) three heavy chain CDRs and three light chain CDRs of the V_{H} region SEQ ID NO: 342, and the V_{L} region SEQ ID NO: 367;
(xxxvii) three heavy chain CDRs and three light chain CDRs of the V_{H} region SEQ ID NO: 343, and the V_{L} region SEQ ID NO: 367;
(xxxviii) three heavy chain CDRs and three light chain CDRs of the V_{H} region SEQ ID NO: 344, and the V_{L} region SEQ ID NO: 367;
(xxxix) three heavy chain CDRs and three light chain CDRs of the V_{H} region SEQ ID NO: 345, and the V_{L} region SEQ ID NO: 367;
(xl) three heavy chain CDRs and three light chain CDRs of the V_{H} region SEQ ID NO: 346, and the V_{L} region SEQ ID NO: 367;
(xli) three heavy chain CDRs and three light chain CDRs of the V_{H} region SEQ ID NO: 347, and the V_{L} region SEQ ID NO: 367;
(xlii) three heavy chain CDRs and three light chain CDRs of the V_{H} region SEQ ID NO: 348, and the V_{L} region SEQ ID NO: 367;
(xliii) three heavy chain CDRs and three light chain CDRs of the V_{H} region SEQ ID NO: 349, and the V_{L} region SEQ ID NO: 367;
(xliv) three heavy chain CDRs and three light chain CDRs of the V_{H} region SEQ ID NO: 350, and the V_{L} region SEQ ID NO: 367;
(xlv) three heavy chain CDRs and three light chain CDRs of the V_{H} region SEQ ID NO: 351, and the V_{L} region SEQ ID NO: 367;
(xlvi) three heavy chain CDRs and three light chain CDRs of the V_{H} region SEQ ID NO: 352, and the V_{L} region SEQ ID NO: 367;
(xlvii) three heavy chain CDRs and three light chain CDRs of the V_{H} region SEQ ID NO: 353, and the V_{L} region SEQ ID NO: 367;
(xlviii) three heavy chain CDRs and three light chain CDRs of the V_{H} region SEQ ID NO: 354, and the V_{L} region SEQ ID NO: 367;
(xlix) three heavy chain CDRs and three light chain CDRs of the V_{H} region SEQ ID NO: 355, and the V_{L} region SEQ ID NO: 367;
(1) three heavy chain CDRs and three light chain CDRs of the V_{H} region SEQ ID NO: 356, and the V_{L} region SEQ ID NO: 367;
(li) three heavy chain CDRs and three light chain CDRs of the V_{H} region SEQ ID NO: 357, and the V_{L} region SEQ ID NO: 367;
(lii) three heavy chain CDRs and three light chain CDRs of the V_{H} region SEQ ID NO: 358, and the V_{L} region SEQ ID NO: 367;
(liii) three heavy chain CDRs and three light chain CDRs of the V_{H} region SEQ ID NO: 359, and the V_{L} region SEQ ID NO: 367;
(liv) three heavy chain CDRs and three light chain CDRs of the V_{H} region SEQ ID NO: 360, and the V_{L} region SEQ ID NO: 367;
(lv) three heavy chain CDRs and three light chain CDRs of the V_{H} region SEQ ID NO: 361, and the V_{L} region SEQ ID NO: 367;
(lvi) three heavy chain CDRs and three light chain CDRs of the V_{H} region SEQ ID NO: 363, and the V_{L} region SEQ ID NO: 368;
(lvii) three heavy chain CDRs and three light chain CDRs of the V_{H} region SEQ ID NO: 364, and the V_{L} region SEQ ID NO: 368;
(lviii) three heavy chain CDRs and three light chain CDRs of the V_{H} region SEQ ID NO: 365, and the V_{L} region SEQ ID NO: 369; or
(lix) three heavy chain CDRs and three light chain CDRs of the V_{H} region SEQ ID NO: 366, and the V_{L} region SEQ ID NO: 369.

73. The method of claim 1, wherein the antibody comprises:
(i) a VH comprising: a CDR-H1 comprising one of SEQ ID NOs: 58 and 117; a CDR-H2 comprising one of SEQ ID NOs: 176 and 235; and a CDR-H3 comprising SEQ ID NO: 294;
(ii) a VH comprising: a CDR-H1 comprising one of SEQ ID NOs: 19 and 78; a CDR-H2 comprising one of SEQ ID NOs: 137 and 196; and a CDR-H3 comprising SEQ ID NO: 255;
(iii) a VH comprising: a CDR-H1 comprising one of SEQ ID NOs: 4 and 63; a CDR-H2 comprising one of SEQ ID NOs: 122 and 181; and a CDR-H3 comprising SEQ ID NO: 240;
(iv) a VH comprising: a CDR-H1 comprising one of SEQ ID NOs: 5 and 64; a CDR-H2 comprising one of SEQ ID NOs: 123 and 182; and a CDR-H3 comprising SEQ ID NO: 241;
(v) a VH comprising: a CDR-H1 comprising one of SEQ ID NOs: 6 and 65; a CDR-H2 comprising one of SEQ ID NOs: 124 and 183; and a CDR-H3 comprising SEQ ID NO: 242;
(vi) a VH comprising: a CDR-H1 comprising one of SEQ ID NOs: 7 and 66; a CDR-H2 comprising one of SEQ ID NOs: 125 and 184; and a CDR-H3 comprising SEQ ID NO: 243;
(vii) a VH comprising: a CDR-H1 comprising one of SEQ ID NOs: 8 and 67; a CDR-H2 comprising one of SEQ ID NOs: 126 and 185; and a CDR-H3 comprising SEQ ID NO: 244;
(viii) a VH comprising: a CDR-H1 comprising one of SEQ ID NOs: 9 and 68; a CDR-H2 comprising one of SEQ ID NOs: 127 and 186; and a CDR-H3 comprising SEQ ID NO: 245;
(ix) a VH comprising: a CDR-H1 comprising one of SEQ ID NOs: 10 and 69; a CDR-H2 comprising one of SEQ ID NOs: 128 and 187; and a CDR-H3 comprising SEQ ID NO: 246;
(x) a VH comprising: a CDR-H1 comprising one of SEQ ID NOs: 11 and 70; a CDR-H2 comprising one of SEQ ID NOs: 129 and 188; and a CDR-H3 comprising SEQ ID NO: 247;
(xi) a VH comprising: a CDR-H1 comprising one of SEQ ID NOs: 12 and 71; a CDR-H2 comprising one of SEQ ID NOs: 130 and 189; and a CDR-H3 comprising SEQ ID NO: 248;
(xii) a VH comprising: a CDR-H1 comprising one of SEQ ID NOs: 13 and 72; a CDR-H2 comprising one of SEQ ID NOs: 131 and 190; and a CDR-H3 comprising SEQ ID NO: 249;
(xiii) a VH comprising: a CDR-H1 comprising one of SEQ ID NOs: 14 and 73; a CDR-H2 comprising one of SEQ ID NOs: 132 and 191; and a CDR-H3 comprising SEQ ID NO: 250;
(xiv) a VH comprising: a CDR-H1 comprising one of SEQ ID NOs: 15 and 74; a CDR-H2 comprising one of SEQ ID NOs: 133 and 192; and a CDR-H3 comprising SEQ ID NO: 251;
(xv) a VH comprising: a CDR-H1 comprising one of SEQ ID NOs: 16 and 75; a CDR-H2 comprising one of SEQ ID NOs: 134 and 193; and a CDR-H3 comprising SEQ ID NO: 252;
(xvi) a VH comprising: a CDR-H1 comprising one of SEQ ID NOs: 17 and 76; a CDR-H2 comprising one of SEQ ID NOs: 135 and 194; and a CDR-H3 comprising SEQ ID NO: 253;
(xvii) a VH comprising: a CDR-H1 comprising one of SEQ ID NOs: 18 and 77; a CDR-H2 comprising one of SEQ ID NOs: 136 and 195; and a CDR-H3 comprising SEQ ID NO: 254;
(xviii) a VH comprising: a CDR-H1 comprising one of SEQ ID NOs: 20 and 79; a CDR-H2 comprising one of SEQ ID NOs: 138 and 197; and a CDR-H3 comprising SEQ ID NO: 256;
(xix) a VH comprising: a CDR-H1 comprising one of SEQ ID NOs: 21 and 80; a CDR-H2 comprising one of SEQ ID NOs: 139 and 198; and a CDR-H3 comprising SEQ ID NO: 257;
(xx) a VH comprising: a CDR-H1 comprising one of SEQ ID NOs: 22 and 81; a CDR-H2 comprising one of SEQ ID NOs: 140 and 199; and a CDR-H3 comprising SEQ ID NO: 258;
(xxi) a VH comprising: a CDR-H1 comprising one of SEQ ID NOs: 23 and 82; a CDR-H2 comprising one of SEQ ID NOs: 141 and 200; and a CDR-H3 comprising SEQ ID NO: 259;
(xxii) a VH comprising: a CDR-H1 comprising one of SEQ ID NOs: 24 and 83; a CDR-H2 comprising one of SEQ ID NOs: 142 and 201; and a CDR-H3 comprising SEQ ID NO: 260;
(xxiii) a VH comprising: a CDR-H1 comprising one of SEQ ID NOs: 25 and 84; a CDR-H2 comprising one of SEQ ID NOs: 143 and 202; and a CDR-H3 comprising SEQ ID NO: 261;
(xxiv) a VH comprising: a CDR-H1 comprising one of SEQ ID NOs: 26 and 85; a CDR-H2 comprising one of SEQ ID NOs: 144 and 203; and a CDR-H3 comprising SEQ ID NO: 262;
(xxv) a VH comprising: a CDR-H1 comprising one of SEQ ID NOs: 27 and 86; a CDR-H2 comprising one of SEQ ID NOs: 145 and 204; and a CDR-H3 comprising SEQ ID NO: 263;
(xxvi) a VH comprising: a CDR-H1 comprising one of SEQ ID NOs: 28 and 87; a CDR-H2 comprising one of SEQ ID NOs: 146 and 205; and a CDR-H3 comprising SEQ ID NO: 264;
(xxvii) a VH comprising: a CDR-H1 comprising one of SEQ ID NOs: 29 and 88; a CDR-H2 comprising one of SEQ ID NOs: 147 and 206; and a CDR-H3 comprising SEQ ID NO: 265;
(xxviii) a VH comprising: a CDR-H1 comprising one of SEQ ID NOs: 30 and 89; a CDR-H2 comprising one of SEQ ID NOs: 148 and 207; and a CDR-H3 comprising SEQ ID NO: 266;
(xxix) a VH comprising: a CDR-H1 comprising one of SEQ ID NOs: 31 and 90; a CDR-H2 comprising one of SEQ ID NOs: 149 and 208; and a CDR-H3 comprising SEQ ID NO: 267;
(xxx) a VH comprising: a CDR-H1 comprising one of SEQ ID NOs: 32 and 91; a CDR-H2 comprising one of SEQ ID NOs: 150 and 209; and a CDR-H3 comprising SEQ ID NO: 268;
(xxxi) a VH comprising: a CDR-H1 comprising one of SEQ ID NOs: 33 and 92; a CDR-H2 comprising one of SEQ ID NOs: 151 and 210; and a CDR-H3 comprising SEQ ID NO: 269;
(xxxii) a VH comprising: a CDR-H1 comprising one of SEQ ID NOs: 34 and 93; a CDR-H2 comprising one of SEQ ID NOs: 152 and 211; and a CDR-H3 comprising SEQ ID NO: 270;
(xxxiii) a VH comprising: a CDR-H1 comprising one of SEQ ID NOs: 35 and 94; a CDR-H2 comprising one of SEQ ID NOs: 153 and 212; and a CDR-H3 comprising SEQ ID NO: 271;
(xxxiv) a VH comprising: a CDR-H1 comprising one of SEQ ID NOs: 36 and 95; a CDR-H2 comprising one of SEQ ID NOs: 154 and 213; and a CDR-H3 comprising SEQ ID NO: 272;
(xxxv) a VH comprising: a CDR-H1 comprising one of SEQ ID NOs: 37 and 96; a CDR-H2 comprising one of SEQ ID NOs: 155 and 214; and a CDR-H3 comprising SEQ ID NO: 273;
(xxxvi) a VH comprising: a CDR-H1 comprising one of SEQ ID NOs: 38 and 97; a CDR-H2 comprising one of SEQ ID NOs: 156 and 215; and a CDR-H3 comprising SEQ ID NO: 274;
(xxxvii) a VH comprising: a CDR-H1 comprising one of SEQ ID NOs: 39 and 98; a CDR-H2 comprising one of SEQ ID NOs: 157 and 216; and a CDR-H3 comprising SEQ ID NO: 275;
(xxxviii) a VH comprising: a CDR-H1 comprising one of SEQ ID NOs: 40 and 99; a CDR-H2 comprising one of SEQ ID NOs: 158 and 217; and a CDR-H3 comprising SEQ ID NO: 276;
(xxxix) a VH comprising: a CDR-H1 comprising one of SEQ ID NOs: 41 and 100; a CDR-H2 comprising one of SEQ ID NOs: 159 and 218; and a CDR-H3 comprising SEQ ID NO: 277;
(xl) a VH comprising: a CDR-H1 comprising one of SEQ ID NOs: 42 and 101; a CDR-H2 comprising one of SEQ ID NOs: 160 and 219; and a CDR-H3 comprising SEQ ID NO: 278;
(xli) a VH comprising: a CDR-H1 comprising one of SEQ ID NOs: 43 and 102; a CDR-H2 comprising one of SEQ ID NOs: 161 and 220; and a CDR-H3 comprising SEQ ID NO: 279;
(xlii) a VH comprising: a CDR-H1 comprising one of SEQ ID NOs: 44 and 103; a CDR-H2 comprising one of SEQ ID NOs: 162 and 221; and a CDR-H3 comprising SEQ ID NO: 280;
(xliii) a VH comprising: a CDR-H1 comprising one of SEQ ID NOs: 45 and 104; a CDR-H2 comprising one of SEQ ID NOs: 163 and 222; and a CDR-H3 comprising SEQ ID NO: 281;
(xliv) a VH comprising: a CDR-H1 comprising one of SEQ ID NOs: 46 and 105; a CDR-H2 comprising one of SEQ ID NOs: 164 and 223; and a CDR-H3 comprising SEQ ID NO: 282;
(xlv) a VH comprising: a CDR-H1 comprising one of SEQ ID NOs: 47 and 106; a CDR-H2 comprising one of SEQ ID NOs: 165 and 224; and a CDR-H3 comprising SEQ ID NO: 283;
(xlvi) a VH comprising: a CDR-H1 comprising one of SEQ ID NOs: 48 and 107; a CDR-H2 comprising one of SEQ ID NOs: 166 and 225; and a CDR-H3 comprising SEQ ID NO: 284;
(xlvii) a VH comprising: a CDR-H1 comprising one of SEQ ID NOs: 49 and 108; a CDR-H2 comprising one of SEQ ID NOs: 167 and 226; and a CDR-H3 comprising SEQ ID NO: 285;
(xlviii) a VH comprising: a CDR-H1 comprising one of SEQ ID NOs: 50 and 109; a CDR-H2 comprising one of SEQ ID NOs: 168 and 227; and a CDR-H3 comprising SEQ ID NO: 286;
(xlix) a VH comprising: a CDR-H1 comprising one of SEQ ID NOs: 51 and 110; a CDR-H2 comprising one of SEQ ID NOs: 169 and 228; and a CDR-H3 comprising SEQ ID NO: 287;
(l) a VH comprising: a CDR-H1 comprising one of SEQ ID NOs: 52 and 111; a CDR-H2 comprising one of SEQ ID NOs: 170 and 229; and a CDR-H3 comprising SEQ ID NO: 288;
(ii) a VH comprising: a CDR-H1 comprising one of SEQ ID NOs: 53 and 112; a CDR-H2 comprising one of SEQ ID NOs: 171 and 230; and a CDR-H3 comprising SEQ ID NO: 289;
(lii) a VH comprising: a CDR-H1 comprising one of SEQ ID NOs: 54 and 113; a CDR-H2 comprising one of SEQ ID NOs: 172 and 231; and a CDR-H3 comprising SEQ ID NO: 290;
(liii) a VH comprising: a CDR-H1 comprising one of SEQ ID NOs: 55 and 114; a CDR-H2 comprising one of SEQ ID NOs: 173 and 232; and a CDR-H3 comprising SEQ ID NO: 291;
(liv) a VH comprising: a CDR-H1 comprising one of SEQ ID NOs: 56 and 115; a CDR-H2 comprising one of SEQ ID NOs: 174 and 233; and a CDR-H3 comprising SEQ ID NO: 292;
(lv) a VH comprising: a CDR-H1 comprising one of SEQ ID NOs: 57 and 116; a CDR-H2 comprising one of SEQ ID NOs: 175 and 234; and a CDR-H3 comprising SEQ ID NO: 293;
(lvi) a VH comprising: a CDR-H1 comprising one of SEQ ID NOs: 59 and 118; a CDR-H2 comprising one of SEQ ID NOs: 177 and 236; and a CDR-H3 comprising SEQ ID NO: 295;
(lvii) a VH comprising: a CDR-H1 comprising one of SEQ ID NOs: 60 and 119; a CDR-H2 comprising one of SEQ ID NOs: 178 and 237; and a CDR-H3 comprising SEQ ID NO: 296;
(lviii) a VH comprising: a CDR-H1 comprising one of SEQ ID NOs: 61 and 120; a CDR-H2 comprising one of SEQ ID NOs: 179 and 238; and a CDR-H3 comprising SEQ ID NO: 297; or
(lix) a VH comprising: a CDR-H1 comprising one of SEQ ID NOs: 62 and 121; a CDR-H2 comprising one of SEQ ID NOs: 180 and 239; and a CDR-H3 comprising SEQ ID NO: 298.

74. The method of any of the previous claims claim 1, wherein the antibody comprises:
(a) a VL comprising: a CDR-L1 comprising SEQ ID NO: 300; a CDR-L2 comprising SEQ ID NO: 303; and a CDR-L3 comprising SEQ ID NO: 306; or
(b) a VL comprising: a CDR-L1 comprising SEQ ID NO: 301; a CDR-L2 comprising SEQ ID NO: 304; and a CDR-L3 comprising SEQ ID NO: 307.

75. The method of claim 1, wherein the antibody comprises:
(i) the VH region SEQ ID NO: 362, or a variant thereof, and the VL region SEQ ID NO: 367, or a variant thereof,
(ii) the VH region SEQ ID NO: 323, or a variant thereof, and the VL region SEQ ID NO: 367, or a variant thereof,
(iii) the VH region SEQ ID NO: 308, or a variant thereof, and the VL region SEQ ID NO: 367, or a variant thereof,
(iv) the VH region SEQ ID NO: 309, or a variant thereof, and the VL region SEQ ID NO: 367, or a variant thereof,
(v) the VH region SEQ ID NO: 310, or a variant thereof, and the VL region SEQ ID NO: 367, or a variant thereof,
(vi) the VH region SEQ ID NO: 311, or a variant thereof, and the VL region SEQ ID NO: 367, or a variant thereof,
(vii) the VH region SEQ ID NO: 312, or a variant thereof, and the VL region SEQ ID NO: 367, or a variant thereof;
(viii) the VH region SEQ ID NO: 313, or a variant thereof, and the VL region SEQ ID NO: 367, or a variant thereof;
(ix) the VH region SEQ ID NO: 314, or a variant thereof, and the VL region SEQ ID NO: 367, or a variant thereof,
(x) the VH region SEQ ID NO: 315, or a variant thereof, and the VL region SEQ ID NO: 367, or a variant thereof,
(xi) the VH region SEQ ID NO: 316, or a variant thereof, and the VL region SEQ ID NO: 367, or a variant thereof,
(xii) the VH region SEQ ID NO: 317, or a variant thereof, and the VL region SEQ ID NO: 367, or a variant thereof;
(xiii) the VH region SEQ ID NO: 318, or a variant thereof, and the VL region SEQ ID NO: 367, or a variant thereof;
(xiv) the VH region SEQ ID NO: 319, or a variant thereof, and the VL region SEQ ID NO: 367, or a variant thereof;
(xv) the VH region SEQ ID NO: 320, or a variant thereof, and the VL region SEQ ID NO: 367, or a variant thereof;
(xvi) the VH region SEQ ID NO: 321, or a variant thereof, and the VL region SEQ ID NO: 367, or a variant thereof;
(xvii) the VH region SEQ ID NO: 322, or a variant thereof, and the VL region SEQ ID NO: 367, or a variant thereof;
(xviii) the VH region SEQ ID NO: 324, or a variant thereof, and the VL region SEQ ID NO: 367, or a variant thereof;
(xix) the VH region SEQ ID NO: 325, or a variant thereof, and the VL region SEQ ID NO: 367, or a variant thereof;
(xx) the VH region SEQ ID NO: 326, or a variant thereof, and the VL region SEQ ID NO: 367, or a variant thereof; or
(xxi) the VH region SEQ ID NO: 327, or a variant thereof, and the VL region SEQ ID NO: 367, or a variant thereof;
(xxii) the VH region SEQ ID NO: 328, or a variant thereof, and the VL region SEQ ID NO: 367, or a variant thereof;
(xxiii) the VH region SEQ ID NO: 329, or a variant thereof, and the VL region SEQ ID NO: 367, or a variant thereof;
(xxiv) the VH region SEQ ID NO: 330, or a variant thereof, and the VL region SEQ ID NO: 367, or a variant thereof;
(xxv) the VH region SEQ ID NO: 331, or a variant thereof, and the VL region SEQ ID NO: 367, or a variant thereof;
(xxvi) the VH region SEQ ID NO: 332, or a variant thereof, and the VL region SEQ ID NO: 367, or a variant thereof;
(xxvii) the VH region SEQ ID NO: 333, or a variant thereof, and the VL region SEQ ID NO: 367, or a variant thereof;
(xxviii) the VH region SEQ ID NO: 334, or a variant thereof, and the VL region SEQ ID NO: 367, or a variant thereof;
(xxix) the VH region SEQ ID NO: 335, or a variant thereof, and the VL region SEQ ID NO: 367, or a variant thereof;
(xxx) the VH region SEQ ID NO: 336, or a variant thereof, and the VL region SEQ ID NO: 367, or a variant thereof;
(xxxi) the VH region SEQ ID NO: 337, or a variant thereof, and the VL region SEQ ID NO: 367, or a variant thereof;
(xxxii) the VH region SEQ ID NO: 338, or a variant thereof, and the VL region SEQ ID NO: 367, or a variant thereof;
(xxxiii) the VH region SEQ ID NO: 339, or a variant thereof, and the VL region SEQ ID NO: 367, or a variant thereof;
(xxxiv) the VH region SEQ ID NO: 340, or a variant thereof, and the VL region SEQ ID NO: 367, or a variant thereof;
(xxxv) the VH region SEQ ID NO: 341, or a variant thereof, and the VL region SEQ ID NO: 367, or a variant thereof;
(xxxvi) the VH region SEQ ID NO: 342, or a variant thereof, and the VL region SEQ ID NO: 367, or a variant thereof;
(xxxvii) the VH region SEQ ID NO: 343, or a variant thereof, and the VL region SEQ ID NO: 367, or a variant thereof;
(xxxviii) the VH region SEQ ID NO: 344, or a variant thereof, and the VL region SEQ ID NO: 367, or a variant thereof;
(xxxix) the VH region SEQ ID NO: 345, or a variant thereof, and the VL region SEQ ID NO: 367, or a variant thereof;
(xl) the VH region SEQ ID NO: 346, or a variant thereof, and the VL region SEQ ID NO: 367, or a variant thereof,
(xli) the VH region SEQ ID NO: 347, or a variant thereof, and the VL region SEQ ID NO: 367, or a variant thereof;
(xlii) the VH region SEQ ID NO: 348, or a variant thereof, and the VL region SEQ ID NO: 367, or a variant thereof;
(xliii) the VH region SEQ ID NO: 349, or a variant thereof, and the VL region SEQ ID NO: 367, or a variant thereof;
(xliv) the VH region SEQ ID NO: 350, or a variant thereof, and the VL region SEQ ID NO: 367, or a variant thereof;
(xlv) the VH region SEQ ID NO: 351, or a variant thereof, and the VL region SEQ ID NO: 367, or a variant thereof;
(xlvi) the VH region SEQ ID NO: 352, or a variant thereof, and the VL region SEQ ID NO: 367, or a variant thereof;
(xlvii) the VH region SEQ ID NO: 353, or a variant thereof, and the VL region SEQ ID NO: 367, or a variant thereof;
(xlviii) the VH region SEQ ID NO: 354, or a variant thereof, and the VL region SEQ ID NO: 367, or a variant thereof;
(xlix) the VH region SEQ ID NO: 355, or a variant thereof, and the VL region SEQ ID NO: 367, or a variant thereof;
(l) the VH region SEQ ID NO: 356, or a variant thereof, and the VL region SEQ ID NO: 367, or a variant thereof,
(li) the VH region SEQ ID NO: 357, or a variant thereof, and the VL region SEQ ID NO: 367, or a variant thereof,
(lii) the VH region SEQ ID NO: 358, or a variant thereof, and the VL region SEQ ID NO: 367, or a variant thereof,
(liii) the VH region SEQ ID NO: 359, or a variant thereof, and the VL region SEQ ID NO: 367, or a variant thereof;
(liv) the VH region SEQ ID NO: 360, or a variant thereof, and the VL region SEQ ID NO: 367, or a variant thereof;
(lv) the VH region SEQ ID NO: 361, or a variant thereof, and the VL region SEQ ID NO: 367, or a variant thereof,
(lvi) the VH region SEQ ID NO: 363, or a variant thereof, and the VL region SEQ ID NO: 368, or a variant thereof;
(lvii) the VH region SEQ ID NO: 364, or a variant thereof, and the VL region SEQ ID NO: 368, or a variant thereof;
(lviii) the VH region SEQ ID NO: 365, or a variant thereof, and the VL region SEQ ID NO: 369, or a variant thereof; or
(lix) the VH region SEQ ID NO: 366, or a variant thereof, and the VL region SEQ ID NO: 369, or a variant thereof.

76. The method of claim 1, wherein the antibody conjugate has the structure of Conjugate P: 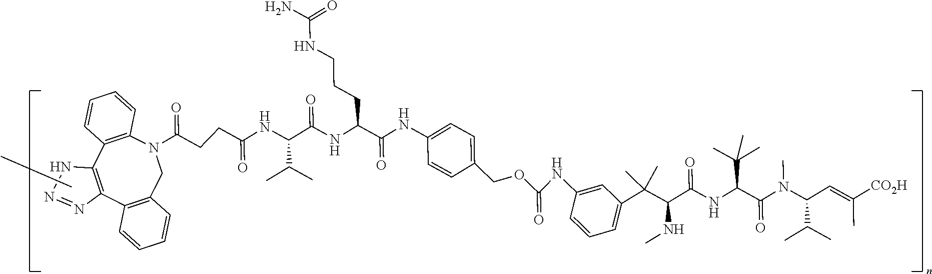
wherein n is an integer from 1 to 6.

77. The method of claim 1, wherein the antibody conjugate has the structure of Conjugate M: 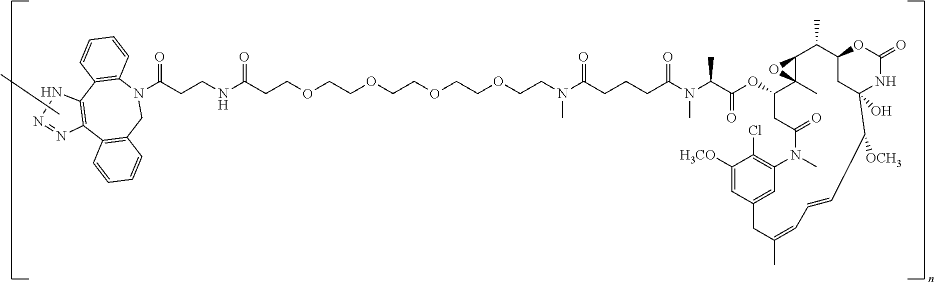
wherein n is an integer from 1 to 6.

78. The method of claim 1, wherein the antibody conjugate has the structure of Conjugate Q: 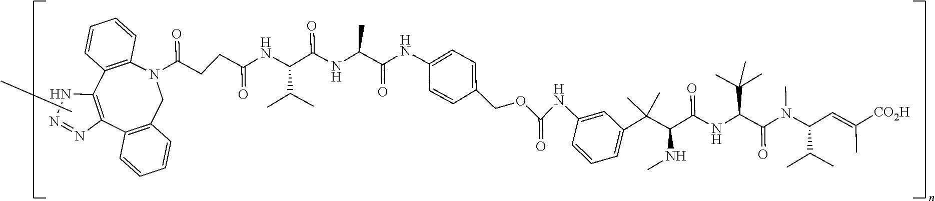
wherein n is an integer from 1 to 6.

79. The method of claim 1, wherein the antibody comprises:
a VH region of SEQ ID NO: 362, or a variant thereof 7 or fewer amino acid substitutions, and a VL region of SEQ ID NO: 367, or a variant thereof having 7 or fewer amino acid substitutions, or
a VH region of SEQ ID NO: 323, or a variant thereof 7 or fewer amino acid substitutions, and a VL region of SEQ ID NO: 367, or a variant thereof having 7 or fewer amino acid substitutions.

80. (canceled)

81. The method of claim 1, wherein the amino acid substitutions are conservative amino acid substitutions.

82. The method of claim 1, wherein the antibody further comprises at least one constant region domain.

83. The method of claim 82, wherein the constant region domain comprises a sequence selected from SEQ ID NOs: 370, 371, and 372.

84. (canceled)

85. (canceled)

86. (canceled)

87. The method of claim 1, wherein the antibody is:
a monoclonal antibody,
an IgA, an IgD, an IgE, an IgG, or an IgM,
humanized or human,
aglycosylated, and/or
an antibody fragment.

88. (canceled)

89. (canceled)

90. (canceled)

91. (canceled)

92. The method of claim 87, wherein the antibody fragment is selected from an Fv fragment, a Fab fragment, a F(ab′)2 fragment, a Fab′ fragment, an scFv (sFv) fragment, and an scFv-Fc fragment.

93. (canceled)

94. (canceled)

95. The method of claim 1 wherein the antibody conjugate has the structure of Conjugate P: 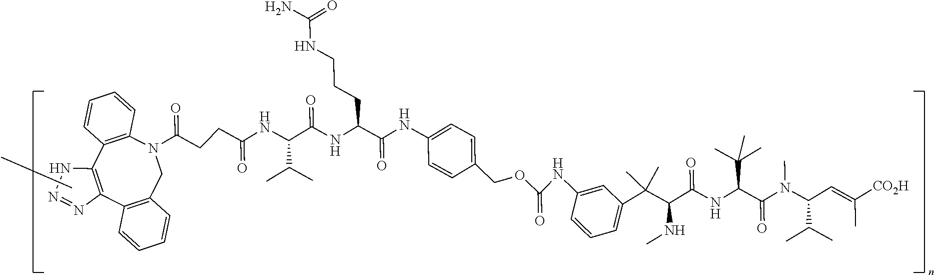
wherein
n is 4;
the antibody is an IgG antibody comprising VH regions according to SEQ ID NO: 362, and VL regions according SEQ ID NO: 367;
the antibody further comprises para-azidomethylphenylalanine residues substituting for Y180 and F404 of each VH region; and
each bond in the structure to the antibody is to the side chain of one of the para-azidomethylphenylalanine residues.


SEQUENCE LISTING (444 sequences)

SEQ ID NO: 1 - protein, 257 aa, Homo sapiens
  REGION 1..257: note = misc_feature - Human folate receptor alpha (hFOLR1)
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - protein, 257 aa, Macaca fascicularis
  REGION 1..257: note = misc_feature - Cynomolgus folate receptor alpha
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

SEQ ID NO: 3 - protein, 255 aa, Mus musculus
  REGION 1..255: note = misc_feature - Murine folate receptor alpha
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 4 - protein, 7 aa, synthetic construct
  REGION 1..7: note = Synthetic: SRP1848-A01
GFNITRY

SEQ ID NO: 5 - protein, 7 aa, synthetic construct
  REGION 1..7: note = Synthetic: SRP1848-A02
GFNISGF

SEQ ID NO: 6 - protein, 7 aa, synthetic construct
  REGION 1..7: note = Synthetic: SRP1848-A04
GFNIDQS

SEQ ID NO: 7 - protein, 7 aa, synthetic construct
  REGION 1..7: note = Synthetic: SRP1848-A06
GFNIGNS

SEQ ID NO: 8 - protein, 7 aa, synthetic construct
  REGION 1..7: note = Synthetic: SRP1848-A07
GFNIGYH

SEQ ID NO: 9 - protein, 7 aa, synthetic construct
  REGION 1..7: note = Synthetic: SRP1848-A08
GSNIRKH

SEQ ID NO: 10 - protein, 7 aa, synthetic construct
  REGION 1..7: note = Synthetic: SRP1848-A09
GFNIRKQ

SEQ ID NO: 11 - protein, 7 aa, synthetic construct
  REGION 1..7: note = Synthetic: SRP1848-A10
GFNIRKY

SEQ ID NO: 12 - protein, 7 aa, synthetic construct
  REGION 1..7: note = Synthetic: SRP1848-B01
GFNIRNY

SEQ ID NO: 13 - protein, 7 aa, synthetic construct
  REGION 1..7: note = Synthetic: SRP1848-B03
GFNISMK

SEQ ID NO: 14 - protein, 7 aa, synthetic construct
  REGION 1..7: note = Synthetic: SRP1848-B04
SFNISNH

SEQ ID NO: 15 - protein, 7 aa, synthetic construct
  REGION 1..7: note = Synthetic: SRP1848-B05
GFNISNY

SEQ ID NO: 16 - protein, 7 aa, synthetic construct
  REGION 1..7: note = Synthetic: SRP1848-B06
GFNISNY

SEQ ID NO: 17 - protein, 7 aa, synthetic construct
  REGION 1..7: note = Synthetic: SRP1848-B07
GFNISRF

SEQ ID NO: 18 - protein, 7 aa, synthetic construct
  REGION 1..7: note = Synthetic: SRP1848-B09
GFNITNY

SEQ ID NO: 19 - protein, 7 aa, synthetic construct
  REGION 1..7: note = Synthetic: SRP1848-B10
GFNTTTK

SEQ ID NO: 20 - protein, 7 aa, synthetic construct
  REGION 1..7: note = Synthetic: SRP1848-B11
GFNIGNN

SEQ ID NO: 21 - protein, 7 aa, synthetic construct
  REGION 1..7: note = Synthetic: SRP1848-C01
GFNIGNS

SEQ ID NO: 22 - protein, 7 aa, synthetic construct
  REGION 1..7: note = Synthetic: SRP1848-C03
GFNIGVY

SEQ ID NO: 23 - protein, 7 aa, synthetic construct
  REGION 1..7: note = Synthetic: SRP1848-C04
GFNIRHY

SEQ ID NO: 24 - protein, 7 aa, synthetic construct
  REGION 1..7: note = Synthetic: SRP1848-C05
GFNIRKY

SEQ ID NO: 25 - protein, 7 aa, synthetic construct
  REGION 1..7: note = Synthetic: SRP1848-C07
GFNIRKY

SEQ ID NO: 26 - protein, 7 aa, synthetic construct
  REGION 1..7: note = Synthetic: SRP1848-C10
GFNIRTY

SEQ ID NO: 27 - protein, 7 aa, synthetic construct
  REGION 1..7: note = Synthetic: SRP1848-D02
GFNISHN

SEQ ID NO: 28 - protein, 7 aa, synthetic construct
  REGION 1..7: note = Synthetic: SRP1848-D03
GFNIRYF

SEQ ID NO: 29 - protein, 7 aa, synthetic construct
  REGION 1..7: note = Synthetic: SRP1848-D04
GFNISHY

SEQ ID NO: 30 - protein, 7 aa, synthetic construct
  REGION 1..7: note = Synthetic: SRP1848-D05
GFNISIS

SEQ ID NO: 31 - protein, 7 aa, synthetic construct
  REGION 1..7: note = Synthetic: SRP1848-D07
GFNISKY

SEQ ID NO: 32 - protein, 7 aa, synthetic construct
  REGION 1..7: note = Synthetic: SRP1848-D09
GFNISNY

SEQ ID NO: 33 - protein, 7 aa, synthetic construct
  REGION 1..7: note = Synthetic: SRP1848-D10
GFNISRN

SEQ ID NO: 34 - protein, 7 aa, synthetic construct
  REGION 1..7: note = Synthetic: SRP1848-E01
GFNITNK

SEQ ID NO: 35 - protein, 7 aa, synthetic construct
  REGION 1..7: note = Synthetic: SRP1848-E02
GFNIGKY

SEQ ID NO: 36 - protein, 7 aa, synthetic construct
  REGION 1..7: note = Synthetic: SRP1848-E03
GFNIGNY

SEQ ID NO: 37 - protein, 7 aa, synthetic construct
  REGION 1..7: note = Synthetic: SRP1848-E05
GFNIGVY

SEQ ID NO: 38 - protein, 7 aa, synthetic construct
  REGION 1..7: note = Synthetic: SRP1848-E06
GFNINRY

SEQ ID NO: 39 - protein, 7 aa, synthetic construct
  REGION 1..7: note = Synthetic: SRP1848-E07
GFNIRKS

SEQ ID NO: 40 - protein, 7 aa, synthetic construct
  REGION 1..7: note = Synthetic: SRP1848-F01
GFNIRTY

SEQ ID NO: 41 - protein, 7 aa, synthetic construct
  REGION 1..7: note = Synthetic: SRP1848-F02
GFNIRTY

SEQ ID NO: 42 - protein, 7 aa, synthetic construct
  REGION 1..7: note = Synthetic: SRP1848-F04
GFNISNY

SEQ ID NO: 43 - protein, 7 aa, synthetic construct
  REGION 1..7: note = Synthetic: SRP1848-F05
GFNISKS

SEQ ID NO: 44 - protein, 7 aa, synthetic construct
  REGION 1..7: note = Synthetic: SRP1848-F06
GFNISLS

SEQ ID NO: 45 - protein, 7 aa, synthetic construct
  REGION 1..7: note = Synthetic: SRP1848-F07
GFNISNH

SEQ ID NO: 46 - protein, 7 aa, synthetic construct
  REGION 1..7: note = Synthetic: SRP1848-F08
GFNISNH

SEQ ID NO: 47 - protein, 7 aa, synthetic construct
  REGION 1..7: note = Synthetic: SRP1848-F09
GFNISNH

SEQ ID NO: 48 - protein, 7 aa, synthetic construct
  REGION 1..7: note = Synthetic: SRP1848-F10
GFNISNN

SEQ ID NO: 49 - protein, 7 aa, synthetic construct
  REGION 1..7: note = Synthetic: SRP1848-F11
GFNISNN

SEQ ID NO: 50 - protein, 7 aa, synthetic construct
  REGION 1..7: note = Synthetic: SRP1848-G01
GFNISRH

SEQ ID NO: 51 - protein, 7 aa, synthetic construct
  REGION 1..7: note = Synthetic: SRP1848-G03
GFNISTY

SEQ ID NO: 52 - protein, 7 aa, synthetic construct
  REGION 1..7: note = Synthetic: SRP1848-G04
GFNIHST

SEQ ID NO: 53 - protein, 7 aa, synthetic construct
  REGION 1..7: note = Synthetic: SRP1848-G06
GFNIRST

SEQ ID NO: 54 - protein, 7 aa, synthetic construct
  REGION 1..7: note = Synthetic: SRP1848-G07
GFNIHST

SEQ ID NO: 55 - protein, 7 aa, synthetic construct
  REGION 1..7: note = Synthetic: SRP1848-G09
GFNIRGT

SEQ ID NO: 56 - protein, 7 aa, synthetic construct
  REGION 1..7: note = Synthetic: SRP1848-G10
GFNIRST

SEQ ID NO: 57 - protein, 7 aa, synthetic construct
  REGION 1..7: note = Synthetic: SRP1848-G11
GFNISST

SEQ ID NO: 58 - protein, 7 aa, synthetic construct
  REGION 1..7: note = Synthetic: SRP1848-H01
GFNIRTQ

SEQ ID NO: 59 - protein, 9 aa, synthetic construct
  REGION 1..9: note = Synthetic: SRP2060-E10
GFSLSTFGM

SEQ ID NO: 60 - protein, 9 aa, synthetic construct
  REGION 1..9: note = Synthetic: SRP2060-E05
GFSLSTFGM

SEQ ID NO: 61 - protein, 9 aa, synthetic construct
  REGION 1..9: note = Synthetic: SRP2060-B01
GFSLSTFGM

SEQ ID NO: 62 - protein, 9 aa, synthetic construct
  REGION 1..9: note = Synthetic: SRP2060-A06
GFSLSTFGM

SEQ ID NO: 63 - protein, 5 aa, synthetic construct
  REGION 1..5: note = Synthetic: SRP1848-A01
RYSIH

SEQ ID NO: 64 - protein, 5 aa, synthetic construct
  REGION 1..5: note = Synthetic: SRP1848-A02
GFRIH

SEQ ID NO: 65 - protein, 5 aa, synthetic construct
  REGION 1..5: note = Synthetic: SRP1848-A04
QSSIH

SEQ ID NO: 66 - protein, 5 aa, synthetic construct
  REGION 1..5: note = Synthetic: SRP1848-A06
NSYIH

SEQ ID NO: 67 - protein, 5 aa, synthetic construct
  REGION 1..5: note = Synthetic: SRP1848-A07
YHSIH

SEQ ID NO: 68 - protein, 5 aa, synthetic construct
  REGION 1..5: note = Synthetic: SRP1848-A08
KHSIH

SEQ ID NO: 69 - protein, 5 aa, synthetic construct
  REGION 1..5: note = Synthetic: SRP1848-A09
KQSIH

SEQ ID NO: 70 - protein, 5 aa, synthetic construct
  REGION 1..5: note = Synthetic: SRP1848-A10
KYSIH

SEQ ID NO: 71 - protein, 5 aa, synthetic construct
  REGION 1..5: note = Synthetic: SRP1848-B01
NYSIH

SEQ ID NO: 72 - protein, 5 aa, synthetic construct
  REGION 1..5: note = Synthetic: SRP1848-B03
MKYIH

SEQ ID NO: 73 - protein, 5 aa, synthetic construct
  REGION 1..5: note = Synthetic: SRP1848-B04
NHSIH

SEQ ID NO: 74 - protein, 5 aa, synthetic construct
  REGION 1..5: note = Synthetic: SRP1848-B05
NYYIH

SEQ ID NO: 75 - protein, 5 aa, synthetic construct
  REGION 1..5: note = Synthetic: SRP1848-B06
NYYIH

SEQ ID NO: 76 - protein, 5 aa, synthetic construct
  REGION 1..5: note = Synthetic: SRP1848-B07
RFYIH

SEQ ID NO: 77 - protein, 5 aa, synthetic construct
  REGION 1..5: note = Synthetic: SRP1848-B09
NYYIH

SEQ ID NO: 78 - protein, 5 aa, synthetic construct
  REGION 1..5: note = Synthetic: SRP1848-B10
TKSIH

SEQ ID NO: 79 - protein, 5 aa, synthetic construct
  REGION 1..5: note = Synthetic: SRP1848-B11
NNSIH

SEQ ID NO: 80 - protein, 5 aa, synthetic construct
  REGION 1..5: note = Synthetic: SRP1848-C01
NSYIH

SEQ ID NO: 81 - protein, 5 aa, synthetic construct
  REGION 1..5: note = Synthetic: SRP1848-C03
VYSIH

SEQ ID NO: 82 - protein, 5 aa, synthetic construct
  REGION 1..5: note = Synthetic: SRP1848-C04
HYSIH

SEQ ID NO: 83 - protein, 5 aa, synthetic construct
  REGION 1..5: note = Synthetic: SRP1848-C05
KYSIH

SEQ ID NO: 84 - protein, 5 aa, synthetic construct
  REGION 1..5: note = Synthetic: SRP1848-C07
KYSIH

SEQ ID NO: 85 - protein, 5 aa, synthetic construct
  REGION 1..5: note = Synthetic: SRP1848-C10
TYYIH

SEQ ID NO: 86 - protein, 5 aa, synthetic construct
  REGION 1..5: note = Synthetic: SRP1848-D02
HNYIH

SEQ ID NO: 87 - protein, 5 aa, synthetic construct
  REGION 1..5: note = Synthetic: SRP1848-D03
YFSIH

SEQ ID NO: 88 - protein, 5 aa, synthetic construct
  REGION 1..5: note = Synthetic: SRP1848-D04
HYSIH

SEQ ID NO: 89 - protein, 5 aa, synthetic construct
  REGION 1..5: note = Synthetic: SRP1848-D05
ISYIH

SEQ ID NO: 90 - protein, 5 aa, synthetic construct
  REGION 1..5: note = Synthetic: SRP1848-D07
KYYIH

SEQ ID NO: 91 - protein, 5 aa, synthetic construct
  REGION 1..5: note = Synthetic: SRP1848-D09
NYYIH

SEQ ID NO: 92 - protein, 5 aa, synthetic construct
  REGION 1..5: note = Synthetic: SRP1848-D10
RNSIH

SEQ ID NO: 93 - protein, 5 aa, synthetic construct
  REGION 1..5: note = Synthetic: SRP1848-E01
NKYIH

SEQ ID NO: 94 - protein, 5 aa, synthetic construct
  REGION 1..5: note = Synthetic: SRP1848-E02
KYSIH

SEQ ID NO: 95 - protein, 5 aa, synthetic construct
  REGION 1..5: note = Synthetic: SRP1848-E03
NYYIH

SEQ ID NO: 96 - protein, 5 aa, synthetic construct
  REGION 1..5: note = Synthetic: SRP1848-E05
VYYIH

SEQ ID NO: 97 - protein, 5 aa, synthetic construct
  REGION 1..5: note = Synthetic: SRP1848-E06
RYYIH

SEQ ID NO: 98 - protein, 5 aa, synthetic construct
  REGION 1..5: note = Synthetic: SRP1848-E07
KSSIH

SEQ ID NO: 99 - protein, 5 aa, synthetic construct
  REGION 1..5: note = Synthetic: SRP1848-F01
TYSIH

SEQ ID NO: 100 - protein, 5 aa, synthetic construct
  REGION 1..5: note = Synthetic: SRP1848-F02
TYSIH

SEQ ID NO: 101 - protein, 5 aa, synthetic construct
  REGION 1..5: note = Synthetic: SRP1848-F04
NYSIH

SEQ ID NO: 102 - protein, 5 aa, synthetic construct
  REGION 1..5: note = Synthetic: SRP1848-F05
KSSIH

SEQ ID NO: 103 - protein, 5 aa, synthetic construct
  REGION 1..5: note = Synthetic: SRP1848-F06
LSYIH

SEQ ID NO: 104 - protein, 5 aa, synthetic construct
  REGION 1..5: note = Synthetic: SRP1848-F07
NHSIH

SEQ ID NO: 105 - protein, 5 aa, synthetic construct
  REGION 1..5: note = Synthetic: SRP1848-F08
NHSIH

SEQ ID NO: 106 - protein, 5 aa, synthetic construct
  REGION 1..5: note = Synthetic: SRP1848-F09
NHYIH

SEQ ID NO: 107 - protein, 5 aa, synthetic construct
  REGION 1..5: note = Synthetic: SRP1848-F10
NNSIH

SEQ ID NO: 108 - protein, 5 aa, synthetic construct
  REGION 1..5: note = Synthetic: SRP1848-F11
NNYIH

SEQ ID NO: 109 - protein, 5 aa, synthetic construct
  REGION 1..5: note = Synthetic: SRP1848-G01
RHSIH

SEQ ID NO: 110 - protein, 5 aa, synthetic construct
  REGION 1..5: note = Synthetic: SRP1848-G03
TYYIH

SEQ ID NO: 111 - protein, 5 aa, synthetic construct
  REGION 1..5: note = Synthetic: SRP1848-G04
STDIH

SEQ ID NO: 112 - protein, 5 aa, synthetic construct
  REGION 1..5: note = Synthetic: SRP1848-G06
STDIH

SEQ ID NO: 113 - protein, 5 aa, synthetic construct
  REGION 1..5: note = Synthetic: SRP1848-G07
STDIH

SEQ ID NO: 114 - protein, 5 aa, synthetic construct
  REGION 1..5: note = Synthetic: SRP1848-G09
GTDIH

SEQ ID NO: 115 - protein, 5 aa, synthetic construct
  REGION 1..5: note = Synthetic: SRP1848-G10
STDIH

SEQ ID NO: 116 - protein, 5 aa, synthetic construct
  REGION 1..5: note = Synthetic: SRP1848-G11
STDIH

SEQ ID NO: 117 - protein, 5 aa, synthetic construct
  REGION 1..5: note = Synthetic: SRP1848-H01
TQSIH

SEQ ID NO: 118 - protein, 7 aa, synthetic construct
  REGION 1..7: note = Synthetic: SRP2060-E10
TFGMGVG

SEQ ID NO: 119 - protein, 7 aa, synthetic construct
  REGION 1..7: note = Synthetic: SRP2060-E05
TFGMGVG

SEQ ID NO: 120 - protein, 7 aa, synthetic construct
  REGION 1..7: note = Synthetic: SRP2060-B01
TFGMGVG

SEQ ID NO: 121 - protein, 7 aa, synthetic construct
  REGION 1..7: note = Synthetic: SRP2060-A06
TFGMGVG

SEQ ID NO: 122 - protein, 6 aa, synthetic construct
  REGION 1..6: note = Synthetic: SRP1848-A01
LPESGG

SEQ ID NO: 123 - protein, 6 aa, synthetic construct
  REGION 1..6: note = Synthetic: SRP1848-A02
YPESGA

SEQ ID NO: 124 - protein, 6 aa, synthetic construct
  REGION 1..6: note = Synthetic: SRP1848-A04
YPVDGT

SEQ ID NO: 125 - protein, 6 aa, synthetic construct
  REGION 1..6: note = Synthetic: SRP1848-A06
TPIDGN

SEQ ID NO: 126 - protein, 6 aa, synthetic construct
  REGION 1..6: note = Synthetic: SRP1848-A07
FPVDGT

SEQ ID NO: 127 - protein, 6 aa, synthetic construct
  REGION 1..6: note = Synthetic: SRP1848-A08
YPNDGT

SEQ ID NO: 128 - protein, 6 aa, synthetic construct
  REGION 1..6: note = Synthetic: SRP1848-A09
FPNDGT

SEQ ID NO: 129 - protein, 6 aa, synthetic construct
  REGION 1..6: note = Synthetic: SRP1848-A10
FPIDDI

SEQ ID NO: 130 - protein, 6 aa, synthetic construct
  REGION 1..6: note = Synthetic: SRP1848-B01
YPVDGI

SEQ ID NO: 131 - protein, 6 aa, synthetic construct
  REGION 1..6: note = Synthetic: SRP1848-B03
TPIDGM

SEQ ID NO: 132 - protein, 6 aa, synthetic construct
  REGION 1..6: note = Synthetic: SRP1848-B04
YPVDGI

SEQ ID NO: 133 - protein, 6 aa, synthetic construct
  REGION 1..6: note = Synthetic: SRP1848-B05
SPIDGY

SEQ ID NO: 134 - protein, 6 aa, synthetic construct
  REGION 1..6: note = Synthetic: SRP1848-B06
TPIDGY

SEQ ID NO: 135 - protein, 6 aa, synthetic construct
  REGION 1..6: note = Synthetic: SRP1848-B07
SPYDGF

SEQ ID NO: 136 - protein, 6 aa, synthetic construct
  REGION 1..6: note = Synthetic: SRP1848-B09
TPVDGY

SEQ ID NO: 137 - protein, 6 aa, synthetic construct
  REGION 1..6: note = Synthetic: SRP1848-B10
YPRDGI

SEQ ID NO: 138 - protein, 6 aa, synthetic construct
  REGION 1..6: note = Synthetic: SRP1848-B11
SPIDGF

SEQ ID NO: 139 - protein, 6 aa, synthetic construct
  REGION 1..6: note = Synthetic: SRP1848-C01
TPNDGY

SEQ ID NO: 140 - protein, 6 aa, synthetic construct
  REGION 1..6: note = Synthetic: SRP1848-C03
YPIDGN

SEQ ID NO: 141 - protein, 6 aa, synthetic construct
  REGION 1..6: note = Synthetic: SRP1848-C04
YPGPGN

SEQ ID NO: 142 - protein, 6 aa, synthetic construct
  REGION 1..6: note = Synthetic: SRP1848-C05
FPIDGI

SEQ ID NO: 143 - protein, 6 aa, synthetic construct
  REGION 1..6: note = Synthetic: SRP1848-C07
FPIDGI

SEQ ID NO: 144 - protein, 6 aa, synthetic construct
  REGION 1..6: note = Synthetic: SRP1848-C10
SPIDGY

SEQ ID NO: 145 - protein, 6 aa, synthetic construct
  REGION 1..6: note = Synthetic: SRP1848-D02
TPQDGY

SEQ ID NO: 146 - protein, 6 aa, synthetic construct
  REGION 1..6: note = Synthetic: SRP1848-D03
FPNDGS

SEQ ID NO: 147 - protein, 6 aa, synthetic construct
  REGION 1..6: note = Synthetic: SRP1848-D04
YPRDGI

SEQ ID NO: 148 - protein, 6 aa, synthetic construct
  REGION 1..6: note = Synthetic: SRP1848-D05
SPIDGY

SEQ ID NO: 149 - protein, 6 aa, synthetic construct
  REGION 1..6: note = Synthetic: SRP1848-D07
SPNDGY

SEQ ID NO: 150 - protein, 6 aa, synthetic construct
  REGION 1..6: note = Synthetic: SRP1848-D09
SPNDGY

SEQ ID NO: 151 - protein, 6 aa, synthetic construct
  REGION 1..6: note = Synthetic: SRP1848-D10
SPNDGT

SEQ ID NO: 152 - protein, 6 aa, synthetic construct
  REGION 1..6: note = Synthetic: SRP1848-E01
TPFDGF

SEQ ID NO: 153 - protein, 6 aa, synthetic construct
  REGION 1..6: note = Synthetic: SRP1848-E02
YPNDGN

SEQ ID NO: 154 - protein, 6 aa, synthetic construct
  REGION 1..6: note = Synthetic: SRP1848-E03
TPRDGF

SEQ ID NO: 155 - protein, 6 aa, synthetic construct
  REGION 1..6: note = Synthetic: SRP1848-E05
TPNDGY

SEQ ID NO: 156 - protein, 6 aa, synthetic construct
  REGION 1..6: note = Synthetic: SRP1848-E06
TPNDGY

SEQ ID NO: 157 - protein, 6 aa, synthetic construct
  REGION 1..6: note = Synthetic: SRP1848-E07
FPYDGS

SEQ ID NO: 158 - protein, 6 aa, synthetic construct
  REGION 1..6: note = Synthetic: SRP1848-F01
FPNDGT

SEQ ID NO: 159 - protein, 6 aa, synthetic construct
  REGION 1..6: note = Synthetic: SRP1848-F02
FPNDGT

SEQ ID NO: 160 - protein, 6 aa, synthetic construct
  REGION 1..6: note = Synthetic: SRP1848-F04
YPIDGI

SEQ ID NO: 161 - protein, 6 aa, synthetic construct
  REGION 1..6: note = Synthetic: SRP1848-F05
YPNDGS

SEQ ID NO: 162 - protein, 6 aa, synthetic construct
  REGION 1..6: note = Synthetic: SRP1848-F06
SPIDGN

SEQ ID NO: 163 - protein, 6 aa, synthetic construct
  REGION 1..6: note = Synthetic: SRP1848-F07
YPNDGI

SEQ ID NO: 164 - protein, 6 aa, synthetic construct
  REGION 1..6: note = Synthetic: SRP1848-F08
YPVDGI

SEQ ID NO: 165 - protein, 6 aa, synthetic construct
  REGION 1..6: note = Synthetic: SRP1848-F09
SPLDGY

SEQ ID NO: 166 - protein, 6 aa, synthetic construct
  REGION 1..6: note = Synthetic: SRP1848-F10
FPNDGY

SEQ ID NO: 167 - protein, 6 aa, synthetic construct
  REGION 1..6: note = Synthetic: SRP1848-F11
TPIDGN

SEQ ID NO: 168 - protein, 6 aa, synthetic construct
  REGION 1..6: note = Synthetic: SRP1848-G01
APNDGS

SEQ ID NO: 169 - protein, 6 aa, synthetic construct
  REGION 1..6: note = Synthetic: SRP1848-G03
TPSDGF

SEQ ID NO: 170 - protein, 6 aa, synthetic construct
  REGION 1..6: note = Synthetic: SRP1848-G04
TPAGGA

SEQ ID NO: 171 - protein, 6 aa, synthetic construct
  REGION 1..6: note = Synthetic: SRP1848-G06
TPAGGA

SEQ ID NO: 172 - protein, 6 aa, synthetic construct
  REGION 1..6: note = Synthetic: SRP1848-G07
TPAGGA

SEQ ID NO: 173 - protein, 6 aa, synthetic construct
  REGION 1..6: note = Synthetic: SRP1848-G09
TPAGGA

SEQ ID NO: 174 - protein, 6 aa, synthetic construct
  REGION 1..6: note = Synthetic: SRP1848-G10
TPAGGA

SEQ ID NO: 175 - protein, 6 aa, synthetic construct
  REGION 1..6: note = Synthetic: SRP1848-G11
TPAGGA

SEQ ID NO: 176 - protein, 6 aa, synthetic construct
  REGION 1..6: note = Synthetic: SRP1848-H01
FPIDGI

SEQ ID NO: 177 - protein, 5 aa, synthetic construct
  REGION 1..5: note = Synthetic: SRP2060-E10
WWDDD

SEQ ID NO: 178 - protein, 5 aa, synthetic construct
  REGION 1..5: note = Synthetic: SRP2060-E05
WWDDD

SEQ ID NO: 179 - protein, 5 aa, synthetic construct
  REGION 1..5: note = Synthetic: SRP2060-B01
WWDDD

SEQ ID NO: 180 - protein, 5 aa, synthetic construct
  REGION 1..5: note = Synthetic: SRP2060-A06
WWDDD

SEQ ID NO: 181 - protein, 17 aa, synthetic construct
  REGION 1..17: note = Synthetic: SRP1848-A01
GILPESGGTSYADSVKG

SEQ ID NO: 182 - protein, 17 aa, synthetic construct
  REGION 1..17: note = Synthetic: SRP1848-A02
GIYPESGATYYADSVKG

SEQ ID NO: 183 - protein, 17 aa, synthetic construct
  REGION 1..17: note = Synthetic: SRP1848-A04
VIYPVDGTTDYADSVKG

SEQ ID NO: 184 - protein, 17 aa, synthetic construct
  REGION 1..17: note = Synthetic: SRP1848-A06
GITPIDGNTDYADSVKG

SEQ ID NO: 185 - protein, 17 aa, synthetic construct
  REGION 1..17: note = Synthetic: SRP1848-A07
EIFPVDGTTDYADSVKG

SEQ ID NO: 186 - protein, 17 aa, synthetic construct
  REGION 1..17: note = Synthetic: SRP1848-A08
SIYPNDGTTDYADSVKG

SEQ ID NO: 187 - protein, 17 aa, synthetic construct
  REGION 1..17: note = Synthetic: SRP1848-A09
SIFPNDGTTDYADSVKG

SEQ ID NO: 188 - protein, 17 aa, synthetic construct
  REGION 1..17: note = Synthetic: SRP1848-A10
DIFPIDDITDYADSVKG

SEQ ID NO: 189 - protein, 17 aa, synthetic construct
  REGION 1..17: note = Synthetic: SRP1848-B01
EIYPVDGITDYADSVKG

SEQ ID NO: 190 - protein, 17 aa, synthetic construct
  REGION 1..17: note = Synthetic: SRP1848-B03
GITPIDGMTDYADSVKG

SEQ ID NO: 191 - protein, 17 aa, synthetic construct
  REGION 1..17: note = Synthetic: SRP1848-B04
EIYPVDGITDYADSVKG

SEQ ID NO: 192 - protein, 17 aa, synthetic construct
  REGION 1..17: note = Synthetic: SRP1848-B05
GISPIDGYTDYADSMKG

SEQ ID NO: 193 - protein, 17 aa, synthetic construct
  REGION 1..17: note = Synthetic: SRP1848-B06
GITPIDGYTDYADSVKG

SEQ ID NO: 194 - protein, 17 aa, synthetic construct
  REGION 1..17: note = Synthetic: SRP1848-B07
GISPYDGFTDYADSVKG

SEQ ID NO: 195 - protein, 17 aa, synthetic construct
  REGION 1..17: note = Synthetic: SRP1848-B09
GITPVDGYTDYADRVKG

SEQ ID NO: 196 - protein, 17 aa, synthetic construct
  REGION 1..17: note = Synthetic: SRP1848-B10
EIYPRDGITDYADSVKG

SEQ ID NO: 197 - protein, 17 aa, synthetic construct
  REGION 1..17: note = Synthetic: SRP1848-B11
DISPIDGFTDYADSVKG

SEQ ID NO: 198 - protein, 17 aa, synthetic construct
  REGION 1..17: note = Synthetic: SRP1848-C01
GVTPNDGYTDYADSVKG

SEQ ID NO: 199 - protein, 17 aa, synthetic construct
  REGION 1..17: note = Synthetic: SRP1848-C03
EIYPIDGNTDYADSVKG

SEQ ID NO: 200 - protein, 17 aa, synthetic construct
  REGION 1..17: note = Synthetic: SRP1848-C04
EIYPGPGNTDYADSVKG

SEQ ID NO: 201 - protein, 17 aa, synthetic construct
  REGION 1..17: note = Synthetic: SRP1848-C05
DIFPIDGINDYADSVKG

SEQ ID NO: 202 - protein, 17 aa, synthetic construct
  REGION 1..17: note = Synthetic: SRP1848-C07
DIFPIDGITDYADSVKG

SEQ ID NO: 203 - protein, 17 aa, synthetic construct
  REGION 1..17: note = Synthetic: SRP1848-C10
GISPIDGYTDYADSMKG

SEQ ID NO: 204 - protein, 17 aa, synthetic construct
  REGION 1..17: note = Synthetic: SRP1848-D02
GITPQDGYTDYADSVKG

SEQ ID NO: 205 - protein, 17 aa, synthetic construct
  REGION 1..17: note = Synthetic: SRP1848-D03
DIFPNDGSTDYADSVKG

SEQ ID NO: 206 - protein, 17 aa, synthetic construct
  REGION 1..17: note = Synthetic: SRP1848-D04
EIYPRDGITDYADSVKG

SEQ ID NO: 207 - protein, 17 aa, synthetic construct
  REGION 1..17: note = Synthetic: SRP1848-D05
GISPIDGYTDYADSVKG

SEQ ID NO: 208 - protein, 17 aa, synthetic construct
  REGION 1..17: note = Synthetic: SRP1848-D07
GISPNDGYTDYADSVKG

SEQ ID NO: 209 - protein, 17 aa, synthetic construct
  REGION 1..17: note = Synthetic: SRP1848-D09
GISPNDGYTDYADSVKG

SEQ ID NO: 210 - protein, 17 aa, synthetic construct
  REGION 1..17: note = Synthetic: SRP1848-D10
WISPNDGTTDYADSVKG

SEQ ID NO: 211 - protein, 17 aa, synthetic construct
  REGION 1..17: note = Synthetic: SRP1848-E01
GITPFDGFTDYADSVKG

SEQ ID NO: 212 - protein, 17 aa, synthetic construct
  REGION 1..17: note = Synthetic: SRP1848-E02
EIYPNDGNTDYADSVKG

SEQ ID NO: 213 - protein, 17 aa, synthetic construct
  REGION 1..17: note = Synthetic: SRP1848-E03
GITPRDGFTDYADSVKG

SEQ ID NO: 214 - protein, 17 aa, synthetic construct
  REGION 1..17: note = Synthetic: SRP1848-E05
GITPNDGYTDYADSVKG

SEQ ID NO: 215 - protein, 17 aa, synthetic construct
  REGION 1..17: note = Synthetic: SRP1848-E06
GITPNDGYTDYADSVEG

SEQ ID NO: 216 - protein, 17 aa, synthetic construct
  REGION 1..17: note = Synthetic: SRP1848-E07
EIFPYDGSTDYADNVKG

SEQ ID NO: 217 - protein, 17 aa, synthetic construct
  REGION 1..17: note = Synthetic: SRP1848-F01
SIFPNDGTTDYADSVKG

SEQ ID NO: 218 - protein, 17 aa, synthetic construct
  REGION 1..17: note = Synthetic: SRP1848-F02
SIFPNDGTTDYADSVKG

SEQ ID NO: 219 - protein, 17 aa, synthetic construct
  REGION 1..17: note = Synthetic: SRP1848-F04
EIYPIDGITDYADSVKG

SEQ ID NO: 220 - protein, 17 aa, synthetic construct
  REGION 1..17: note = Synthetic: SRP1848-F05
EIYPNDGSTDYADSVKG

SEQ ID NO: 221 - protein, 17 aa, synthetic construct
  REGION 1..17: note = Synthetic: SRP1848-F06
GISPIDGNTDYADSVKG

SEQ ID NO: 222 - protein, 17 aa, synthetic construct
  REGION 1..17: note = Synthetic: SRP1848-F07
EIYPNDGITDYADSVKG

SEQ ID NO: 223 - protein, 17 aa, synthetic construct
  REGION 1..17: note = Synthetic: SRP1848-F08
EIYPVDGITDYADSVKG

SEQ ID NO: 224 - protein, 17 aa, synthetic construct
  REGION 1..17: note = Synthetic: SRP1848-F09
GISPLDGYTDYADSVKG

SEQ ID NO: 225 - protein, 17 aa, synthetic construct
  REGION 1..17: note = Synthetic: SRP1848-F10
SIFPNDGYTDYADSVKG

SEQ ID NO: 226 - protein, 17 aa, synthetic construct
  REGION 1..17: note = Synthetic: SRP1848-F11
GITPIDGNTDYADSVKG

SEQ ID NO: 227 - protein, 17 aa, synthetic construct
  REGION 1..17: note = Synthetic: SRP1848-G01
WIAPNDGSTDYADSVKG

SEQ ID NO: 228 - protein, 17 aa, synthetic construct
  REGION 1..17: note = Synthetic: SRP1848-G03
GITPSDGFTDYADSVKG

SEQ ID NO: 229 - protein, 17 aa, synthetic construct
  REGION 1..17: note = Synthetic: SRP1848-G04
YITPAGGATFYADSVKG

SEQ ID NO: 230 - protein, 17 aa, synthetic construct
  REGION 1..17: note = Synthetic: SRP1848-G06
YITPAGGATYYADNVKG

SEQ ID NO: 231 - protein, 17 aa, synthetic construct
  REGION 1..17: note = Synthetic: SRP1848-G07
YITPAGGATWYADSVKG

SEQ ID NO: 232 - protein, 17 aa, synthetic construct
  REGION 1..17: note = Synthetic: SRP1848-G09
YITPAGGATFYADSVKG

SEQ ID NO: 233 - protein, 17 aa, synthetic construct
  REGION 1..17: note = Synthetic: SRP1848-G10
YITPAGGATYYADSVKG

SEQ ID NO: 234 - protein, 17 aa, synthetic construct
  REGION 1..17: note = Synthetic: SRP1848-G11
YITPAGGATWYADSVKG

SEQ ID NO: 235 - protein, 17 aa, synthetic construct
  REGION 1..17: note = Synthetic: SRP1848-H01
DIFPIDGITDYADSVKG

SEQ ID NO: 236 - protein, 16 aa, synthetic construct
  REGION 1..16: note = Synthetic: SRP2060-E10
HIWWDDDKYYHPALKG

SEQ ID NO: 237 - protein, 16 aa, synthetic construct
  REGION 1..16: note = Synthetic: SRP2060-E05
HIWWDDDKYYHPALKG

SEQ ID NO: 238 - protein, 16 aa, synthetic construct
  REGION 1..16: note = Synthetic: SRP2060-B01
HIWWDDDKYYHPALKG

SEQ ID NO: 239 - protein, 16 aa, synthetic construct
  REGION 1..16: note = Synthetic: SRP2060-A06
HIWWDDDKYYYPALKG

SEQ ID NO: 240 - protein, 15 aa, synthetic construct
  REGION 1..15: note = Synthetic: SRP1848-A01
HIYPWDWFSNYVLDY

SEQ ID NO: 241 - protein, 15 aa, synthetic construct
  REGION 1..15: note = Synthetic: SRP1848-A02
HLYVWDWVLDHVLDY

SEQ ID NO: 242 - protein, 15 aa, synthetic construct
  REGION 1..15: note = Synthetic: SRP1848-A04
GAWSWRSGYGYYIDY

SEQ ID NO: 243 - protein, 15 aa, synthetic construct
  REGION 1..15: note = Synthetic: SRP1848-A06
GAWSWRSGYGYYIDY

SEQ ID NO: 244 - protein, 15 aa, synthetic construct
  REGION 1..15: note = Synthetic: SRP1848-A07
GFWAWRSGYGYYLDY

SEQ ID NO: 245 - protein, 15 aa, synthetic construct
  REGION 1..15: note = Synthetic: SRP1848-A08
GSWFWRAGYGYYLDY

SEQ ID NO: 246 - protein, 15 aa, synthetic construct
  REGION 1..15: note = Synthetic: SRP1848-A09
GSWFWRSGYGYFLEY

SEQ ID NO: 247 - protein, 15 aa, synthetic construct
  REGION 1..15: note = Synthetic: SRP1848-A10
GSWSWPSGHSYYLDY

SEQ ID NO: 248 - protein, 15 aa, synthetic construct
  REGION 1..15: note = Synthetic: SRP1848-B01
GFWSWPSGYSYFLDY

SEQ ID NO: 249 - protein, 15 aa, synthetic construct
  REGION 1..15: note = Synthetic: SRP1848-B03
GSWSWPSGYSYYLDY

SEQ ID NO: 250 - protein, 15 aa, synthetic construct
  REGION 1..15: note = Synthetic: SRP1848-B04
GRYSWRAGYSYYLDY

SEQ ID NO: 251 - protein, 15 aa, synthetic construct
  REGION 1..15: note = Synthetic: SRP1848-B05
GSWFWQSGYGYYLDY

SEQ ID NO: 252 - protein, 15 aa, synthetic construct
  REGION 1..15: note = Synthetic: SRP1848-B06
GFWSWPSGYGYYQDY

SEQ ID NO: 253 - protein, 15 aa, synthetic construct
  REGION 1..15: note = Synthetic: SRP1848-B07
GSWSWPAGYGYYQDY

SEQ ID NO: 254 - protein, 15 aa, synthetic construct
  REGION 1..15: note = Synthetic: SRP1848-B09
GAWSWRSGYGYYMDY

SEQ ID NO: 255 - protein, 15 aa, synthetic construct
  REGION 1..15: note = Synthetic: SRP1848-B10
GGWHWRSGYSYYLDY

SEQ ID NO: 256 - protein, 15 aa, synthetic construct
  REGION 1..15: note = Synthetic: SRP1848-B11
GSWSWRAGYGYYLDY

SEQ ID NO: 257 - protein, 15 aa, synthetic construct
  REGION 1..15: note = Synthetic: SRP1848-C01
GSWFWRAGYGYYLDY

SEQ ID NO: 258 - protein, 15 aa, synthetic construct
  REGION 1..15: note = Synthetic: SRP1848-C03
GSWAWRSGYSYYLDY

SEQ ID NO: 259 - protein, 15 aa, synthetic construct
  REGION 1..15: note = Synthetic: SRP1848-C04
GSLSWRAGYGYYLDY

SEQ ID NO: 260 - protein, 15 aa, synthetic construct
  REGION 1..15: note = Synthetic: SRP1848-C05
GSWSWKAGYGYYLDY

SEQ ID NO: 261 - protein, 15 aa, synthetic construct
  REGION 1..15: note = Synthetic: SRP1848-C07
GSWSWPAGYGYYQDY

SEQ ID NO: 262 - protein, 15 aa, synthetic construct
  REGION 1..15: note = Synthetic: SRP1848-C10
GSWSWPAGYGYYLDY

SEQ ID NO: 263 - protein, 15 aa, synthetic construct
  REGION 1..15: note = Synthetic: SRP1848-D02
GAWSWRAGYGYYLDY

SEQ ID NO: 264 - protein, 15 aa, synthetic construct
  REGION 1..15: note = Synthetic: SRP1848-D03
GHWSWPSGYWYYLDY

SEQ ID NO: 265 - protein, 15 aa, synthetic construct
  REGION 1..15: note = Synthetic: SRP1848-D04
GYWFWRSGYGYYLDY

SEQ ID NO: 266 - protein, 15 aa, synthetic construct
  REGION 1..15: note = Synthetic: SRP1848-D05
GSWSWRAGYGYYLDY

SEQ ID NO: 267 - protein, 15 aa, synthetic construct
  REGION 1..15: note = Synthetic: SRP1848-D07
GFWAWRSGYGYYLDY

SEQ ID NO: 268 - protein, 15 aa, synthetic construct
  REGION 1..15: note = Synthetic: SRP1848-D09
GSWSWRHGYGYYLDY

SEQ ID NO: 269 - protein, 15 aa, synthetic construct
  REGION 1..15: note = Synthetic: SRP1848-D10
GAWSWRSGYGYYIDY

SEQ ID NO: 270 - protein, 15 aa, synthetic construct
  REGION 1..15: note = Synthetic: SRP1848-E01
GSWSWPAGYGYYQDY

SEQ ID NO: 271 - protein, 15 aa, synthetic construct
  REGION 1..15: note = Synthetic: SRP1848-E02
GSWSWRSGYGYYLDY

SEQ ID NO: 272 - protein, 15 aa, synthetic construct
  REGION 1..15: note = Synthetic: SRP1848-E03
GSWSWPAGHSYYLDY

SEQ ID NO: 273 - protein, 15 aa, synthetic construct
  REGION 1..15: note = Synthetic: SRP1848-E05
GFWAWRSGYGYYLDY

SEQ ID NO: 274 - protein, 15 aa, synthetic construct
  REGION 1..15: note = Synthetic: SRP1848-E06
GTWSWPSGHSYYLDY

SEQ ID NO: 275 - protein, 15 aa, synthetic construct
  REGION 1..15: note = Synthetic: SRP1848-E07
GAWSWRSGYGYYIDY

SEQ ID NO: 276 - protein, 15 aa, synthetic construct
  REGION 1..15: note = Synthetic: SRP1848-F01
GSWAWRAGYSYYLDY

SEQ ID NO: 277 - protein, 15 aa, synthetic construct
  REGION 1..15: note = Synthetic: SRP1848-F02
GSWSWQAGYGYYLDY

SEQ ID NO: 278 - protein, 15 aa, synthetic construct
  REGION 1..15: note = Synthetic: SRP1848-F04
GSWFWRSGYGYYLDY

SEQ ID NO: 279 - protein, 15 aa, synthetic construct
  REGION 1..15: note = Synthetic: SRP1848-F05
GSWAWRSGYSYFLDY

SEQ ID NO: 280 - protein, 15 aa, synthetic construct
  REGION 1..15: note = Synthetic: SRP1848-F06
GFWAWRSGYGYYLDY

SEQ ID NO: 281 - protein, 15 aa, synthetic construct
  REGION 1..15: note = Synthetic: SRP1848-F07
GSWDWRSGYSYYLDY

SEQ ID NO: 282 - protein, 15 aa, synthetic construct
  REGION 1..15: note = Synthetic: SRP1848-F08
GSWYWQSGYSYYLDY

SEQ ID NO: 283 - protein, 15 aa, synthetic construct
  REGION 1..15: note = Synthetic: SRP1848-F09
GAWSWRSGYGYYIDY

SEQ ID NO: 284 - protein, 15 aa, synthetic construct
  REGION 1..15: note = Synthetic: SRP1848-F10
GSWFWRSGYGYYLDY

SEQ ID NO: 285 - protein, 15 aa, synthetic construct
  REGION 1..15: note = Synthetic: SRP1848-F11
GSWYWRAGYGYYLDY

SEQ ID NO: 286 - protein, 15 aa, synthetic construct
  REGION 1..15: note = Synthetic: SRP1848-G01
GSWAWRSGYSYFLDY

SEQ ID NO: 287 - protein, 15 aa, synthetic construct
  REGION 1..15: note = Synthetic: SRP1848-G03
GSWSWPSGHGYFLDY

SEQ ID NO: 288 - protein, 11 aa, synthetic construct
  REGION 1..11: note = Synthetic: SRP1848-G04
YPYWFAGYMDY

SEQ ID NO: 289 - protein, 11 aa, synthetic construct
  REGION 1..11: note = Synthetic: SRP1848-G06
QPYWFAGYMDY

SEQ ID NO: 290 - protein, 11 aa, synthetic construct
  REGION 1..11: note = Synthetic: SRP1848-G07
YPFWFAGYMDY

SEQ ID NO: 291 - protein, 11 aa, synthetic construct
  REGION 1..11: note = Synthetic: SRP1848-G09
HEYWFSGYMDY

SEQ ID NO: 292 - protein, 11 aa, synthetic construct
  REGION 1..11: note = Synthetic: SRP1848-G10
YPYWFAGYIDY

SEQ ID NO: 293 - protein, 11 aa, synthetic construct
  REGION 1..11: note = Synthetic: SRP1848-G11
YPYWFSGYMDY

SEQ ID NO: 294 - protein, 15 aa, synthetic construct
  REGION 1..15: note = Synthetic: SRP1848-H01
GSWSWPSGMDYYLDY

SEQ ID NO: 295 - protein, 16 aa, synthetic construct
  REGION 1..16: note = Synthetic: SRP2060-E10
NHFPHYYGSSHWYFNV

SEQ ID NO: 296 - protein, 16 aa, synthetic construct
  REGION 1..16: note = Synthetic: SRP2060-E05
NHFPHYYGSSHWYFNV

SEQ ID NO: 297 - protein, 16 aa, synthetic construct
  REGION 1..16: note = Synthetic: SRP2060-B01
NHFPHYYGSSHWYFNV

SEQ ID NO: 298 - protein, 16 aa, synthetic construct
  REGION 1..16: note = Synthetic: SRP2060-A06
NHFPHYYGSSHWYFDV

SEQ ID NO: 299 - protein, 11 aa, synthetic construct
  REGION 1..11: note = Synthetic: trastuzumab
RASQDVNTAVA

SEQ ID NO: 300 - protein, 11 aa, synthetic construct
  REGION 1..11: note = Synthetic: H6D1-LC4
KASQDINSYLS

SEQ ID NO: 301 - protein, 11 aa, synthetic construct
  REGION 1..11: note = Synthetic: H6D1-LC5
KASQDINSYLS

SEQ ID NO: 302 - protein, 7 aa, synthetic construct
  REGION 1..7: note = Synthetic: trastuzumab
SASFLYS

SEQ ID NO: 303 - protein, 7 aa, synthetic construct
  REGION 1..7: note = Synthetic: H6D1-LC4
RANRLVD

SEQ ID NO: 304 - protein, 7 aa, synthetic construct
  REGION 1..7: note = Synthetic: H6D1-LC5
RANRLVD

SEQ ID NO: 305 - protein, 9 aa, synthetic construct
  REGION 1..9: note = Synthetic: trastuzumab
QQHYTTPPT

SEQ ID NO: 306 - protein, 9 aa, synthetic construct
  REGION 1..9: note = Synthetic: H6D1-LC4
LQYDEFPYT

SEQ ID NO: 307 - protein, 9 aa, synthetic construct
  REGION 1..9: note = Synthetic: H6D1-LC5
LQYDEFPYT

SEQ ID NO: 308 - protein, 124 aa, synthetic construct
  REGION 1..124: note = Synthetic: SRP1848-A01
EVQLVESGGGLVQPGGSLRLSCAASGFNITRYSIHWVRQAPGKGLEWVAGILPESGGTSYADSVKGRFTISADTSKNTAYLQMNSLRAEDTAVYYCARHIYPWDWFSNYVLDYWGQGTLVTVSS

SEQ ID NO: 309 - protein, 124 aa, synthetic construct
  REGION 1..124: note = Synthetic: SRP1848-A02
EVQLVESGGGLVQPGGSLRLSCAASGFNISGFRIHWVRQAPGKGLEWVAGIYPESGATYYADSVKGRFTISADTSKNTAYLQMNSLRAEDTAVYYCARHLYVWDWVLDHVLDYWGQGTLVTVSS

SEQ ID NO: 310 - protein, 124 aa, synthetic construct
  REGION 1..124: note = Synthetic: SRP1848-A04
EVQLVESGGGLVQPGGSLRLSCAASGFNIDQSSIHWVRQAPGKGLEWVGVIYPVDGTTDYADSVKGRFTISADTSKNTAYLQMNSLRAEDTAVYYCARGAWSWRSGYGYYIDYWGQGTLVTVSS

SEQ ID NO: 311 - protein, 124 aa, synthetic construct
  REGION 1..124: note = Synthetic: SRP1848-A06
EVQLVESGGGLVQPGGSLRLSCAASGFNIGNSYIHWVRQAPGKGLEWVGGITPIDGNTDYADSVKGRFTISADTSKNTAYLQMNSLRAEDTAVYYCARGAWSWRSGYGYYIDYWGQGTLVTVSS

SEQ ID NO: 312 - protein, 124 aa, synthetic construct
  REGION 1..124: note = Synthetic: SRP1848-A07
EVQLVESGGGLVQPGGSLRLSCAASGFNIGYHSIHWVRQAPGKGLEWVGEIFPVDGTTDYADSVKGRFTISADTSKNTAYLHMNSLRAEDTAVYYCARGFWAWRSGYGYYLDYWGQGTLVTVSS

SEQ ID NO: 313 - protein, 124 aa, synthetic construct
  REGION 1..124: note = Synthetic: SRP1848-A08
EVQLVESGGGLVQPGGSLRLSCAASGSNIRKHSIHWVRQAPGKGLEWVGSIYPNDGTTDYADSVKGRFTISADTSKNTAYLQMNSLRAEDTAVYYCARGSWFWRAGYGYYLDYWGQGTLVTVSS

SEQ ID NO: 314 - protein, 124 aa, synthetic construct
  REGION 1..124: note = Synthetic: SRP1848-A09
EVQLVESGGGLVQPGGSLRLSCAASGFNIRKQSIHWVRQAPGKGLEWVGSIFPNDGTTDYADSVKGRFTISADTSKNTAYLQVNSLRAEDTAVYYCARGSWFWRSGYGYFLEYWGQGTLVTVSS

SEQ ID NO: 315 - protein, 124 aa, synthetic construct
  REGION 1..124: note = Synthetic: SRP1848-A10
EVQLVESGGGLVQPGGSLRLSCAASGFNIRKYSIHWARQAPGKGLEWVGDIFPIDDITDYADSVKGRFTISADTSKNTAYLQMNSLRAEDTAVYYCARGSWSWPSGHSYYLDYWGQGTLVTVSS

SEQ ID NO: 316 - protein, 124 aa, synthetic construct
  REGION 1..124: note = Synthetic: SRP1848-B01
EVQLVESGGGLVQPGGSLRLSCAASGFNIRNYSIHWVRQAPGKGLEWVGEIYPVDGITDYADSVKGRFTISADTSKNTAYLQMNSLRAEDTAVYYCARGFWSWPSGYSYFLDYWGQGTLVTVSS

SEQ ID NO: 317 - protein, 124 aa, synthetic construct
  REGION 1..124: note = Synthetic: SRP1848-B03
EVQLVESGGGLVQPGGSLRLSCAASGFNISMKYIHWVRQAPGKGLEWVGGITPIDGMTDYADSVKGRFTISADTSKNTAYLQMNSLRAEDTAVYYCARGSWSWPSGYSYYLDYWGQGTLVTVSS

SEQ ID NO: 318 - protein, 124 aa, synthetic construct
  REGION 1..124: note = Synthetic: SRP1848-B04
EVQLVESGGGLVQPGGSLRLSCAASSFNISNHSIHWVRQAPGKGLEWVGEIYPVDGITDYADSVKGRFTISADTSKNTAYLQMNSLRAEDTAVYYCARGRYSWRAGYSYYLDYWGQGTLVTVSS

SEQ ID NO: 319 - protein, 124 aa, synthetic construct
  REGION 1..124: note = Synthetic: SRP1848-B05
EVQLVESGGGLVQPGGSLRLSCAASGFNISNYYIHWVRQAPGKGLEWVGGISPIDGYTDYADSMKGRFTISADTSKNTAYLQMSSLRAEDTAVYYCARGSWFWQSGYGYYLDYWGQGTLVTVSS

SEQ ID NO: 320 - protein, 124 aa, synthetic construct
  REGION 1..124: note = Synthetic: SRP1848-B06
EVQLVESGGGLVQPGGSLRLSCAASGFNISNYYIHWVRQAPGKGLEWVGGITPIDGYTDYADSVKGRFTISADTSKNTAYLQMNSLRAEDTAVYYCARGFWSWPSGYGYYQDYWGQGTLVTVSS

SEQ ID NO: 321 - protein, 124 aa, synthetic construct
  REGION 1..124: note = Synthetic: SRP1848-B07
EVQLVESGGGLVQPGGSLRLSCAASGFNISRFYIHWVRQAPGKGLEWVGGISPYDGFTDYADSVKGRFTISADTSKNTAYLQMNSLRAEDTAVYYCARGSWSWPAGYGYYQDYWGQGTLVTVSS

SEQ ID NO: 322 - protein, 124 aa, synthetic construct
  REGION 1..124: note = Synthetic: SRP1848-B09
EVQLVESGGGLVQPGGSLRLSCAAGGFNITNYYIHWVRQAPGKGLEWVGGITPVDGYTDYADRVKGRFTISADTSKNTAYLQMNSLRAEDTAVYYCARGAWSWRSGYGYYMDYWGQGTLVTVSS

SEQ ID NO: 323 - protein, 124 aa, synthetic construct
  REGION 1..124: note = Synthetic: SRP1848-B10
EVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSS

SEQ ID NO: 324 - protein, 124 aa, synthetic construct
  REGION 1..124: note = Synthetic: SRP1848-B11
EVQLVESGGGLVQPGGSLRLSCAASGFNIGNNSIHWVRQAPGKGLEWVGDISPIDGFTDYADSVKGRFTISADTSKNTAYLQMNSLRAEDTAVYYCARGSWSWRAGYGYYLDYWGQGTLVTVSS

SEQ ID NO: 325 - protein, 124 aa, synthetic construct
  REGION 1..124: note = Synthetic: SRP1848-C01
EVQLVESGGGLVQPGGSLRLSCAASGFNIGNSYIHWVRQAPGKGLEWVGGVTPNDGYTDYADSVKGRFTISADTSKNTTYLQMNSLRAEDTAVYYCARGSWFWRAGYGYYLDYWGQGALVTVSS

SEQ ID NO: 326 - protein, 124 aa, synthetic construct
  REGION 1..124: note = Synthetic: SRP1848-C03
EVQLVESGGGLVQPGGSLRLSCAASGFNIGVYSIHWVRQAPGKGLEWVGEIYPIDGNTDYADSVKGRFTISADTSKNTAYLQMNSLRAEDTAVYYCARGSWAWRSGYSYYLDYWGQGTLVTVSS

SEQ ID NO: 327 - protein, 124 aa, synthetic construct
  REGION 1..124: note = Synthetic: SRP1848-C04
EVQLVESGGGLVQPGGSLRLSCAASGFNIRHYSIHWVRQAPGKGLEWVGEIYPGPGNTDYADSVKGRFTISADTSKNTAYLQMNSLRAEDTAVYYCARGSLSWRAGYGYYLDYWGQGTLVTVSS

SEQ ID NO: 328 - protein, 124 aa, synthetic construct
  REGION 1..124: note = Synthetic: SRP1848-C05
EVQLVESGGGLVQPGGSLRLSCAASGFNIRKYSIHWVRQAPGKGLEWVGDIFPIDGINDYADSVKGRFTISADTSKNTAYLQMNSLRAEDTAVYYCARGSWSWKAGYGYYLDYWGQGTLVTVSS

SEQ ID NO: 329 - protein, 124 aa, synthetic construct
  REGION 1..124: note = Synthetic: SRP1848-C07
EVQLVESGGGLVQPGGSLRLSCAASGFNIRKYSIHWVRQAPGKGLEWVGDIFPIDGITDYADSMKGRFTISADTSKNTAYLQMNSLRAEDTAVYYCARGSWSWPAGYGYYQDYWGQGTLVTVSS

SEQ ID NO: 330 - protein, 124 aa, synthetic construct
  REGION 1..124: note = Synthetic: SRP1848-C10
EVQLVESGGGLVQPGGSLRLSCAASGFNIRTYYIHWVRQAPGKGLEWVGGISPIDGYTDYADSVKGRFTISADTSKNTAYLQMNSLRAEDTAVYYCARGSWSWPAGYGYYLDYWGQGTLVTVSS

SEQ ID NO: 331 - protein, 124 aa, synthetic construct
  REGION 1..124: note = Synthetic: SRP1848-D02
EVQLVESGGGLVQPGGSLRLSCAASGFNISHNYIHWVRQAPGKGLEWVGGITPQDGYTDYADSVKGRFTISADTSKNTAYLQMNRLRAEDTAVYYCARGAWSWRAGYGYYLDYWGQGTLVTVSS

SEQ ID NO: 332 - protein, 124 aa, synthetic construct
  REGION 1..124: note = Synthetic: SRP1848-D03
EVQLVESGGGVVQPGGSLRLSCAASGFNIRYFSIHWVRQAPGKGLEWVGDIFPNDGSTDYADSVKGRFTISADTSKNTAYLQMNSLRAEETAVYYCARGHWSWPSGYWYYLDYWGQGTLVTVSS

SEQ ID NO: 333 - protein, 124 aa, synthetic construct
  REGION 1..124: note = Synthetic: SRP1848-D04
EVQLVESGGGLVQPGGSLRLSCAASGFNISHYSIHWVRQAPGKGLEWVGEIYPRDGITDYADSVKGRFTISADTSKNTAYLQMNSLSAEDTAVYYCARGYWFWRSGYGYYLDYWGQGTLVTVSS

SEQ ID NO: 334 - protein, 124 aa, synthetic construct
  REGION 1..124: note = Synthetic: SRP1848-D05
EVQLVESGGGLVQPGGSLRLSCAASGFNISISYIHWVRQAPGKGLEWVGGISPIDGYTDYADSVKGRFTISADTSKNTAYLQMNSLRAEDTAVYYCARGSWSWRAGYGYYLDYWGQGTLVTVSS

SEQ ID NO: 335 - protein, 124 aa, synthetic construct
  REGION 1..124: note = Synthetic: SRP1848-D07
EVQLVESGGGLVQPGGSLRLSCAASGFNISKYYIHWVRQAPGKGLEWVGGISPNDGYTDYADSVKGRFAISADTSKNTAYLQMNSLRAEDTAVYYCARGFWAWRSGYGYYLDYWGQGTLVTVSS

SEQ ID NO: 336 - protein, 124 aa, synthetic construct
  REGION 1..124: note = Synthetic: SRP1848-D09
EVQLVESGGGLVQPGGSLRLSCAASGFNISNYYIHWVRQAPGKGLEWVGGISPNDGYTDYADSVKGRFTISADTSKNTAYLQMNSLRAEDTAVYYCARGSWSWRHGYGYYLDYWGQGTLVTVSS

SEQ ID NO: 337 - protein, 124 aa, synthetic construct
  REGION 1..124: note = Synthetic: SRP1848-D10
EVQLVESGGGLVQPGGSLRLSCAASGFNISRNSIHWVRQAPGKGLEWVGWISPNDGTTDYADSVKGRFTISADGSKNTAYLQMNSLRAEDTAVYYCARGAWSWRSGYGYYIDYWGQGTLVTVSS

SEQ ID NO: 338 - protein, 124 aa, synthetic construct
  REGION 1..124: note = Synthetic: SRP1848-E01
EVQLVESGGGLVQPGGSLRLSCAASGFNITNKYIHWVRQAPGKGLEWVGGITPFDGFTDYADSVKGRFTISADTSKNTAYLQMNSLRAEDTAVYYCARGSWSWPAGYGYYQDYWGQGTLVTVSS

SEQ ID NO: 339 - protein, 124 aa, synthetic construct
  REGION 1..124: note = Synthetic: SRP1848-E02
EVQLVESGGGLVQPGGSLRLSCAASGFNIGKYSIHWVRQAPGKGLEWVGEIYPNDGNTDYADSVKGRFTISADTSKNTAYLQMNSLRAEDTAVYYCARGSWSWRSGYGYYLDYWGQGTLVTVSS

SEQ ID NO: 340 - protein, 124 aa, synthetic construct
  REGION 1..124: note = Synthetic: SRP1848-E03
EVQLVESGGGLAQPGGSLRLSCAASGFNIGNYYIHWVRQAPGKGLEWVGGITPRDGFTDYADSVKGRFTISADTSKNTAYLQVNSLRAEDTAVYYCARGSWSWPAGHSYYLDYWGQGTLVTVSS

SEQ ID NO: 341 - protein, 124 aa, synthetic construct
  REGION 1..124: note = Synthetic: SRP1848-E05
EVQLVESGGGLVQPGGSLRVSCAASGFNIGVYYIHWVRQAPGKGLEWVGGITPNDGYTDYADSVKGRFTISADTSKNTAYLQMNSLRAEDTAVYYCARGFWAWRSGYGYYLDYWGQGTLVTVSS

SEQ ID NO: 342 - protein, 124 aa, synthetic construct
  REGION 1..124: note = Synthetic: SRP1848-E06
EVQLVESGGGLVQPSGSLRLSCAASGFNINRYYIHWVRQAPGKGLEWVGGITPNDGYTDYADSVEGRFTTSADTSKNTAYLQMNSLRAEDTAVYYCARGTWSWPSGHSYYLDYWGQGTLVTVSS

SEQ ID NO: 343 - protein, 124 aa, synthetic construct
  REGION 1..124: note = Synthetic: SRP1848-E07
EVQLVESGGGLVQPGGSLRLSCAASGFNIRKSSIHWVRQAPGKGLEWVGEIFPYDGSTDYADNVKGRFTISADTSKNTAYLQMNSLRAEDTAVYYCARGAWSWRSGYGYYIDYWGQGTLVTVSS

SEQ ID NO: 344 - protein, 124 aa, synthetic construct
  REGION 1..124: note = Synthetic: SRP1848-F01
EVQLVESGGGLVQPGGSLRLSCAASGFNIRTYSIHWVRQAPGKGLEWVGSIFPNDGTTDYADSVKGRFTISADTSKNTAYLQMNSLRAEDTAVYYCARGSWAWRAGYSYYLDYWGQGTLVTVSS

SEQ ID NO: 345 - protein, 124 aa, synthetic construct
  REGION 1..124: note = Synthetic: SRP1848-F02
EVQLVESGGGLVQPGGSLRLSCAASGFNIRTYSIHWVRQAPGKGLEWVGSIFPNDGTTDYADSVKGRLTISADTSKNTAYLQMNSLRAEDTAVYYCARGSWSWQAGYGYYLDYWGQGTLVTVSS

SEQ ID NO: 346 - protein, 124 aa, synthetic construct
  REGION 1..124: note = Synthetic: SRP1848-F04
EVQLVESGGGLVQPGGSLRLSCAASGFNISNYSIHWVRQAPGKGLEWVGEIYPIDGITDYADSVKGRFTISADTSKNTAYLQMNSLRAEDTAVYYCARGSWFWRSGYGYYLDYWGQGTLVTVSS

SEQ ID NO: 347 - protein, 124 aa, synthetic construct
  REGION 1..124: note = Synthetic: SRP1848-F05
EVQLVESGGGLVQPGGSLRLSCAASGFNISKSSIHWVRQAPGKGLEWVGEIYPNDGSTDYADSVKGRFTISADTSKNTAYLQMNSLRAEDTAVYYCARGSWAWRSGYSYFLDYWGQGTLVTVSS

SEQ ID NO: 348 - protein, 124 aa, synthetic construct
  REGION 1..124: note = Synthetic: SRP1848-F06
EVQLVESGGGLVQPGGSLRLSCAASGFNISLSYIHWVRQAPGKGLEWVGGISPIDGNTDYADSVKGRFTISADTSKNTAYLQMNSLRAEDTAVYYCARGFWAWRSGYGYYLDYWGQGTLVTVSS

SEQ ID NO: 349 - protein, 124 aa, synthetic construct
  REGION 1..124: note = Synthetic: SRP1848-F07
EVQLVESGGGLVQPGGSLRLSCAASGFNISNHSIHWVRQAPGKGLEWVGEIYPNDGITDYADSVKGRFTISADTSKNTAYLQMNSLSAEDTAVYYCARGSWDWRSGYSYYLDYWGQGTLVTVSS

SEQ ID NO: 350 - protein, 124 aa, synthetic construct
  REGION 1..124: note = Synthetic: SRP1848-F08
EVQLVESGGGLVQPGGSLRLSCAAGGFNISNHSIHWVRQAPGKGVEWVGEIYPVDGITDYADSVKGRFTISADTSKNTAYLRMNSLRAEDTAVYYCARGSWYWQSGYSYYLDYWGQGTLVTVSS

SEQ ID NO: 351 - protein, 124 aa, synthetic construct
  REGION 1..124: note = Synthetic: SRP1848-F09
EVQLVESGGGLVQPGGSLRLSCAASGFNISNHYIHWVRQAPGKGLEWVGGISPLDGYTDYADSVKGRFTISADTSKNTAYLQMNSLRAEDTAVYYCARGAWSWRSGYGYYIDYWGQGTLVTVSS

SEQ ID NO: 352 - protein, 124 aa, synthetic construct
  REGION 1..124: note = Synthetic: SRP1848-F10
EVQLVESGGGLVQPGGSLRLSCAASGFNISNNSIHWVRQAPGKGLEWVGSIFPNDGYTDYADSVKGRFTISADTSKNTAYLQMNSLRAEDTAVYYCARGSWFWRSGYGYYLDYWGQGTLVTVSS

SEQ ID NO: 353 - protein, 124 aa, synthetic construct
  REGION 1..124: note = Synthetic: SRP1848-F11
EVQLVESGGGLVQPGGSLRLSCAASGFNISNNYIHWVRQAPGKGLEWVGGITPIDGNTDYADSVKGRFTISADTSMNTAYLQMNSLRAEDTAVYYCARGSWYWRAGYGYYLDYWGQGALVTVSS

SEQ ID NO: 354 - protein, 124 aa, synthetic construct
  REGION 1..124: note = Synthetic: SRP1848-G01
EVQLVESGGGLVQPGGSLRLSCAASGFNISRHSIHWVRQAPGKGLEWVGWIAPNDGSTDYADSVKGRFTISADTSKNTAYLQMNSLRAEDTAVYYCARGSWAWRSGYSYFLDYWGQGTLVTVSS

SEQ ID NO: 355 - protein, 124 aa, synthetic construct
  REGION 1..124: note = Synthetic: SRP1848-G03
EVQLVESGGGLVQPGGSLRLSCAASGFNISTYYIHWVRQAPGKGLEWVGGITPSDGFTDYADSVKGRSTISADTSKNTAYLQMNSLRAEDTAVYYCARGSWSWPSGHGYFLDYWGQGTLVTVSS

SEQ ID NO: 356 - protein, 120 aa, synthetic construct
  REGION 1..120: note = Synthetic: SRP1848-G04
EVQLVESGGGLVQPGGSLRLSCAASGFNIHSTDIHWVRQAPGKGLEWVAYITPAGGATFYADSVKGRFTISADTSKNTAYLQMNSLRAEDTAVYYCARYPYWFAGYMDYWGQGTLVTVSS

SEQ ID NO: 357 - protein, 120 aa, synthetic construct
  REGION 1..120: note = Synthetic: SRP1848-G06
EVQLVESGGGLVQPGGSLRLSCAASGFNIRSTDIHWVRQAPGKGLEWVAYITPAGGATYYADNVKGRFTISADTSKNTAYLQMNSLRAEDTAVYYCARQPYWFAGYMDYWGQGTLVTVSS

SEQ ID NO: 358 - protein, 120 aa, synthetic construct
  REGION 1..120: note = Synthetic: SRP1848-G07
EVQLVESGGGLVQPGGSLRLSCAASGFNIHSTDIHWVRQAPGKGLEWVAYITPAGGATWYADSVKGRFTISADTSKNTAYLQMNSLRAEDTAVYYCARYPFWFAGYMDYWGQGTLVTVSS

SEQ ID NO: 359 - protein, 120 aa, synthetic construct
  REGION 1..120: note = Synthetic: SRP1848-G09
EVQLVESGGGLVQPGGSLRLSCAASGFNIRGTDIHWVRQAPGKGLEWVAYITPAGGATFYADSVKGRFTISADTSKNTAYLQMNSLRAEDTAVYYCARHEYWFSGYMDYWGQGTLVTVSS

SEQ ID NO: 360 - protein, 120 aa, synthetic construct
  REGION 1..120: note = Synthetic: SRP1848-G10
EVQLVESGGGLVQPGSSLRLSCAASGFNIRSTDIHWVRQAPGKGLEWVAYITPAGGATYYADSVKGRFTISADTSKNTAYLQMNSLRAEDTAVYYCARYPYWFAGYIDYWGQGTLVTVSS

SEQ ID NO: 361 - protein, 120 aa, synthetic construct
  REGION 1..120: note = Synthetic: SRP1848-G11
EVQLVESGGGLVQPGGSLRLSCAASGFNISSTDIHWVRQAPGKGLEWVAYITPAGGATWYADSVKGRFTISADTSKNTAYLQMNSLRAEDTAVYYCARYPYWFSGYMDYWGQGTLVTVSS

SEQ ID NO: 362 - protein, 124 aa, synthetic construct
  REGION 1..124: note = Synthetic: SRP1848-H01
EVQLVESGGGLVQPGGSLRLSCAASGFNIRTQSIHWVRQAPGKGLEWIGDIFPIDGITDYADSVKGRFTISADTSKNTAYLQMNSLRAEDTAVYYCARGSWSWPSGMDYYLDYWGQGTLVTVSS

SEQ ID NO: 363 - protein, 126 aa, synthetic construct
  REGION 1..126: note = Synthetic: SRP2060-E10
EVQLLESGGGLVQPGGSLRLSCAFSGFSLSTFGMGVGWVRQAPGKGLEWVSHIWWDDDKYYHPALKGRFTISKDNSKNTVYLQMNSLRAEDTAVYYCGRNHFPHYYGSSHWYFNVWGQGTTVTVSS

SEQ ID NO: 364 - protein, 126 aa, synthetic construct
  REGION 1..126: note = Synthetic: SRP2060-E05
EVQLLESGGGLVQPGGSLRLSCAFSGFSLSTFGMGVGWVRQAPGKGLEWVSHIWWDDDKYYHPALKGRFTVSKDNSKNTVYLQMNSLRAEDTAVYYCGRNHFPHYYGSSHWYFNVWGQGTTVTVSS

SEQ ID NO: 365 - protein, 126 aa, synthetic construct
  REGION 1..126: note = Synthetic: SRP2060-B01
EVQLLESGGGLVQPGGSLRLSCALSGFSLSTFGMGVGWVRQATGKGLEWVSHIWWDDDKYYHPALKGRFTISKDNSKNTVHLQMNSLRAEDTAVYYCGRNHFPHYYGSSHWYFNVWGQGTTVTVSS

SEQ ID NO: 366 - protein, 126 aa, synthetic construct
  REGION 1..126: note = Synthetic: SRP2060-A06
EVQLLESGGGLVQPGGSLRLSCAFSGFSLSTFGMGVGWVRQAPGKGLEWVGHIWWDDDKYYYPALKGRFTISKDNSKNTVYLQMNSLRAEDTAVYYCGRNHFPHYYGSSHWYFDVWGQGTTVTVSS

SEQ ID NO: 367 - protein, 107 aa, synthetic construct [sequence omitted]

SEQ ID NO: 368 - protein, 107 aa, synthetic construct
  REGION 1..107: note = Synthetic: H6D1-LC4
EIVMTQSPATLSLSPGERATLSCKASQDINSYLSWYQQKPGQAPRLLIYRANRLVDGIPARFSGSGSGTDYTLTISSLEPEDFAVYYCLQYDEFPYTFGGGTKVEIK

SEQ ID NO: 369 - protein, 107 aa, synthetic construct
  REGION 1..107: note = Synthetic: H6D1-LC5
DIQMTQSPSTLSASVGDRVTITCKASQDINSYLSWYQQKPGKAPKLLIYRANRLVDGVPSRFSGSGSGTEFTLTISSLQPDDFATYYCLQYDEFPYTFGGGTKVEIK

SEQ ID NO: 370 - protein, 330 aa, Homo sapiens
  REGION 1..330: note = misc_feature - Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 107 aa, Homo sapiens [sequence omitted]

SEQ ID NO: 372 - protein, 323 aa, synthetic construct
  REGION 1..323: note = Synthetic: Mouse IgG1 HC Constant
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 373 - protein, 107 aa, synthetic construct
  REGION 1..107: note = Synthetic: Mouse IgG LC Constant Ckappa
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 374 - protein, 108 aa, synthetic construct
  REGION 1..108: note = Synthetic: Kappa LC
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 106 aa, synthetic construct
  REGION 1..106: note = Synthetic: Lambda LD
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 376 - protein, 20 aa, synthetic construct
  REGION 1..20: note = Synthetic: FlagHis Tag
GSGDYKDDDDKGSGHHHHHH

SEQ ID NO: 377 - protein, 15 aa, synthetic construct
  REGION 1..15: note = Synthetic: Linker
GGGGSGGGGSGGGGS

SEQ ID NO: 378 - protein, 11 aa, synthetic construct
  REGION 1..11: note = Synthetic: Linker
AAGSDQEPKSS

SEQ ID NO: 379 - protein, 247 aa, synthetic construct
  REGION 1..247: note = Synthetic: 1848-B10-VH-(G4S)3-VL
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 380 - protein, 247 aa, synthetic construct
  REGION 1..247: note = Synthetic: 1848-B10-VL-(G4S)3-VH
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSS

SEQ ID NO: 381 - protein, 485 aa, synthetic construct
  REGION 1..485: note = Synthetic: 1848-B10-VH-(G4S)3-VL
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 485 aa, synthetic construct
  REGION 1..485: note = Synthetic: 1848-B10-VL-(G4S)3-VH
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 249 aa, synthetic construct
  REGION 1..249: note = Synthetic: Farletuzumab
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKGGGGSGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 384 - protein, 249 aa, synthetic construct
  REGION 1..249: note = Synthetic: Farletuzumab
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSGGGGSGGGGSGGGGSGGGGSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 385 - protein, 5 aa, synthetic construct
  REGION 1..5: note = Synthetic: Farletuzumab
GYGLS

SEQ ID NO: 386 - protein, 17 aa, synthetic construct
  REGION 1..17: note = Synthetic: Farletuzumab
MISSGGSYTYYADSVKG

SEQ ID NO: 387 - protein, 10 aa, synthetic construct
  REGION 1..10: note = Synthetic: Farletuzumab
HGDDPAWFAY

SEQ ID NO: 388 - protein, 12 aa, synthetic construct
  REGION 1..12: note = Synthetic: Farletuzumab
SVSSSISSNNLH

SEQ ID NO: 389 - protein, 7 aa, synthetic construct
  REGION 1..7: note = Synthetic: Farletuzumab
GTSNLAS

SEQ ID NO: 390 - protein, 11 aa, synthetic construct
  REGION 1..11: note = Synthetic: Farletuzumab
QQWSSYPYMYT

SEQ ID NO: 391 - protein, 119 aa, synthetic construct
  REGION 1..119: note = Synthetic: Farletuzumab
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 392 - protein, 110 aa, synthetic construct
  REGION 1..110: note = Synthetic: Farletuzumab
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 393 - DNA, 357 bp, synthetic construct
  misc_feature 1..357: note = Synthetic
  source 1..357: mol_type = other DNA
gaggtacagcttgtcgagagcggtggtggagtagtccaaccgggtcgaagtcttaggctttcctgtagcgcatctgggttcacttttagtggctacggcctctcctgggtgagacaggcgcctgggaaggggctggagtgggtagccatgatttcatctggtggctcatatacttattatgccgactccgtaaagggaagattcgcaatatcacgcgataacgctaaaaatacactcttcttgcagatggattctttgagacctgaggataccggggtttacttttgcgccagacacggggatgaccccgcctggtttgcctattggggacagggaacccctgtgacggtatcctct

SEQ ID NO: 394 - DNA, 330 bp, synthetic construct
  misc_feature 1..330: note = Synthetic
  source 1..330: mol_type = other DNA
gatattcagcttactcaaagtccgagtagtctgtctgcctcagttggcgatagggtgaccatcacttgctccgtaagtagttctatttcttccaacaacctgcattggtatcaacagaaaccaggtaaagcacctaagccgtggatctacggaacgtccaaccttgcgtctggcgtaccaagccggttctccgggagtgggagtggtacagattacacatttactatcagttctcttcaaccggaagacattgccacatattattgccagcaatggtcatcttacccctatatgtacacatttggtcagggtacaaaggttgaaataaaa

SEQ ID NO: 395 - protein, 5 aa, synthetic construct
  REGION 1..5: note = Synthetic: huMOV19
GYFMN

SEQ ID NO: 396 - protein, 17 aa, synthetic construct
  REGION 1..17: note = Synthetic: huMOV19
RIHPYDGDTFYNQKFQG

SEQ ID NO: 397 - protein, 9 aa, synthetic construct
  REGION 1..9: note = Synthetic: huMOV19
YDGSRAMDY

SEQ ID NO: 398 - protein, 15 aa, synthetic construct
  REGION 1..15: note = Synthetic: huMOV19
KASQSVSFAGTSLMH

SEQ ID NO: 399 - protein, 7 aa, synthetic construct
  REGION 1..7: note = Synthetic: huMOV19
RASNLEA

SEQ ID NO: 400 - protein, 9 aa, synthetic construct
  REGION 1..9: note = Synthetic: huMOV19
QQSREYPYT

SEQ ID NO: 401 - protein, 118 aa, synthetic construct
  REGION 1..118: note = Synthetic: huMOV19 1.00
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 402 - protein, 112 aa, synthetic construct
  REGION 1..112: note = Synthetic: huMOV19 1.00
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKR

SEQ ID NO: 403 - protein, 118 aa, synthetic construct; identical to SEQ ID NO: 401
  REGION 1..118: note = Synthetic: huMOV19 1.60

SEQ ID NO: 404 - protein, 112 aa, synthetic construct
  REGION 1..112: note = Synthetic: huMOV19 1.60
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 405 - protein, 5 aa, synthetic construct
  REGION 1..5: note = Synthetic: RA15-7
DFYMN

SEQ ID NO: 406 - protein, 19 aa, synthetic construct
  REGION 1..19: note = Synthetic: RA15-7
FIRNKANGYTTEFNPSVKG

SEQ ID NO: 407 - protein, 13 aa, synthetic construct
  REGION 1..13: note = Synthetic: RA15-7
TLYGYAYYYVMDA

SEQ ID NO: 408 - protein, 11 aa, synthetic construct
  REGION 1..11: note = Synthetic: RA15-7
RTSEDIFRNLA

SEQ ID NO: 409 - protein, 7 aa, synthetic construct
  REGION 1..7: note = Synthetic: RA15-7
DTNRLAD

SEQ ID NO: 410 - protein, 9 aa, synthetic construct
  REGION 1..9: note = Synthetic: RA15-7
QQYDNYPLT

SEQ ID NO: 411 - protein, 124 aa, synthetic construct
  REGION 1..124: note = Synthetic: RA15-7
EVQLVESGGGLVQPGGSLRLSCAASGFTFTDFYMNWVRQPPGKAPEWLGFIRNKANGYTTEFNPSVKGRFTISRDNSKNSLYLQMNSLKTEDTATYYCARTLYGYAYYYVMDAWGQGTLVTVSS

SEQ ID NO: 412 - protein, 107 aa, synthetic construct
  REGION 1..107: note = Synthetic: RA15-7
DIQMTQSPSSLSASLGDRVTITCRTSEDIFRNLAWYQQKPGKAPKLLIYDTNRLADGVPSRFSGSGSGTDYTLTISSLQPEDFATYFCQQYDNYPLTFGQGTKLEIK

SEQ ID NO: 413 - protein, 5 aa, synthetic construct
  REGION 1..5: note = Synthetic: huFR1-48
NYWMQ

SEQ ID NO: 414 - protein, 17 aa, synthetic construct
  REGION 1..17: note = Synthetic: huFR1-48
AIYPGNGDSRYTQKFQG

SEQ ID NO: 415 - protein, 8 aa, synthetic construct
  REGION 1..8: note = Synthetic: huFR1-48
RDGNYAAY

SEQ ID NO: 416 - protein, 11 aa, synthetic construct
  REGION 1..11: note = Synthetic: huFR1-48
RASENIYSNLA

SEQ ID NO: 417 - protein, 7 aa, synthetic construct
  REGION 1..7: note = Synthetic: huFR1-48
AATNLAD

SEQ ID NO: 418 - protein, 9 aa, synthetic construct
  REGION 1..9: note = Synthetic: huFR1-48
QHFWASPYT

SEQ ID NO: 419 - protein, 117 aa, synthetic construct
  REGION 1..117: note = Synthetic: huFR1-48
QVQLVQSGAEVAKPGASVKLSCKASGYTFTNYWMQWIKQRPGQGLEWIGAIYPGNGDSRYTQKFQGKATLTADKSSSTAYMQVSSLTSEDSAVYYCARRDGNYAAYWGQGTLVTVSA

SEQ ID NO: 420 - protein, 108 aa, synthetic construct
  REGION 1..108: note = Synthetic: huFR1-48
DIQMTQSPSSLSVSVGERVTITCRASENIYSNLAWYQQKPGKSPKLLVYAATNLADGVPSRFSGSESGTDYSLKINSLQPEDFGSYYCQHFWASPYTFGQGTKLEIKR

SEQ ID NO: 421 - protein, 5 aa, synthetic construct
  REGION 1..5: note = Synthetic: huFR1-49
NYWMY

SEQ ID NO: 422 - protein, 17 aa, synthetic construct
  REGION 1..17: note = Synthetic: huFR1-49
AIYPGNSDTTYNQKFQG

SEQ ID NO: 423 - protein, 9 aa, synthetic construct
  REGION 1..9: note = Synthetic: huFR1-49
RHDYGAMDY

SEQ ID NO: 424 - protein, 11 aa, synthetic construct
  REGION 1..11: note = Synthetic: huFR1-49
RASENIYTNLA

SEQ ID NO: 425 - protein, 7 aa, synthetic construct
  REGION 1..7: note = Synthetic: huFR1-49
TASNLAD

SEQ ID NO: 426 - protein, 9 aa, synthetic construct
  REGION 1..9: note = Synthetic: huFR1-49
QHFWVSPYT

SEQ ID NO: 427 - protein, 118 aa, synthetic construct
  REGION 1..118: note = Synthetic: huFR1-49
QVQLQQSGAVVAKPGASVKMSCKASGYTFTNYWMYWIKQRPGQGLELIGAIYPGNSDTTYNQKFQGKATLTAVTSANTVYMEVSSLTSEDSAVYYCTKRHDYGAMDYWGQGTSVTVSS

SEQ ID NO: 428 - protein, 108 aa, synthetic construct
  REGION 1..108: note = Synthetic: huFR1-49
DIQMTQSPSSLSVSVGERVTITCRASENIYTNLAWYQQKPGKSPKLLVYTASNLADGVPSRFSGSGSGTDYSLKINSLQPEDFGTYYCQHFWVSPYTFGQGTKLEIKR

SEQ ID NO: 429 - protein, 5 aa, synthetic construct
  REGION 1..5: note = Synthetic: huFR1-57
SFGMH

SEQ ID NO: 430 - protein, 17 aa, synthetic construct
  REGION 1..17: note = Synthetic: huFR1-57
YISSGSSTISYADSVKG

SEQ ID NO: 431 - protein, 9 aa, synthetic construct
  REGION 1..9: note = Synthetic: huFR1-57
EAYGSSMEY

SEQ ID NO: 432 - protein, 11 aa, synthetic construct
  REGION 1..11: note = Synthetic: huFR1-57
RASQNINNNLH

SEQ ID NO: 433 - protein, 7 aa, synthetic construct
  REGION 1..7: note = Synthetic: huFR1-57
YVSQSVS

SEQ ID NO: 434 - protein, 10 aa, synthetic construct
  REGION 1..10: note = Synthetic: huFR1-57
QQSNSWPHYT

SEQ ID NO: 435 - protein, 118 aa, synthetic construct
  REGION 1..118: note = Synthetic: huFR1-57
EVQLVESGGGLVQPGGSRRLSCAASGFTFSSFGMHWVRQAPGKGLEWVAYISSGSSTISYADSVKGRFTISRDNSKKTLLLQMTSLRAEDTAMYYCAREAYGSSMEYWGQGTLVTVSS

SEQ ID NO: 436 - protein, 109 aa, synthetic construct
  REGION 1..109: note = Synthetic: huFR1-57
EIVLTQSPATLSVTPGDRVSLSCRASQNINNNLHWYQQKPGQSPRLLIKYVSQSVSGIPDRFSGSGSGTDFTLSISSVEPEDFGMYFCQQSNSWPHYTFGQGTKLEIKR

SEQ ID NO: 437 - protein, 5 aa, synthetic construct
  REGION 1..5: note = Synthetic: huFR1-65
SYTMH

SEQ ID NO: 438 - protein, 17 aa, synthetic construct
  REGION 1..17: note = Synthetic: huFR1-65
YINPISGYTNYNQKFQG

SEQ ID NO: 439 - protein, 11 aa, synthetic construct
  REGION 1..11: note = Synthetic: huFR1-65
GGAYGRKPMDY

SEQ ID NO: 440 - protein, 11 aa, synthetic construct
  REGION 1..11: note = Synthetic: huFR1-65
KASQNVGPNVA

SEQ ID NO: 441 - protein, 7 aa, synthetic construct
  REGION 1..7: note = Synthetic: huFR1-65
SASYRYS

SEQ ID NO: 442 - protein, 9 aa, synthetic construct
  REGION 1..9: note = Synthetic: huFR1-65
QQYNSYPYT

SEQ ID NO: 443 - protein, 120 aa, synthetic construct
  REGION 1..120: note = Synthetic: huFR1-65
QVQLVQSGAEVAKPGASVKMSCKASGYTFTSYTMHWVKQRPGQGLAWIGYINPISGYTNYNQKFQGKATLTADKSSSTAYMQLNSLTSEDSAVYYCASGGAYGRKPMDYWGQGTSVTVSS

SEQ ID NO: 444 - protein, 108 aa, synthetic construct
  REGION 1..108: note = Synthetic: huFR1-65
EIVMTQSPATMSTSPGDRVSVTCKASQNVGPNVAWYQQKPGQSPRALIYSASYRYSGVPARFTGSGSGTDFTLTISNMQSEDLAEYFCQQYNSYPYTFGQGTKLEIKR